(19) United States
(12) Patent Application Publication
(10) Pub. No.: US 2009/0111791 A1
(43) Pub. Date: Apr. 30, 2009

(54) Soluble Epoxide Hydrolase Inhibitors and Methods of Using Same

(75) Inventors: Stephane De Lombaert, Madison, CT (US); Anne Bettina Eldrup, Danbury, CT (US); Jennifer A. Kowalski, New Milford, CT (US); Ingo Andreas Mugge, New Haven, CT (US); Fariba Soleymanzadeh, Danbury, CT (US); Alan David Swinamer, Southbury, CT (US); Steven John Taylor, Southbury, CT (US)

Correspondence Address: Micheal P. Morris;Boehringer Ingelheim USA Corporation, 900 Ridgebury Road, Ridgefield, CT, 06877-0368, US

(73) Assignee: BOEHRINGER INGELHEIM INTERNATIONAL GMBH, Ingelheim (DE)

(21) Appl. No.: 12/281,065

(22) PCT Filed: Mar. 8, 2007
(86) PCT No.: PCT/US2007/063544

§ 371 Date: Aug. 28, 2008

Related U.S. Application Data
(60) Provisional application No. 60/743,452, filed on Mar. 10, 2006.

(51) Int. Cl. A61K 31/506; C07D 211/52; A61K 31/445; A61K 31/40; A61P 3/10; C07D 207/16; C07D 403/12

(52) U.S. Cl. 514/210.18; 546/221; 514/327; 514/423; 548/538; 544/316

(57) ABSTRACT

Disclosed are compounds active against soluble epoxide hydrolase (sEH), compositions thereof and methods of using and making same.

### APPLICATION DATA

This application claims benefit to U.S. provisional application Ser. No. 60/743,452 filed Mar. 10, 2006.

### BACKGROUND OF THE INVENTION

1. Technical Field

This invention relates to compounds possessing anti-sEH activity and methods of using soluble epoxide hydrolase (sEH) inhibitors for diseases related to cardiovascular disease.

2. Background Information

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems and their diol products are frequently found as intermediates in the metabolic pathway of xenobiotics. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are four regioisomeric cis epoxides of arachidonic acid, 5,6-, 8,9-, 11,12-, and 14,15-epoxyeicosatrienoic acid, also known as epoxyeicosatrienoic acids or EETs. Also known to be substrates for sEH are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

EETs function as chemical autocrine and paracrine mediators in the cardiovascular and renal systems (Spector, et al, Progress in Lipid Research, 43: 55-90 (2004); Newman, et al., Progress in Lipid Research 44: 1-51 (2005)). EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lead to activation of BK_{Ca2+} channels (big Ca²⁺ activated potassium channels) and inhibition of L-type Ca²⁺ channels, ultimately resulting in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37[part 2]:744-748 (2001)). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In angiotensin II infused rats the treatment with a selective sEH inhibitor attenuated the afferent arteriolar diameter in the kidney and lowered urinary albumin secretion, a marker of compromised renal function, suggesting antihypertensive and renal vascular protective effects of increased EETs levels (Zhao, et al, 15: 1244-1253 (2004)). In the spontaneously hypertensive rat model, both cytochrome P450 and sEH activities were found to increase (Yu et al., Molecular Pharmacology, 57: 1011-1020 (2000)). Addition of a known sEH inhibitor was shown to decrease the blood pressure to normal levels. Furthermore, administration of a selective sEH inhibitor to angiotensin II treated rats was demonstrated to lower systolic blood pressure (Imig, et al, Hypertension, 39: 690-694 (2002)). Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition. In addition, the administration of EETs and/or the administration of a selective sEH inhibitor was demonstrated to attenuate tobacco smoke induced inflammation, as assessed by total bronchoalveolar lavage cell numbers and concomittant reduction in neutrophils, alveolar macrophages, and lymphocytes (Smith, et al, 102: 2186-2191 (2005)).

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm.& Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20): 15265-15270 (2000); Nakagawa et al. Bioorg. Med. Chem., 8: 2663-2673 (2000); US 2005/0026844 and Kim, et al., J. Med. Chem. 47(8): 2110-2122 (2004) some of which describe inhibitors with additional, tethered oxo pharmacophores).

WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of inhibiting an epoxide hydrolase, using compounds having the structure

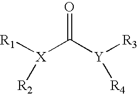

wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

Ashwell, M. A. et al., Bioorganic & Medicical Chemistry Letters, 11: 3123-3127 (2001) describes particular 4-aminopiperidine ureas which are alleged to possess selective activity towards the human beta 3-adrenergic receptor. The compounds described in this application are structurally distinct from the compounds disclosed in the Ashwell paper.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### BRIEF SUMMARY OF THE INVENTION

It is therefore an object of the invention to provide compounds active as sEH inhibitors described herein below.

It is a further object of the invention to provide a method of treating hypertension by administering to a patient a compound as described herein below which modulates sEH.

It is yet a further object to provide methods of making the compounds described herein below.

### DETAILED DESCRIPTION OF THE INVENTION

In one generic aspect of the invention, there is provided a compound of the formula (I):

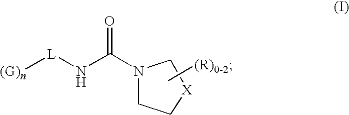

wherein:
G is carbocycle, heteroaryl or heterocyclyl optionally substituted by one or more Y;
n is 1 or 2 such that L can be substituted with one to two G;
L is a methylene or ethylene linking group optionally substituted by hydroxy, amino, lower alkoxy, lower alkylamino, lower alkylthio or 1-3 fluorine atoms;
X is a bond, methylene or ethylene;
R if present is chosen from:

### i) —C(O)—R₁;

R₁ is chosen from —OH, —O(CH₂)₀₋₅—CH₃, —NR₂R₃, carbocycle, heteroaryl or heterocyclyl;
ii) carbocycle, heteroaryl or heterocyclyl optionally substituted by one or more R₄;
iii) —W-Q, wherein:
W is chosen from alkylene, O, S, NH—S(O)₂— and NH;
Q is chosen from OH, alkyl, carbocycle, heteroaryl and heterocyclyl optionally substituted by one or more R₅;
iv) lower alkyl;
Y is chosen from
halogen, lower alkyl, lower alkoxy each optionally halogenated, aryloxy, sulfone, nitrile, or Y is carbocycle optionally substituted by one to three oxo, lower acyl, halogen, nitrile, lower alkylS(O)ₘ—, lower alkylS(O)ₘ—NH—, lower alkoxycarbonyl, NR₂R₃—C(O)—, —NR₂R₃, lower alkyl, C₃₋₆ cycloalkylC₀₋₂alkyl, hydroxy, lower alkoxy or arylC₀₋₄ alkyl the aryl group being optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl or lower alkyl optionally substituted by lower alkylS(O)ₘ—, lower alkoxy, hydroxy or mono or diC₁₋₃ alkyl amino; or R₂ and R₃ optionally combine with the nitrogen atom to which they are attached to form a heterocyclic ring;
each R₄ and R₅ are independently nitrile, hydroxy, lower alkylS(O)ₘ—, carboxy, halogen, lower alkoxy, arylC₀₋₄ alkyl, heteroaryl C₀₋₄ alkyl, heterocycle C₀₋₄alkyl, C₁₋₂ acyl, aroyl, lower alkyl optionally substituted by lower alkylS(O)ₘ—, lower alkoxy or hydroxy, —C(O)—NH₂ or —S(O)ₘ—NH₂ wherein each case the N atom is optionally substituted by lower-alkyl; each R₄ and R₅ are optionally halogenated;
m is 0, 1 or 2;
or the pharmaceutically acceptable salts thereof.

In another embodiment of the invention there is provided compounds of the formula (I) as described immediately above, and wherein:

X is ethylene;
R if present is chosen from:

### i) —C(O)—R₁;

R₁ is chosen from —OH, —NR₂R₃, phenyl, C₃₋₆ cycloalkyl and heteroaryl chosen from pyrimidinyl, pyridinyl, pyridazinyl, pyrazinyl, pyranyl, pyrrolyl, pyrazolyl, imidazolyl, furanyl, oxazolyl, thienyl and thiazolyl;
ii) phenyl, heteroaryl or heterocyclyl optionally substituted by one or more R₄;
iii) —W-Q, wherein:
W is chosen from methylene, ethylene and O;
Q is chosen from OH, —O(CH₂)₀₋₂—CH₃, methyl, phenyl, heteroaryl chosen from pyrimidinyl, pyridinyl, pyridazinyl, pyrazinyl, pyranyl, pyrrolyl, pyrazolyl, imidazolyl, furanyl, oxazolyl, thienyl and thiazolyl, optionally substituted by one or more R₅;
iv) lower alkyl;
Y is chosen from
aryloxy, sulfone, nitrile, halogen, lower alkyl, lower alkoxy each optionally halogenated or Y is phenyl or C₃₋₆ cycloalkyl each optionally substituted by C₃₋₆ cycloalkylC₀₋₂alkyl or arylC₀₋₄ alkyl the cycloalkyl or aryl group being optionally substituted by one to three hydroxy, lower alkyl or lower alkoxy;
each R₂ and R₃ are independently hydrogen, phenylC₀₋₂ alkyl, heteroaryl C₀₋₂ alkyl, heterocycle C₀₋₂alkyl or lower alkyl optionally substituted by lower alkylS(O)ₘ—, lower alkoxy or hydroxy;
each R₄ and R₅ are independently nitrile, hydroxy, lower alkylS(O)ₘ—, carboxy, halogen, lower alkoxy, phenylC₀₋₂ alkyl, heteroaryl C₀₋₂ alkyl, heterocycle C₀₋₂alkyl, lower alkyl optionally substituted by lower alkylS(O)ₘ—, lower alkoxy or hydroxyl or hydroxy, —C(O)—NH₂ or —S(O)ₘ—NH₂ wherein each case the N atom is optionally substituted by lower-alkyl; each R₄ and R₅ are optionally halogenated;

In another embodiment of the invention there is provided compounds of the formula (I) as described immediately above, and wherein:

G is phenyl, C₃₋₈ cycloalkyl, bicycloheptane [2.2.1], bicyclo[2.2.1]5-heptene or adamantyl optionally substituted by one or more Y;
L is a methylene linking group optionally substituted by hydroxy, amino, lower alkoxy, lower alkylamino, lower alkylthio or 1-3 fluorine atoms;
R if present is chosen from:

### i) —C(O)—R₁;

R₁ is chosen from —OH, —NR₂R₃, phenyl, C₃₋₆ cycloalkyl and heteroaryl chosen from pyrimidinyl, pyridinyl, pyridazinyl and pyrazinyl;
ii) phenyl, morpholino, piperidinyl, benzimidazolyl or pyridinyl optionally substituted by one or more R₄;
iii) —W-Q, wherein:
W is chosen from methylene, ethylene and 0;
Q is chosen from OH, —O(CH₂)₀₋₂—CH₃, methyl, phenyl, heteroaryl chosen from pyrimidinyl, pyridinyl, pyridazinyl and pyrazinyl, optionally substituted by one or more R₅;
iv) lower alkyl;
Y is chosen from
Cl, F, —CH₃, —O—CF₃, —O—CH₃, phenoxy or phenyl;
each R₂ and R₃ are independently hydrogen, pyridinylmethyl, tetrahydropyranylethyl, pyrrolidinylethyl, benzodioxanylmethyl, or lower alkyl optionally substituted by lower alkylS(O)ₘ— or lower alkoxy;
each R₄ and R₅ are independently Cl, F, lower alkoxy, phenyl and —CF₃.

In another generic aspect of the invention, there is provided a compound of the formula (Ia):

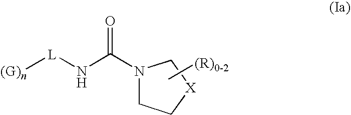

wherein for the Formula (Ia), the component

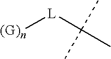

is chosen from A1-A67 in the table I below; in combination with any component

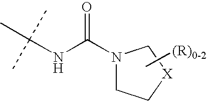

chosen from B1-B97 in the table I below;

TABLE I

| | |
|---|---|
| A | 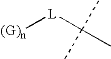 |
| A1 | 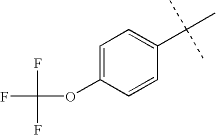 |
| A2 | 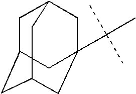 |
| A3 | 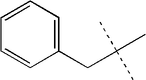 |
| A4 | 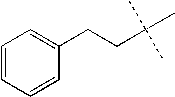 |
| A5 | 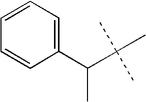 |
| A6 | 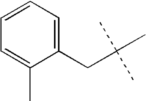 |
| A7 | 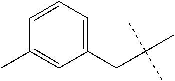 |
| A8 | 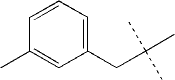 |
| A9 | 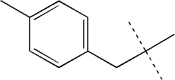 |
| A10 | 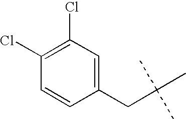 |
| A11 | 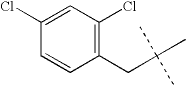 |
| A12 | 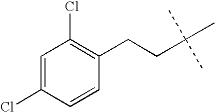 |
| A13 | 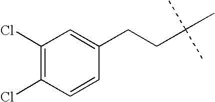 |
| A14 | 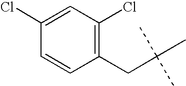 |
| A15 | 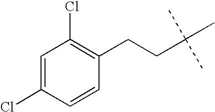 |
| A16 | 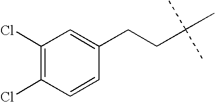 |
| A17 | 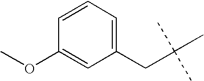 |
| A18 | 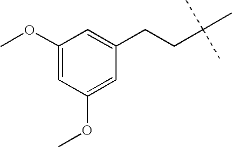 |
| A19 | 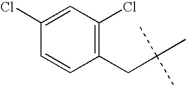 |
| A20 | 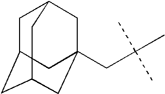 |
| A21 | 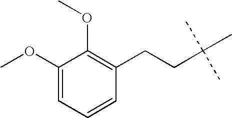 |
| A22 | 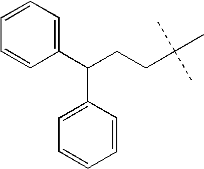 |
| A22 | 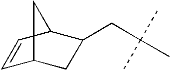 |
| A23 | 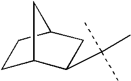 |
| A24 | 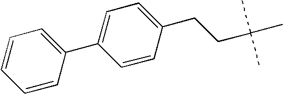 |
| A25 | 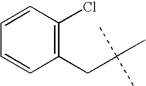 |
| A26 | 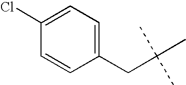 |
| A27 | 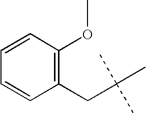 |
| A28 | 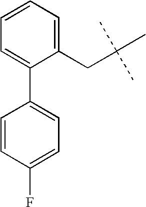 |
| A29 | 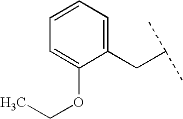 |
| A30 | 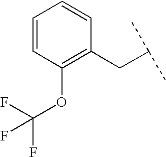 |
| A31 | 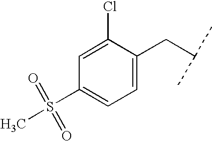 |
| A32 | 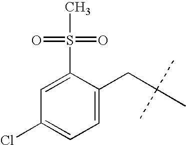 |
| A33 | 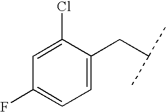 |
| A34 | 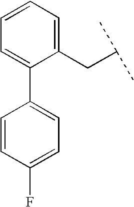 |
| A35 | 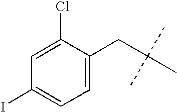 |
| A36 | 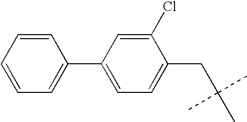 |
| A37 | 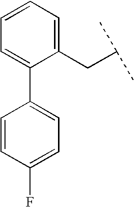 |
| A38 | 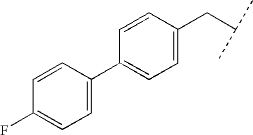 |
| A39 | 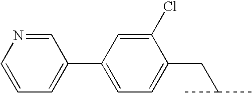 |
| A40 | 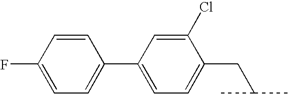 |
| A41 | 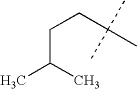 |
| A42 | 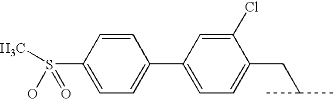 |
| A43 | 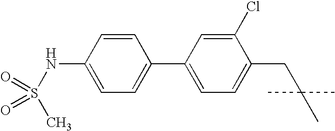 |
| A44 | 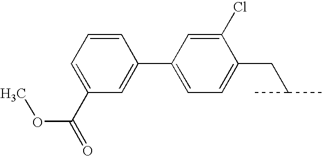 |
| A45 | 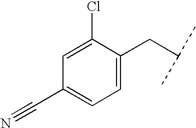 |
| A46 | 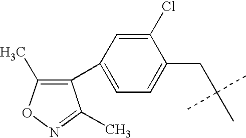 |
| A47 | 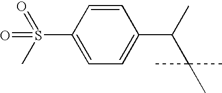 |
| A48 | 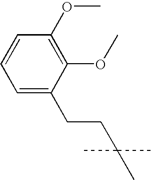 |
| A49 | 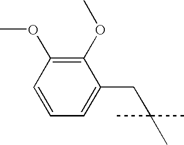 |
| A50 | 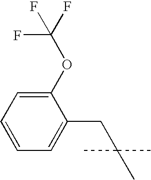 |
| A51 | 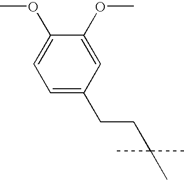 |
| A52 | 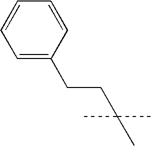 |
| A53 | 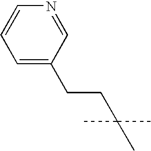 |
| A54 | 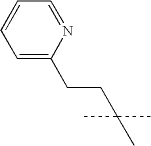 |
| A55 | 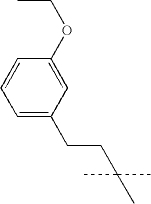 |
| A56 | 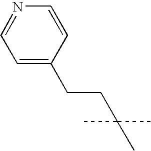 |
| A57 | 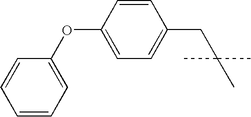 |
| A58 | 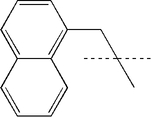 |
| A59 | 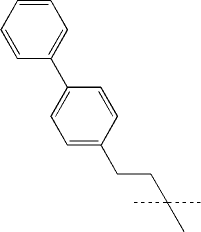 |
| A60 | 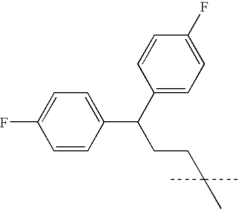 |
| A61 | 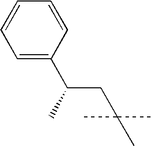 |
| A62 | 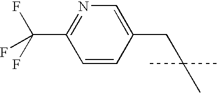 |
| A63 | 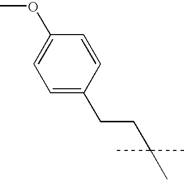 |
| A64 | 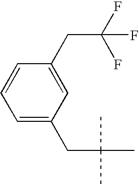 |
| A65 | 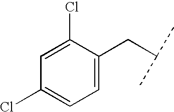 |
| A66 | 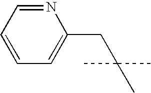 |
| A67 | 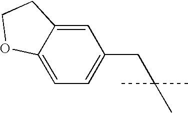 |
| B | 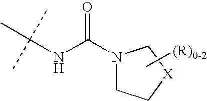 |
| B1 | 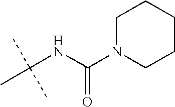 |
| B2 | 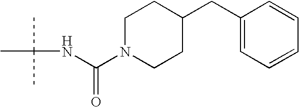 |
| B3 | 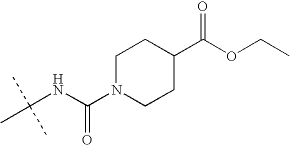 |
| B4 | 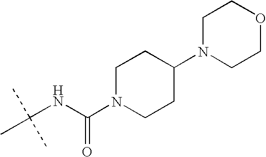 |
| B5 | 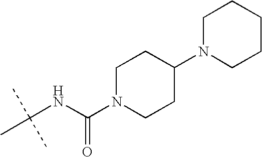 |
| B6 | 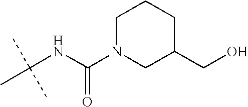 |
| B7 | 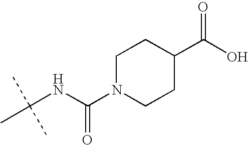 |
| B8 | 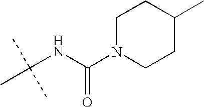 |
| B9 | 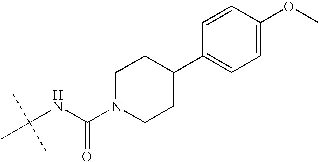 |
| B10 | 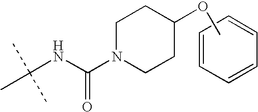 |
| B11 | 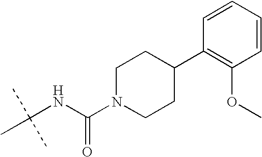 |
| B12 | 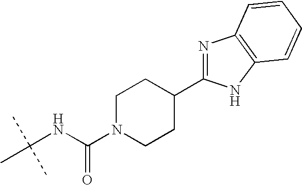 |
| B13 | 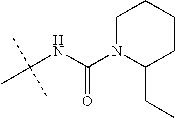 |
| B14 | 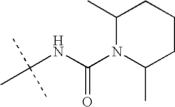 |
| B15 | 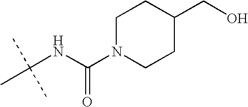 |
| B16 | 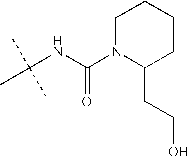 |
| B17 | 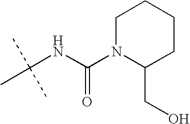 |
| B18 | 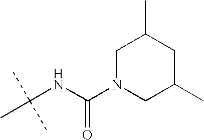 |
| B19 | 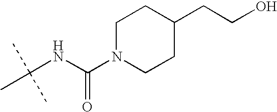 |
| B20 | 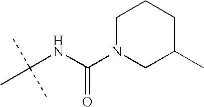 |
| B21 | 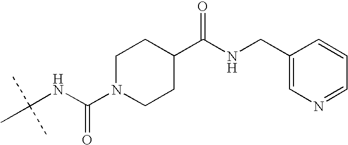 |
| B22 | 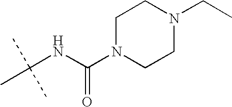 |
| B22 | 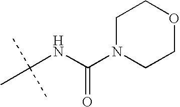 |
| B23 | 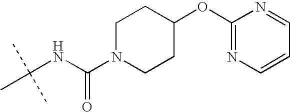 |
| B24 | 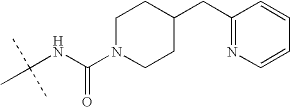 |
| B25 | 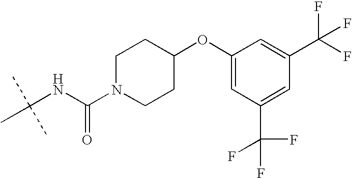 |
| B26 | 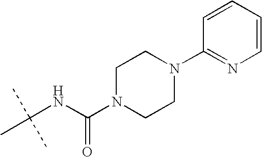 |
| B27 | 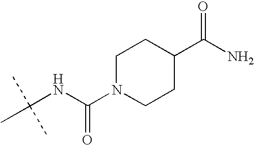 |
| B28 | 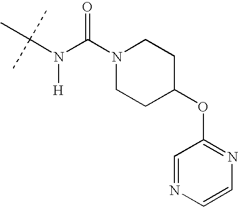 |
| B29 | 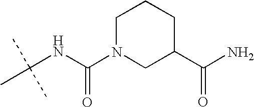 |
| B30 | 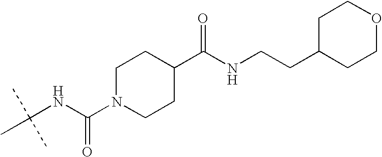 |
| B31 | 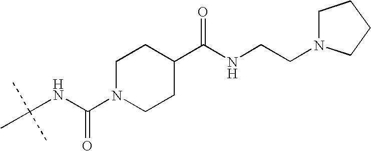 |
| B32 | 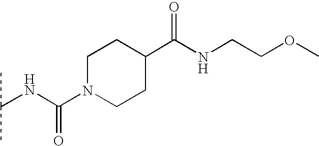 |
| B33 | 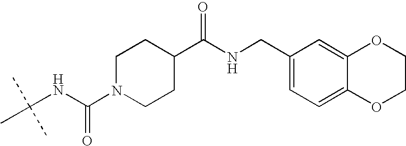 |
| B34 | 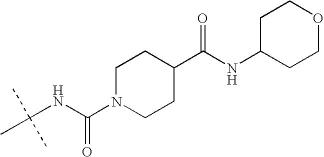 |
| B35 | 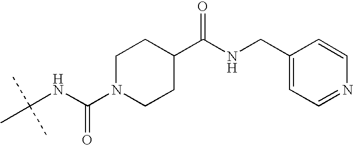 |
| B36 | 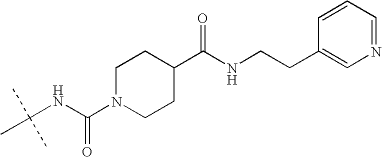 |
| B37 | 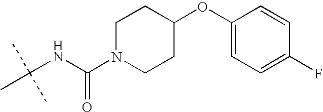 |
| B38 | 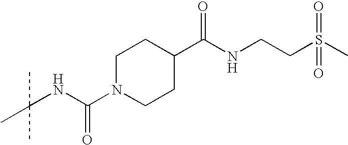 |
| B39 | 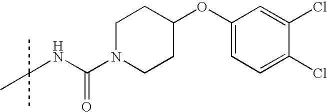 |
| B40 | 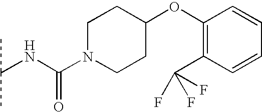 |
| B41 | 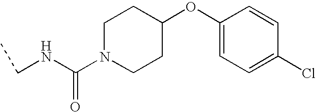 |
| B42 | 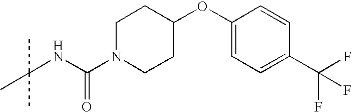 |
| B43 | 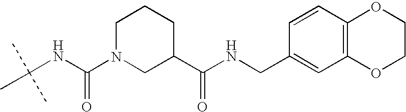 |
| B44 | 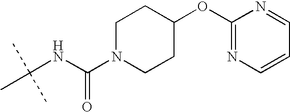 |
| B45 | 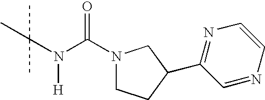 |
| B46 | 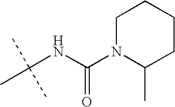 |
| B47 | 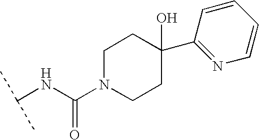 |
| B48 | 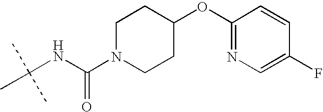 |
| B49 | 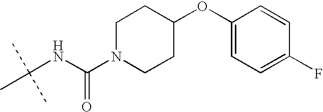 |
| B50 | 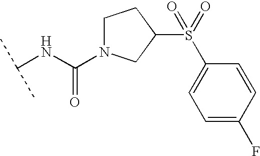 |
| B51 | 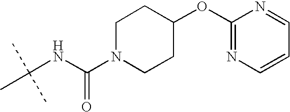 |
| B52 | 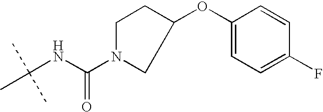 |
| B53 | 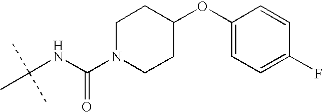 |
| B54 | 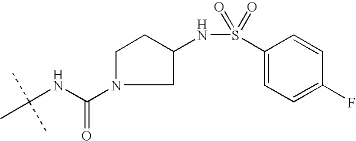 |
| B55 | 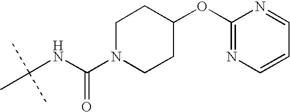 |
| B56 | 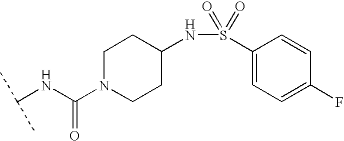 |
| B57 | 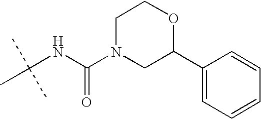 |
| B58 | 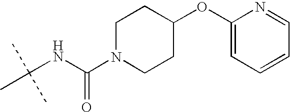 |
| B59 | 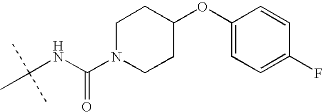 |
| B60 | 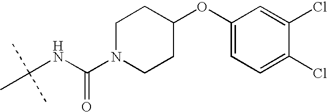 |
| B61 | 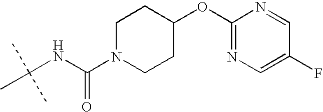 |
| B62 | 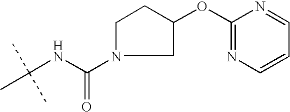 |
| B63 | 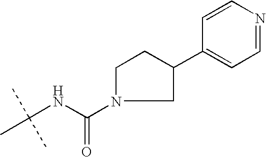 |
| B64 | 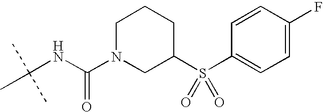 |
| B65 | 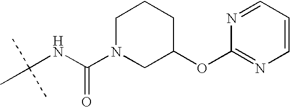 |
| B66 | 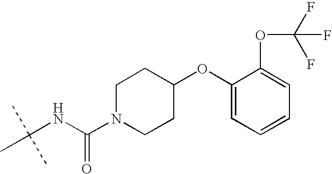 |
| B67 | 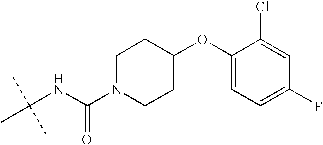 |
| B68 | 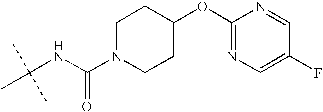 |
| B69 | 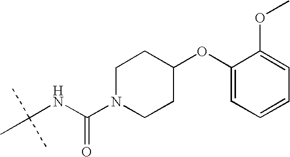 |
| B70 | 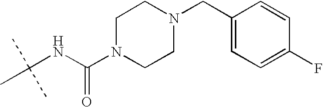 |
| B71 | 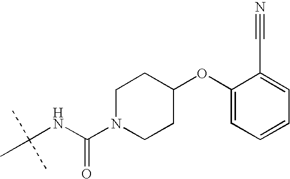 |
| B72 | 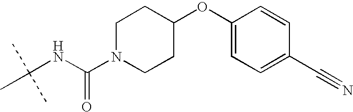 |
| B73 | 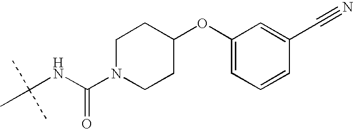 |
| B74 | 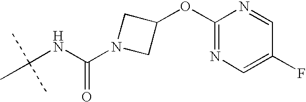 |
| B75 | 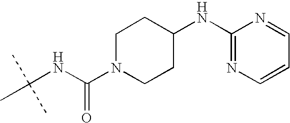 |
| B76 | 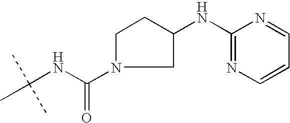 |
| B77 | 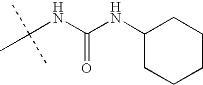 |
| B78 | 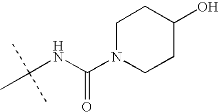 |
| B79 | 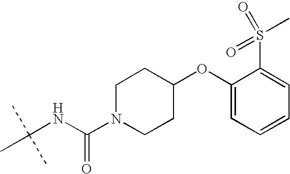 |
| B80 | 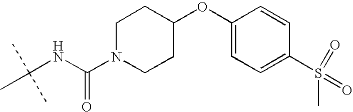 |
| B81 | 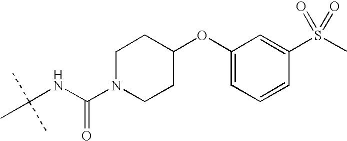 |
| B82 | 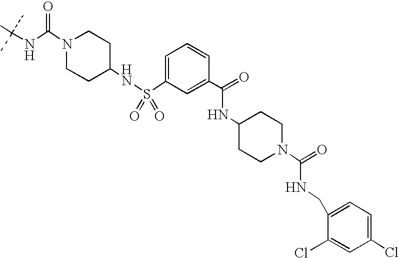 |
| B83 | 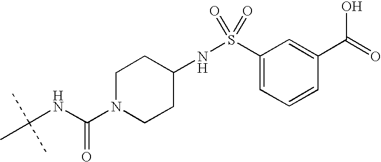 |
| B84 | 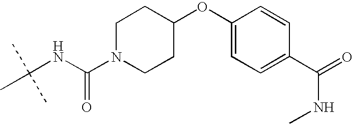 |
| B85 | 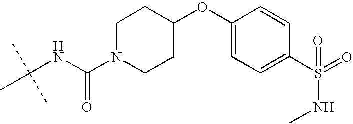 |
| B86 | 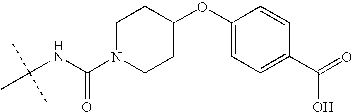 |
| B87 | 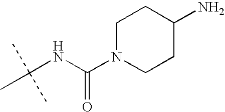 |
| B88 | 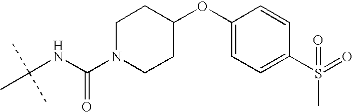 |
| B89 | 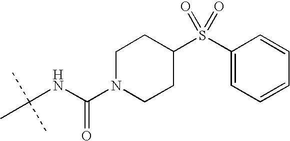 |
| B90 | 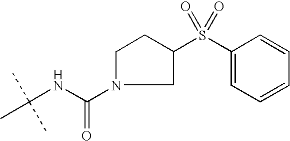 |
| B91 | 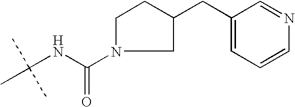 |
| B92 | 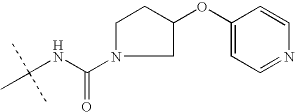 |
| B93 | 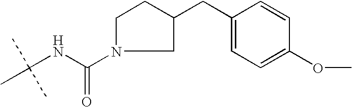 |
| B94 | 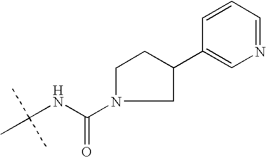 |
| B95 | 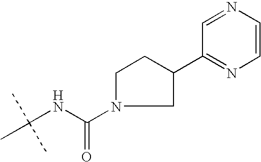 |
| B96 | 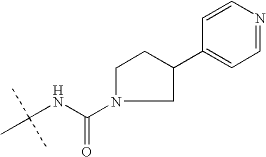 |
| B97 | 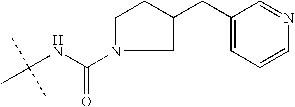 |

or the pharmaceutically acceptable salts thereof.

In another embodiment of the invention there is provided compounds of the formula (Ia) as described immediately above, and wherein:

wherein for the Formula (Ia), the component

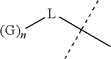

is chosen from A1-A41 in the table II below; in combination with any component

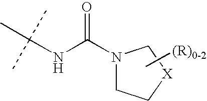

chosen from B1-B97 in the table II below;

| | |
|---|---|
| A | 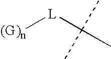 |
| A1 | 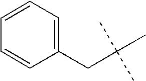 |
| A2 | 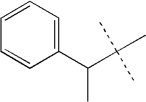 |
| A3 | 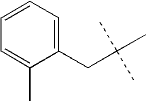 |
| A4 | 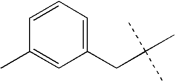 |
| A5 | 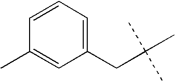 |
| A6 | 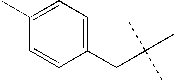 |
| A7 | 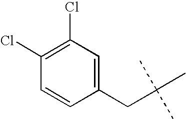 |
| A8 | 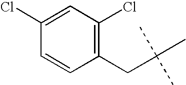 |
| A9 | 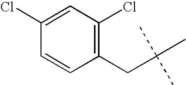 |
| A10 | 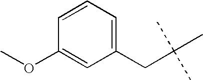 |
| A11 | 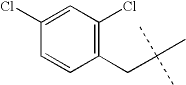 |
| A12 | 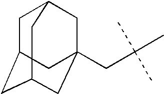 |
| A13 | 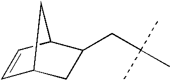 |
| A14 | 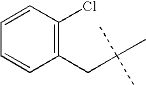 |
| A15 | 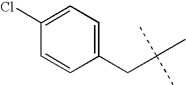 |
| A16 | 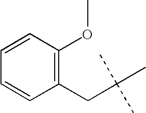 |
| A17 | 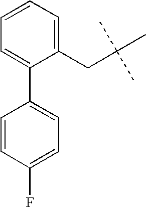 |
| A18 | 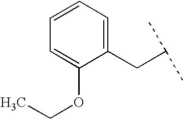 |
| A19 | 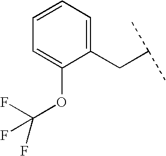 |
| A20 | 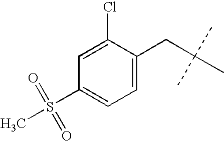 |
| A21 | 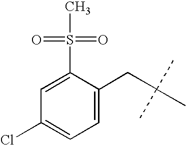 |
| A22 | 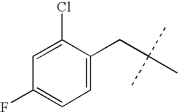 |
| A22 | 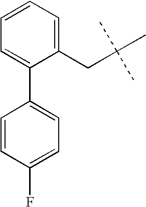 |
| A23 | 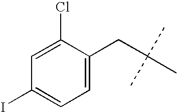 |
| A24 | 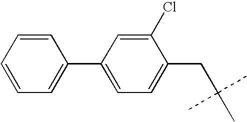 |
| A25 | 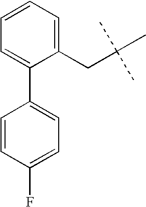 |
| A26 | 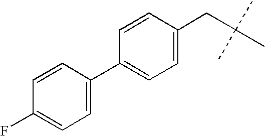 |
| A27 | 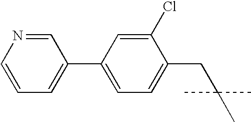 |
| A28 | 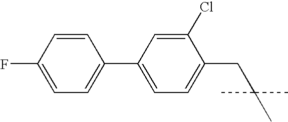 |
| A29 | 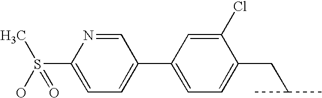 |
| A30 | 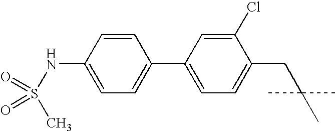 |
| A31 | 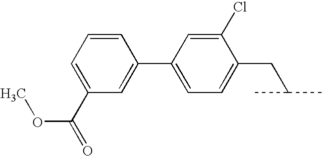 |
| A32 | 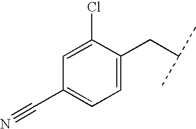 |
| A33 | 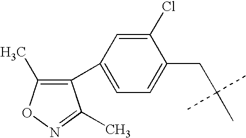 |
| A34 | 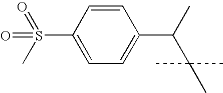 |
| A35 | 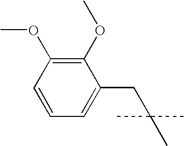 |
| A36 | 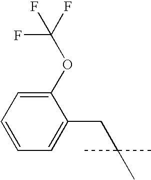 |
| A37 | 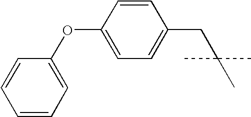 |
| A38 | 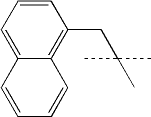 |
| A39 | 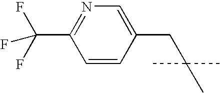 |
| A40 | 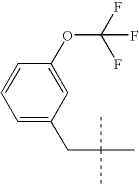 |
| A41 | 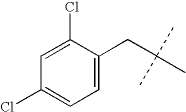 |
| B | 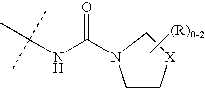 |
| B1 | 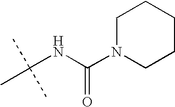 |
| B2 | 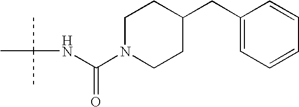 |
| B3 | 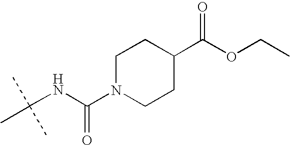 |
| B4 | 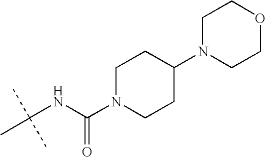 |
| B5 | 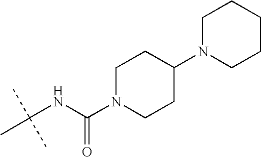 |
| B6 | 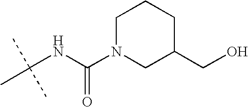 |
| B7 | 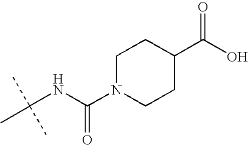 |
| B8 | 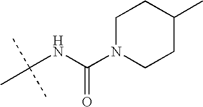 |
| B9 | 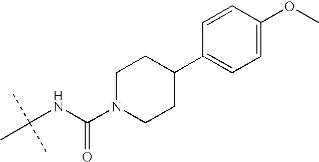 |
| B10 | 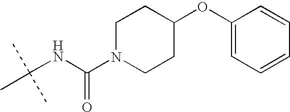 |
| B11 | 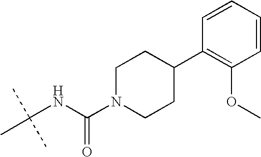 |
| B12 | 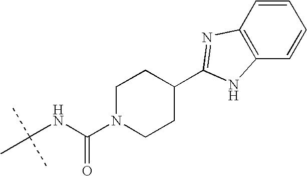 |
| B13 | 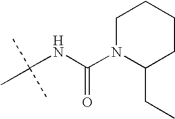 |
| B14 | 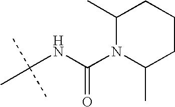 |
| B15 | 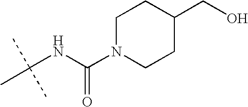 |
| B16 | 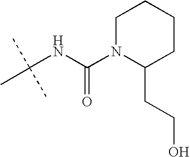 |
| B17 | 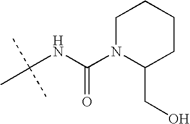 |
| B18 | 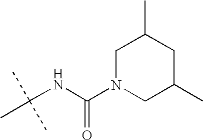 |
| B19 | 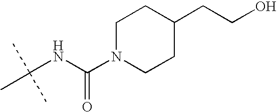 |
| B20 | 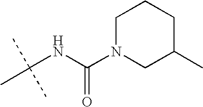 |
| B21 | 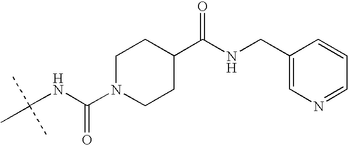 |
| B22 | 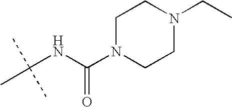 |
| B22 | 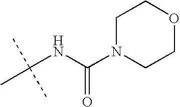 |
| B23 | 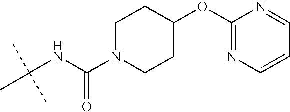 |
| B24 | 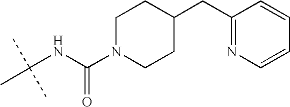 |
| B25 | 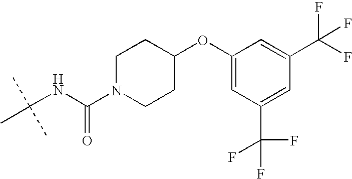 |
| B26 | 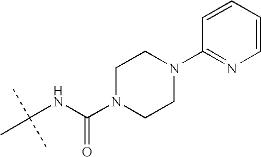 |
| B27 | 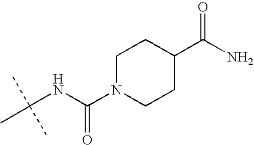 |
| B28 | 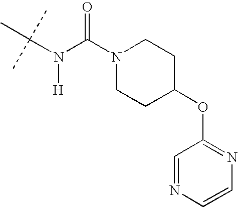 |
| B29 | 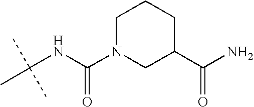 |
| B30 | 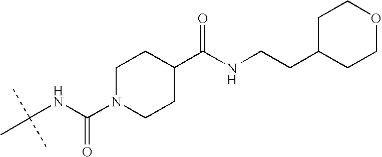 |
| B31 | 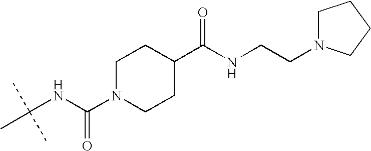 |
| B32 | 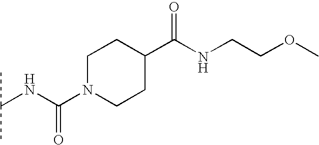 |
| B33 | 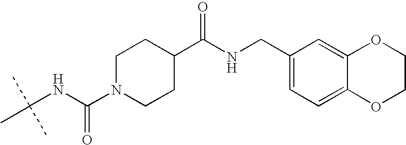 |
| B34 | 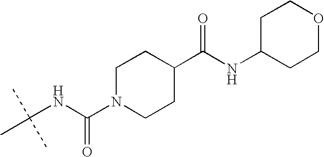 |
| B35 | 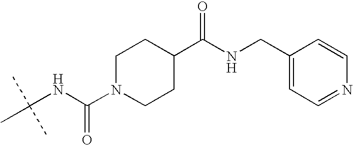 |
| B36 | 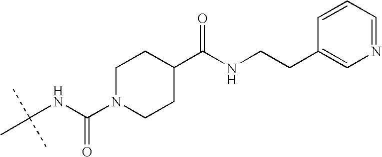 |
| B37 | 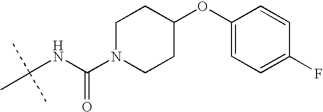 |
| B38 | 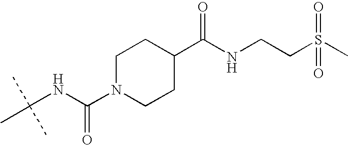 |
| B39 | 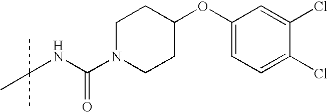 |
| B40 | 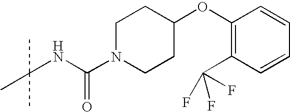 |
| B41 | 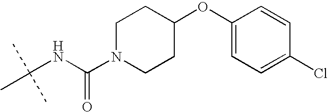 |
| B42 | 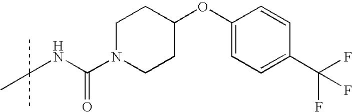 |
| B43 | 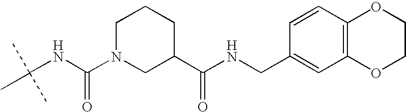 |
| B44 | 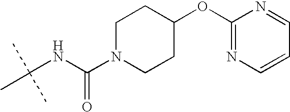 |
| B45 | 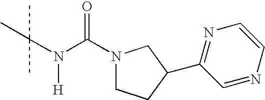 |
| B46 | 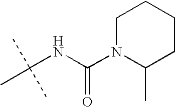 |
| B47 | 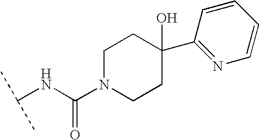 |
| B48 | 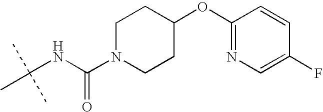 |
| B49 | 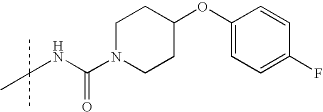 |
| B50 | 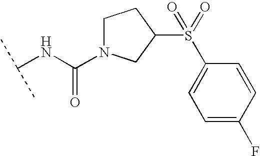 |
| B51 | 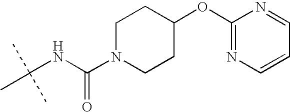 |
| B52 | 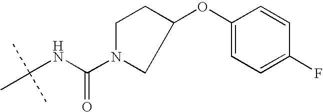 |
| B53 | 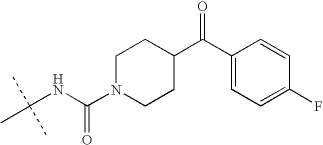 |
| B54 | 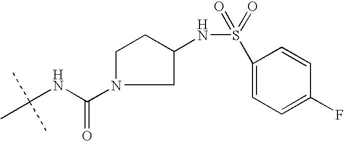 |
| B55 | 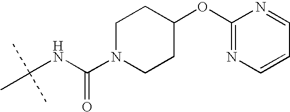 |
| B56 | 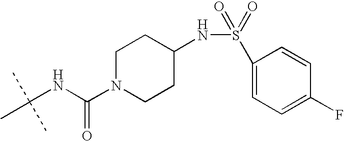 |
| B57 | 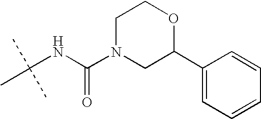 |
| B58 | 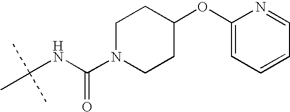 |
| B59 | 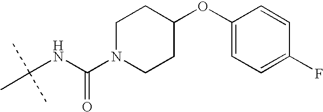 |
| B60 | 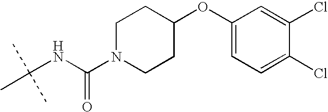 |
| B61 | 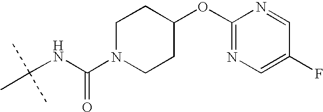 |
| B62 | 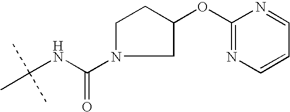 |
| B63 | 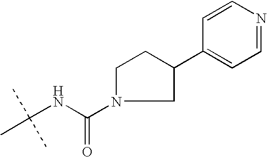 |
| B64 | 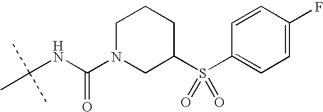 |
| B65 | 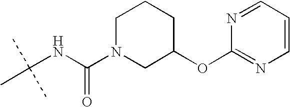 |
| B66 | 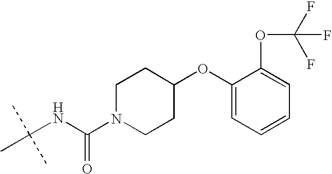 |
| B67 | 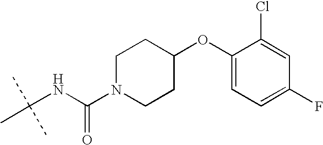 |
| B68 | 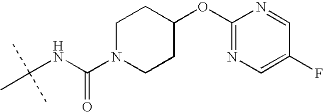 |
| B69 | 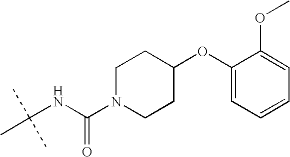 |
| B70 | 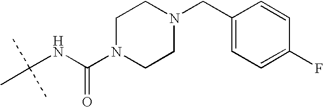 |
| B71 | 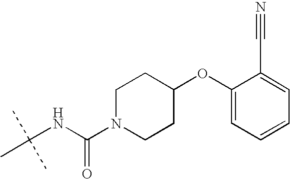 |
| B72 | 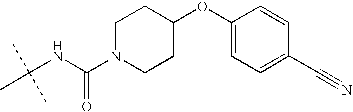 |
| B73 | 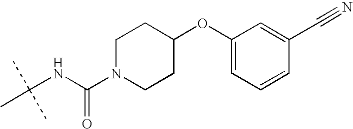 |
| B74 | 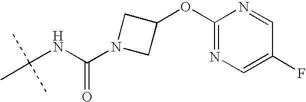 |
| B75 | 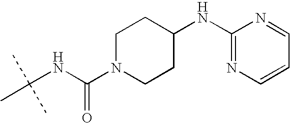 |
| B76 | 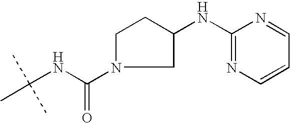 |
| B77 | 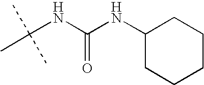 |
| B78 | 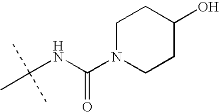 |
| B79 | 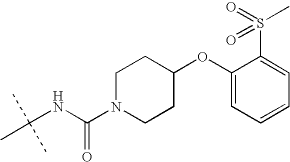 |
| B80 | 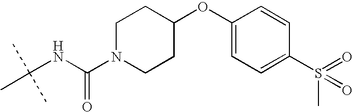 |
| B81 | 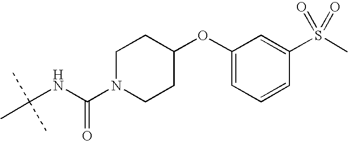 |
| B82 | 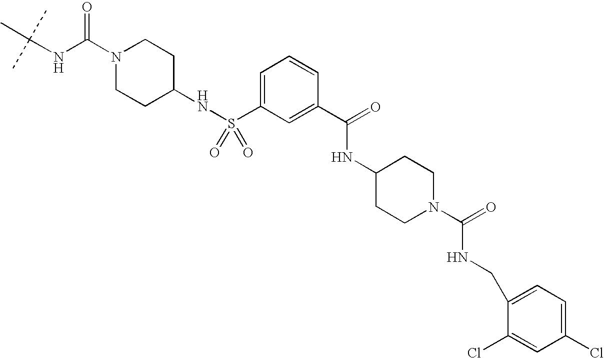 |
| B83 | 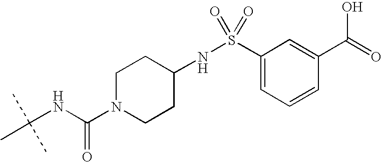 |
| B84 | 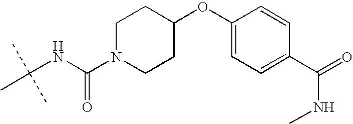 |
| B85 | 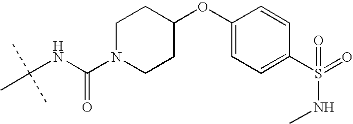 |
| B86 | 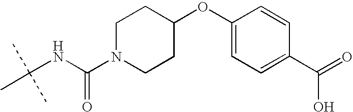 |
| B87 | 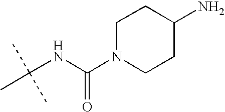 |
| B88 | 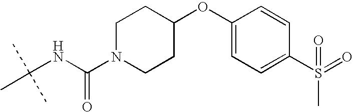 |
| B89 | 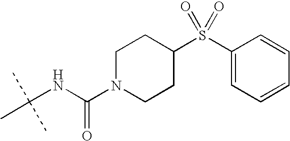 |
| B90 | 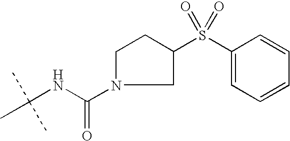 |
| B91 | 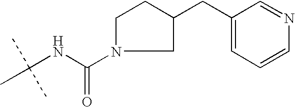 |
| B92 | 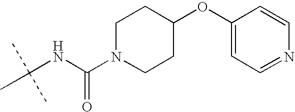 |
| B93 | 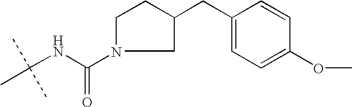 |
| B94 | 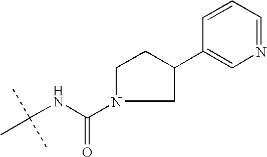 |
| B95 | 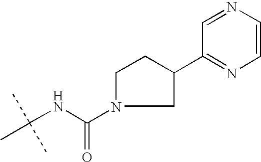 |
| B96 | 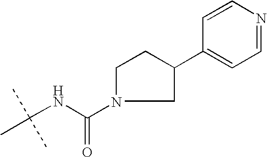 |
| B97 | 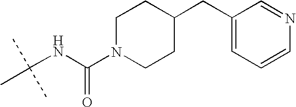 |

In a preferred embodiment of the invention there is provided compounds of the formula (Ia) as described immediately above, and wherein column B of table II is:

| | |
|---|---|
| B | 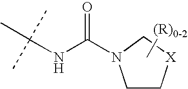 |
| B10 | 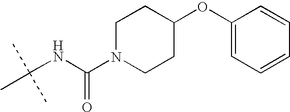 |
| B23 | 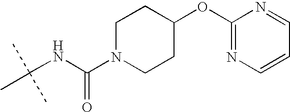 |
| B25 | 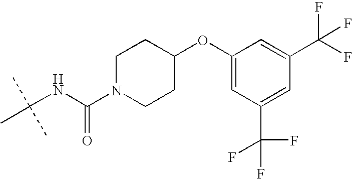 |
| B28 | 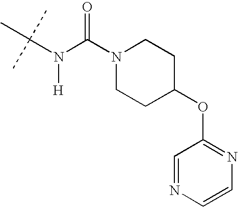 |
| B37 | 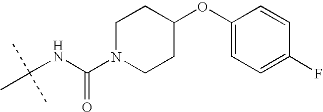 |
| B39 | 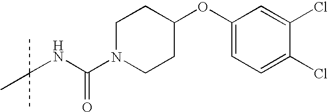 |
| B40 | 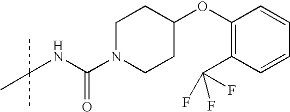 |
| B41 | 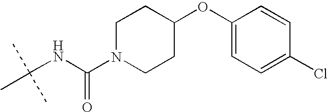 |
| B42 | 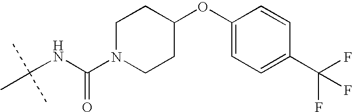 |
| B44 | 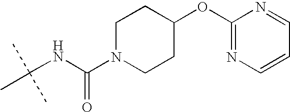 |
| B48 | 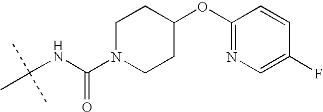 |
| B49 | 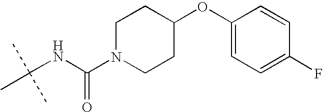 |
| B51 | 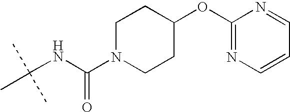 |
| B52 | 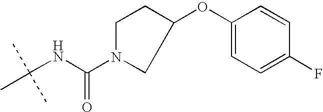 |
| B55 | 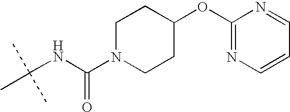 |
| B58 | 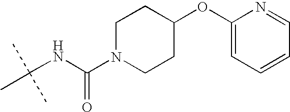 |
| B59 | 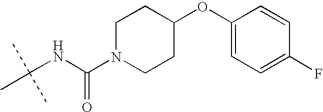 |
| B60 | 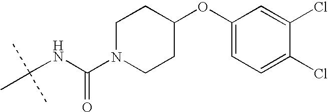 |
| B61 | 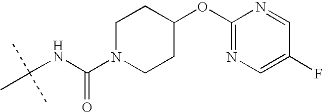 |
| B62 | 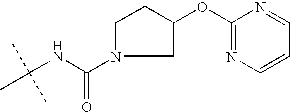 |
| B65 | 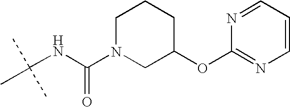 |
| B66 | 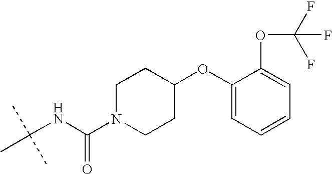 |
| B67 | 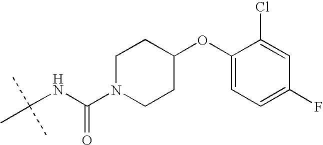 |
| B68 | 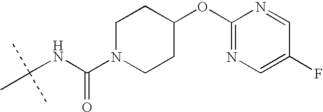 |
| B69 | 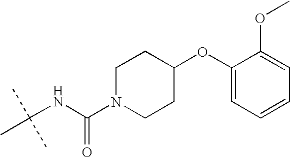 |
| B71 | 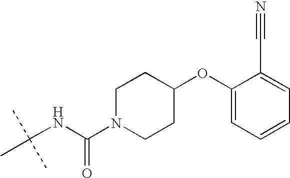 |
| B72 | 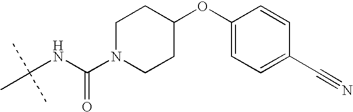 |
| B73 | 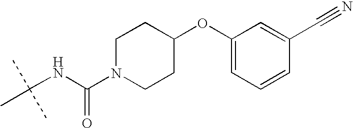 |
| B74 | 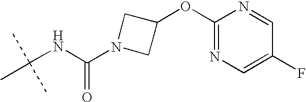 |
| B79 | 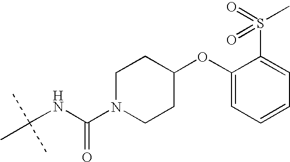 |
| B80 | 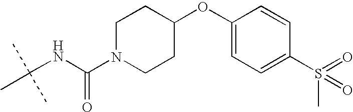 |
| B81 | 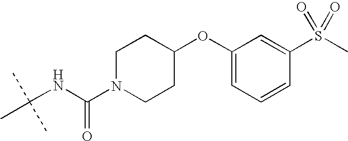 |
| B84 | 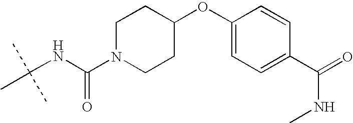 |
| B85 | 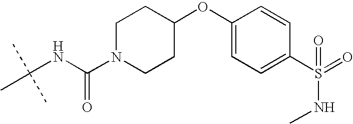 |
| B86 | 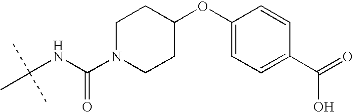 |
| B88 | 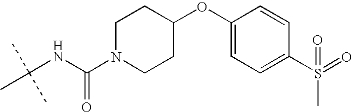 |
| B89 | 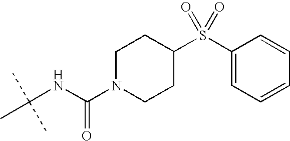 |
| B90 | 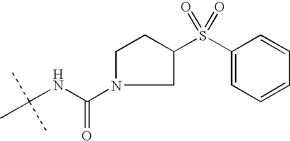 |
| B92 | 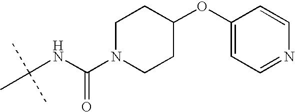 |

The following are representative compounds of the invention which can be made according to the general synthetic procedures and examples which follow:

TABLE III

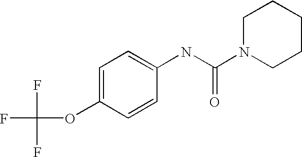

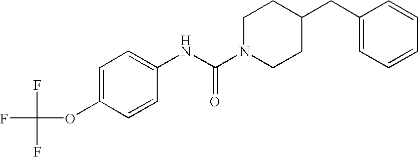

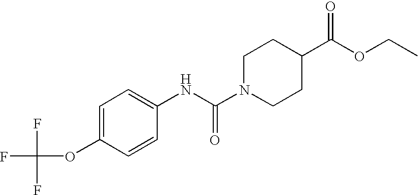

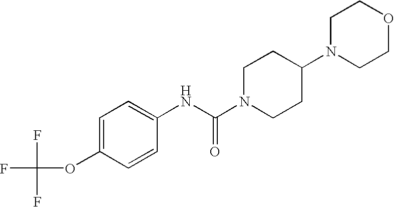

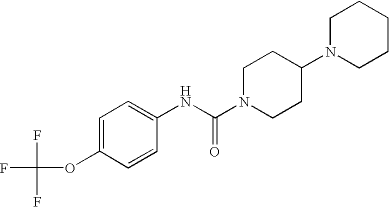

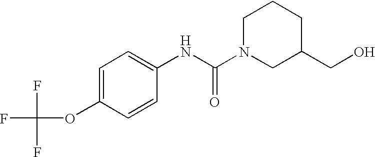

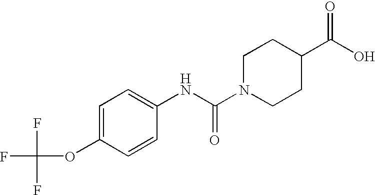

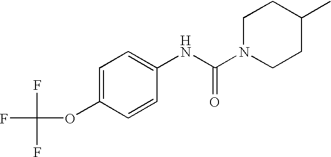

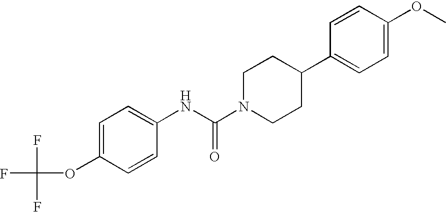

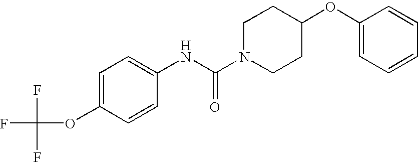

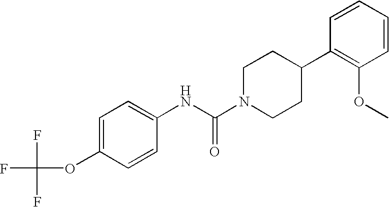

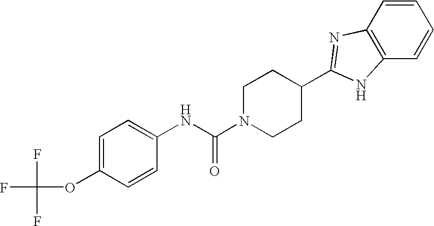

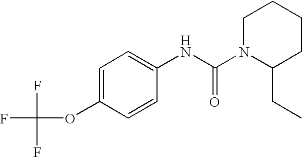

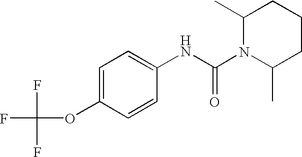

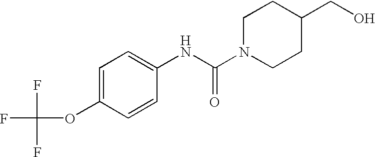

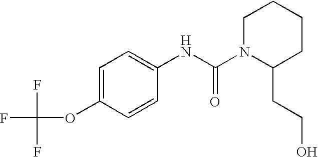

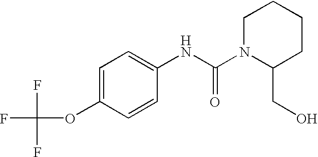

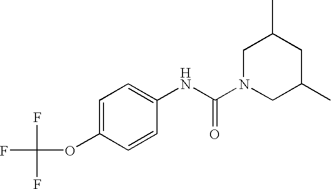

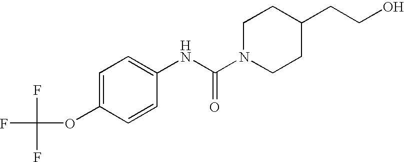

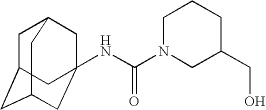

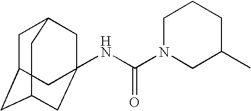

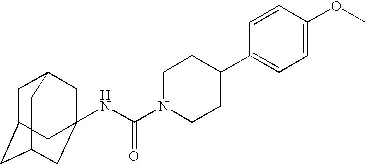

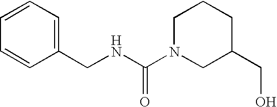

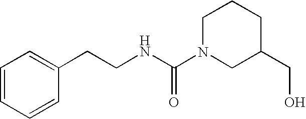

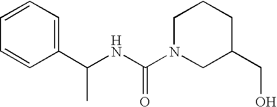

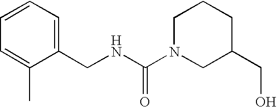

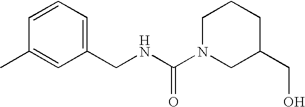

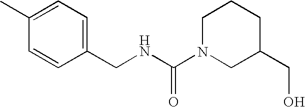

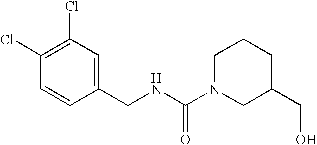

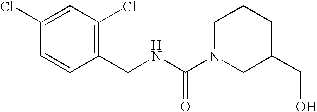

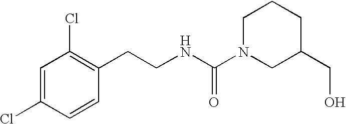

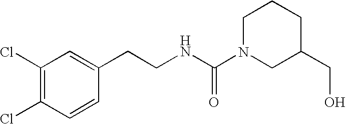

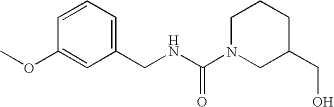

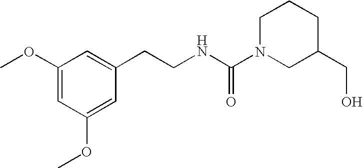

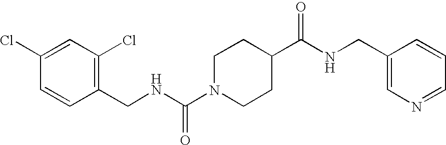

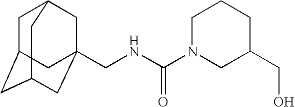

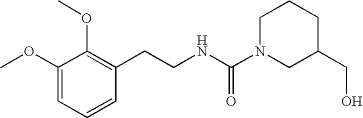

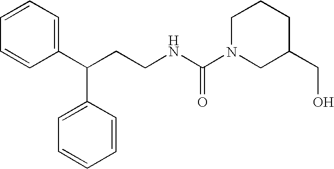

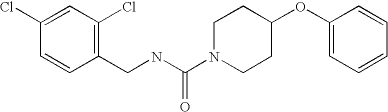

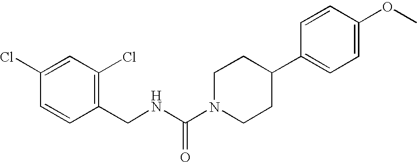

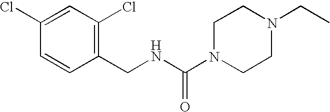

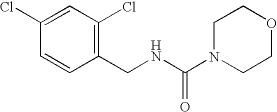

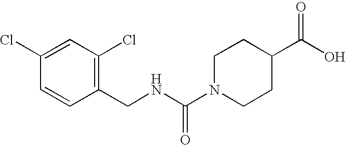

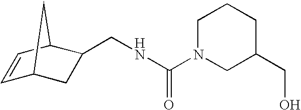

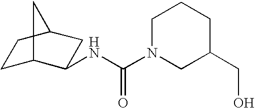

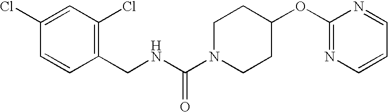

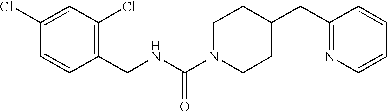

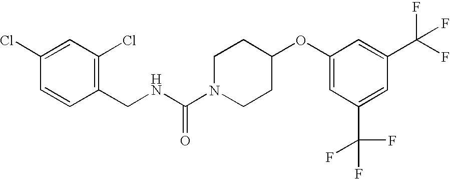

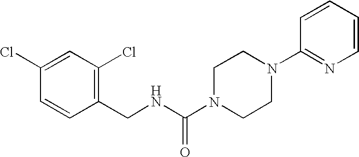

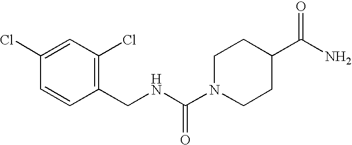

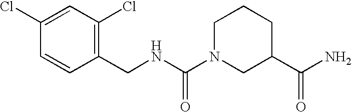

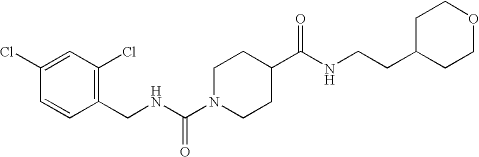

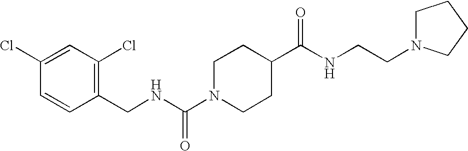

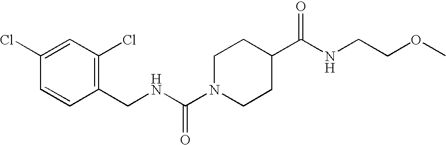

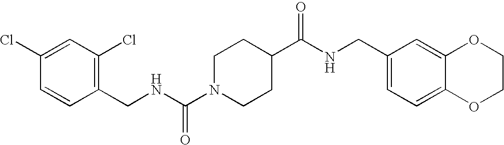

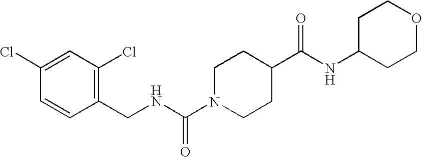

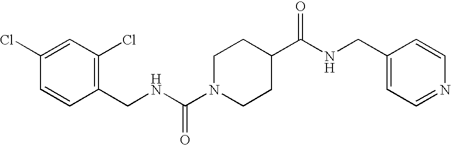

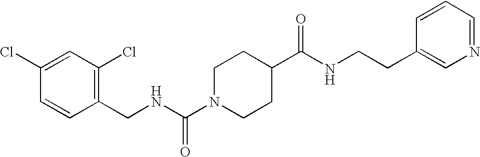

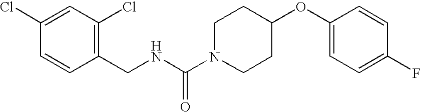

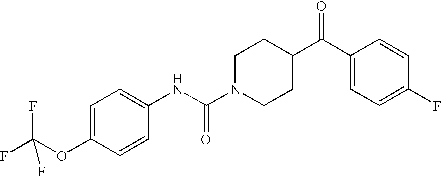

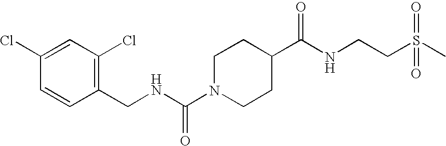

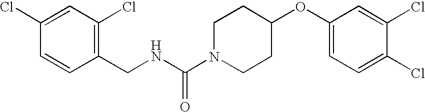

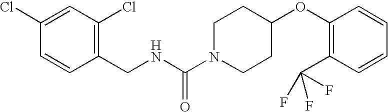

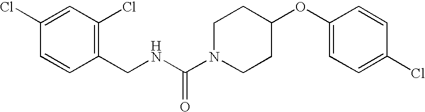

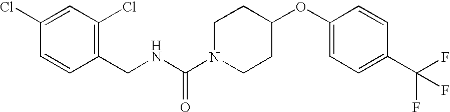

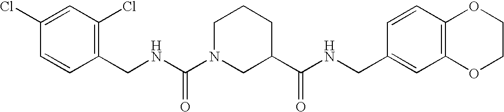

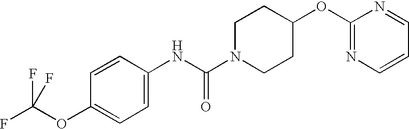

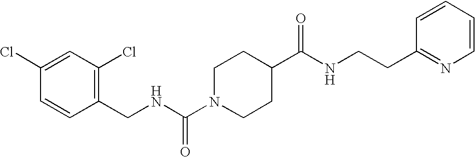

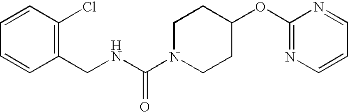

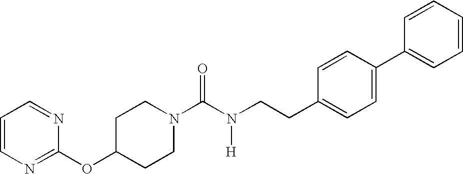

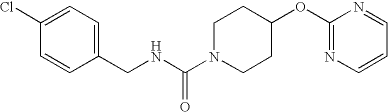

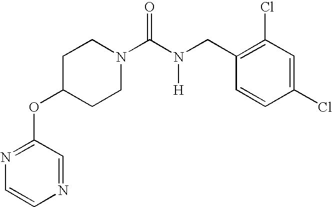

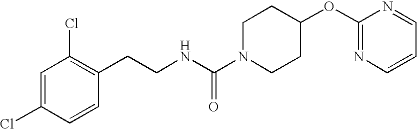

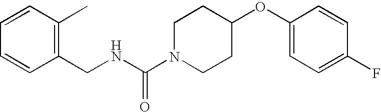

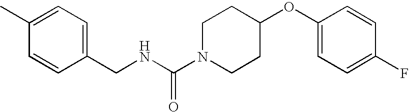

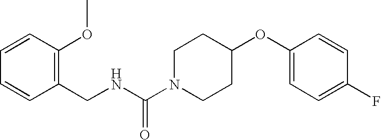

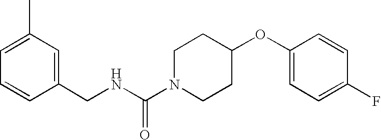

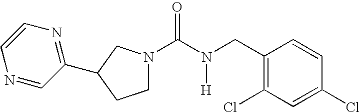

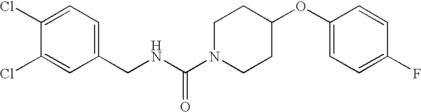

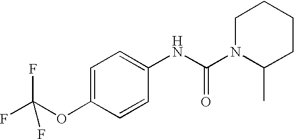

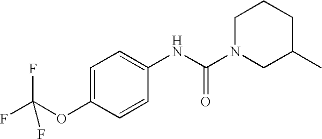

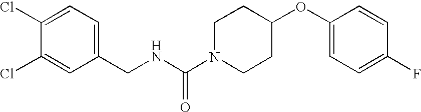

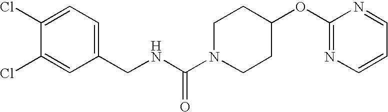

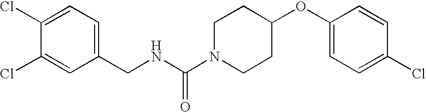

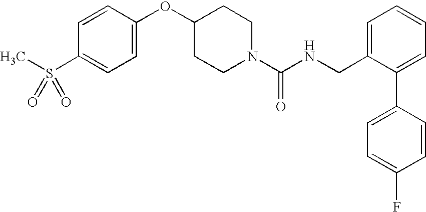

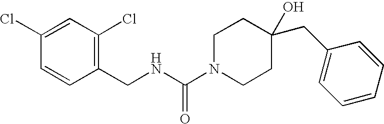

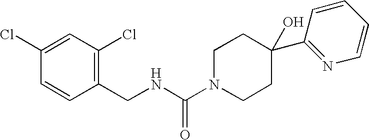

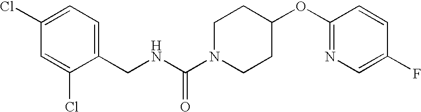

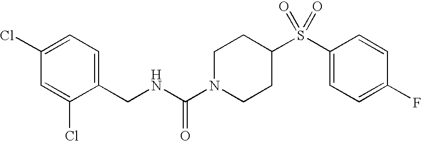

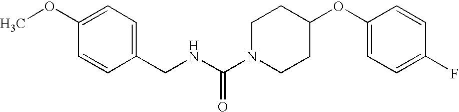

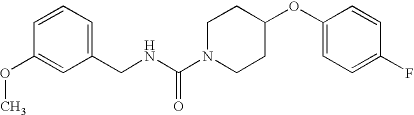

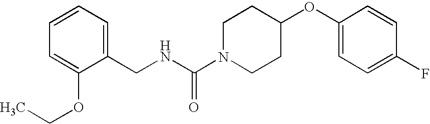

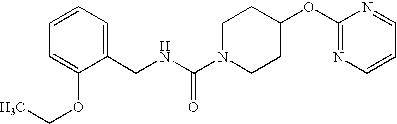

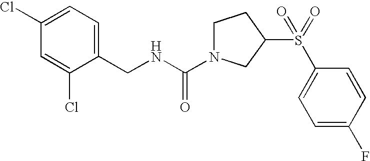

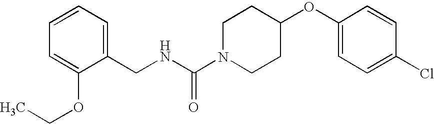

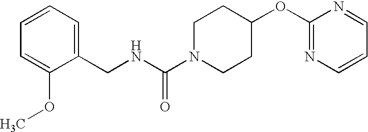

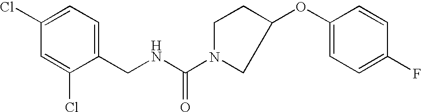

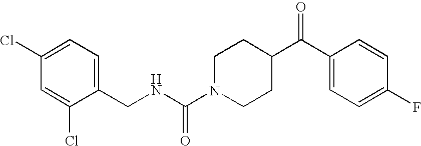

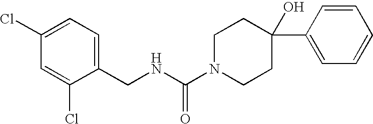

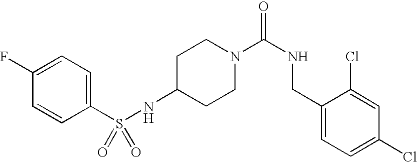

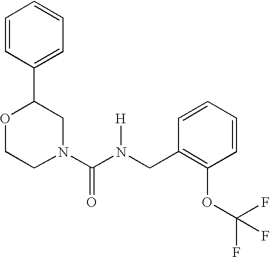

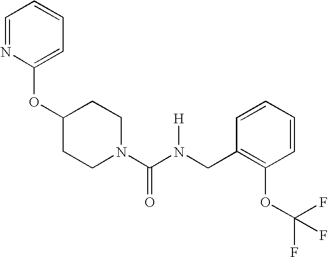

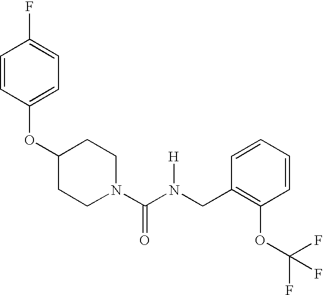

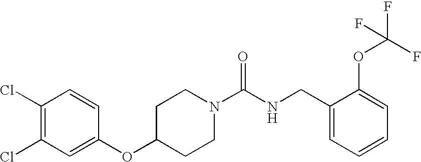

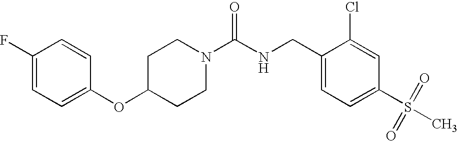

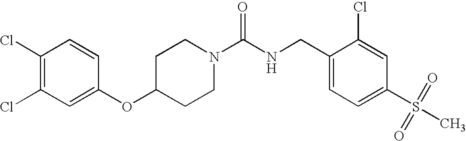

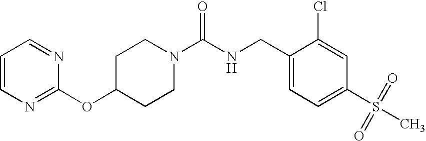

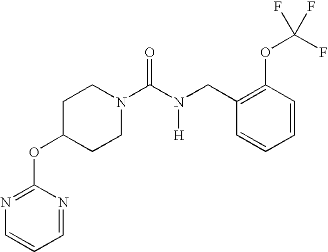

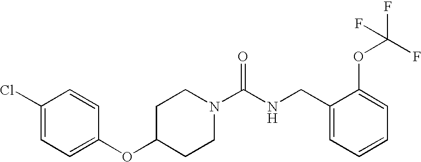

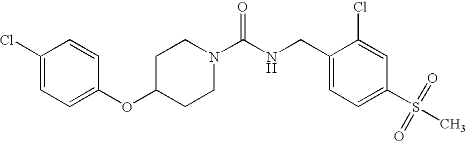

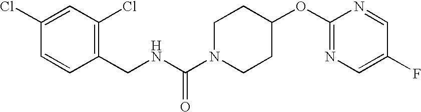

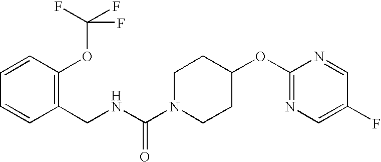

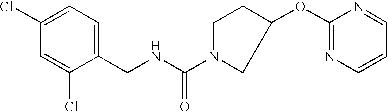

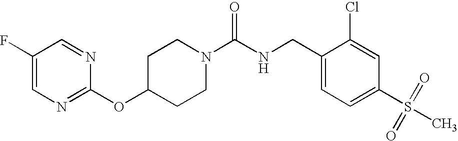

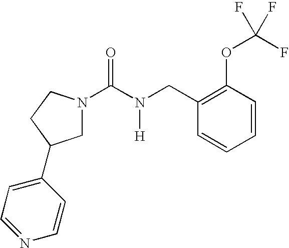

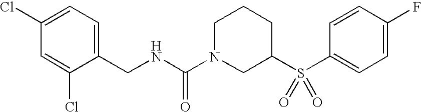

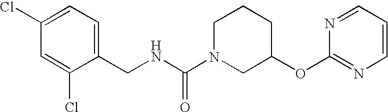

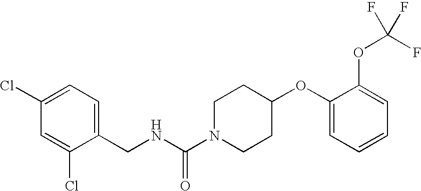

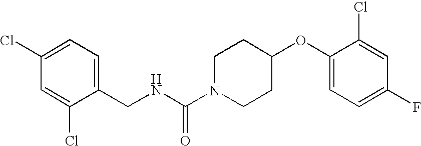

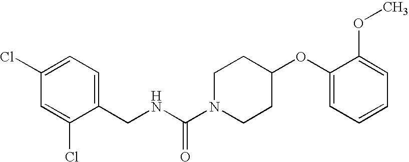

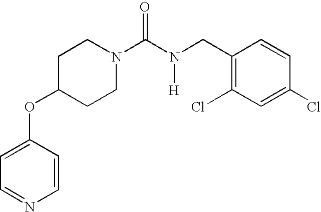

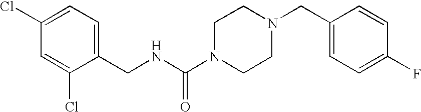

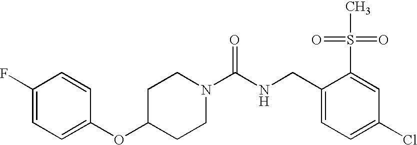

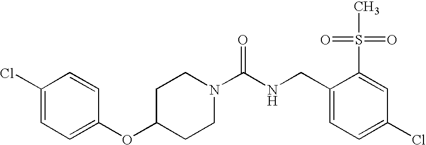

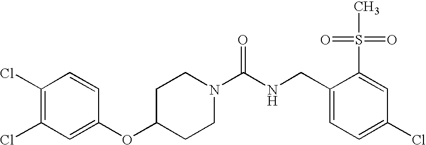

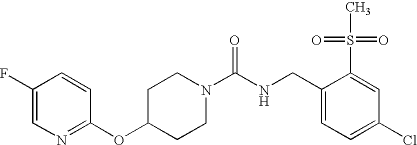

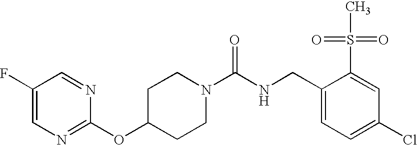

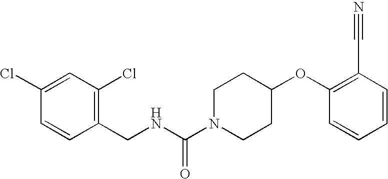

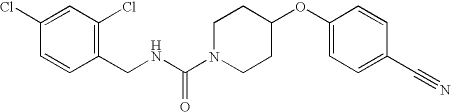

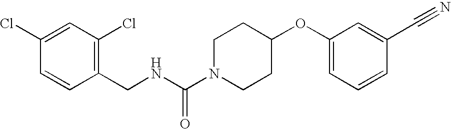

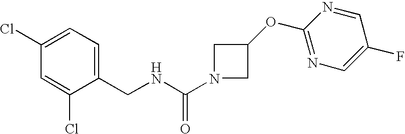

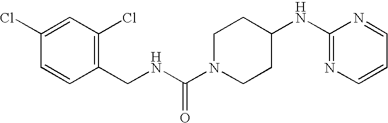

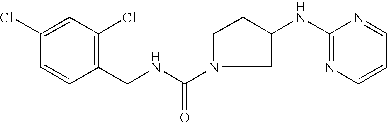

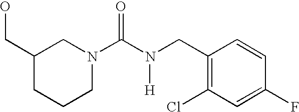

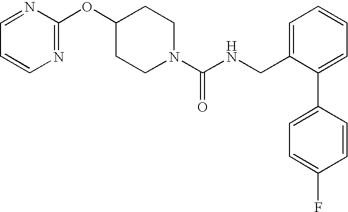

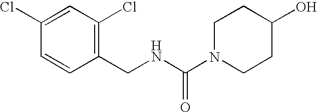

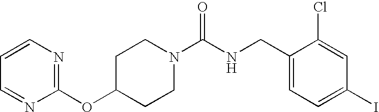

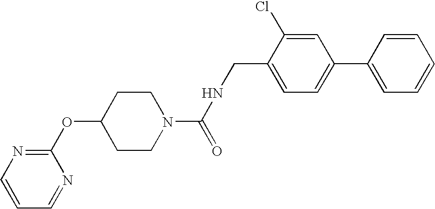

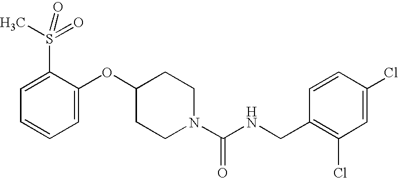

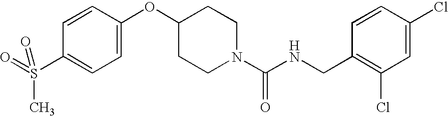

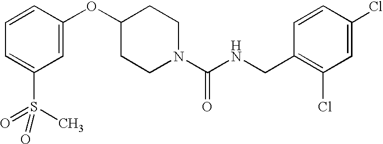

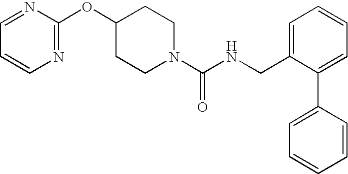

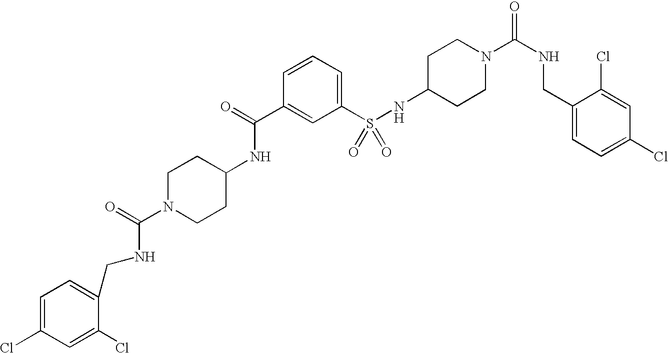

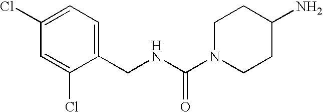

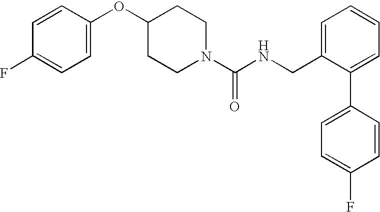

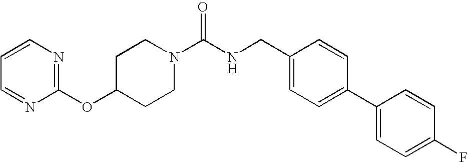

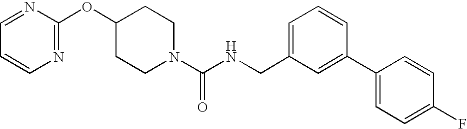

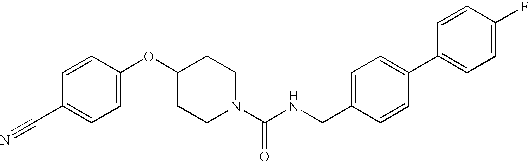

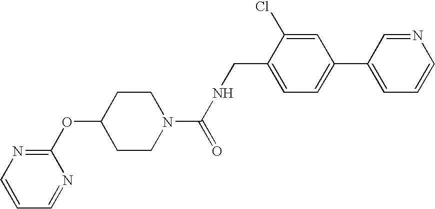

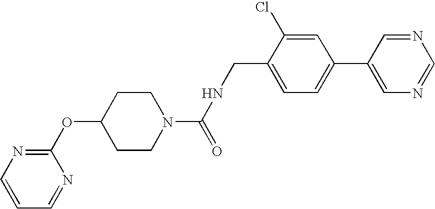

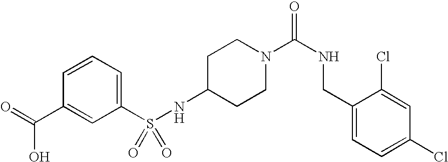

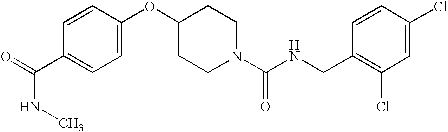

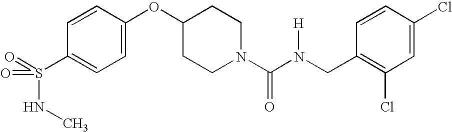

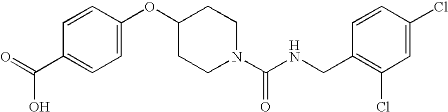

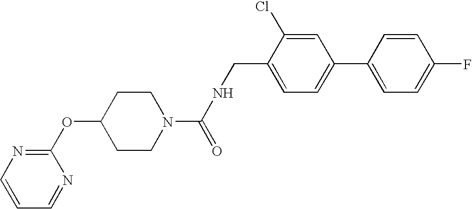

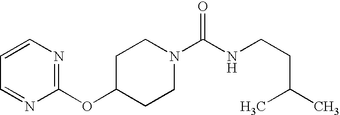

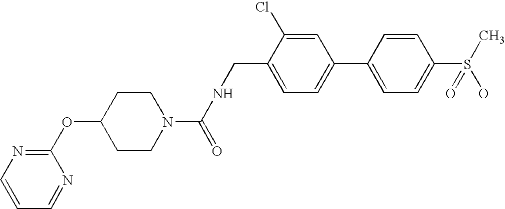

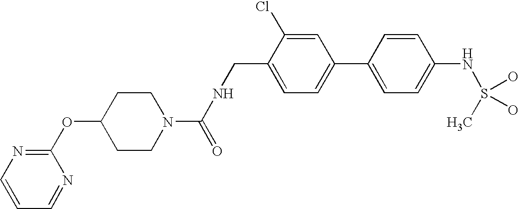

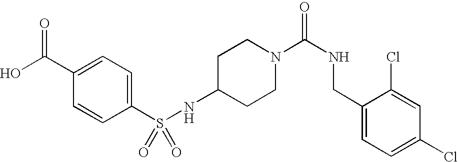

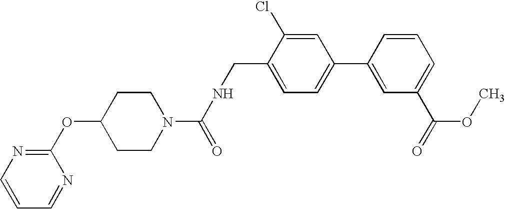

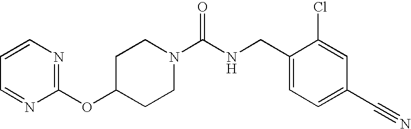

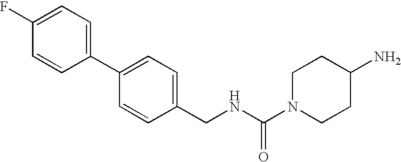

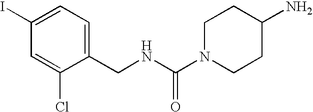

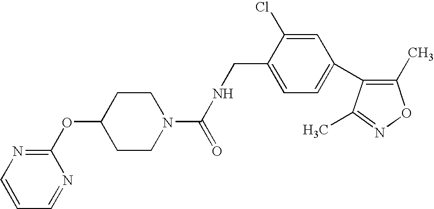

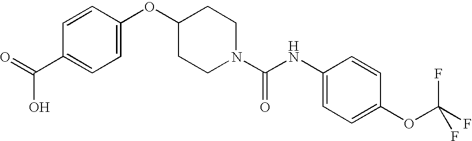

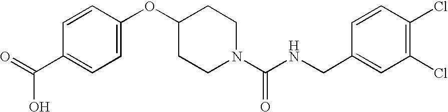

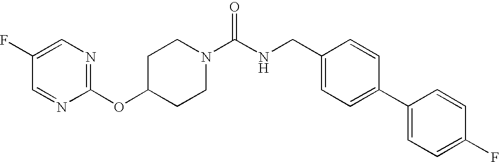

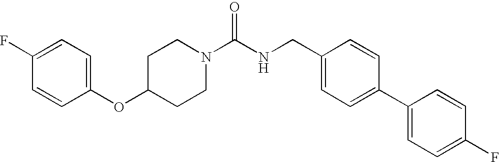

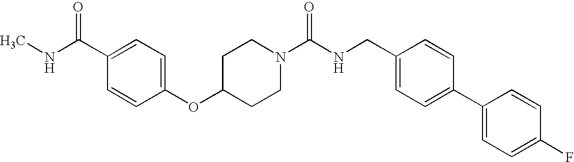

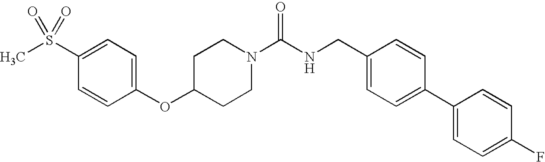

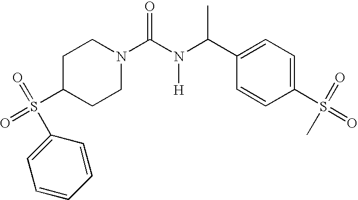

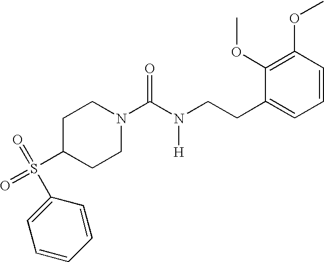

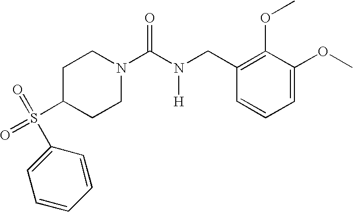

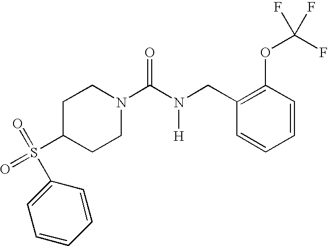

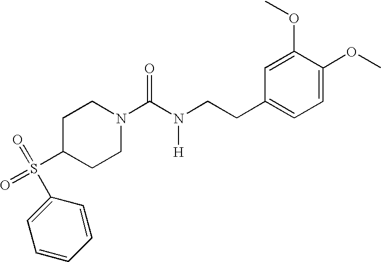

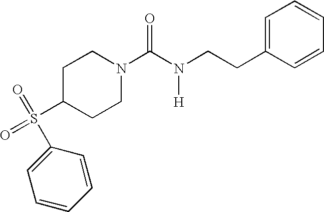

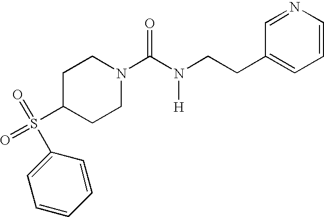

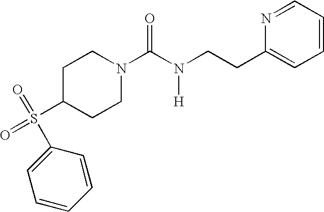

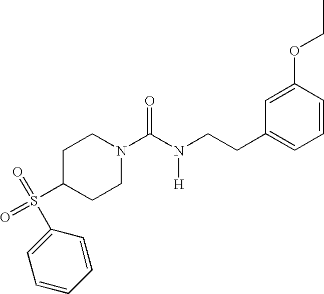

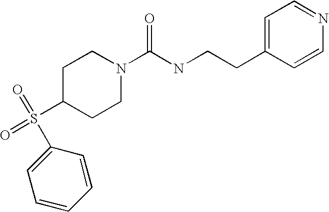

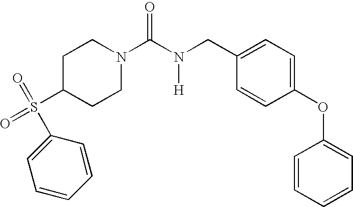

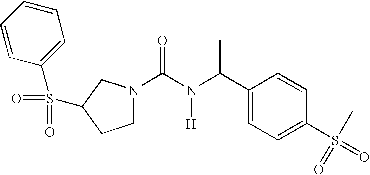

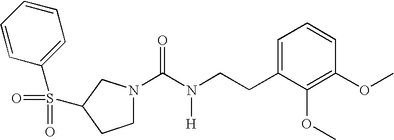

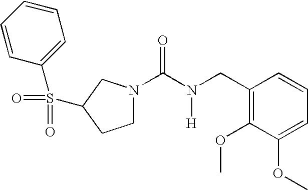

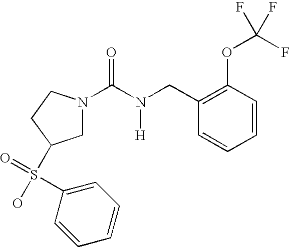

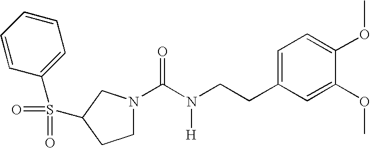

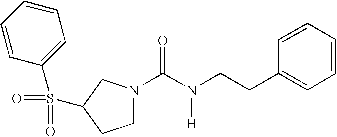

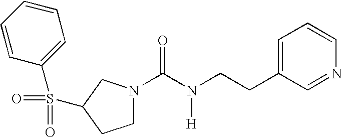

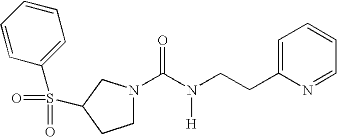

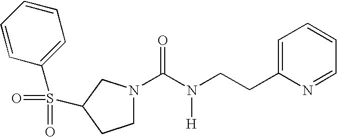

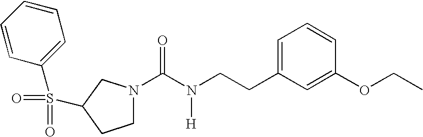

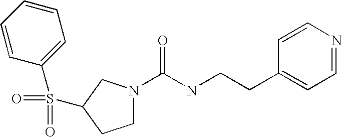

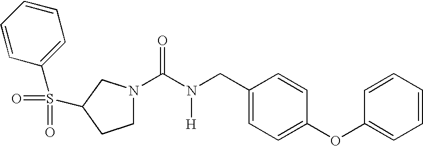

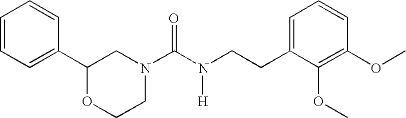

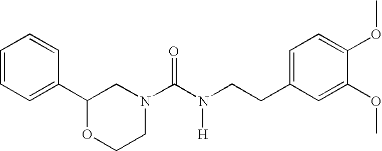

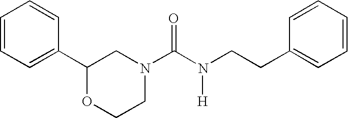

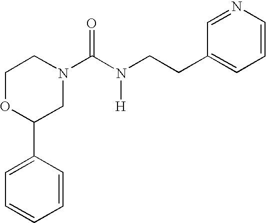

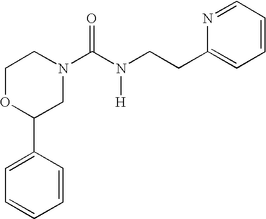

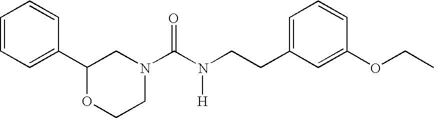

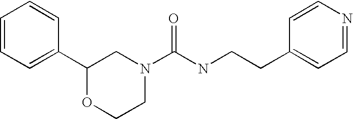

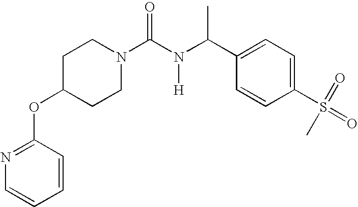

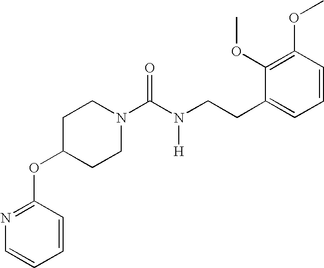

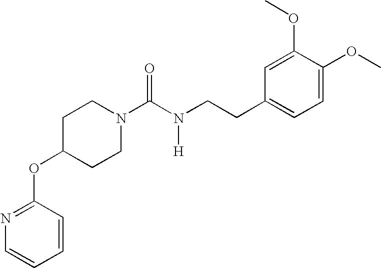

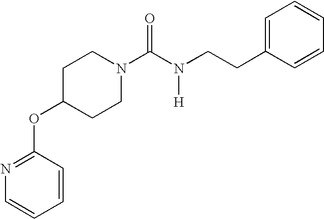

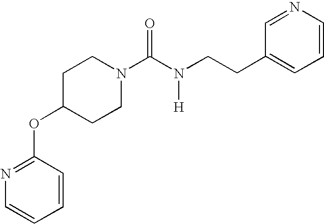

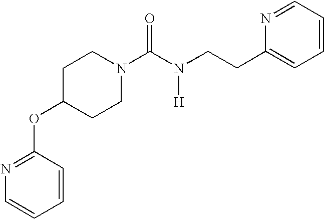

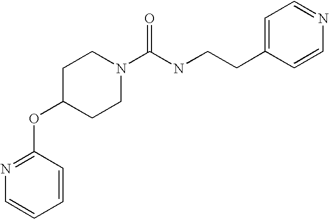

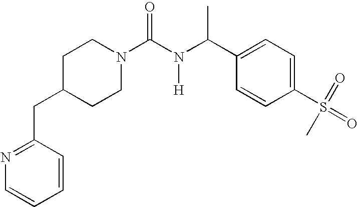

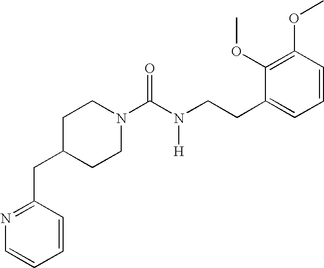

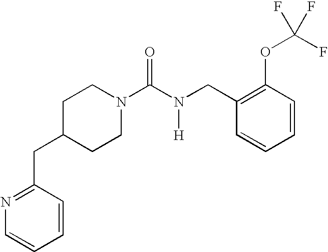

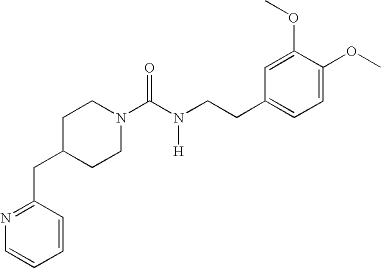

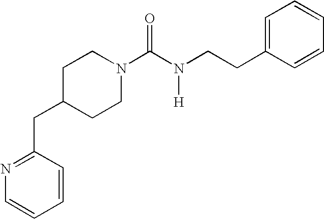

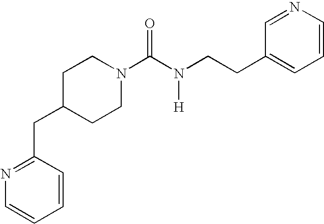

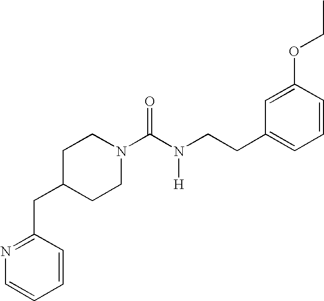

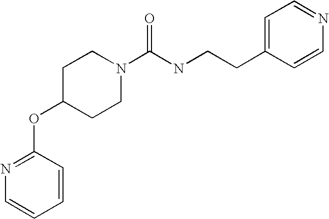

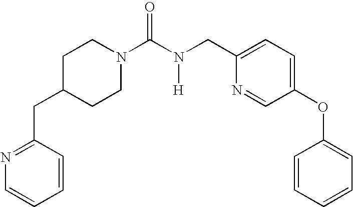

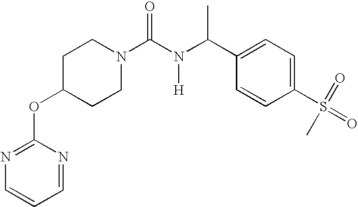

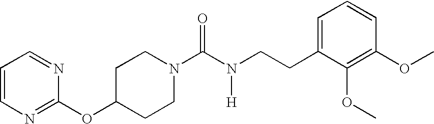

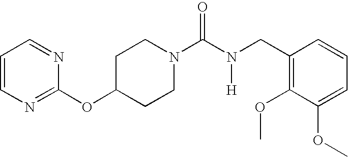

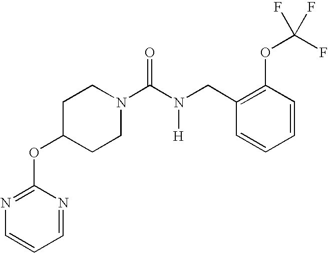

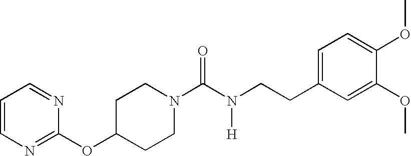

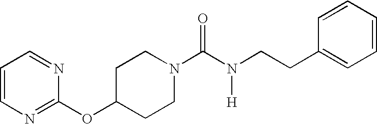

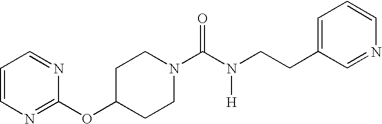

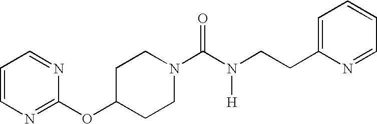

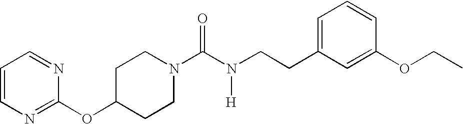

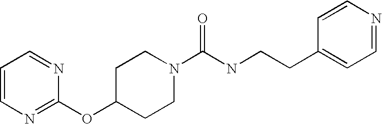

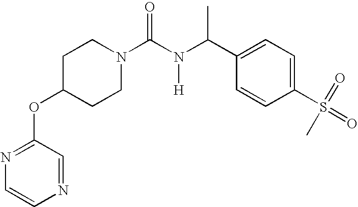

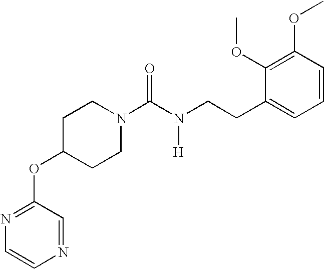

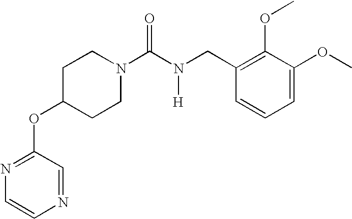

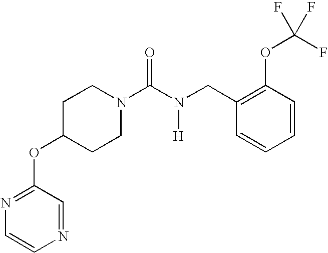

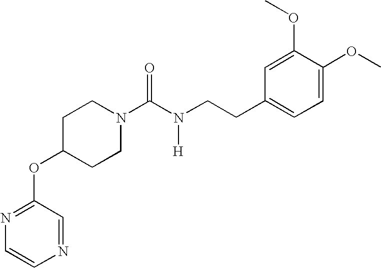

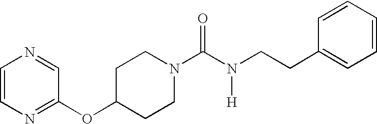

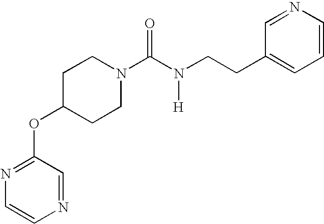

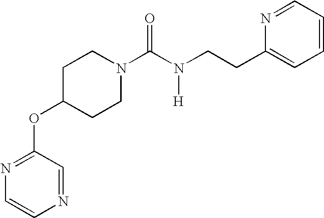

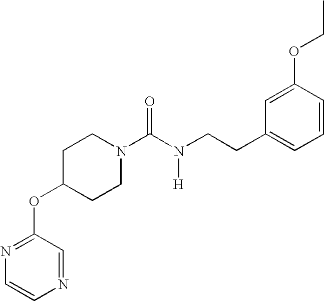

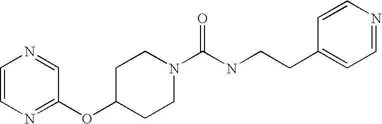

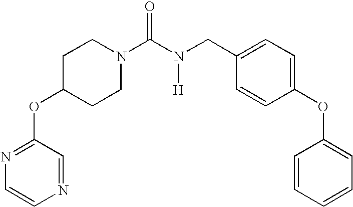

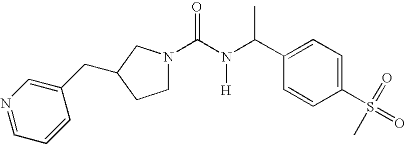

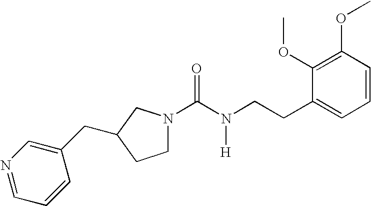

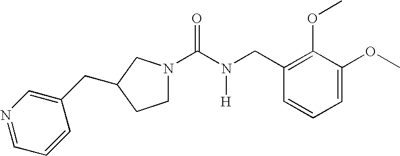

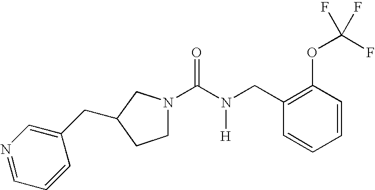

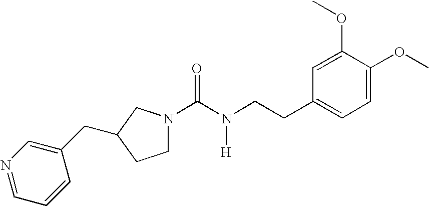

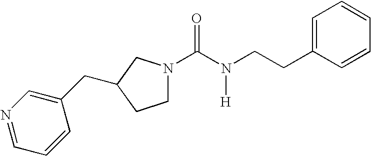

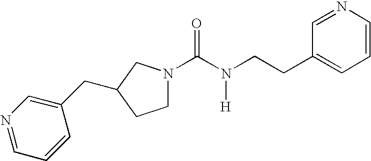

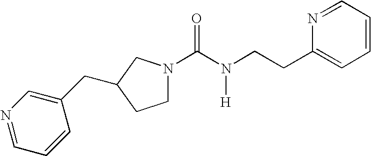

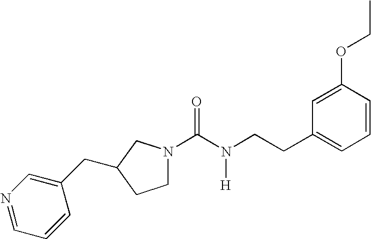

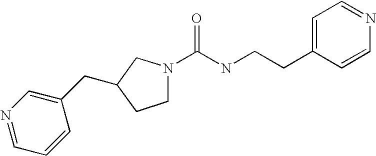

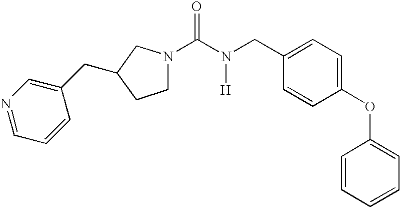

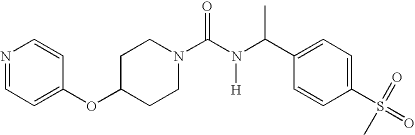

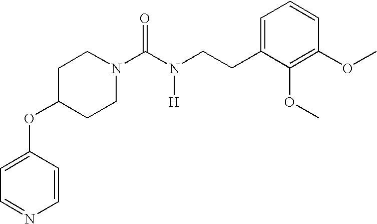

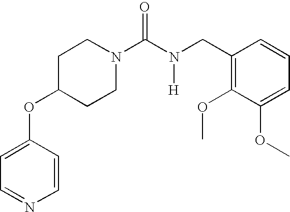

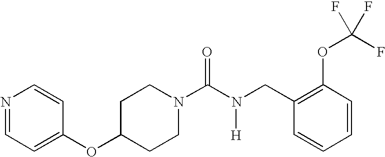

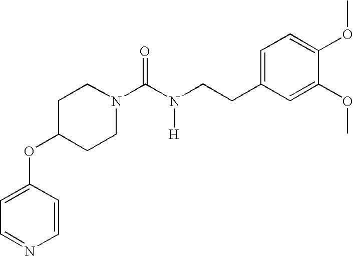

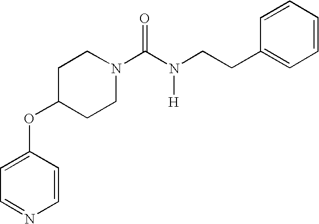

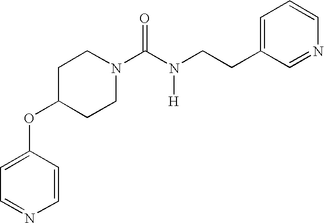

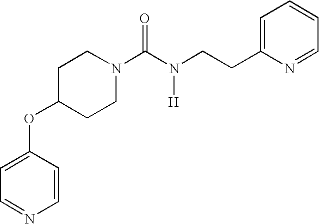

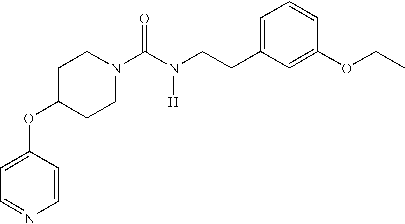

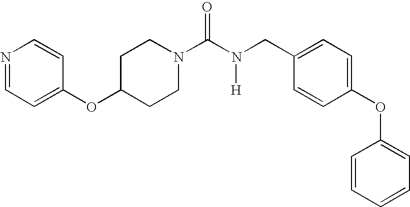

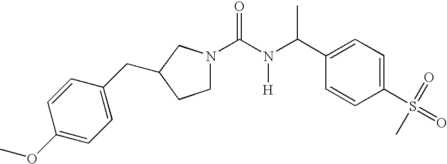

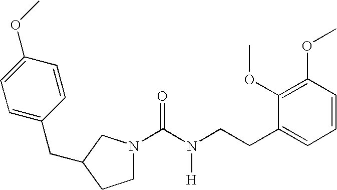

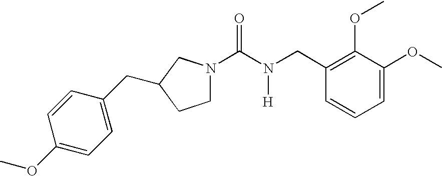

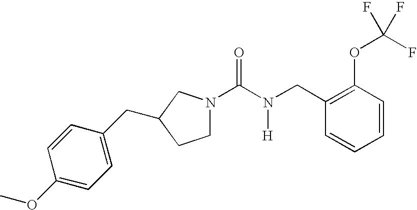

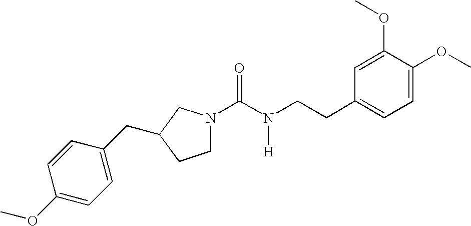

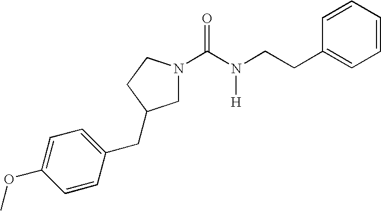

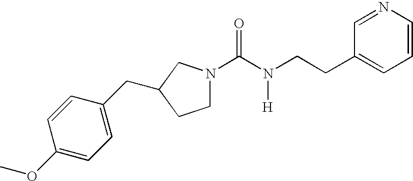

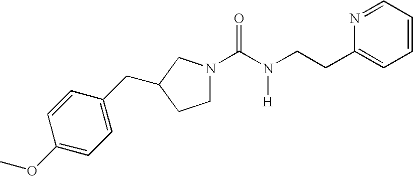

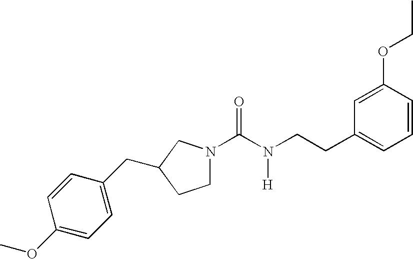

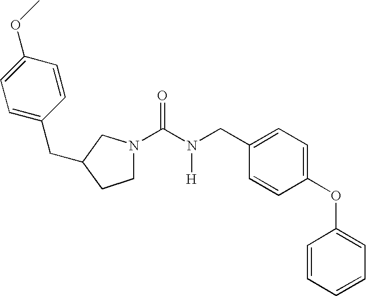

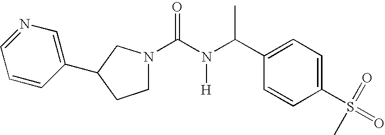

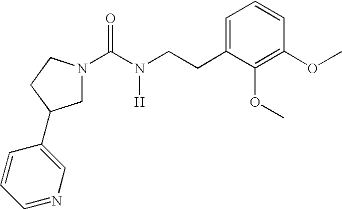

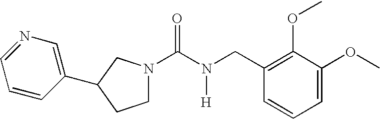

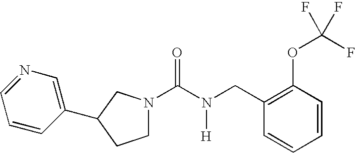

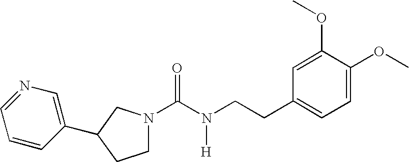

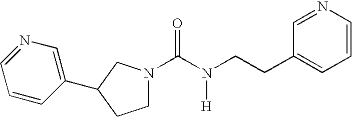

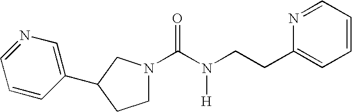

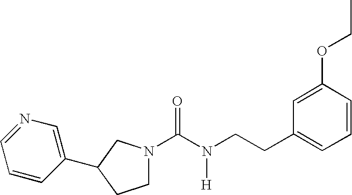

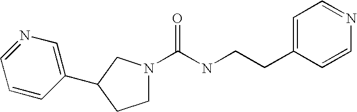

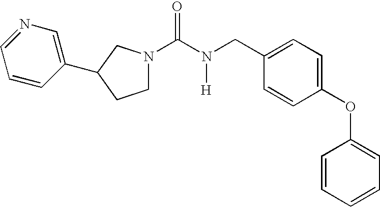

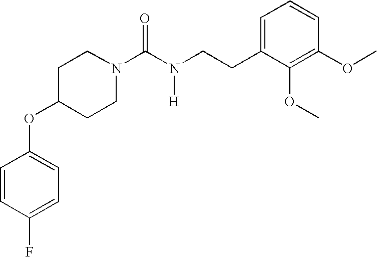

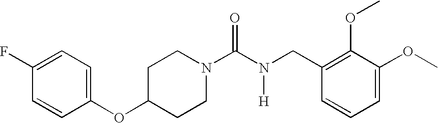

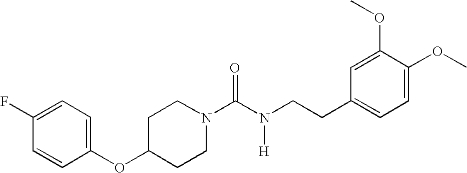

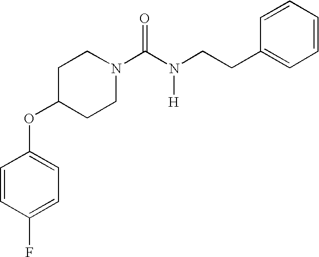

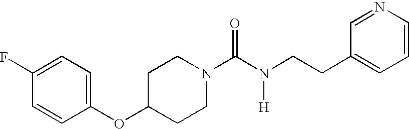

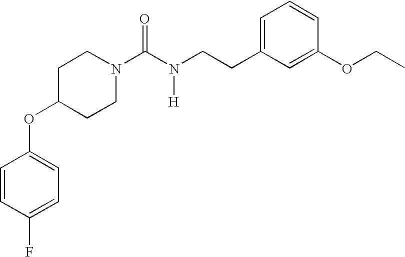

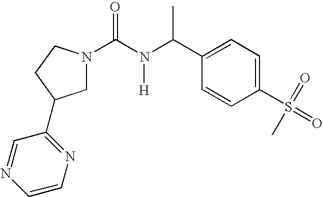

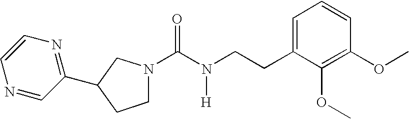

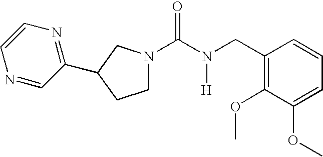

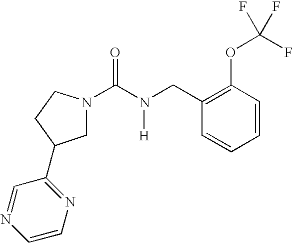

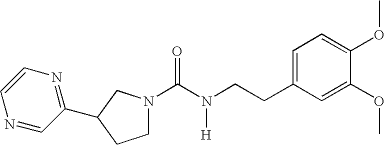

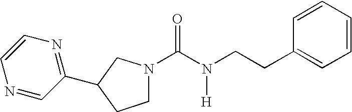

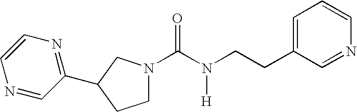

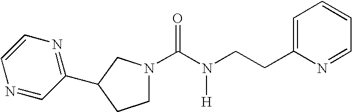

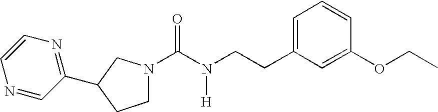

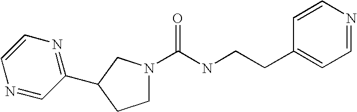

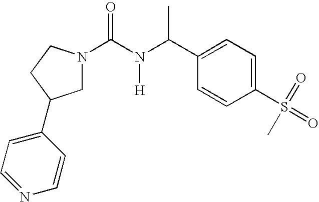

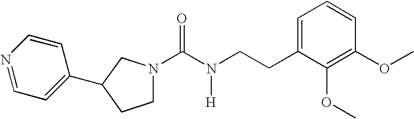

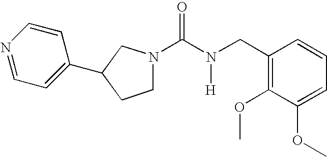

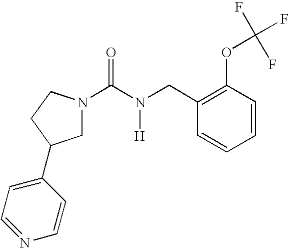

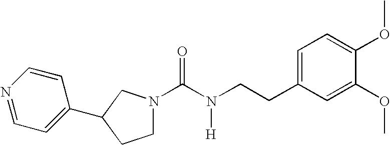

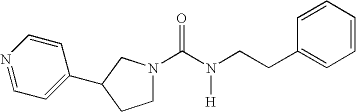

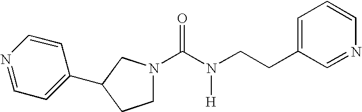

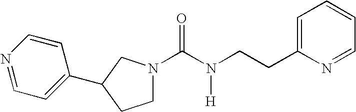

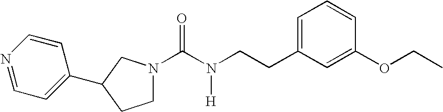

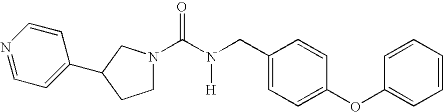

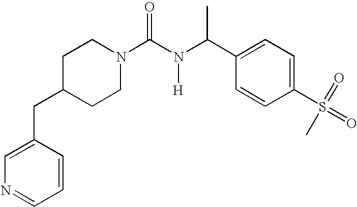

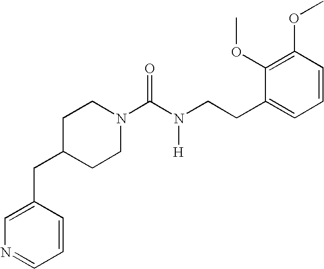

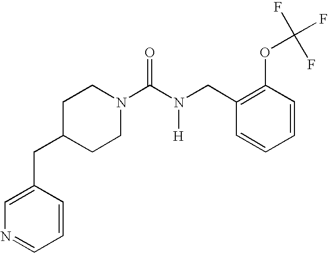

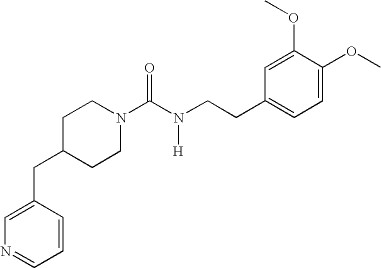

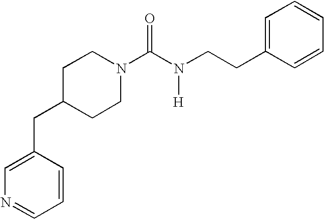

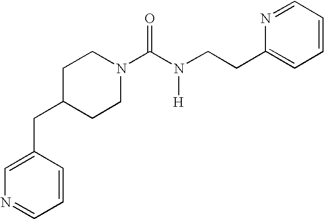

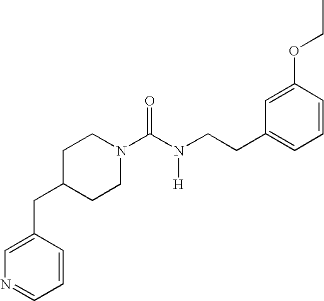

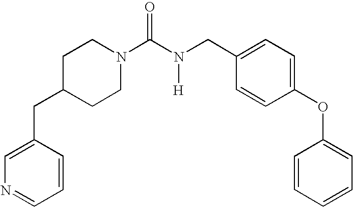

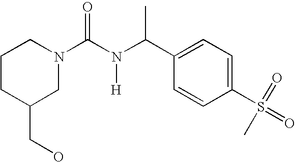

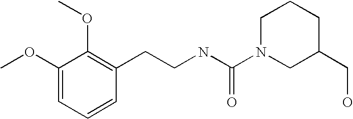

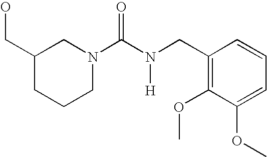

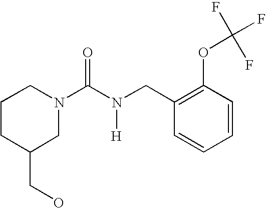

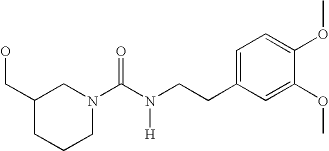

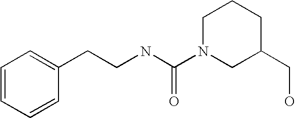

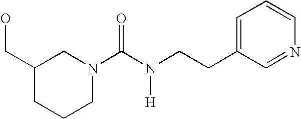

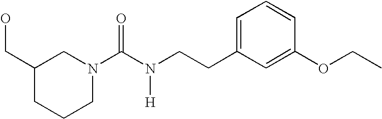

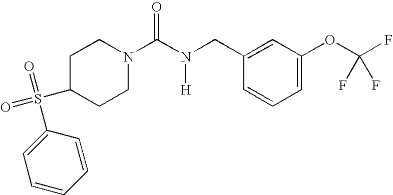

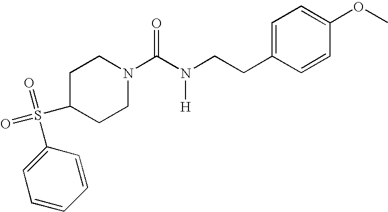

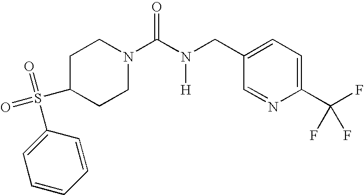

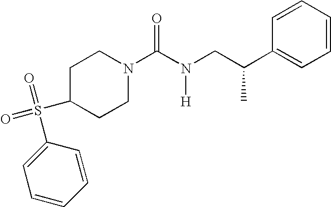

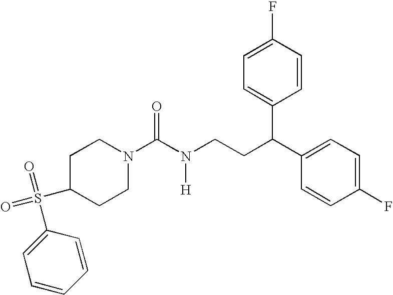

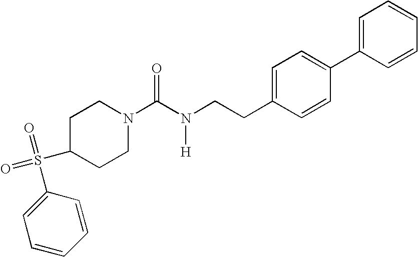

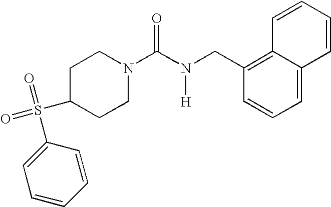

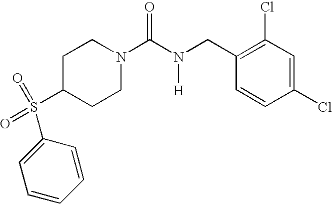

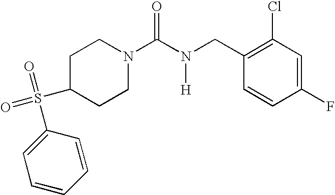

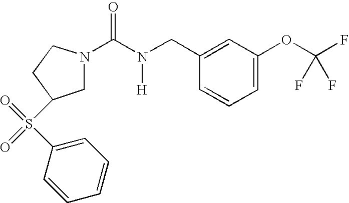

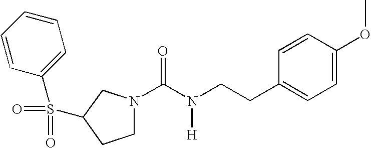

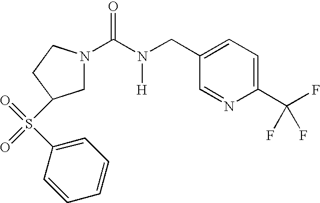

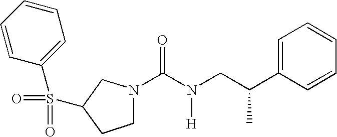

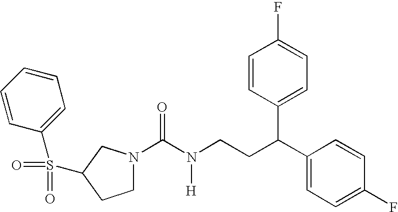

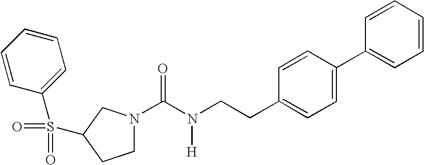

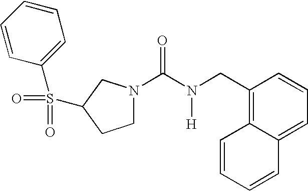

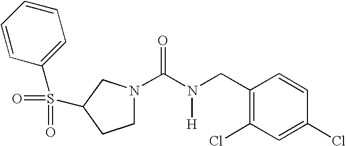

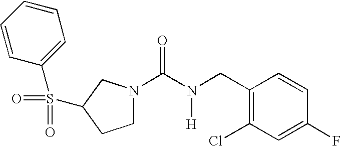

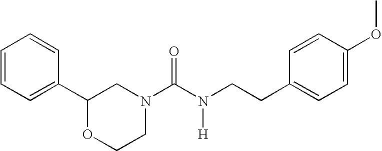

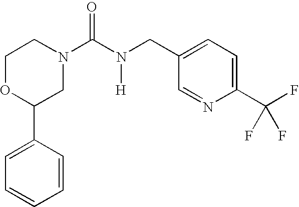

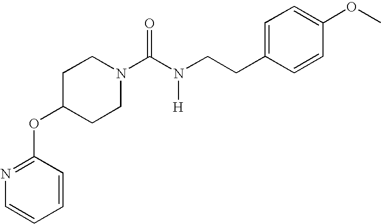

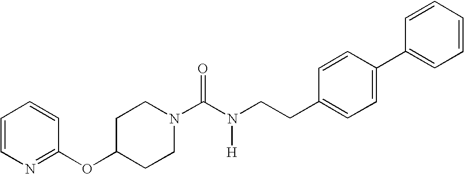

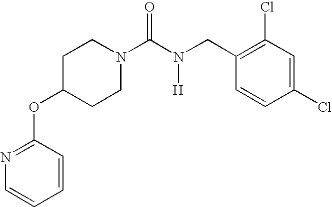

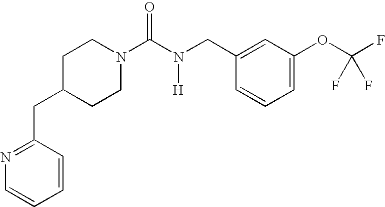

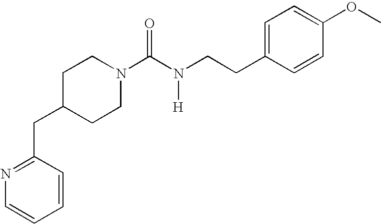

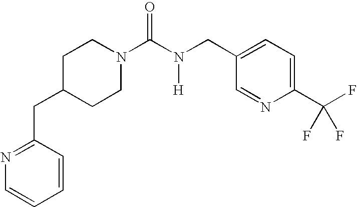

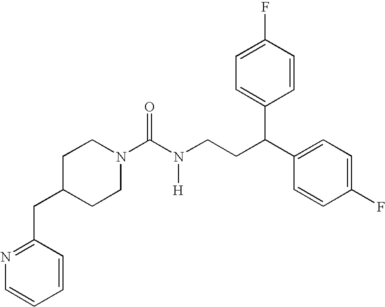

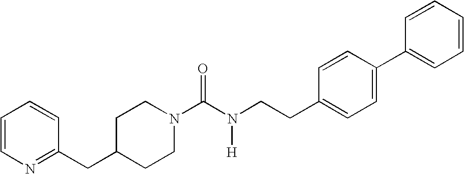

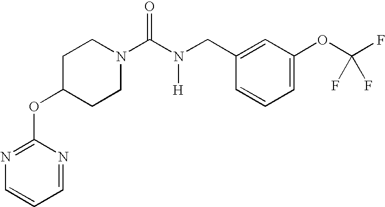

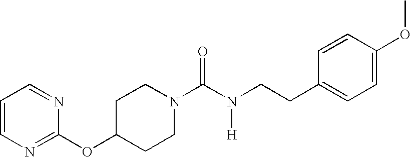

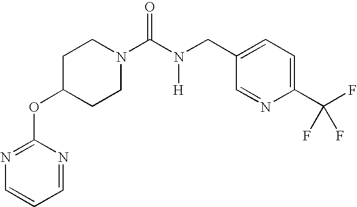

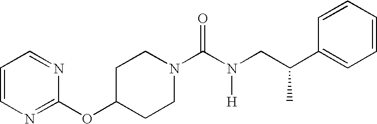

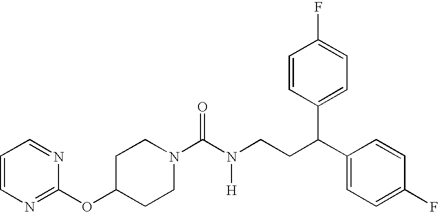

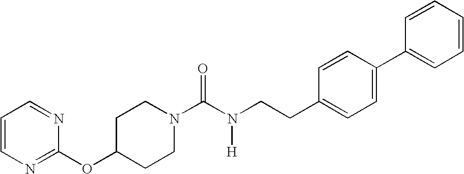

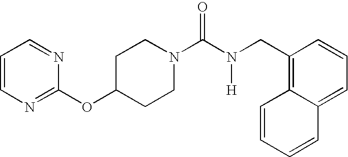

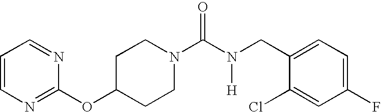

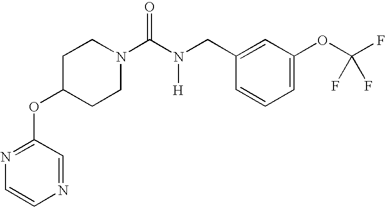

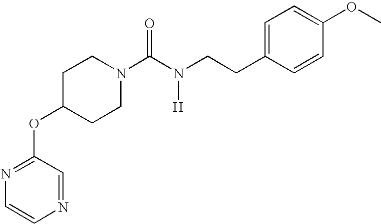

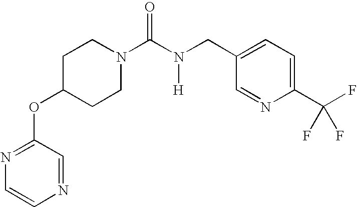

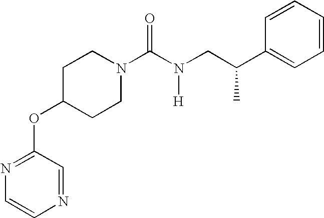

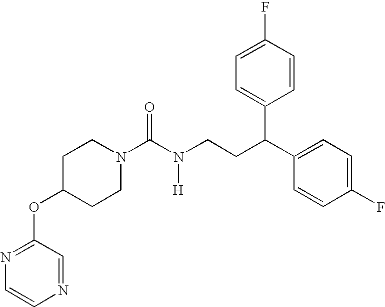

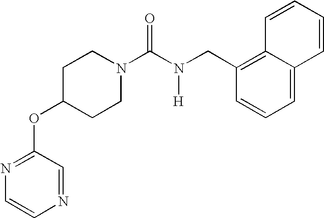

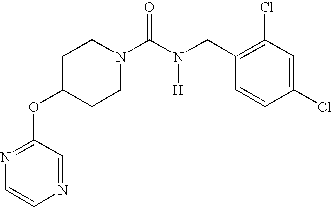

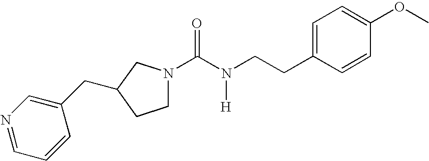

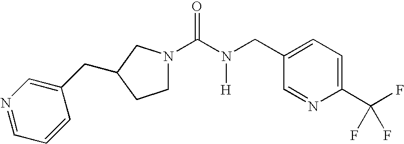

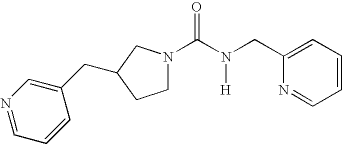

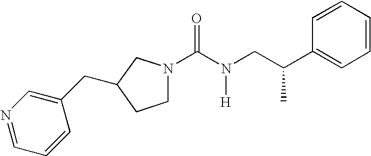

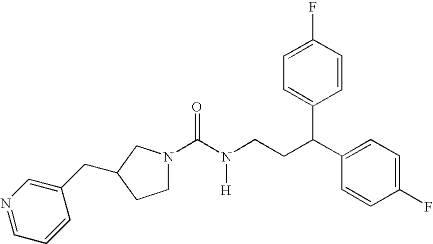

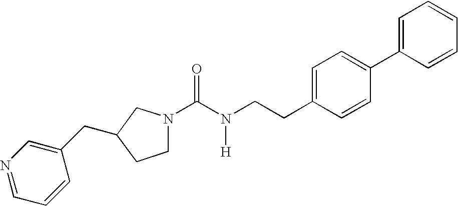

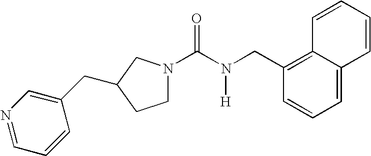

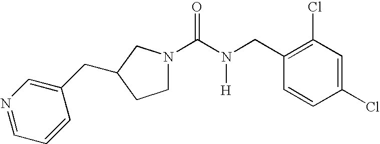

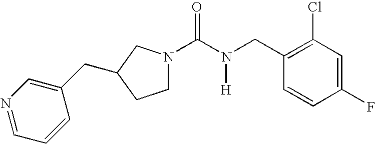

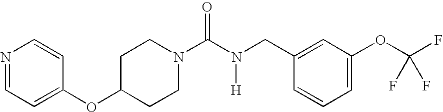

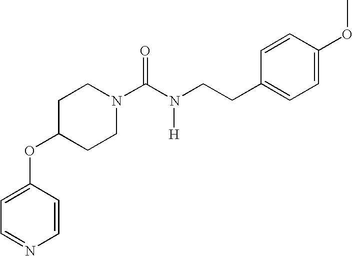

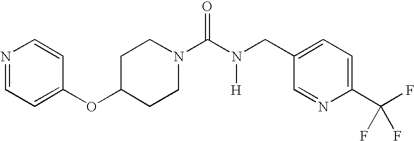

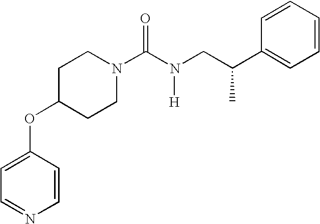

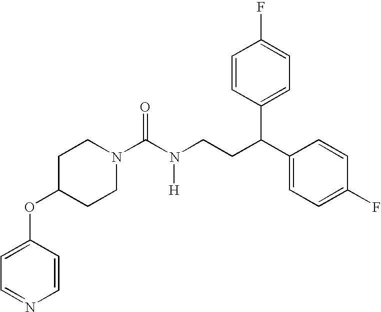

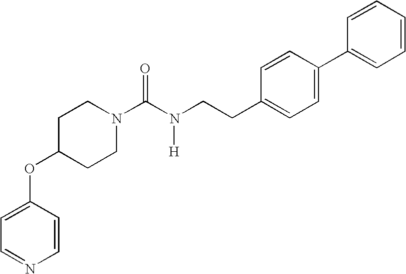

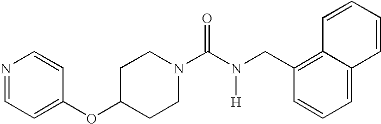

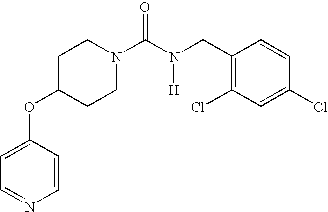

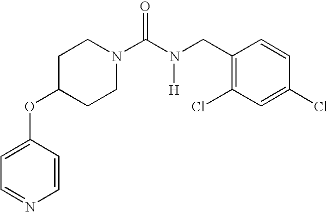

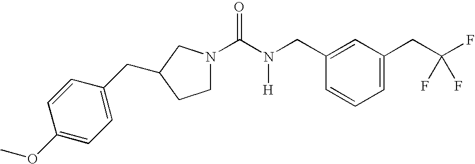

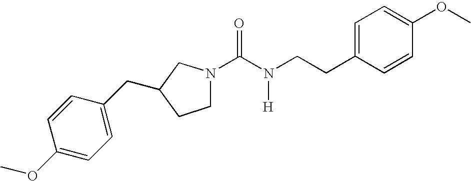

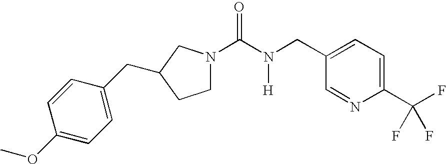

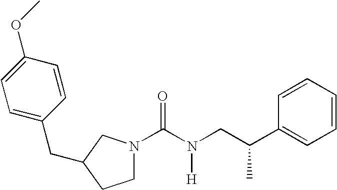

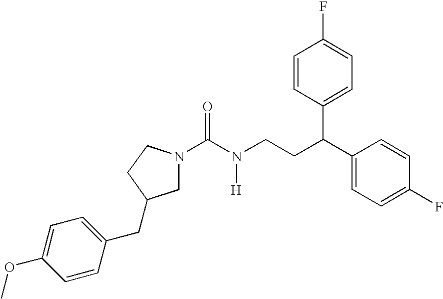

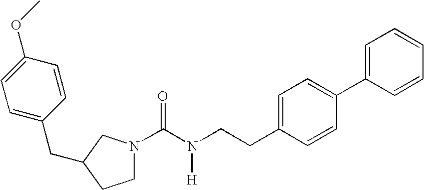

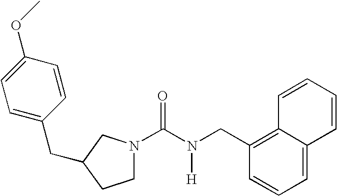

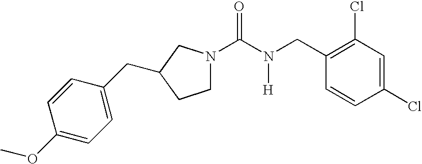

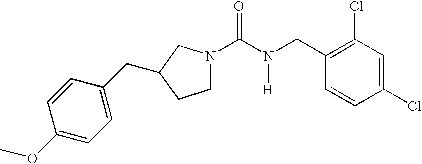

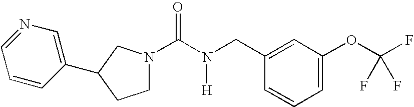

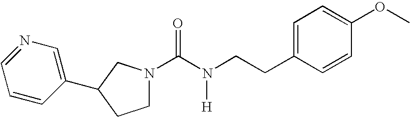

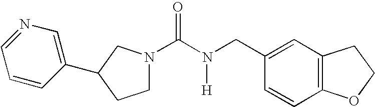

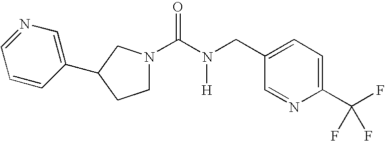

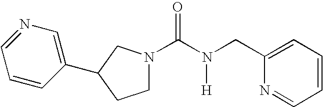

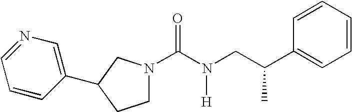

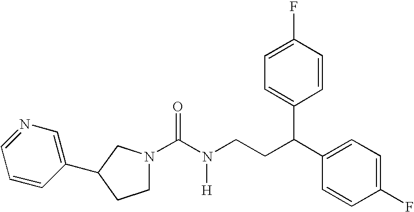

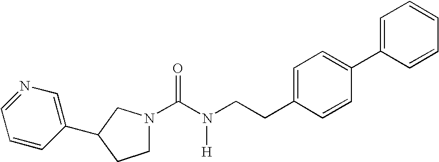

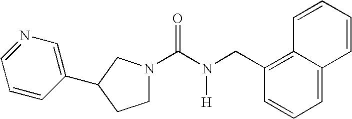

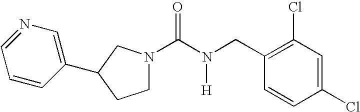

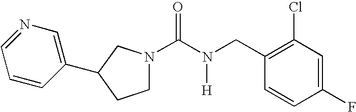

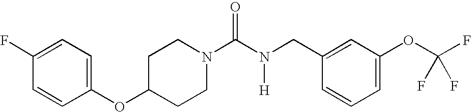

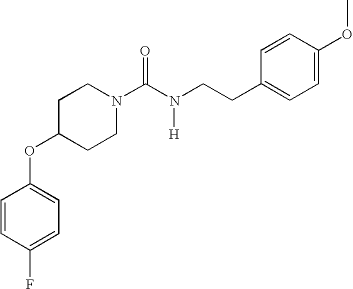

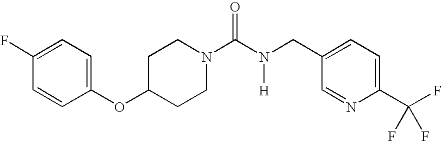

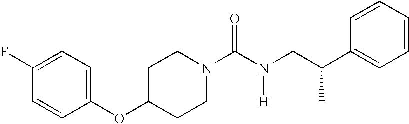

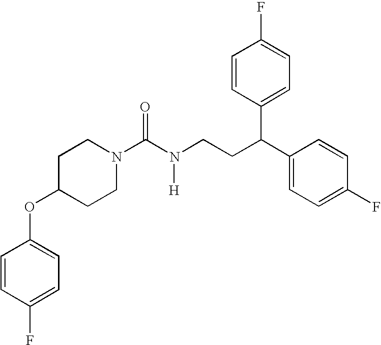

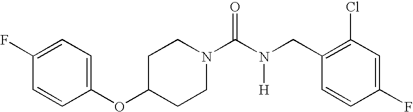

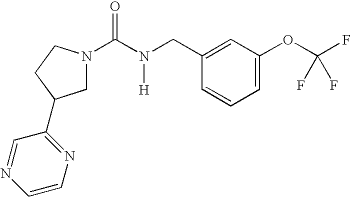

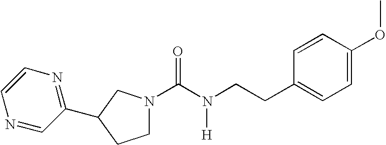

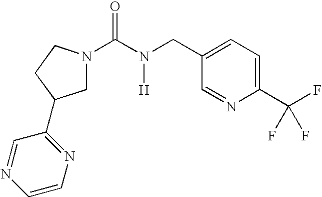

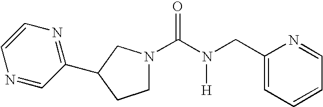

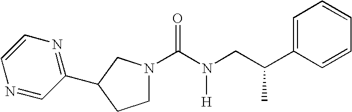

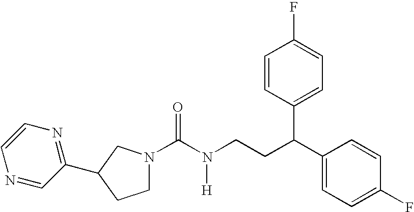

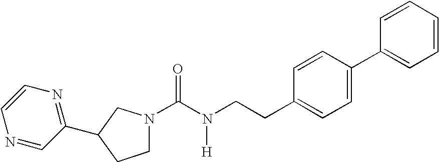

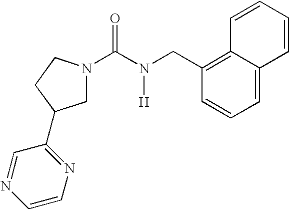

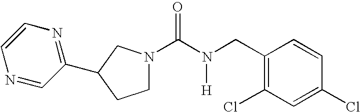

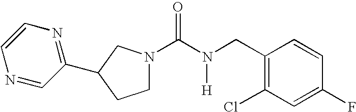

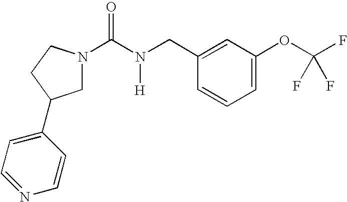

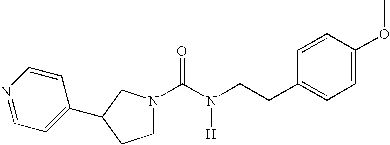

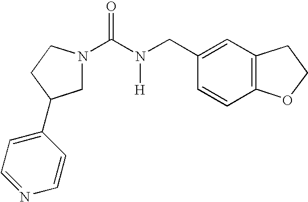

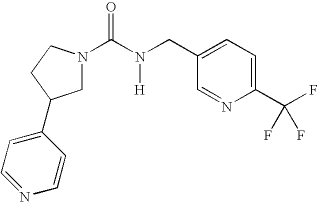

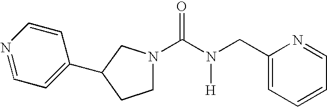

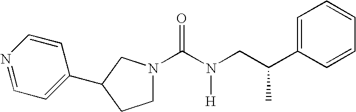

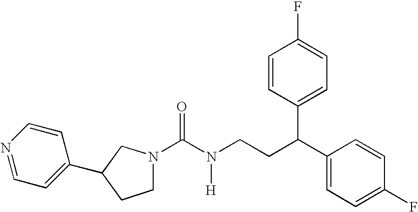

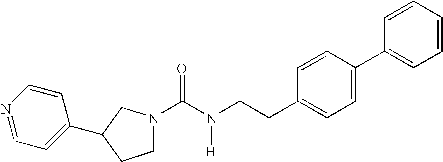

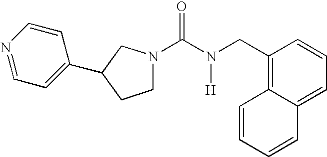

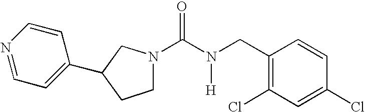

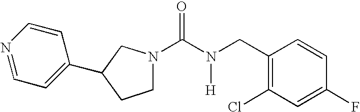

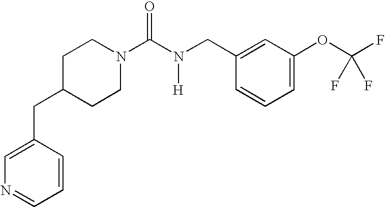

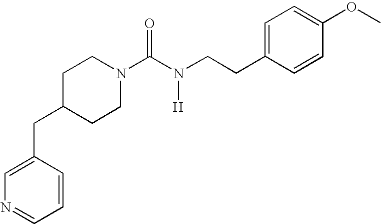

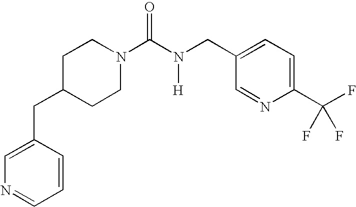

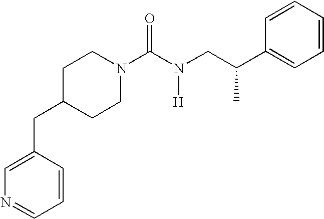

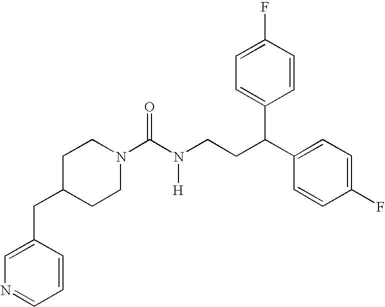

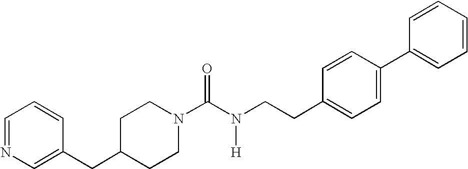

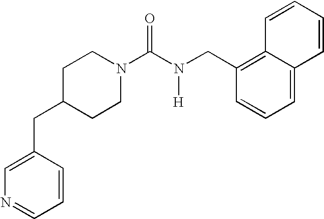

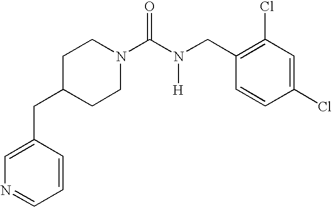

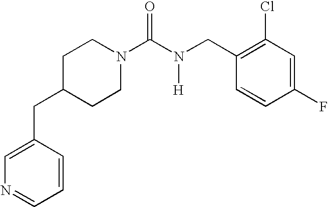

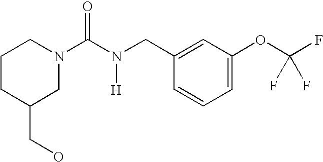

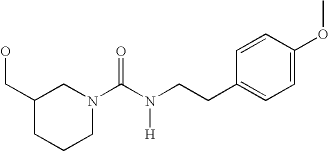

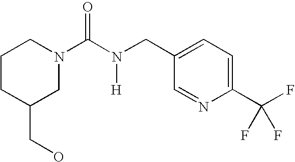

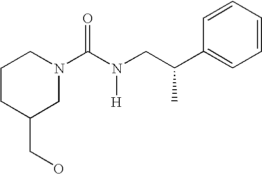

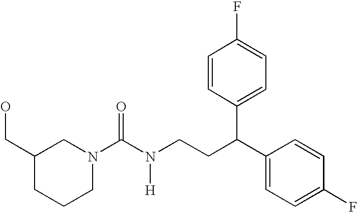

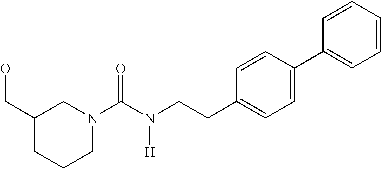

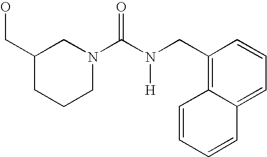

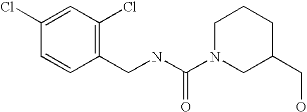

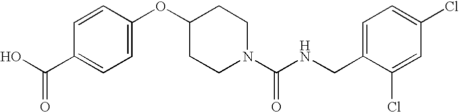

or the pharmaceutically acceptable salts thereof.

In all the compounds disclosed hereinabove in this application, in the event the nomenclature is in conflict with the structure, it shall be understood that the compound is defined by the structure.

The invention includes the use of any compounds of described above which may contain one or more asymmetric carbon atoms and may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of formula (I) can exist in more than one tautomeric form. The invention includes methods using all such tautomers.

All terms as used herein in this specification, unless otherwise stated, shall be understood in their ordinary meaning as known in the art. For example, C₁₋₄alkoxy includes the organic radical C₁₋₄alkyl with a terminal oxygen, such as methoxy, ethoxy, propoxy, butoxy.

All organic radicals: alkyl, alkenyl and alkynyl groups, or such groups which are incorporated in other radicals such as acyl and alkoxy, shall be understood as being branched or unbranched where structurally possible and unless otherwise specified, and may be partially or fully halogenated.

The term “lower” referred to above and hereinafter in connection with organic radicals or compounds respectively defines such as branched or unbranched with up to and including 7, preferably up to and including 4 and advantageously one or two carbon atoms.

A cyclic group shall be understood to mean carbocycle, heterocycle or heteroaryl, each may be partially or fully halogenated.

An acyl group is a radical defined as —C(O)—R, where R is an organic radical or a cyclic group. Acyl represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocycles include hydrocarbon rings containing from three to fourteen carbon atoms. These carbocycles may be either aromatic either aromatic or non-aromatic ring systems. The non-aromatic ring systems may be mono- or polyunsaturated, monocyclic, bicyclic or tricyclic and may be bridged. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, benzyl, indanyl, indenyl, benzocyclobutanyl, dihydronaphthyl, tetrahydronaphthyl, naphthyl, decahydronaphthyl, benzocycloheptanyl, fluorene, and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl and imidazo[4,5-b]pyridinyl.

The term “heteroatom” as used herein shall be understood to mean atoms other than carbon such as oxygen, nitrogen, sulfur and phosphorous.

As used herein, “nitrogen” and “sulfur” include any oxidized form of nitrogen and sulfur and the quaternized form of any basic nitrogen. All heteroatoms in open chain or cyclic radicals include all oxidized forms.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative and/or is partially or fully halogenated. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts.

Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

### General Synthetic Methods

The invention also provides processes for making compounds of Formula (I). In all schemes, unless specified otherwise, G, L, n, R, and X in the formulas below shall have the meaning of G, L, n, R, and X in Formula (I) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization.

The appropriately substituted starting materials and intermediates used in the preparation of compounds of the invention are either commercially available or readily prepared by methods known in the literature to those skilled in the art, and are illustrated in the synthetic examples below.

Compounds of Formula (I) may be synthesized by the method illustrated in Scheme 1

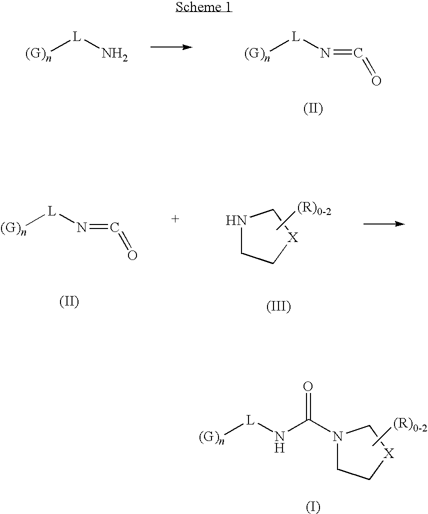

Reaction of the starting amine with a reagent such as triphosgene, in a suitable solvent, provides an isocyanate of formula (II). The isocyanate may also be commercially available. Reacting the isocyanate of formula (II) with a secondary amine of formula (III), in a suitable solvent, in the presence of a suitable base, provides the desired compound of formula (I).

Alternatively, reaction of the starting amine with secondary amine of formula (III), in the presence of a coupling agent such as carbonyldiimidazole, in a suitable solvent, provides the desired compound of formula (I).

Further modification of the initial product of formula (I) by methods known in the art and illustrated in the Examples below, may be used to prepare additional compounds of this invention.

Intermediate (III) may be synthesized by methods outlined in Schemes 2, 3, or 4, when R=—W-Q, wherein W and Q are as defined in Formula (I) described herein above.

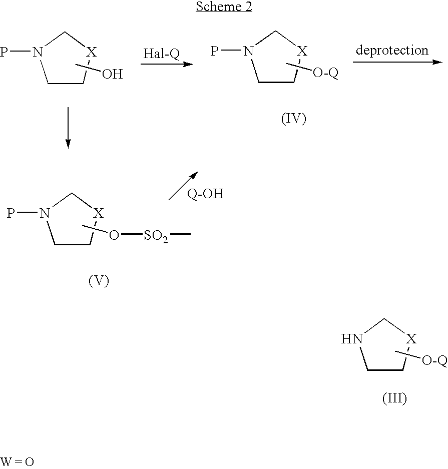

As illustrated in Scheme 2, reaction of an N-protected hydroxyl compound, wherein P is a protecting group, with Hal-Q (wherein Hal is F, Cl, Br or I), in a suitable solvent, in the presence of a suitable base, provides a compound of formula (IV). Protecting groups for amines are well known in the art. N-deprotection of the compound of formula (IV), in a suitable solvent, under standard conditions, depending on the protecting group, provides an amine of formula (III).

Alternatively, reaction of the starting N-protected hydroxyl compound with a reagent such as methanesulfonyl chloride, in a suitable solvent, in the presence of a suitable base, provides a compound of formula (V). Reaction of the compound of formula (V) with Q-OH, in a suitable solvent, in the presence of a suitable base, provides a compound of formula (IV) which may be deprotected, as above, to give the amine of formula (III).

The starting N-protected hydroxyl compound may also be reacted with Q-OH, in a suitable solvent, in the presence of reagents such as diisopropyl azodicarboxylate and triphenyl phosphine to provide the intermediate compound of formula (IV). N-deprotection of the compound of formula (IV), in a suitable solvent, under standard conditions provides an amine of formula (III).

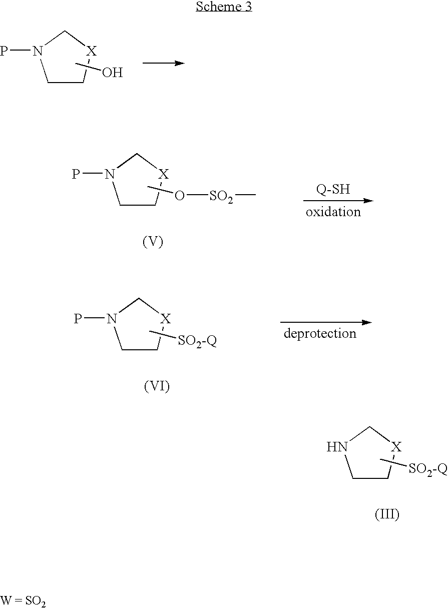

As illustrated in Scheme 3, reaction of the starting N-protected hydroxyl compound, wherein P is a protecting group, with a reagent such as methanesulfonyl chloride, in a suitable solvent, in the presence of a suitable base, provides a compound of formula (V). Reaction of the compound of formula (V) with Q-SH, in a suitable solvent, in the presence of a suitable base, followed by oxidation with a suitable reagent, provides a sulfone of formula (VI). N-deprotection of the compound of formula (VI), in a suitable solvent, under standard conditions, provides an amine formula (III).

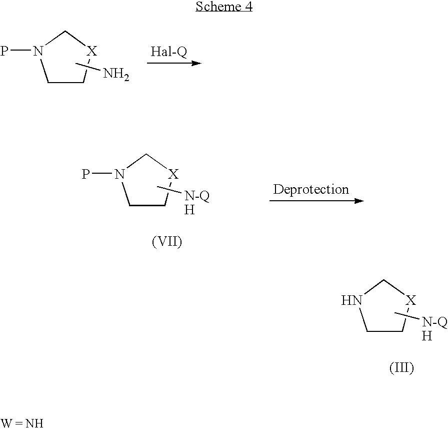

As illustrated in Scheme 4, reaction of a starting amine, wherein P is a protecting group, with Hal-Q (wherein Hal is Cl, Br or I), in a suitable solvent, in the presence of a suitable base, provides a compound of formula (VII). N-deprotection of the compound of formula (IV), in a suitable solvent, under standard conditions provides an amine of formula (III).

### EXAMPLE 1

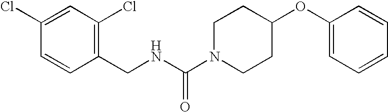

### 4-Phenoxy-piperidine-1-carboxylic Acid 2,4-dichloro-benzylamide

To a solution of 4-phenoxy-piperidine hydrochloride (0.213 g, 1.00 mmol) in acetonitrile (3 mL) is added 2,4-dichloro-1-isocyanato-benzene (0.202 g, 1.00 mmol) followed by the addition of triethylamine (0.101 g, 1.00 mmol) and the mixture is stirred overnight. The solvent is evaporated in vacuo and the resulting solid is purified on silica gel using hexane/ethyl acetate (1:1) as the eluent, to give the desired compound (0.211 g, 55.6%). LCMS: 378.9 (M+H⁺).

### EXAMPLE 2

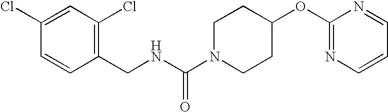

### 4-(Pyrimidin-2-yloxy)-piperidine-1-carboxylic Acid 2,4-dichloro-benzylamide

The compound is prepared using the procedure from Example 1, starting from 2-(piperidin-4-yloxy)-pyrimidine dihydrochloride (0.252 g, 1.00 mmol), diisopropylethylamine (0.258 g, 2.00 mmol) and 2,4-dichloro-1-isocyanato-benzene (0.202 g, 1.00 mmol), and is purified on silica gel using methanol/dichloromethane (5:95) as the eluent to give the desired product (0.282 g, 73.1%). LCMS: 381.0 (M+H⁺).

### EXAMPLE 3

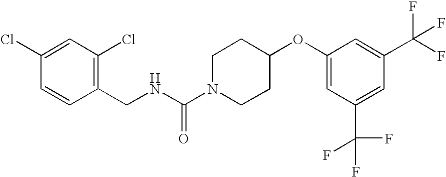

### 4-(3,5-Bis-trifluoromethyl-phenoxy)-piperidine-1-carboxylic Acid 2,4-dichloro-benzylamide

The compound is prepared using the procedure from Example 1, starting from 4-(3,5-bis-trifluoromethyl-phenoxy)-piperidine hydrochloride (0.313 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane to give the desired product (0.315 g, 61.1%). LCMS: 515.00 (M+H⁺).

### EXAMPLE 4

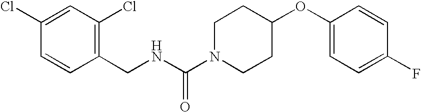

### 4-(4-Fluoro-phenoxy)-piperidine-1-carboxylic Acid 2,4-dichloro-benzylamide

The compound is prepared using the procedure from Example 1, starting from 4-(4-fluoro-phenoxy)-piperidine hydrochloride (0.231 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (5:95) as the eluent, to give the desired product (0.137 g, 34.5%). LCMS: 397.00 (M+H⁺).

### EXAMPLE 5

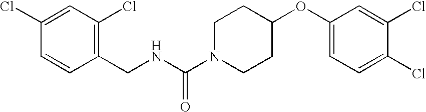

### 4-(3,4-Dichloro-phenoxy)-piperidine-1-carboxylic Acid 2,4-dichloro-benzylamide

The compound is prepared using the procedure from Example 1, starting from 4-(3,4-dichloro-phenoxy)-piperidine hydrochloride (0.282 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (2.5:97.5), to give the desired product (0.383 g, 85.5%). LCMS: 446.9 (M+H⁺).

### EXAMPLE 6

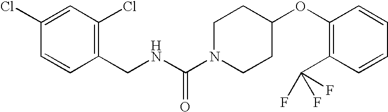

### 4-(2-Trifluoromethyl-phenoxy)-piperidine-1-carboxylic Acid 2,4-dichloro-benzylamide

The compound is prepared using the procedure from Example 1, starting from 4-(2-trifluoromethyl-phenoxy)-piperidine hydrochloride (0.281 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (2.5:97.5) as the eluent, to give the desired product (0.395 g, 88.3%). LCMS: 446.97 (M+H⁺).

### EXAMPLE 7

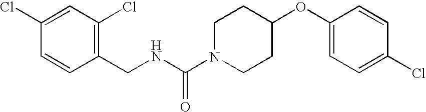

### 4-(4-Chloro-phenoxy)-piperidine-1-carboxylic Acid 2,4-dichloro-benzylamide

The compound is prepared using the procedure from Example 1, starting from 4-(4-chloro-phenoxy)-piperidine hydrochloride (0.248 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (2.5:97.5) as the eluent, to give the desired product (0.197 g, 47.6%). LCMS: 412.94 (M+H⁺).

### EXAMPLE 8

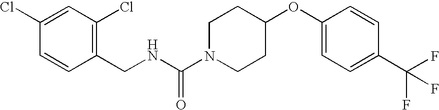

### 4-(4-Trifluoromethyl-phenoxy)-piperidine-1-carboxylic Acid 2,4-dichloro-benzylamide

The compound is prepared using the procedure from Example 1, starting from 4-(4-trifluoromethyl-phenoxy)-piperidine hydrochloride (0.282 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (2.5:97.5) as the eluent, to give the desired product (0.205 g, 45.8%). LCMS: 446.98 (M+H⁺).

### EXAMPLE 9

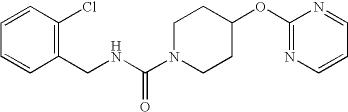

### 4-(Pyrimidin-2-yloxy)-piperidine-1-carboxylic Acid 2-chloro-benzylamide

The compound is prepared using the procedure from Example 1, starting from 2-(piperidin-4-yloxy)-pyrimidine dihydrochloride (0.126 g, 0.50 mmol), diisopropylethylamine (0.129 g, 1.00 mmol) and 1-chloro-2-isocyanatomethyl-benzene (0.084 g, 0.50 mmol), and is purified on silica gel using methanol/dichloromethane (2.5:97.5) as the eluent, to give the desired product (0.111 g, 64.0%). LCMS: 347.30 (M+H⁺).

### EXAMPLE 10

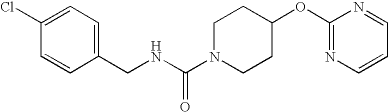

### 4-(Pyrimidin-2-yloxy)-piperidine-1-carboxylic Acid 4-chloro-benzylamide

The compound is prepared using the procedure from Example 1, starting from 2-(piperidin-4-yloxy)-pyrimidine dihydrochloride (0.126 g, 0.50 mmol), diisopropylethylamine (0.129 g, 1.00 mmol) and 4-chloro-2-isocyanatomethyl-benzene (0.084 g, 0.50 mmol) and is purified on silica gel using ethyl acetate as the eluent, to give the desired product (0.098 g, 56.5%). LCMS: 347.29 (M+H⁺).

### EXAMPLE 11

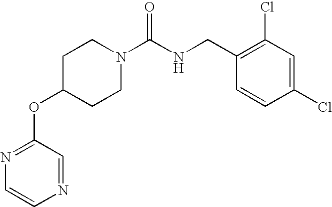

### 4-(Pyrazin-2-yloxy)-piperidine-1-carboxylic Acid 2,4-dichloro-benzylamide

The compound is prepared using the procedure from Example 1, starting from 2-(piperidin-4-yloxy)-pyrazine dihydrochloride (0.252 g, 1.00 mmol), diisopropylethylamine (0.129 g, 1.00 mmol) and 2,4-dichloro-1-isocyanato-benzene (0.202 g, 1.00 mmol), and is purified on silica gel using methanol/dichloromethane (2.5:97.5), to give the desired product (0.205 g, 45.8%). LCMS: 381.2, 381.00 (M+H⁺).

### EXAMPLE 12

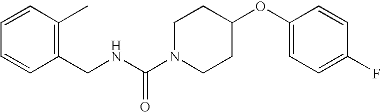

### 4-(4-Fluoro-phenoxy)-piperidine-1-carboxylic Acid 2-methyl-benzylamide

To a solution of 4-(4-fluoro-phenoxy)-piperidine hydrochloride (0.116 g, 0.50 mmol) in acetonitrile (2 mL) is added diisopropylethylamine (0.065 g, 0.50 mmol) and 1-isocyanatomethyl-2-methyl-benzene (0.073 g, 0.50 mmol). The mixture is stirred overnight and the solid is filtered off, washed several times with hexane, and dried in vacuo to give the desired compound (0.077 g, 45.0%). LCMS: 343.11 (M+H⁺).

### EXAMPLE 13

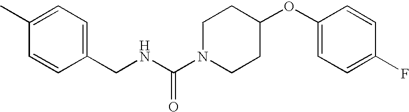

### 4-(4-Fluoro-phenoxy)-piperidine-1-carboxylic Acid 4-methyl-benzylamide

The compound is prepared using the procedure from Example 12, starting from 4-(4-fluoro-phenoxy)-piperidine hydrochloride (0.116 g, 0.50 mmol), diisopropylethylamine (0.065 g, 0.50 mmol) and 1-isocyanatomethyl-4-methyl-benzene (0.073 g, 0.50 mmol), to give the desired product (0.107 g, 62.5%). LCMS: 343.11 (M+H⁺).

### EXAMPLE 14

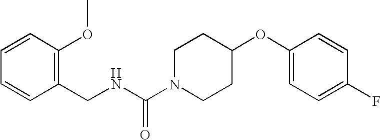

### 4-(4-Fluoro-phenoxy)-piperidine-1-carboxylic Acid 2-methoxy-benzylamide

The compound is prepared and purified using the procedure from Example 12, starting from 4-(4-fluoro-phenoxy)-piperidine hydrochloride (0.116 g, 0.50 mmol), diisopropylethylamine (0.065 g, 0.50 mmol) and 1-isocyanatomethyl-2-methoxy-benzene (0.081 g, 0.50 mmol), to give the desired product (0.137 g, 76.4%). LCMS: 359.07 (M+H⁺).

### EXAMPLE 15

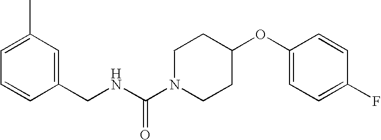

### 4-(4-Fluoro-phenoxy)-piperidine-1-carboxylic Acid 3-methyl-benzylamide

The compound is prepared using the procedure from Example 1, starting from 4-(4-fluoro-phenoxy)-piperidine hydrochloride (0.116 g, 0.50 mmol), diisopropylethylamine (0.065 g, 0.50 mmol) and 1-isocyanatomethyl-3-methyl-benzene (0.074 g, 0.5 mmol) and purified on silica gel using ethyl acetate as the eluent, to give the desired product (0.103 g, 60.2%). LCMS: 343.11 (M+H⁺).

### EXAMPLE 16

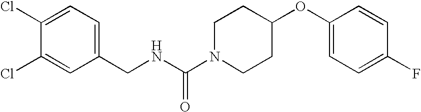

### 4-(4-Fluoro-phenoxy)-piperidine-1-carboxylic Acid 3,4-dichloro-benzylamide

The compound is prepared using the procedure from Example 1, starting from 4-(4-fluoro-phenoxy)-piperidine hydrochloride (0.230 g, 1.00 mmol), diisopropylethylamine (0.129 g, 1.00 mmol) 1,2-dichloro-4-isocyanatomethyl-benzene (0.202 g, 1.00 mmol) and purified on silica gel using ethyl acetate as the eluent, to give the desired product (0.256 g, 64.4%). LCMS: 396.97 (M+H⁺).

### EXAMPLE 17

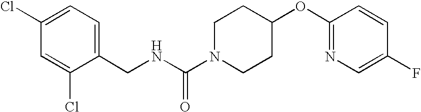

### 4-(5-Fluoro-pyridin-2-yloxy)-piperidine-1-carboxylic Acid 2,4-dichloro-benzylamide

### Step A: tert-Butyl 4-(5-fluoro-pyridin-2-yloxy)-1-piperidinecarboxylate

A mixture of tert-butyl 4-hydroxy-1-piperidinecarboxylate (3.02 g, 15.0 mmol), 5-fluoro-2 hydroxypyridine (0.85 g, 7.50 mmol), diisopropyl-azodicarboxylate (3.03 g, 15.00 mmol), triphenylphosphine (3.90 g, 15.00 mmol) and tetrahydrofuran (100 mL) is stirred at 0° C., allowed to come to room temperature and stirred overnight. The reaction mixture is evaporated in vacuo and purified on silica gel using ethyl acetate/hexane as the eluent. Fractions containing the product are pooled and evaporated to give the desired product (2.12 g, 95%) as a colorless solid.

### Step B: 4-(5-Fluoro-pyridin-2-yloxy)-1-piperidine

To the compound from Step B is added a mixture of 1,2-dichloroethane and TFA (1:1). The mixture is stirred for 45 minutes, evaporated in vacuo and triturated with diethyl ether/hexane to give the TFA salt of the desired compound (0.92 g, 88%) as a colorless solid.

### Step C: 4-(5-Fluoro-pyridin-2-yloxy)-piperidine-1-carboxylic Acid 2,4-dichloro-benzylamide

To the solution of the compound from Step B (0.232 g, 1.00 mmol) and diisopropylethylamine (0.129 g, 1.00 mmol) in acetonitrile (3 ml) is added 2,4-dichloro-1-isocyanatomethyl-benzene (0.202 g, 1.00 mmol). The mixture is stirred at room temperature for 3 hours and evaporated in vacuo. The resulting residue is purified on silica gel using methanol/methylene chloride (5:95) as the eluent, to give the desired product (0.311 g, 78.1%). LCMS: 397.95 (M+H⁺).

### EXAMPLE 18

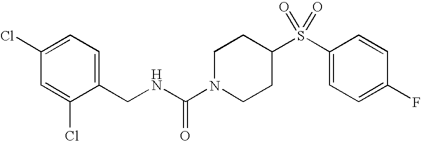

### 4-(4-Fluoro-benzenesulfonyl)-piperidine-1-carboxylic Acid 2,4-dichloro-benzylamide

### Step A: 4-Methanesulfonyloxy-piperidine-1-carboxylic Acid tert-butyl Ester

To a solution of the tert-butyl 4-hydroxy-piperidine-1-carboxylate (5.00 g, 24.9 mmol) in dichloromethane (50 mL) is added pyridine (10.00 mL, 122.6 mmol) and DMAP (0.56 g, 4.60 mmol). The mixture is then cooled to 0° C. and methanesulfonyl chloride (4.5 g, 39.30 mmol) is added over 10 minutes. The reaction mixture is stirred for 24 hours, evaporated in vacuo and the solid residue obtained is triturated with ethyl acetate (100 mL) and filtered. The filterate is evaporated in vacuo and the residue obtained is purified by flash chromatography (25-100% ethyl acetate in hexanes) to obtain the title compound (5.36 g, 77%).

### Step B: tert-Butyl 4-(4-fluoro-phenylsulfanyl)-piperidine-1-carboxylate

To a solution of the product from Step A (5.30 g, 18.99 mmol) in acetonitrile (100 mL) is added 4-fluorobenzenethiol (2.95 g, 23.00 mmol) and potassium carbonate (4.11 g, 29.78 mmol). The mixture is heated at reflux for 18 hours. The mixture is diluted with water and extracted with ethyl acetate. Organic phase is evaporated in vacuo and purified by flash chromatography (5-100% ethyl acetate in hexanes) to give the title compound (6.00 g, 100%).

### Step C: tert-Butyl 4-(4-fluoro-benzenesulfonyl)-piperidine-1-carboxylate

Water (3 mL) is added to alumina (15.0 g) and stirred for 5 minutes. A solution of the product from Step B (6.0 g, 18.99 mmol) in chloroform (100 mL) is added followed by the addition of oxone and the temperature of the mixture is brought up to the reflux. After 18 hours the reaction mixture is cooled to room temperature, diluted and filtered. The insoluble materials are washed with chloroform. The organic layers are combined and evaporated in vacuo to give the title compound (6.00 g, 92%).

### Step D: 4-(4-Fluoro-phenylsulfonyl)-piperidine Hydrochloride

To the solution of the product from Step C (6.0 g, 17.49 mmol) in methanol (100 mL) is added HCl (5 N, 25 mL). The mixture is heated under reflux for 3 hour. Solvents are evaporated in vacuo and the residue is triturated with ether, filtered and dried in vacuo to give the desired compound (3.80 g, 78%).

### Step E: 4-(4-Fluoro-benzenesulfonyl)-piperidine-1-carboxylic Acid 2,4-dichloro-benzylamide

The compound is prepared using the procedure from Example 17, starting from the compound from Step D (0.279 g, 1.00 mmol), diisopropylethylamine (0.129 g, 1.00 mmol) and 2,4-dichloro-1-isocyanatomethyl-benzene (0.202 g, 1.00 mmol), and purified on silica gel using methanol/methylene chloride (5:95) as the eluent, to give the desired product (0.32 g, 72%). LCMS: 444.87 (M+H⁺).

### EXAMPLE 19

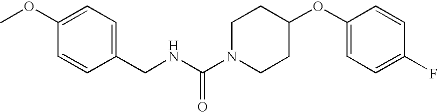

### 4-(4-Fluoro-phenoxy)-piperidine-1-carboxylic Acid 4-methoxy-benzylamide

The compound is prepared and purified using the procedure from Example 17, starting from 4-(4-fluoro-phenoxy)-piperidine hydrochloride (0.115 g, 0.50 mmol), diisopropylethylamine (0.129 g, 1.00 mmol) in acetonitrile and 1-isocyanatomethyl-4-methoxy-benzene (0.081, 0.50 mmol), to give the desired product (0.045 g, 25.1%). LCMS: 359.03 (M+H⁺).

### EXAMPLE 20

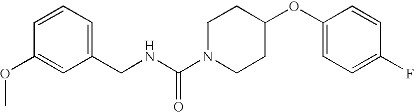

### 4-(4-Fluoro-phenoxy)-piperidine-1-carboxylic Acid 3-methoxy-benzylamide

The compound is prepared and purified using the procedure from Example 17, starting from 4-(4-fluoro-phenoxy)-piperidine hydrochloride (0.115 g, 0.50 mmol), diisopropylethylamine (0.129 g, 1.00 mmol) and 1-isocyanatomethyl-4-methoxy-benzene (0.081, 0.50 mmol), to give the desired product (0.115 g, 64.2%). LCMS: 359.07 (M+H⁺).

### EXAMPLE 21

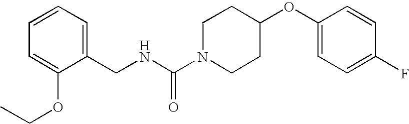

### 4-(4-Fluoro-phenoxy)-piperidine-1-carboxylic Acid 2-ethoxy-benzylamide

The compound is prepared and purified using the procedure from Example 17, starting from 4-(4-fluoro-phenoxy)-piperidine hydrochloride (0.231 g, 1.00 mmol), diisopropylethylamine (0.129 g, 1.00 mmol) acetonitrile and 1-isocyanatomethyl-2-ethoxy-benzene (0.177, 1.00 mmol), to give the desired product (0.316 g, 84.2%). LCMS: 373.05 (M+H⁺).

### EXAMPLE 22

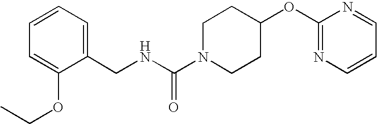

### 4-(Pyrimidin-2-yloxy)-piperidine-1-carboxylic Acid 2-ethoxy-benzylamide

The compound is prepared using the procedure from Example 17, starting from 2-(piperidin-4-yloxy)-pyrimidine dihydrochloride (0.252 g, 1 mmol), diisopropylethylamine (0.129 g, 1.00 mmol) and 1-isocyanatomethyl-2-ethoxy-benzene (0.177, 1 mmol), to give the desired product (0.298 g, 83.6%). LCMS: 357.07 (M+H⁺).

### EXAMPLE 23

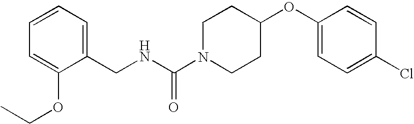

### 4-(4-Chloro-phenoxy)-piperidine-1-carboxylic Acid 2-ethoxy-benzylamide

The compound is prepared using the procedure from Example 17, starting from 4-(4-chloro-phenoxy)-piperidine hydrochloride (0.248 mg, 1.00 mmol), diisopropylethyl amine (0.129 g, 1.00 mmol) and 1-isocyanatomethyl-2-ethoxy-benzene (0.177, 1.00 mmol), to give the desired product (0.389 g, 82.5%). LCMS: 389.82 (M+H⁺).

### EXAMPLE 24

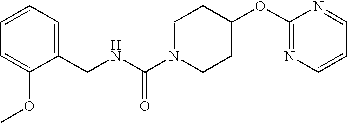

### 4-(Pyrimidin-2-yloxy)-piperidine-1-carboxylic Acid 2-methoxy-benzylamide

The compound is prepared using the procedure from Example 17, starting from 2-(piperidin-4-yloxy)-pyrimidine dihydrochloride (0.252 g, 1.00 mmol), diisopropylethylamine (0.258 g, 2.00 mmol) and 1-isocyanatomethyl-2-methoxy-benzene (0.163, 1.00 mmol), to give the desired product (0.266 g, 77.7%). LCMS: 343.41 (M+H⁺).

### EXAMPLE 25

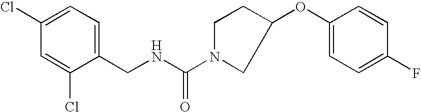

### 3-(4-Fluoro-phenoxy)-pyrrolidine-1-carboxylic Acid 2,4-dichloro-benzylamide

### Step A: 3-Methanesulfonyloxy-pyrrolidine-1-carboxylic Acid tert-butyl Ester

To a solution of the tert-butyl 3-hydroxy-1-pyrrolidinecarboxylate (5.00 g, 26.7 mmol) in dichloromethane (50 mL) is added pyridine (10 mL, 122.6 mmol) and DMAP (0.56 g, 4.6 mmol). The mixture is then cooled to 0° C. and methanesulfonyl chloride (3 mL, 38.8 mmol) is added over 10 minutes. The reaction mixture is then stirred for 18 hours, evaporated in vacuo, and the solid residue obtained triturated with ethyl acetate (500 mL) and filtered. The filterate is evaporated in vacuo and the residue is purified by flash chromatography to obtain the title compound (6.35 g, 90%).

### Step B: 3-(4-Fluoro-phenoxy)-pyrrolidine-1-carboxylic Acid tert-butyl Ester

To a solution of the product from Step A (1.06 g, 8.60 mmol) in acetonitrile (25 mL) is added 4-fluorophenol (0.55 g, 8.98 mmol) and potassium carbonate (0.86 g, 6.23 mmol). The mixture is heated at 85° C. for 5 days. Analysis of the reaction by TLC shows the formation of the product. The mixture is then diluted with water and extracted with ethyl acetate. Organic layer is then condensed in vacuo and purified by flash chromatography (20-100% ethyl acetate in heptanes) to give the product (0.72 g, 64%).

### Step C: 3-(4-Fluoro-phenoxy)-pyrrolidine Tosylate

To the solution of the product from Step B (3.44 g, 12.2 mmol) in dichloromethane is added water (1 mL) and TFA (5 mL) at room temperature. The mixture is stirred for 4 hours. The solvents are removed in vacuo and the residue is taken in dichloroethane (25 mL) and PTSA (2.3 g, 13.35 mmol) added. The mixture is stirred for overnight and then solvents are evaporated in vacuo. The residue obtained is triturated with ether, filtered and dried to give the desired product (3.3 g, 76%).

### Step D: 3-(4-Fluoro-phenoxy)-pyrrolidine-1-carboxylic Acid 2,4-dichloro-benzylamide

The compound is prepared using the procedure from Example 17, starting from the compound from Step C (0.353 g, 1.00 mmol), diisopropylethylamine (0.129 g, 1.00 mmol) and 2,4-dichloro-1-isocyanatomethyl-benzene (0.202 g, 1.00 mmol), to give the desired product (0.254 g, 66.3%). LCMS: 382.93 (M+H⁺).

### EXAMPLE 26

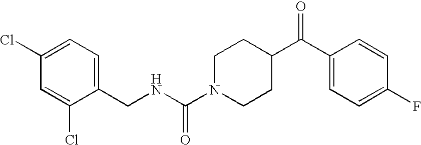

### 4-(4-Fluoro-benzoyl)-piperidine-1-carboxylic Acid 2,4-dichloro-benzylamide

The compound is prepared using the procedure from Example 17, starting from (4-fluoro-phenyl)-piperidin-4-yl-methanone (0.244 g, 1.00 mmol), diisopropylethylamine (0.129 g, 1.00 mmol) and 2,4-dichloro-1-isocyanatomethyl-benzene (0.202 g, 1.00 mmol), to give the desired product (0.221 g, 54.0%). LCMS: 409.25 (M+H⁺).

### EXAMPLE 27

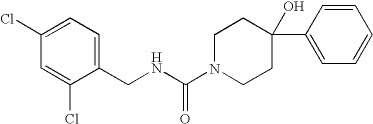

### 4-Hydroxy-4-phenyl-piperidine-1-carboxylic Acid 2,4-dichloro-benzylamide

The compound is prepared using the procedure from Example 17, starting from 4-hydroxy-4-phenyl-piperidine (0.177 g, 1.00 mmol) and 2,4-dichloro-1-isocyanatomethyl-benzene (0.202 g, 1.00 mmol). The solid is filtered off, washed several times with hexane, and dried in vacuo to give the desired compound (0.285 g, 75.1%). LCMS: 379.01 (M+H⁺).

### EXAMPLE 28

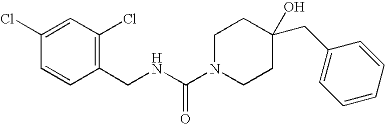

### 4-Benzyl-4-hydroxy-piperidine-1-carboxylic Acid 2,4-dichloro-benzylamide

The compound is prepared using the procedure from Example 17, starting from 4-hydroxy-4-benzyl-piperidine (0.191 g, 1.00 mmol) and 2,4-dichloro-1-isocyanatomethyl-benzene (0.202 g, 1.00 mmol). The solid is filtered off, washed several times with hexane, and dried in vacuo to give the desired compound (0.350 g, 89.0%). LCMS: 393.32 (M+H⁺).

### EXAMPLE 29

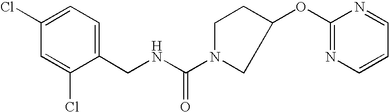

### 3-(Pyrimidin-2-yloxy)-pyrrolidine-1-carboxylic Acid 2,4-dichloro-benzylamide

### Step A: tert-Butyl-3-(pyrimidin-2-yloxy)-pyrrolidine-1-carboxylate

To a solution of tert-butyl-3-hydroxy-1-pyrrolidinecarboxylate (1.87 g, 10.00 mmol) in DMF (20 mL) is added sodium hydride (60% suspension in mineral oil) (0.50 g, 35 mmol). The mixture is stirred for 15 minutes and 2-chloropyrimidine (1.37 g, 12.00 mmol) is added. The mixture is heated to 80° C. for 4 days and the reaction is quenched with water (25 mL) and extracted with ethyl acetate (2×50 mL). The organic phase is evaporated in vacuo and purified on silica using ethyl acetate/heptane as the eluent. Fractions containing the product are pooled and evaporated to give the desired product (2.20 g, 83%) as a colorless solid.

### Step B: 3-(Pyrimidin-2-yloxy)-pyrrolidine

To the compound from Step B is added methanol (70 mL) and HCl (aqueous, 5N) (10 mL). The mixture was heated under reflux for 1 h, allowed to come to room temperature, and the solvent removed in vacuo. The crude product is purified by preperative HPLC to give the desired compound (0.80 g, 52%).

### Step C: 3-(Pyrimidin-2-yloxy)-pyrrolidine-1-carboxylic Acid 2,4-dichloro-benzylamide

The compound is prepared using the procedure from Example 17, starting from the compound from Step B (0.337 g, 1.00 mmol), diisopropylethylamine (0.129 g, 1.00 mmol) and 2,4-dichloro-1-isocyanatomethyl-benzene (0.202 g, 1.00 mmol) to give the desired compound (0.265 g, 72.2%). LCMS: 366.97 (M+H⁺).

### EXAMPLE 30

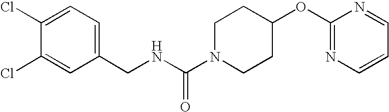

### 4-(Pyrimidin-2-yloxy)-piperidine-1-carboxylic Acid 3,4-dichloro-benzylamide

The compound is prepared using the procedure from Example 17, starting from 2-(piperidin-4-yloxy)-pyrimidine dihydrochloride (0.252 g, 1 mmol), diisopropylethylamine (0.258 g, 2.00 mmol) and 3,4-dichloro-1-isocyanatomethyl-benzene (0.202 g, 1.00 mmol) to give the desired compound (0.179 g, 47.0%). LCMS: 381.29 (M+H⁺).

### EXAMPLE 31

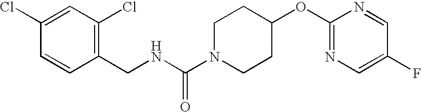

### 4-(5-Fluoro-pyrimidin-2-yloxy)-piperidine-1-carboxylic Acid 2,4-dichloro-benzylamide

### Step A: 2-Chloro-5-fluoropyrimidine

To 2,4-dichloro-5-fluoropyrimidine (15.0 g, 89.8 mmol) is added tetrahydrofuran (100 mL) and zinc powder (17.6 g, 269 mmol). The mixture is heated to 70° C. with vigorous stirring, acetic acid (5.14 mL, 89.8 mmol) is added over 1 h and the mixture is heated at reflux for an additional 5 h. The mixture is diluted with dichloromethane, filtered through celite, evaporated in vacuo and purified on silica gel to give the desired product (6.00 g, 50%).

### Step B: tert-Butyl-4-(5-fluoropyrimidine pyrimidine-2-yloxy)-1-piperidinecarboxylate

To tert-butyl 4-hydroxy-1-piperidinecarboxylate (5.72 g, 28.4 mmol) in tetrahydrofuran (40 mL) is added sodium hydride (60% emulsion in mineral oil) (1.75 g, 43.8 mmol) and the resulting mixture is stirred for 1 h. The mixture was then cooled and the compound from Step A (2.90 g, 21.9 mmol) in tetrahydrofuran (10 mL) was added dropwise. The resulting mixture was allowed to come to rt over a 12 h period, diluted with ethyl acetate, quenched with water and the organic phase dried over sodium sulfate. The crude mixture was purified over silica gel to give the desired compound (5.20 g, 80%)

### Step C: 4-(5-Fluoropyrimidine pyrimidine-2-yloxy)-1-piperidine Hydrochloride

To the compound from Step B (8.00 g, 26.9 mmol) in 1,4-dioxane (60.0 mL) is added HCl (4N, aqueous) (20 mL) and the mixture is stirred at rt for 12 h, evaporated in vacuo, evaporated repeatedly from toluene and triturated with hexane/diethylether to give the desired compound (6.17 g, 98%.

### Step D: 4-(5-Fluoro-pyrimidin-2-yloxy)-piperidine-1-carboxylic Acid 2,4-dichloro-benzylamide

The compound is prepared using the procedure from Example 17, starting from the compound from Step C (0.232 g, 1.00 mmol), diisopropylethylamine (0.129 g, 1.00 mmol) and 2,4-dichloro-1-isocyanatomethyl-benzene (0.202 g, 1.00 mmol) to give the desired compound (0.257 g, 64.4%). LCMS: 399.24 (M+H⁺).

### EXAMPLE 32

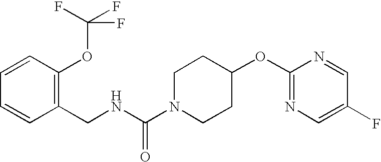

### 4-(5-Fluoro-pyrimidin-2-yloxy)-piperidine-1-carboxylic Acid 2-trifluoromethoxy-benzylamide

The compound is prepared using the procedure from Example 17, starting from the product of Example 31 Step C (0.37 g, 1.00 mmol), diisopropylethylamine (0.129 g, 1.00 mmol) and 2-trifluoromethoxy-1-isocyanatomethyl-benzene (0.217 g, 1.00 mmol) to give the desired compound (0.343 g, 82.8%).

### EXAMPLE 33

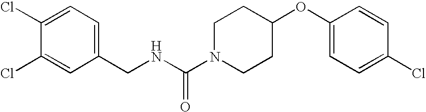

### 4-(4-Chloro-phenoxy)-piperidine-1-carboxylic Acid 3,4-dichloro-benzylamide

The compound is prepared using the procedure from Example 17, starting from 4-(4-chloro-phenoxy)-piperidine hydrochloride (0.248 mg, 1.00 mmol), diisopropylethylamine (0.129 g, 1.00 mmol) and 3,4-dichloro-1-isocyanatomethyl-benzene (0.202 g, 1.00 mmol). The mixture is stirred at room temperature for 3 hours and evaporated in vacuo. The resulting residue is purified on silica gel using methanol/methylene chloride (5:95) as the eluent. Subsequent recrystallization from acetonitrile proceeds to give the desired compound (0.164 g, 39.6%). LCMS: 413.18

### EXAMPLE 34

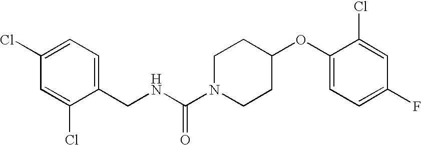

### 4-(2-Chloro-4-fluoro-phenoxy)-piperidine-1-carboxylic Acid 2,4-dichloro-benzylamide

### Step A: 4-(2-Chloro-4-fluoro-phenoxy)-piperidine-1-carboxylic Acid tert-butyl Ester

tert-Butyl 4-hydroxy-piperidine-1-carboxylate (2.74 g, 13.6 mmol), 2-chloro-4-fluoro-phenol (1.00 g, 6.80 mmol), diisopropyl azo-dicarboxylate (2.75 g, 13.6 mmol), triphenylphosphine (3.6 g, 13.6) and anhydrous tetrahydrofuran (100 mL) are stirred at 0° C. and allowed to warm to room temperature overnight. After the completion of the reaction, the mixture is condensed in vacuo and purified by flash chromatography (ethyl acetate/heptane) to give the title compound (1.63 g, 73%).

### Step B: 4-(2-Chloro-4-fluoro-phenoxy)-piperidine Hydrochloride

The compound from Step A (1.57 g, 4.8 mmol) is dissolved in dioxane (15 mL) and 4N HCl (5 mL) added. The mixture is stirred at room temperature for overnight. The reaction is then condensed in vacuo dissolved in EtOAc and condensed again. The resulting residue is triturated with hexanes/ether to give the title compound (1.001 g, 83%).

### Step C: 4-(2-Chloro-4-fluoro-phenoxy)-piperidine-1-carboxylic Acid 2,4-dichloro-benzylamide

The compound is prepared using the procedure from Example 17, the compound from Step B (0.266 mg, 1.00 mmol), diisopropylethylamine (0.129 g, 1.00 mmol) and 2,4-dichloro-1-isocyanatomethyl-benzene (0.202 g, 1.00 mmol), to give the desired compound (0.298 g, 69.0%). LCMS: 430.84 (M+H⁺).

### EXAMPLE 35

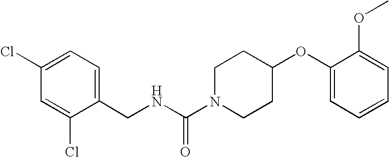

### 4-(2-Methoxy-phenoxy)-piperidine-1-carboxylic Acid 2,4-dichloro-benzylamide

The compound is prepared using the procedure from Example 17, starting from 4-(2-methoxy-phenoxy)-piperidine hydrochloride (0.244 mg, 1.00 mmol), diisopropylethylamine (0.129 g, 1.00 mmol) and 2,4-dichloro-1-isocyanatomethyl-benzene (0.202 g, 1.00 mmol), to give the desired compound (0.249 g, 60.8%). LCMS: 408.93 (M+H⁺).

### EXAMPLE 36

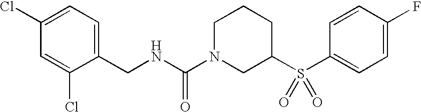

### Step A: tert-Butyl 3-methanesulfonyl-piperidine-1-carboxylate

To a solution of the tert-butyl 3-hydroxy-piperidine-1-carboxylate (5.00 g, 24.9 mmol) in dichloromethane (50 mL) is added pyridine (10 mL, 122.6 mmol) and DMAP (0.56 g, 4.60 mmol). The mixture is then cooled to 0° C. and methanesulfonyl chloride (4.50 g, 39.3 mmol) is added over 10 minutes. The reaction mixture is then stirred for 18 hours, evaporated in vacuo and the solid residue obtained is then triturated with ethyl acetate and then filtered. The filterate is then evaporated in vacuo and the residue obtained is purified by flash chromatography to give the desired compound (6.50 g, 94%).

### Step B: tert-Butyl 3-(4-fluoro-phenylsulfanyl)-piperidine-1-carboxylate

To a solution of the product from Step A (2.40 g, 8.60 mmol) in acetonitrile (50 mL) is added 4-fluorobenzenethiol (1.15 g, 8.98 mmol) and potassium carbonate (1.90 g, 13.76 mmol). The mixture is brought to reflux temperature and stirred for 18 hours. The mixture is diluted with water and extracted with ethyl acetate. The organic layer is condensed in vacuo and purified by flash chromatography (5-100% ethyl acetate in hexanes) to give the desired compound (1.70 g, 64%).

### Step C: tert-Butyl 3-(4-fluoro-benzenesulfonyl)-piperidine-1-carboxylate

Water (0.9 mL) is added to aluminum oxide (4.50 g) and stirred for 5 minutes. A solution of the product from Step B (1.70 g, 5.47 mmol) in chloroform (25 mL) is added to the mixture followed by the addition of oxane (10.35 g) and the mixture is brought to reflux. After 18 hours the reaction mixture is cooled to room temperature and filtered. The insoluble materials are washed with chloroform and the organic layers are combined, evaporated in vacuo and purified by flash chromatography (20-100% ethyl acetate in hexanes) to give the desired compound (1.40 g, 74%).

### Step D: 3-(4-Fluoro-phenylsulfonyl)-piperidine Tosylate

To the solution of the compound from Step C (1.40 g, 4.08 mmol) in methanol (30 mL) is added HCl (5 N, 5 mL). The mixture is heated under reflux for 1 hour. Solvents are evaporated in vacuo and the residue is triturated with ether, evaporated and purified by preparative HPLC. The product taken up in dichloroethane and PTSA (0.56 g, 3.5 mmol) is added. The mixture is stirred for 30 minutes and solvents evaporated in vacuo. The residue is triturated with ether and filtered to give the desired product (1.08 g, 99

### Step E: 3-(4-Fluoro-benzenesulfonyl)-piperidine-1-carboxylic Acid 2,4-dichloro-benzylamide

The compound is prepared using the procedure from Example 17, starting from the product of Step D (0.416 mg, 1.00 mmol), diisopropylethylamine (0.129 g, 1.00 mmol) and 2,4-dichloro-1-isocyanatomethyl-benzene (0.202 g, 1.00 mmol). The mixture is stirred overnight and the solid is filtered off, washed with acetonitrile, and dried in vacuo to give the desired compound (0.409 g, 91.8%). LCMS: 444.84 (M+H⁺).

### EXAMPLE 37

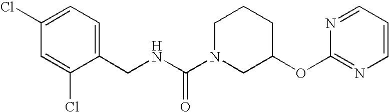

### Step A: tert-Butyl 3-(Pyrimidin-2-yloxy)-piperidine-1-carboxylate

To a solution of tert-butyl-hydroxy-1-pyrrolidinecarboxylate piperidine carboxylate (2.01 g, 10.00 mmol) in dimethylformide is added sodium hydride (60% suspension in mineral oil) (0.50 g, 35 mmol). The mixture is stirred for 15 minutes and 2-chloropyrimidine (1.37 g, 12.00 mmol) is added. The mixture is heated to 80° C. for 4 days and the reaction is quenched with water (25 mL) and extracted with ethyl acetate (2×50 mL). The organic phase is evaporated in vacuo and purified on silica using ethyl acetate/heptane as the eluent. Fractions containing the product are pooled and evaporated to give the desired product (2.40 g, 86%) as a colorless solid.

### Step B: 2-(Piperidin-3-yloxy)-pyrimidine Dihydrochloride

To the compound from Step A (2.40 g, 8.60 mmol) is added methanol (70 mL) and HCl (aqueous, 5N) (10 mL). The mixture is heated under reflux for 1 h, allowed to come to room temperature, and the solvent is removed in vacuo. The crude product is purified by preparative HPLC to give the title compound (0.80 g, 52%).

### Step C: 3-(Pyrimidin-2-yloxy)-piperidine-1-carboxylic Acid 2,4-dichloro-benzylamide

The compound is prepared using the procedure from Example 17, starting from 2-(piperidin-3-yloxy)-pyrimidine hydrochloride (0.252 g, 1.00 mmol), diisopropylethylamine (0.129 g, 1.00 mmol) and 2,4-dichloro-1-isocyanatomethyl-benzene (0.202 g, 1.00 mmol), to give the desired compound (0.23 g, 60.9%).

### EXAMPLE 38

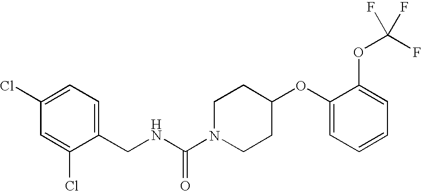

### 4-(2-Trifluoromethoxy-phenoxy)-piperidine-1-carboxylic Acid 2,4-dichloro-benzylamide

### Step A: 4-(2-Trifluoromethoxy-phenoxy)-piperidine-1-carboxylic Acid tert-butyl Ester

tert-Butyl 4-hydroxy-piperidine-1-carboxylic acid (2.26 g, 11.2 mmol), 2-trifluoromethoxyphenol (1 g, 5.61 mmol), diisopropyl azo-dicarboxylate (2.27 g, 11.2 mmol), triphenylphosphine (2.9 g, 11.2) and anhydrous tetrahydrofuran (100 mL) are stirred at 0° C. and allowed to warm to room temperature over night. After the completion of the reaction, the mixture is condensed in vacuo and purified by flash chromatography (ethyl acetate/heptane) to give 4-boc-(2-trifluoromethoxy-phenoxy)-piperidine (1.52 g, 75%).

### Step B: 4-(2-Trifluoromethoxy-phenoxy)-piperidine Hydrochloride

To the compound from Step A is added methanol and HCl (aqueous, 5N). The mixture is heated under reflux for 1 h, allowed to come to room temperature, and the solvent is removed in vacuo. The crude product is purified by preparative HPLC to give the title compound.

### Step C: 4-(2-Trifluoromethoxy-phenoxy)-piperidine-1-carboxylic Acid 2,4-dichloro-benzylamide

The compound is prepared using the procedure from Example 17, starting from 4-(2-trifluoromethoxy-phenoxy)-piperidine hydrochloride (0.266 g, 1.00 mmol), diisopropylethylamine (0.129 g, 1.00 mmol) and 2,4-dichloro-1-isocyanatomethyl-benzene (0.202 g, 1.00 mmol). The mixture is stirred overnight and the solid is filtered off, washed with acetonitrile, and dried in vacuo to give to give the desired compound (0.232 g, 60.9%). LCMS: 462.86 (M+H⁺).

### EXAMPLE 39

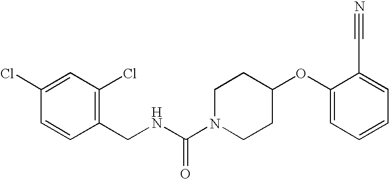

### 4-(2-Cyano-phenoxy)-piperidine-1-carboxylic Acid 2,4-dichloro-benzylamide

### Step A: tert-Butyl 4-(2-cyanophenyloxy-1-piperidinecarboxylate

To 1-N-Boc-4-hydroxy-1-piperidine (2.41 g, 10.0 mmol) in dimethylformamide (25 mL) is added sodium hydride (60% emulsion in mineral oil) (0.80 g, 20.0 mmol) and the mixture is stirred at room temperature for 1 h. 2-Fluorobenzonitrile (1.21 g, 10.0 mmol) is added and the mixture is heated to 60° C. for 30 minutes. The mixture is diluted with ethyl acetate and the reaction is quenched by the addition of water. The organic phase is evaporated in vacuo and purified on silica gel to give the desired product (3.00 g, 99%) as colorless oil.

### Step B: 4-(2-Cyanophenyloxy)piperidine Hydrochloride

To the compound from Step A (3.00 g, 9.90 mmol) in 1,4-dioxane (30 mL) is added HCl (4N, aqueous) (10.0 mL) and the mixture is stirred at room temperature overnight. The reaction is concentrated in vacuo and evaporated repeatedly from toluene to give the desired product (1.30 g) after crystallization from ethyl acetate.

### Step C: 4-(2-Cyano-phenoxy)-piperidine-1-carboxylic Acid 2,4-dichloro-benzylamide

This compound is prepared using the procedure from Example 1, starting from the compound from Step B (0.238 mg, 1.00 mmol), diisopropylethylamine (0.129 g, 1.00 mmol) and 2,4-dichloro-1-isocyanatomethyl-benzene (0.202 g, 1.00 mmol). The mixture is stirred at room temperature overnight, poured into dichloromethane/aqueous sodium bicarbonate (20 mL each), the organic phase is separated, extracted with water (20 mL), dried over magnesium sulphate, filtered, and evaporated in vacuo to give the desired product (0.30 g, 74.5%). LCMS: 404.24 (M+H⁺).

### EXAMPLE 40

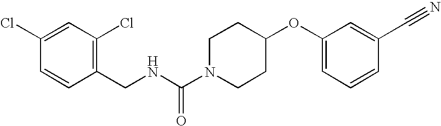

### 4-(3-Cyano-phenoxy)-piperidine-1-carboxylic Acid 2,4-dichloro-benzylamide

### Step A: tert-Butyl 4-(3-cyanophenyloxy)-1-piperidinecarboxylate

To tert-butyl 4-hydroxy-1-piperidinecarboxylate (2.41 g, 12.0 mmol) in dimethylformamide (25 mL) is added sodium hydride (60% emulsion in mineral oil) (0.80 g, 20.0 mmol) and the mixture is stirred at rt for 1 h. 3-Fluorobenzonitrile (1.21 g, 10.0 mmol) is added and the mixture is heated to 60° C. for 30 minutes. The mixture is diluted with ethyl acetate and the reaction is quenched by the addition of water. The organic phase is evaporated in vacuo and purified on silica gel to give the desired product (3.00 g, 99%) as colorless oil.

### Step B: 4-(3-Cyanophenyloxy)piperidine Hydrochloride

To the compound from Step A (3.00 g, 9.90 mmol) in 1,4-dioxane (30 mL) is added HCl (4N, aqueous) (10.0 mL) and the mixture is stirred at rt overnight. The reaction is concentrated in vacuo and evaporated repeatedly from toluene to give the desired product (1.20 g) after trituration with ethyl acetate.

### Step C: 4-(3-Cyano-phenoxy)-piperidine-1-carboxylic Acid 2,4-dichloro-benzylamide

This compound is prepared using the procedure from Example 1, starting from the compound from Step B (0.238 mg, 1.00 mmol), diisopropylethylamine (0.129 g, 1.00 mmol) and 2,4-dichloro-1-isocyanatomethyl-benzene (0.202 g, 1.00 mmol). The mixture is stirred at rt overnight, poured into dichloromethane/aqueous sodium bicarbonate (20 mL each), the organic phase is separated, extracted with water (20 mL), dried over magnesium sulphate, filtered, evaporated in vacuo and purified by filtration through silica using ethyl acetate as the eluent to give the desired product (0.345 g, 85.6%). LCMS: 404.25 (M+H⁺).

### EXAMPLE 41

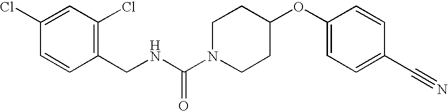

### Step A: tert-Butyl-4-(4-cyanophenyloxy)-1-piperidinecarboxylate

To tert-butyl 4-hydroxy-1-piperidinecarboxylate (24.1 g, 100.0 mmol) in dimethylformamide (250 mL) is added sodium hydride (60% emulsion in mineral oil) (8.00 g, 200.0 mmol) and the mixture is stirred at rt for 1 h. 4-Fluorobenzonitrile (12.1 g, 100.0 mmol) is added, the mixture is heated to 60° C. for 30 minutes, diluted with ethyl acetate and the reaction is quenched by the addition of water. The organic phase is evaporated in vacuo and purified on silica gel to give the desired product (27.5 g, 91%).

### Step B: 4-(4-Cyanophenyloxy)piperidine Hydrochloride

To the compound from Step A (2.20 g, 7.27 mmol) in 1,4-dioxane (30 mL) is added HCl (4N, aqueous) (10.0 mL) and the mixture is stirred at room temperature overnight. The reaction is evaporated in vacuo and evaporated repeatedly from toluene to give the desired product (1.08 g) after trituration with ethyl acetate.

### Step C: 4-(4-Cyano-phenoxy)-piperidine-1-carboxylic Acid 2,4-dichloro-benzylamide

This compound is prepared using the procedure from Example 1, starting from the compound from Step B (0.238 mg, 1.00 mmol), diisopropylethylamine (0.129 g, 1.00 mmol) and 2,4-dichloro-1-isocyanatomethyl-benzene (0.202 g, 1.00 mmol). The mixture is stirred at rt overnight, poured into dichloromethane/aqueous sodium bicarbonate (20 mL each), the organic phase is separated, extracted with water (20 mL), dried over magnesium sulfate, filtered and evaporated in vacuo to give the desired product (0.35 g, 85.6%). LCMS: 404.24 (M+H⁺).

### EXAMPLE 42

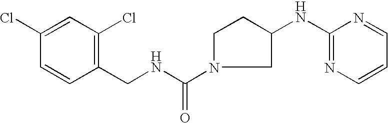

### 3-(Pyrimidin-2-ylamino)-pyrrolidine-1-carboxylic Acid 2,4-dichloro-benzylamide

### Step A: tert-Butyl 3-(pyrimidine-2-ylamino)-1-pyrrolidinecarboxylate

To a solution of 1-N-Boc-3-amino-pyrrolidone (4.00 g, 21.5 mmol) in isopropanol (50 mL) is added 2-chloropyrimidine (2.94 g, 25.8 mmol) and diisopropylethylamine (4.16 g, 32.3 mmol). The mixture is heated at reflux for 3 days, allowed to come to room temperature, evaporated in vacuo and purified on silica gel using ethyl acetate/heptane to give the desired product (4.20 g, 74%).

### Step B: 3-(Pyrimidin-2-ylamino)-pyrrolidine Hydrochloride

To the compound from Step A (4.60 g, 17.42 mmol) in 1,4-dioxane (40 mL) is added water (3.0 mL) and HCl (concentrated, aqueous) (3.0 mL). The mixture is stirred at rt for 19 h, evaporated in vacuo and evaporated to give the desired product (4.00 g, 100

### Step C: 3-(Pyrimidin-2-ylamino)-pyrrolidine-1-carboxylic Acid 2,4-dichloro-benzylamide

This compound is prepared using the procedure from Example 1, starting from the compound from Step B (0.201 mg, 1.00 mmol), diisopropylethylamine (0.129 g, 1.00 mmol) and 2,4-dichloro-1-isocyanatomethyl-benzene (0.202 g, 1.00 mmol), and is purified on silica gel using methanol/dichloromethane (5:95) as the eluent to give the desired product (0.362 g, 64.4%). LCMS: 366.31 (M+H⁺).

### EXAMPLE 43

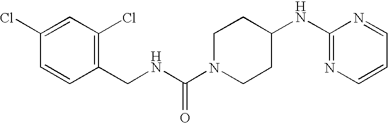

### 4-(Pyrimidin-2-ylamino)-piperidine-1-carboxylic Acid 2,4-dichloro-benzylamide

### Step A: tert-Butyl-4-(pyrimidin-2-ylamino)-1-piperidinecarboxylate

To a solution of 1-N-Boc-4-amino-piperidine (3.20 g, 16.0 mmol) in isopropanol (50 mL) is added 2-chloropyrimidine (2.05 g, 18.0 mmol) and diisopropylethylamine (3.09 g, 24 mmol). The mixture is heated at reflux for 48 h allowed to come to room temperature, evaporated in vacuo and purified on silica gel using ethyl acetate/heptane to give the desired product (2.82 g, 63%).

### Step B: 4-(Pyrimidin-2-ylamino)-piperidine Hydrochloride

To the compound from Step A (2.82 g, 10.2 mmol) in 1,4-dioxane (40 mL) is added water (3.0 mL) and HCl (concentrated, aqueous) (3.0 mL). The mixture is stirred at rt for 4 days, evaporated in vacuo and evaporated repeatedly from toluene to give the desired product (2.40 g) after trituration with diethyl ether.

### Step C: 4-(Pyrimidin-2-ylamino)-piperidine-1-carboxylic Acid 2,4-dichloro-benzylamide

This compound is prepared using the procedure from Example 12, starting from the compound from Step B (0.214 mg, 1.00 mmol), diisopropylethylamine (0.129 g, 1.00 mmol) and 2,4-dichloro-1-isocyanatomethyl-benzene (0.202 g, 1.00 mmol). The mixture is stirred overnight and the solid is filtered off, washed with acetonitrile and dried in vacuo to give the desired compound (0.362 g, 64.4%). LCMS: 380.30 (M+H⁺).

### EXAMPLE 44

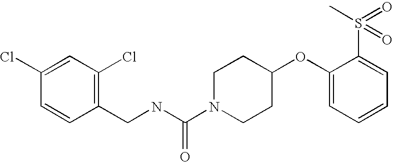

### 4-(2-Methanesulfonyl-phenoxy)-piperidine-1-carboxylic Acid 2,4-dichloro-benzylamide

### Step A: tert-Butyl 4-(2-methylmercaptophenyloxy)-1-piperidinecarboxylate

To a solution of 1-N-Boc-4-hydroxy-piperidine (2.41 g, 12.0 mmol) in dimethylformamide (50 mL) is added sodium hydride (60% emulsion in mineral oil) (0.80 g, 20.0 mmol). The mixture is stirred at rt for 1 h, 2-fluorothioanisole (1.42 g, 100.0 mmol) is added and the mixture is heated to 60° C. for 12 h. The mixture is diluted with ethyl acetate and the reaction quenched by the addition of water. The organic phase is dried over sodium sulfate, evaporated in vacuo and purified on silica gel to give the desired product (2.22 g, 69%).

### Step B: tert-Butyl 4-(2-methanesulfonylphenyloxy)-1-piperidinecarboxylate

To a mixture of aluminium oxide (7.00 g, 69.0 mmol) and water (1.5 mL, 82.8 mmol) is added the compound from Step A (2.22 g, 6.9 mmol) in chloroform (100 mL) followed by the addition of oxone (17.0 g, 27.6 mmol) and the mixture is stirred under reflux for 18 h. The mixture is cooled to room temperature, filtered and the filtrate evaporated in vacuo. The resultant colorless oil is triturated with diethyl ether to give the desired product (2.00 g, 80%) as colorless powder.

### Step C: 4-(2-Methanesulfonylphenyloxy)piperidine Hydrochloride

To the compound from Step B (1.80 g, 5.06 mmol) in 1,4-dioxane (30 mL) is added HCl (4N, aqueous) (10.0 mL) and the mixture is stirred at room temperature for 18 h. The reaction is evaporated in vacuo and evaporated repeatedly from toluene to give the desired product (1.45 g, 98%) after trituration with chloroform.

### Step D: 4-(2-Methanesulfonyl-phenoxy)-piperidine-1-carboxylic Acid 2,4-dichloro-benzylamide

This compound is prepared using the procedure from Example 1, starting from the compound from Step C (0.292 g, 1.00 mmol), diisopropylethylamine (0.129 g, 1.00 mmol) and 2,4-dichloro-1-isocyanatomethyl-benzene (0.202 g, 1.00 mmol), and is purified on silica gel using ethyl acetate as the eluent to give the desired product (0.389 g, 85.1%). LCMS: 457.12 (M+H⁺).

### EXAMPLE 45

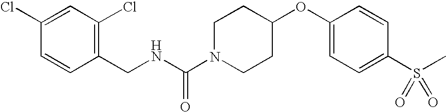

### 4-(4-Methanesulfonyl-phenoxy)-piperidine-1-carboxylic Acid 2,4-dichloro-benzylamide

### Step A: tert-Butyl 4-(2-methylmercaptophenyloxy)-1-piperidinecarboxylate

To a solution of tert-butyl 4-hydroxy-piperidinecarboxylate (2.41 g, 12.0 mmol) in dimethylformamide (25 mL) is added sodium hydride (60% emulsion in mineral oil) (0.80 g, 20.0 mmol). The mixture is stirred at rt for 1 h, 4-fluorothioanisole (1.42 g, 100.0 mmol) is added and the mixture is heated to 60° C. for 12 h. The mixture is diluted with ethyl acetate and the reaction quenched by the addition of water. The organic phase is dried over sodium sulfate, evaporated in vacuo and purified on silica gel to give the desired product (2.25 g, 70%).

### Step B: tert-Butyl 4-(4-methanesulfonylphenyloxy)-1-piperidinecarboxylate

To a mixture of aluminium oxide (7.00 g, 69.0 mmol) and water (1.5 mL, 82.8 mmol) is added the compound from Step A (2.22 g, 6.90 mmol) in chloroform (100 mL) followed by the addition of oxone (17.0 g, 27.6 mmol) and the mixture is stirred under reflux for 18 h. The mixture is cooled to room temperature, filtered and the filtrate evaporated in vacuo. The resultant colorless oil is triturated with diethyl ether to give the desired product (2.20 g, 92%) as colorless powder.

### Step C: 4-(4-Methanesulfonylphenyloxy)piperidine Hydrochloride

To the compound from Step B (2.20 g, 6.19 mmol) in 1,4-dioxane (30 mL) is added HCl (4N, aqueous) (10.0 mL) and the mixture is stirred at room temperature for 18 h. The reaction is evaporated in vacuo and evaporated repeatedly from toluene to give the desired product (1.78 g, 98%) after trituration with chloroform.

### Step D: 4-(4-Methanesulfonyl-phenoxy)-piperidine-1-carboxylic Acid 2,4-dichloro-benzylamide

This compound is prepared using the procedure from Example 1, starting from the compound from Step C (0.292 g, 1.00 mmol), diisopropylethylamine (0.129 g, 1.00 mmol) and 2,4-dichloro-1-isocyanatomethyl-benzene (0.202 g, 1.00 mmol), and is purified on silica gel using ethyl acetate as the eluent to give the desired product (0.394 g, 86.1%). LCMS: 457.12 (M+H⁺).

### EXAMPLE 46

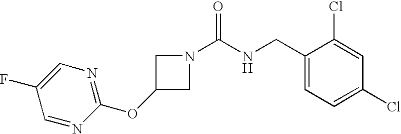

### 3-(5-Fluoro-pyrimidin-2-yloxy)-azetidine-1-carboxylic Acid 2,4-dichloro-benzylamide

### Step A: tert-Butyl 3-hydroxy-azetidine-1-carboxylate

To a suspension of 3-azetidinol hydrochloride (2.50 g, 22.8 mmol) in 33 mL of ethanol is added di-t-butyl dicarbonate (5.47 g, 25.10 mmol) and triethylamine (9.60 mL, 68.5 mmol) and the mixture is stirred at room temperature for 24 h. The solvents are removed in vacuo, and the residue is taken up in ethyl acetate, washed with 10% citric acid, water, and brine. The organic phase is dried over magnesium sulfate, filtered and evaporated to dryness. The resulting white solid is purified on silica gel using hexanes ethyl acetate as the eluent to give the title compound (3.00 g, 69.0%). ¹H NMR (400 MHz): 4.70 (br s, 1H), 4.19 (m, 2H), 3.81 (m, 2H), 1.42 (s, 9H).

### Step B: 2-(Azetidin-3-yloxy)-5-fluoro-pyrimidine Hydrochloride

A suspension of the compound from Step A (0.250 g, 1.44 mmol) in tetrahydrofuran (15 mL) is cooled to 0° C. and treated with potassium tert-butoxide (0.138 g, 1.44 mmol). The reaction is stirred for 10 minutes and 2-chloro-5-fluoropyrimidine (0.192 g, 1.45 mmol) is added and the reaction is warmed to room temperature. After stirring for 3.5 h the solvent is evaporated in vacuo and the residue taken up in 1N HCl and washed with ether. The aqueous solution is made basic and extracted with ethyl acetate. The extracts are washed with water, dried over magnesium sulfate, filtered and evaporated in vacuo to give a clear oil. The oil is taken up in 4N HCl in ether (5 mL) and stirred overnight. After 12 hours the solid precipitate is collected by filtration and dried to give the desired product (0.104 g, 34%) which was used without further purification.

### Step C: 3-(5-Fluoro-pyrimidin-2-yloxy)-azetidine-1-carboxylic Acid 2,4-dichloro-benzylamide

The compound is prepared using the procedure from Example 1 starting from the compound from Step B (0.104 g, 0.57 mmol), diisopropylethylamine (0.174 mL, 1.00 mmol) and 2,4-dichloro-1-isocyanatomethyl-benzene (0.074 mL, 0.50 mmol), and is purified by recrystallization from acetonitrile to give the desired product (0.025 g, 13.3%). LCMS: 372.1 (M+H⁺).

### EXAMPLE 47

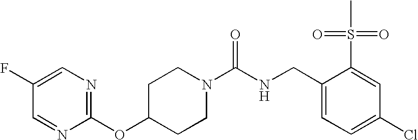

### 4-(5-Fluoro-pyrimidin-2-yloxy)-piperidine-1-carboxylic Acid 4-chloro-2-methanesulfonyl-benzylamide

### Step A: 4-Chloro-2-methylsulfanyl-benzamide

A solution of 2,4-dichlorobenzamide (5.00 g, 26.2 mmol) in dimethylformamide (131 mL) is treated with sodium thiomethoxide (3.20 g, 45.9 mmol) and heated at 60° C. After 2 h the reaction is cooled to room temperature and water is added. The solvent is removed in vacuo to give a white solid that is used without further purification

### Step B: 4-Chloro-2-methylsulfanyl-benzylamine

Borane—THF (40 mL, 40 mmol) is added to the compound from Step A (2.66 g, 13.2 mmol) and allowed to stir for 16 h. The reaction is quenched by the slow addition of methanol. The solvents are removed from the reaction in vacuo, and resulting solid purified on silica gel using ethyl acetate/methanol as the eluent to give the title compound (1.20 g, 48%).

### Step C: 4-Chloro-2-methanesulfinyl-benzylamine

A suspension of the compound from Step B (1.49 g, 7.94 mmol) in dichloromethane (80 mL) is treated with di-tert-butyl dicarbonate (1.73 g, 7.94 mmol) and triethylamine (1.10 mL, 7.94 mmol) and reacted until complete consumption as monitored by LC/MS. The solvents are removed in vacuo and the crude residue treated with dichloromethane (80 mL) and scuba (1.51 g, 8.73 mmol) and reacted until complete consumption as monitored by LC/MS. The solvents are removed in vacuo and the residue taken up in dichloromethane (5 mL) and trifluoroacetic acid (10 mL). After one hour para toluene sulfonic acid is added (7.94 mmol) and the reaction is stirred for 30 minutes, The solvents are evaporated in vacuo to give a white solid (2.01 g, 64%), that was used without further purification.

### Step D: (4-Chloro-2-methanesulfonyl-benzyl)-carbamic Acid tert-butyl Ester

Aluminum oxide (9.68 g, 89.0 mmol) is added to water (2 mL) and stirred for 5 minutes. The compound from Step C (4.61 g, 11.7 mmol) is dissolved in chloroform (185 mL) and added to solution followed by oxone (19.3 g, 30.0 mmol). The reaction is heated at reflux for 16 hour cooled to room temperature, filtered and concentrated to give the title compound as colorless solid (2.01 g, 53.8%) that is in the next step without further purification.

### Step E: 4-Chloro-2-methanesulfonyl-benzylamine

The compound from Step D (2.01 g, 6.3 mmol) is dissolved in dichloromethane (10 mL) and trifluoroacetic acid (20 mL). The reaction is stirred for 1 hour and then the solvent removed in vacuo. Dichloromethane is added and evaporated three times and then the solid is dissolved in dichloromethane (10 mL) and para-toluene sulfonic acid (1.20 g 6.30 mmol) is added. The reaction is stirred for 1 hour then filtered and dried to give the desired compound (2.00 g, 80.1%)

### Step F: 4-(5-Fluoro-pyrimidin-2-yloxy)-piperidine-1-carboxylic Acid 4-chloro-2-methanesulfonyl-benzylamide

To a solution of carbonyldiimidazole (0.151 g, 0.93 mmol) in tetrahydrofuran (5.2 mL) is added the compound from Step E (0.356 g, 0.91 mmol) followed by diisopropylethylamine (0.175 mL, 1.00 mmol) and the mixture is stirred for two hours. The product of Example 31 Step C (0.198 g, 0.85 mmol) is added to the reaction followed by additional diisopropylethylamine (0.175 mL, 1.00 mmol) and the reaction heated at reflux for 16 hours. The reaction is cooled to room temperature, and the solvents evaporated in vacuo. The residue is taken up in ethyl acetate and washed with water and brine, dried over magnesium sulfate and evaporated to dryness. The resulting solid is purified on silica gel using dichloromethane/methanol (10:1) as the eluent to give the desired compound (0.067 g, 16.6%) as the para toluenesulfonic acid salt. LCMS: 443.91 (M+H⁺).

### EXAMPLE 48

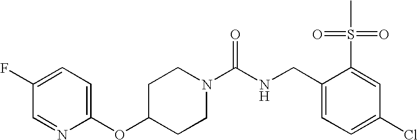

### 4-(5-Fluoro-pyridin-2-yloxy)-piperidine-1-carboxylic Acid 4-chloro-2-methanesulfonyl-benzylamide

This compound is prepared using the procedure from Example 47, starting from carbonyldiimidazole (0.151 g, 0.93 mmol), the compound from Example 47, Step E (0.356 g, 0.91 mmol), diisopropylethylamine (0.175 mL, 1.00 mmol), the compound from Example 17, Step B (0.263 g, 0.850 mmol), diisopropylethylamine (0.175 mL, 1.00 mmol), and is purified on silica gel using dichloromethane/methanol (10:1) as the eluent to give the desired product (0.010 g, 2.5%). LCMS: 441.95 (M+H⁺).

### EXAMPLE 49

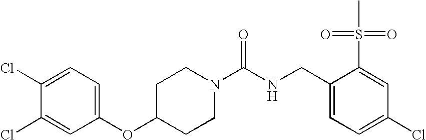

### 4-(3,4-Dichloro-phenoxy)-piperidine-1-carboxylic Acid 4-chloro-2-methanesulfonyl-benzylamide

This compound is prepared using the procedure from Example 47, starting from carbonyldiimidazole (0.151 g, 0.93 mmol), the compound from Example 47, Step E (0.356 g, 0.91 mmol), diisopropylethylamine (0.175 mL, 1.00 mmol), 4-(3,4-dichloro-phenoxy)-piperidine hydrochloride (0.240 g, 0.850 mmol), diisopropylethylamine (0.175 mL, 1.00 mmol), and is purified on silica gel using dichloromethane/methanol (10:1) as the eluent to give the desired product (0.038 g, 8.5%). LCMS: 492.91 (M+H⁺).

### EXAMPLE 50

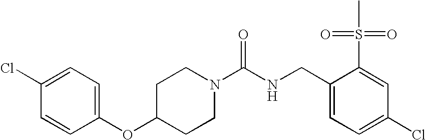

### 4-(4-Chloro-phenoxy)-piperidine-1-carboxylic Acid 4-chloro-2-methanesulfonyl-benzylamide

This compound is prepared using the procedure from Example 47, starting from carbonyldiimidazole (0.151 g, 0.93 mmol), the compound from Example 47, Step E (0.356 g, 0.91 mmol), diisopropylethylamine (0.175 mL, 1.00 mmol), 4-(4-chloro-phenoxy)-piperidine hydrochloride (0.210 g, 0.850 mmol), diisopropylethylamine (0.175 mL, 1.00 mmol), and is purified on silica gel using dichloromethane/methanol (10:1) as the eluent to give the desired product (0.015 g, 3.6%). LCMS: 458.41 (M+H⁺).

### EXAMPLE 51

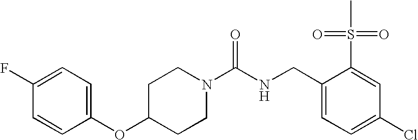

### 4-(4-Fluoro-phenoxy)-piperidine-1-carboxylic Acid 4-chloro-2-methanesulfonyl-benzylamide

This compound is prepared using the procedure from Example 47, starting from carbonyldiimidazole (0.151 g, 0.93 mmol), the compound from Example 47, Step E (0.356 g, 0.91 mmol), diisopropylethylamine (0.175 mL, 1.00 mmol), 4-(4-fluoro-phenoxy)-piperidine hydrochloride (0.196 g, 0.850 mmol), diisopropylethylamine (0.175 mL, 1.00 mmol), and is purified on silica gel using dichloromethane/methanol (10:1) as the eluent to give the desired product (0.049 g, 12.2%). LCMS: 441.10 (M+H⁺).

### EXAMPLE 52

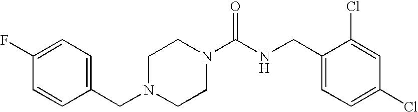

### 4-(4-Fluoro-benzyl)-piperazine-1-carboxylic Acid 2,4-dichloro-benzylamide

This compound is prepared using the procedure from Example 1, starting from 1-(4-fluoro-benzyl)-piperazine (0.194 g, 1.00 mmol), diisopropylethylamine (0.129 g, 1.00 mmol) and 2,4-dichloro-1-isocyanatomethyl-benzene (0.202 g, 1.00 mmol), and is purified on silica gel using ethyl acetate as the eluent to give the desired product (0.220 g, 55.5%). LCMS: 397.4 (M+H⁺).

### EXAMPLE 53

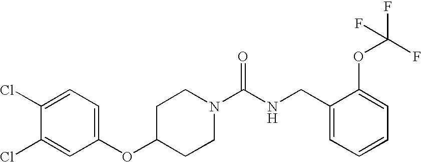

### 4-(3,4-Dichloro-phenoxy)-piperidine-1-carboxylic Acid 2-trifluoromethoxy-benzylamide

To a solution of triphosgene (0.098 g, 0.33 mmol) in dichloromethane (7.0 mL) is added 2-trifluoromethoxy-benzylamine (0.191 mg, 1.00 mmol) and diisopropylethylamine (0.435 mL, 2.50 mmol) in 1.4 mL dichloromethane. The reaction is stirred for 5 minutes and then a solution of 4-(3,4-dichloro-phenoxy)-piperidine hydrochloride (282.5 mg, 1.00 mmol) and diisopropylethylamine (0.191 mL, 1.10 mmol) in is added to the reaction. The reaction is stirred for an additional 30 minutes, diluted with dichloromethane and washed with 1M HCl, saturated sodium bicarbonate and brine. The organic layer is dried over magnesium sulfate, filtered and evaporated to dryness. The crude material is purified on silica gel with dichloromethane/methanol (10:1) as the eluent. The product is further purified by recrystallization from hexanes/ethyl acetate to give the desired product (0.058 g, 12.5%) LCMS: 465.56 (M+H⁺).

### EXAMPLE 54

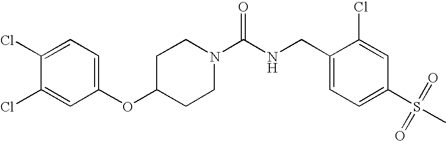

### 4-(3,4-Dichloro-phenoxy)-piperidine-1-carboxylic Acid 2-chloro-4-methanesulfonyl-benzylamide

### Step A: 2-Chloro-4-methanesulfonyl-benzamide

2-Chloro-4-methanesulfonyl-benzoic acid (21.0 g, 89.1 mmol) is suspended in acetonitrile (200 mL) and di-tert-butyl dicarbonate (27.0 g, 124 mmol) is added in one portion. The resulting mixture is stirred for 15 minutes and ammonium bicarbonate (79.10 mmol) followed by pyridine (11.2 mL 124 mmol) are added to the reaction. The reaction is stirred for 16 hours at room temperature and then the solvents evaporated in vacuo. The residue is triturated with 10% NaOH and water until the solutions are clear and the solids washed with water and 5% ether in petroleum ether. The solid is collected and dried in vacuo to give the desired compound (17.6 g, 84%). LCMS: 234.03 (M+H⁺).

### Step B: 2-Chloro-4-methanesulfonyl-benzylamine Hydrochloride

To a solution of borane in THF (1M, 120 mL, 120 mmol) is added the compound from Step A over 5 minutes. The resulting suspension is heated to reflux and reacted for 16 hours. The reaction is cooled with an ice bath and excess borane is quenched by the slow addition of 6N HCl. The addition of HCl is stopped after gas evolution ceases and the resulting white solid precipitate is collected by vacuum filtration. The white solid is washed with 6N HCl and tetrahydrofuran/diethylether (1:1) and dried in vacuo to yield the title compound (8.20 g, 88%) LCMS: 222.23 (M+H⁺).

### Step C: 4-(3,4-Dichloro-phenoxy)-piperidine-1-carboxylic Acid 2-chloro-4-methanesulfonyl-benzylamide

This compound is prepared using the procedure from Example 53, starting from triphosgene (0.098 g, 0.33 mmol) the compound from Step B (0.219 g, 1.00 mmol) diisopropylethylamine (0.435 mL, 2.50 mmol), 4-(2,3-dichlorophenoxy-piperidine hydrochloride (282.5 mg, 1.00 mmol), diisopropylethylamine (0.191 mL, 1.10 mmol), and is purified on silica gel using dichloromethane/methanol (10:1) as the eluent to give the desired product. The product is further purified by recrystallization from hexanes/ethyl acetate to give the desired product (0.012 g, 2.5%). LCMS: 492.88 M+H⁺).

### EXAMPLE 55

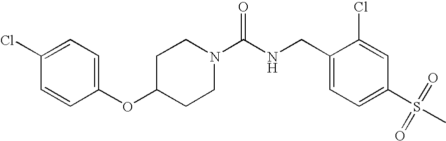

### 4-(4-Chloro-phenoxy)-piperidine-1-carboxylic Acid 2-chloro-4-methanesulfonyl-benzylamide

This compound is prepared using the procedure from Example 47, starting from carbonyldiimidazole (0.151 g, 0.93 mmol), 2-chloro-4-methanesulfonyl-benzylamine (from steps A and B for Example 54) (0.200 g, 0.91 mmol) diisopropylethylamine (0.175 mL, 1.00 mmol), 4-(4-fluoro-phenoxy)-piperidine hydrochloride (0.196 g, 0.850 mmol), diisopropylethylamine (0.175 mL, 1.00 mmol), and is purified on silica gel using dichloromethane/methanol (10:1) as the eluent to give the desired product (0.200 g, 48.2%). LCMS: 458.95 (M+H⁺).

### EXAMPLE 56

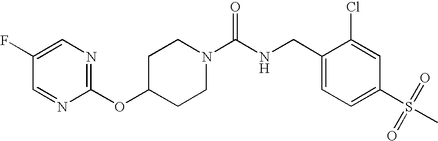

### 4-(5-Fluoro-pyrimidin-2-yloxy)-piperidine-1-carboxylic Acid 2-chloro-4-methanesulfonyl-benzylamide

A solution of the product from Example 31, Step C (1.096 g, 4.28 mmol) and DIPEA (1.49 mL, 8.56 mmol) in DCM (10 mL) is added dropwise to a solution of triphosgene (0.381, 1.284 mmol) in dichloromethane (20 mL). The mixture is stirred for 5 min at room temperature and then a solution of the product from Example 54 Step B (0.94 g, 4.28 mmol) and DIPEA (1.58 mL, 8.56 mmol) in dichloromethane (10 mL) is added.

The mixture is stirred for an additional 40 minutes and subsequently washed with 1N HCl, sodium bicarbonate and brine. The organic phase is dried over magnesium sulfate and the solvent is evaporated in vacuo to give the desired compound (1.70 g, 90.2%).

### EXAMPLE 57

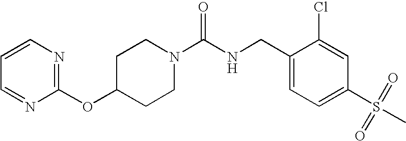

### 4-(Pyrimidin-2-yloxy)-piperidine-1-carboxylic Acid 2-chloro-4-methanesulfonyl-benzylamide

This compound is prepared using the procedure from Example 47, starting from carbonyldiimidazole (0.151 g, 0.937 mmol), the compound from Example 54, Step B (0.200 g, 0.910 mmol) diisopropylethylamine (0.175 mL, 1.00 mmol), 2-(piperidin-4-yloxy)-pyrimidine dihydrochloride (0.183 g, 0.850 mmol), diisopropylethylamine (0.175 mL, 1.00 mmol), and is purified on silica gel using dichloromethane/methanol (10:1) as the eluent to give the desired product (0.204 g, 52.7%). LCMS: 425.88 (M+H⁺).

### EXAMPLE 58

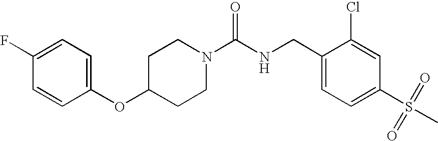

### 4-(4-Fluoro-phenoxy)-piperidine-1-carboxylic Acid 2-chloro-4-methanesulfonyl-benzylamide

This compound is prepared using the procedure from Example 47, starting from carbonyldiimidazole (0.151 g, 0.937 mmol), the compound from Example 54, Step B (0.200 g, 0.910 mmol), diisopropylethylamine (0.175 mL, 1.00 mmol), 4-(4-fluoro-phenoxy)-piperidine hydrochloride (0.196 g, 0.850 mmol), and is purified on silica gel using dichloromethane/methanol (10:1) as the eluent to give the desired product (0.186 g, 46.3%). LCMS: 442.99 (M+H⁺).

### EXAMPLE 59

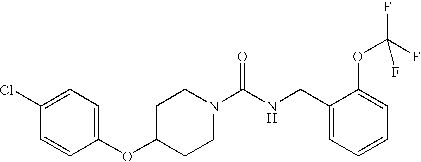

### 4-(4-Chloro-phenoxy)-piperidine-1-carboxylic Acid 2-trifluoromethoxy-benzylamide

This compound is prepared using the procedure from Example 47, starting from carbonyldiimidazole (0.151 g, 0.937 mmol), 2-trifluoromethoxybenzylamine (0.163 g, 0.937 mmol), diisopropylethylamine (0.175 mL, 1.00 mmol), 4-(4-chloro-phenoxy)-piperidine hydrochloride (0.212 g, 0.853 mmol), diisopropylethylamine (0.175 mL, 1.00 mmol), and is purified on silica gel using dichloromethane/methanol (10:1) as the eluent to give the desired product (0.100 g, 27.3%). LCMS: 429.01 (M+H⁺).

### EXAMPLE 60

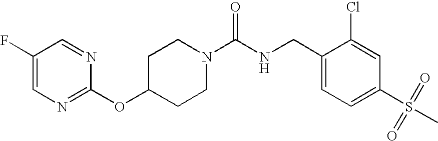

### 4-(Pyrimidin-2-yloxy)-piperidine-1-carboxylic Acid 2-trifluoromethoxy-benzylamide

This compound is prepared using the procedure from Example 53, starting from triphosgene (0.098 g, 0.33 mmol), the compound from Example 54, Step B, (0.191 g, 1.00 mmol), diisopropylethylamine (0.435 mL, 2.50 mmol), 2-(piperidin-4-yloxy)-pyrimidine dihydrochloride (0.251 g, 1.00 mmol), diisopropylethylamine (0.191 mL, 1.10 mmol), and is purified on silica gel using dichloromethane/methanol (10:1) as the eluent to give the desired product. The product is further purified by recrystallization in hexanes/ethyl acetate to give the desired product (0.024 g, 6.1%). LCMS: 397.29 (M+H⁺).

### EXAMPLE 61

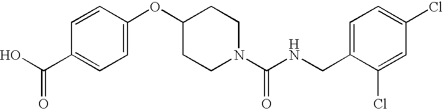

### 4-(4-Carboxyphenoxy)-piperidine-1-carboxylic Acid 2,4-dichloro-benzylamide

### Step A: tert-Butyl-4-(4-carboxyethylphenyloxy)-1-piperidinecarboxylate

To a pre-cooled (0° C.) suspension of ethyl 4-hydroxybenzoate (10.0 g, 60.2, mmol), 1-tert-butoxycarconyl 4-hydroxypiperidine (12.1 g, 60.1 mmol), and triphenylphosphine (15.8 g, 60.2 mmol) in THF is added diisopropyl azodocarboxylate (11.6 g, 60.1 mmol). The mixture is allowed to come to rt, stirred for 16 h, diluted with ethyl acetate, washed with water, sodium bicarbonate (saturated, aqueous), dried over sodium sulfate, filtered and evaporated in vacuo. The crude product is purified on silica to give the desired product (8.27 g, 39%).

### Step B: 4-(4-Carboxyethylphenyloxy)-1-piperidine Hydrochloride

To the compound from Step B (8.27 g, 23.7 mmol) in 1,4-dioxane (25 mL) is added HCl (4N, dioxane) (25.0 mL) and the mixture is stirred at room temperature for 2 h. The The product is filtered off and dried in vacuo to give the desired compound as the hydrochloride (6.27 g, 93%).

### Step C: 4-(4-Carboxyethylphenyloxy)-piperidine-1-carboxylic Acid 2,4-dichloro-benzylamide

To the compound from Step B (8.27 g, 28.9 mmol) and diisopropylethylamine (10.1 mL, 58.0 mmol) in acetonitrile (100 mL) is added 2,4-dichloro-1-isocyanatomethyl-benzene (4.25 mL, 29.2 mmol). The mixture is stirred at rt for 16 h, evaporated in vacuo, dissolved in ethyl acetate, washed repeatedly with water, evaporated in vacuo and triturated with hexane/ethyl acetate to give the desired compound (13.1 g, 99%).

### Step D: 4-(4-Carboxyphenyloxy)-piperidine-1-carboxylic Acid 2,4-dichloro-benzylamide

To the compound from Step C (13.0 g, 28.8 mmol) in dioxane/water (1:1) (100 mL) is added LiOH (2.42 g, 57.7 mmol). The mixture is stirred for 15 minutes, heated to 60° C. and stirred for another 3 h. Dioxane is evaporated in vacuo, the solution is neutralized with 4M HCl in dioxane, dioxane evaporated in vacuo, and product is filtered off, washed with water and then ethyl ether, and dried in vacuo to give the desired compound (12.2 g, 96%).

### EXAMPLE 62

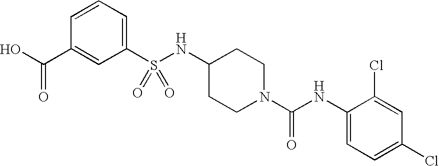

### 3-[1-(2,4-Dichloro-phenylcarbamoyl)-piperidin-4-ylsulfamoyl]-benzoic Acid

### Step A: [1-(2,4-Dichloro-phenylcarbamoyl)-piperidin-4-yl]-carbamic Acid tert-butyl Ester

To a solution of piperidin-4-yl-carbamic acid tert-butyl ester (2.020 g, 10.0 mmol) in acetonitrile (30 mL) is added 2,4-dichloro-1-isocyanatomethyl-benzene (2.00 g, 10.0 mmol). The clear solution is stirred for 2 hours at room temperature. The mixture is filtered and recrystallized from acetonitrile to give the urea product as a white powder. The filtrate is put aside and formed more crystals on it by adding hexanes/ether. The solids were filtered, washed and dried to provide the desired product (4.00 g, 99.4%).

### Step B: 4-Amino-piperidine-1-carboxylic Acid (2,4-dichloro-phenyl)-amide

To the product from Step A product (0.1 g, 0.249 mmol) in dichloromethane, is added 4 N HCl in dioxane (1.00 mL, 4.00 mmol) at room temperature. The mixture is stirred for 4 hours. The resulting solid is removed by filtration and dried to obtain the desired product (0.060 g, 79.9%).

### Step C: 3-[1-(2,4-Dichloro-phenylcarbamoyl)-piperidin-4-ylsulfamoyl]-benzoic Acid

The product from Step B (0.30 g, 0.99 mmol) and 3-chlorosulfonyl-benzoic acid (0.44 g, 1.99 mmol) in pyridine (20 mL) are stirred for 48 hours at 60° C. Pyridine is then removed in vacuo and the viscous liquid is purified by preparative HPLC. Fractions containing the desired product are collected and evaporated in vacuo. The resulting oil is triturated with ether to give the desired product (0.088 g, 18.3%). LCMS: 486.06 (M+H⁺).

### EXAMPLE 63

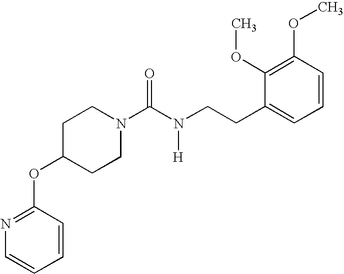

Step A: The polystyrene 4-(4-formyl-3-methoxyphenoxy)butyryl aminomethylated resin (5.00 g, 4.70 mmol; Nova Biochem #01-64-0209; loading 0.94 mmol/g) is suspended in 1,2-dichloroethane (100 mL), followed by the addition of 2,3-dimethoxyphenethylamine (3.95 mL, 23.5 mmol). The suspension is agitated on an orbital shaker at room temperature for approximately 30 minutes. Sodium triacetoxyborohydride (9.96 g, 47.0 mmol) is added and the yellow suspension is agitated on an orbital shaker overnight at room temperature. The resin suspension is diluted with DMA/water (80:20) (25 mL) and the resin is collected by filtration through a sintered glass funnel. The resin is washed with DMA/water (8:2) (3×25 mL), dichloromethane (3×25 mL), methanol (3×50 mL) and dichloromethane (2×50 mL). The resultant pale yellow resin is dried in vacuo.

Step B: The resin from Step A (3.00 g, 2.82 mmol) is suspended in dichloromethane (40 mL) and N-methylmorpholine (0.930 mL, 8.46 mmol) is added, followed by para-nitrophenylchloroformate (1.71 g, 8.46 mmol). The resultant orange suspension is agitated on an orbital shaker overnight at room temperature. The resin is collected on a sintered glass funnel, subsequently washed with dichloromethane (4×50 mL), and dried in vacuo.

Step C: The resin from Step B (100 mg, 0.094 mmol) is placed in a glass reaction tube (Bohdan miniblock reactor equipped with glass reaction tubes and heating jacket). To the resin is added a solution of 2-(piperidin-4-yloxy)pyridine (0.500 mL of a 0.564 mmol solution in DMA, 0.282 mmol, 3 eq), followed by 0.043 mL of DBU (0.282 mmol, 3 eq). The miniblocks are capped and heated at 100° C. After heating for 36 hours, the blocks are cooled to room temperature and the resin filtered, and washed with dichloromethane (2×0.5 mL), DMA (2×0.5 mL), and dichloromethane (2×0.5 mL). To the resin is added a solution of TFA in dichloromethane (20% v/v, 0.500 mL) and the resulting suspension is agitated on an orbital shaker at room temperature for two hours. The resin is filtered and the filtrate collected in a 96-well deep-well plate. The resin is further washed with dichloromethane (2×0.5 mL) and the filtrates are collected in the 96-well deep-well plate. The solution is concentrated and dried in vacuo to give the desired compound (0.018 g, 50%). LCMS: 386 (M+H⁺).

### EXAMPLE 64

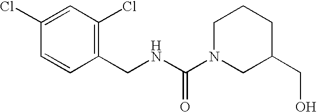

This compound is prepared using the procedure from Example 61, starting from polystyrene 4-(4-formyl-3-methoxyphenoxy)butyryl aminomethylated resin (5.00 g, 4.70 mmol; Nova Biochem #01-64-0209; loading 0.94 mmol/g) in Step A; 2,4-dichlorobenzylamine (3.16 mL, 23.5 mmol) in Step B; and a DMA solution of 3-(t-butylhydroxymethyl)piperidine (0.500 mL of a 0.564 mmol solution in DMA, 0.282 mmol, 3 eq) in Step C. The compound is initially isolated as the trifluoroacetate ester of the hydroxymethylpiperidine and is converted to the desired compound by treatment of the material with 0.500 mL of 10% methanol/DCE (1:9) and Si—CO₃ (150 mg, 0.119 mmol, Silicycle R66030B, loading 0.79 mmol/gram). The suspension is agitated on an orbital shaker overnight at room temperature. The suspension is filtered and the SiCO₃ is washed with methanol DCE (1:9) (2×500 mL). The combined filtrates are concentrated in vacuo and dried to give the desired product (0.010 g, 33%). LCMS: 318 (M+H⁺).

The compounds below are prepared using the procedures from Example 63 and Example 64.

| Structure | Mw | MW from LCMS |
|---|---|---|
| 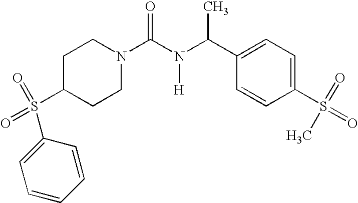 | 450.6 | 451 |
| 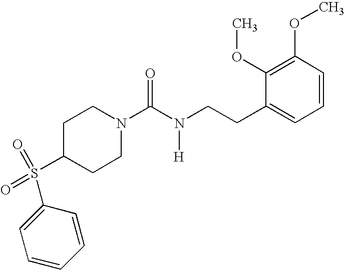 | 432.5 | 433 |
| 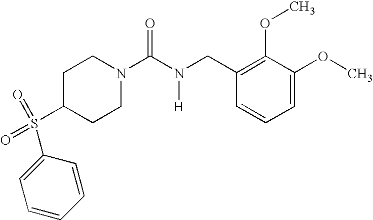 | 418.5 | 419 |
| 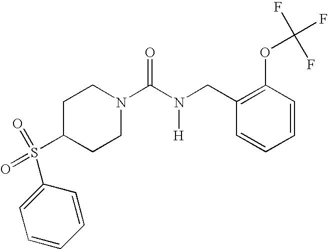 | 442.5 | 443 |
| 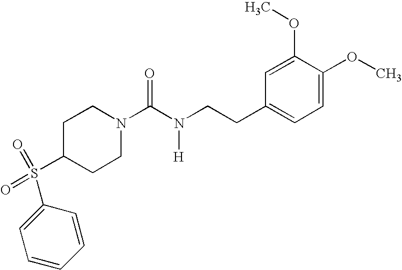 | 432.5 | 433 |
| 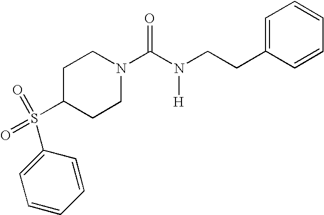 | 372.5 | 373 |
| 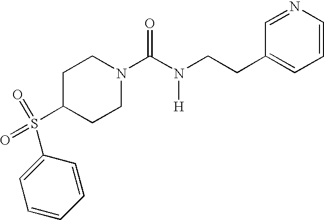 | 373.5 | 374 |
| 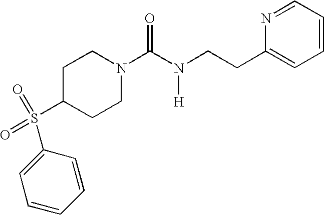 | 373.5 | 374 |
| 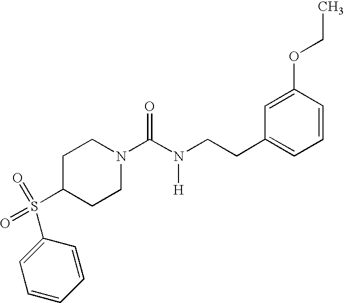 | 416.5 | 417 |
| 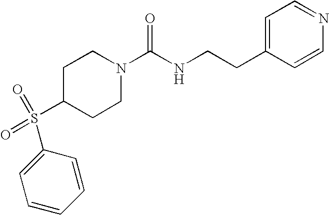 | 373.5 | 374 |
| 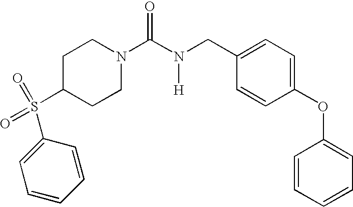 | 450.6 | 451 |
| 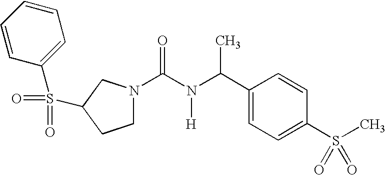 | 436.6 | 437 |
| 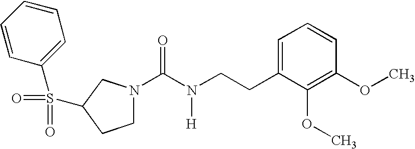 | 418.5 | 419 |
| 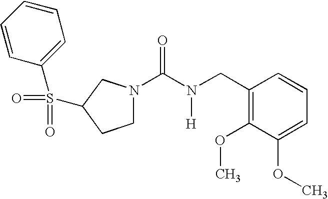 | 404.5 | 405 |
| 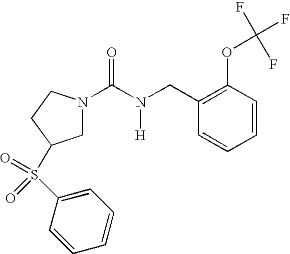 | 428.4 | 429 |
| 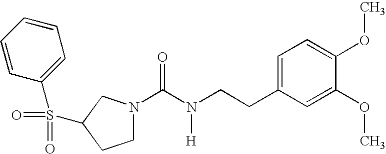 | 418.5 | 419 |
| 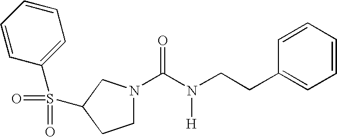 | 358.5 | 359 |
| 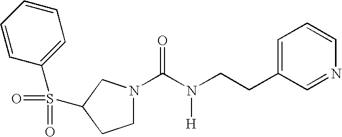 | 359.4 | 360 |
| 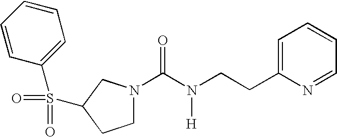 | 359.4 | 360 |
| 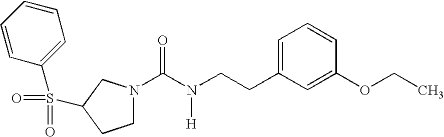 | 402.5 | 403 |
| 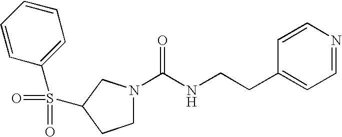 | 359.4 | 360 |
| 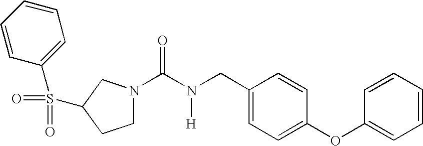 | 436.5 | 437 |
| 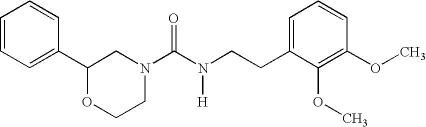 | 370.4 | 371 |
| 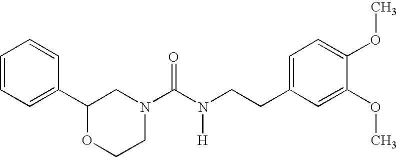 | 370.4 | 371 |
| 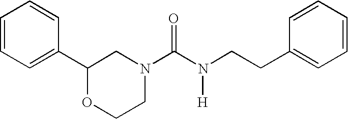 | 310.4 | 311 |
| 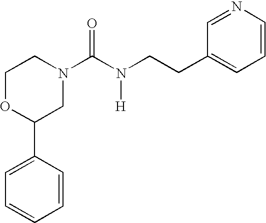 | 311.4 | 312 |
| 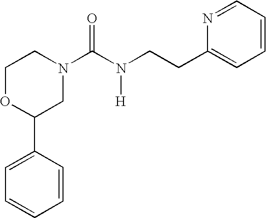 | 311.4 | 312 |
| 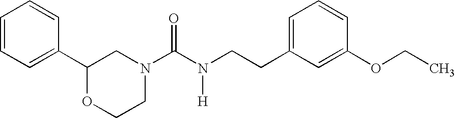 | 354.4 | 355 |
| 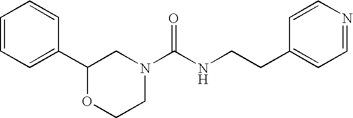 | 311.4 | 312 |
| 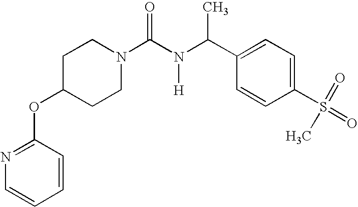 | 403.5 | 404 |
| 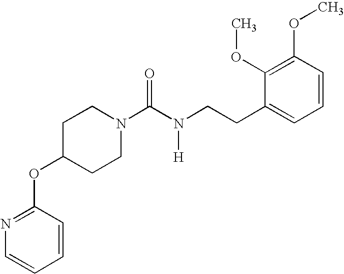 | 385.5 | 386 |
| 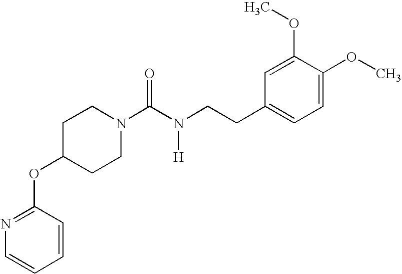 | 385.5 | 386 |
| 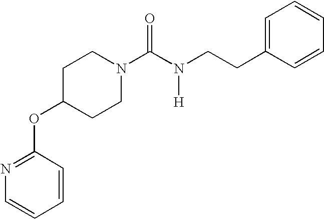 | 325.4 | 326 |
| 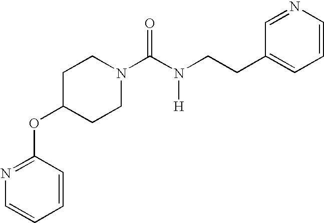 | 326.4 | 327 |
| 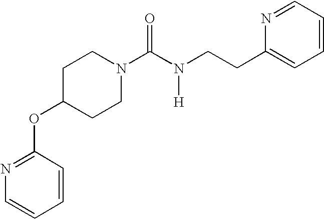 | 326.4 | 327 |
| 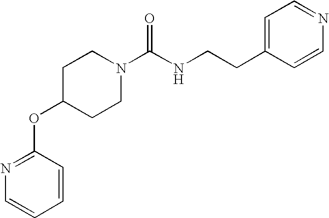 | 326.4 | 327 |
| 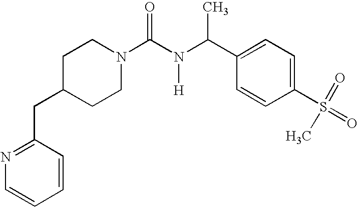 | 401.5 | 402 |
| 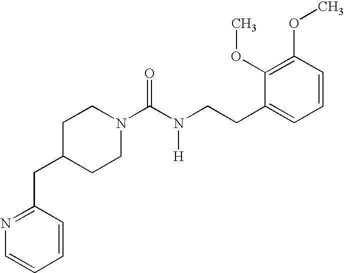 | 383.5 | 384 |
| 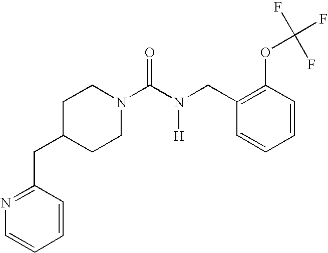 | 393.4 | 394 |
| 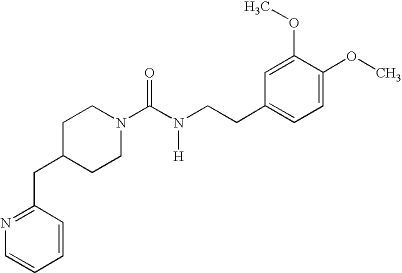 | 383.5 | 384 |
| 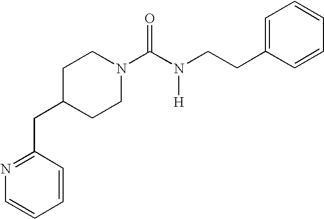 | 323.4 | 324 |
| 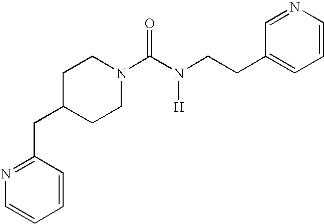 | 324.4 | 325 |
| 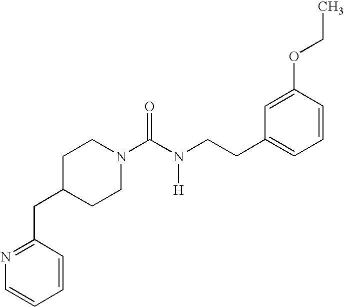 | 367.5 | 368 |
| 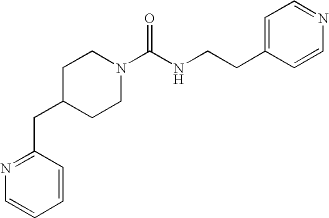 | 324.4 | 325 |
| 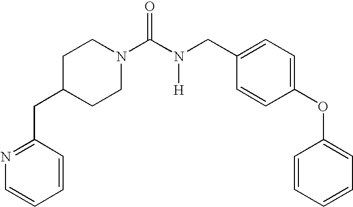 | 401.5 | 402 |
| 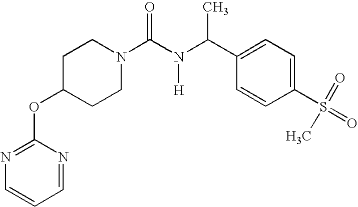 | 404.5 | 405 |
| 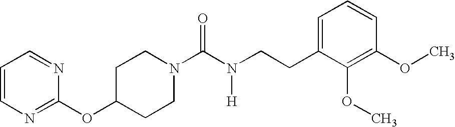 | 386.4 | 387 |
| 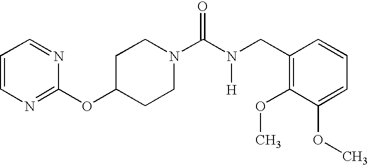 | 372.4 | 373 |
| 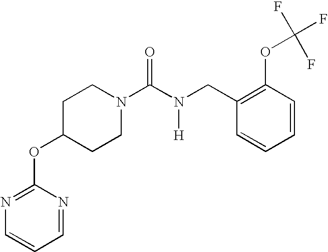 | 396.4 | 397 |
| 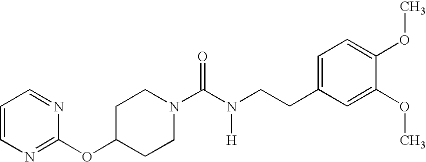 | 386.4 | 387 |
| 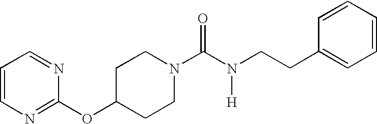 | 326.4 | 327 |
| 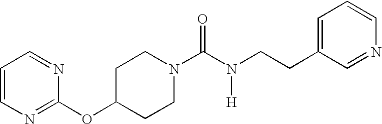 | 327.4 | 328 |
| 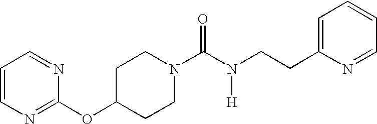 | 327.4 | 328 |
| 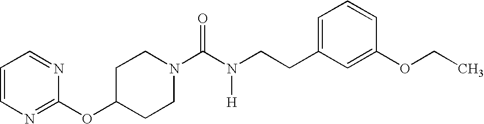 | 370.4 | 371 |
| 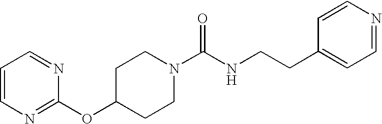 | 327.4 | 328 |
| 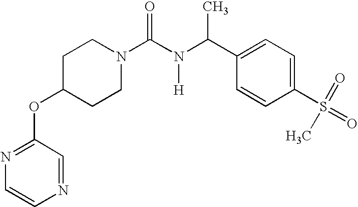 | 404.5 | 405 |
| 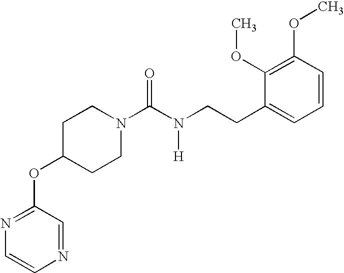 | 386.4 | 387 |
| 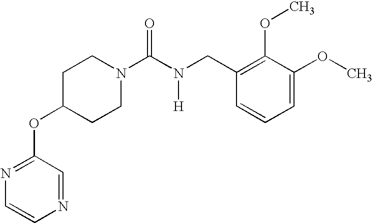 | 372.4 | 373 |
| 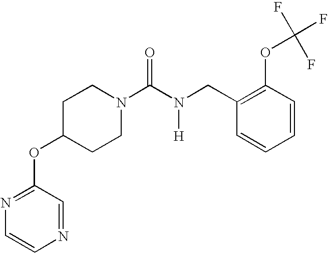 | 396.4 | 397 |
| 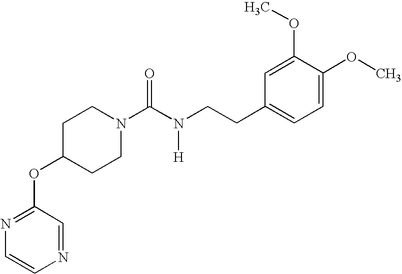 | 386.4 | 387 |
| 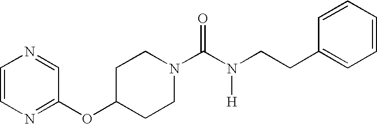 | 326.4 | 327 |
| 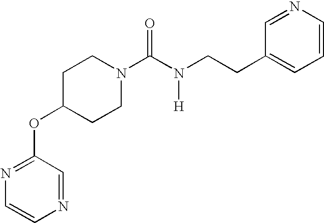 | 327.4 | 328 |
| 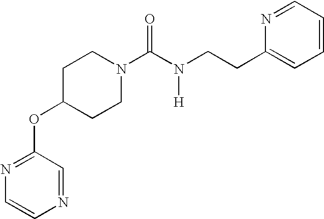 | 327.4 | 328 |
| 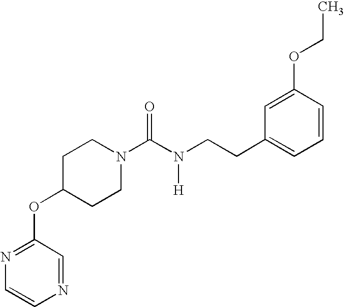 | 370.4 | 371 |
| 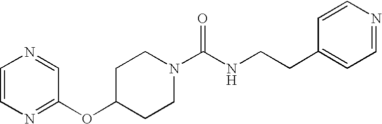 | 327.4 | 328 |
| 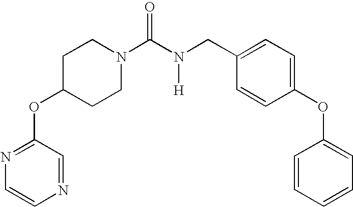 | 404.5 | 405 |
| 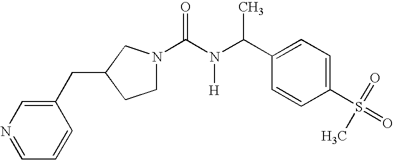 | 387.5 | 388 |
| 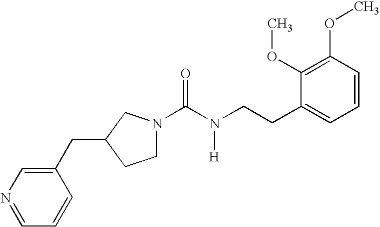 | 369.5 | 370 |
| 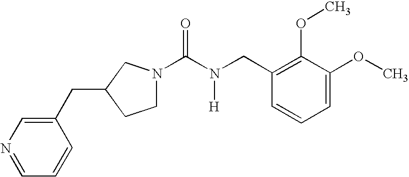 | 355.4 | 356 |
| 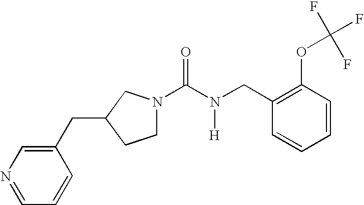 | 379.4 | 380 |
| 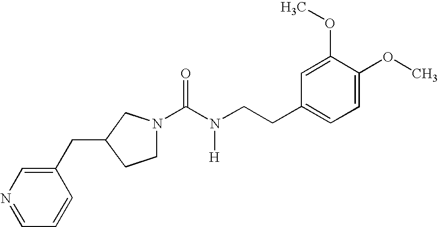 | 369.5 | 370 |
| 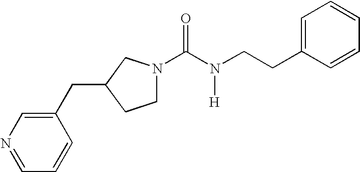 | 309.4 | 310 |
| 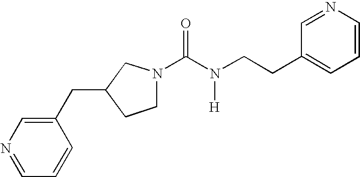 | 310.4 | 311 |
| 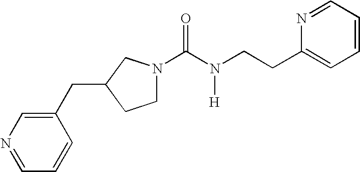 | 310.4 | 311 |
| 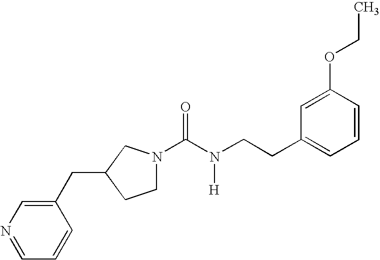 | 353.5 | 354 |
| 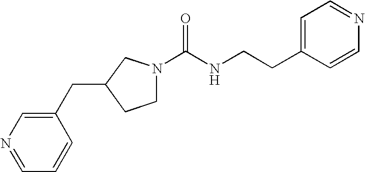 | 310.4 | 311 |
| 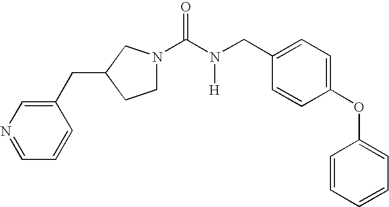 | 387.5 | 388 |
| 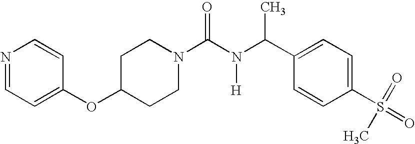 | 403.5 | 404 |
| 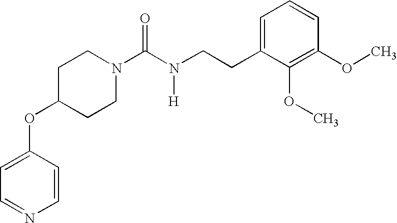 | 385.5 | 386 |
| 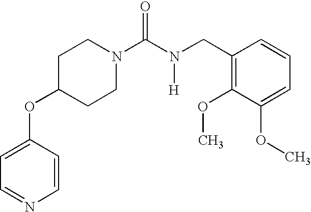 | 371.4 | 372 |
| 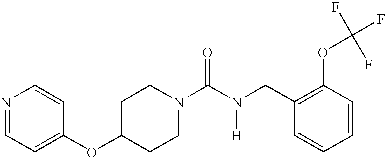 | 395.4 | 396 |
| 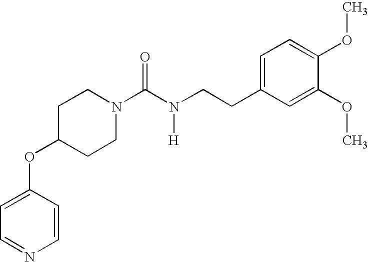 | 385.5 | 386 |
| 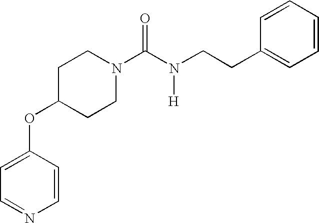 | 325.4 | 326 |
| 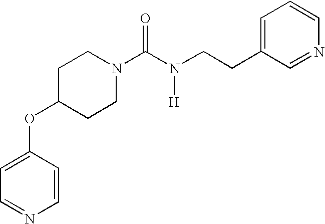 | 326.4 | 327 |
| 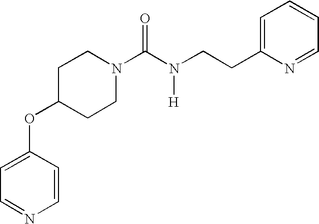 | 326.4 | 327 |
| 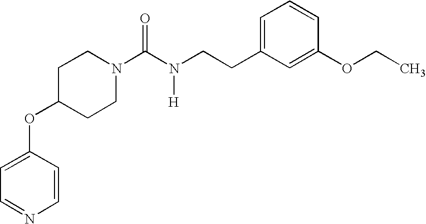 | 369.5 | 370 |
| 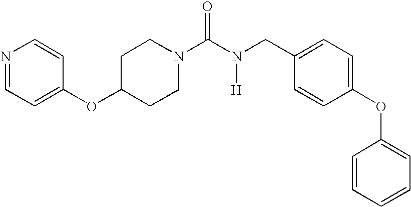 | 403.5 | 404 |
| 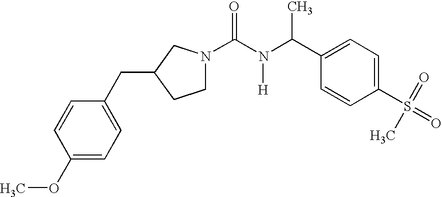 | 416.5 | 417 |
| 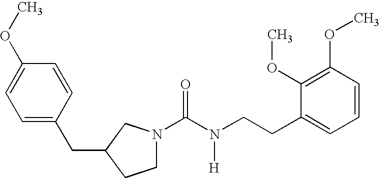 | 398.5 | 399 |
| 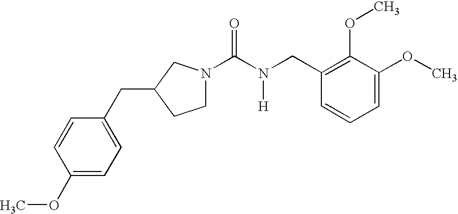 | 384.5 | 385 |
| 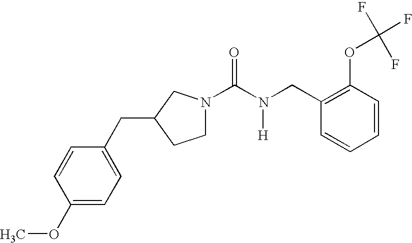 | 408.4 | 409 |
| 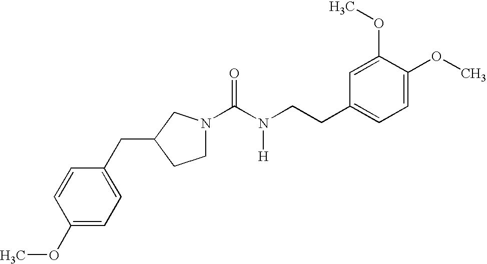 | 398.5 | 399 |
| 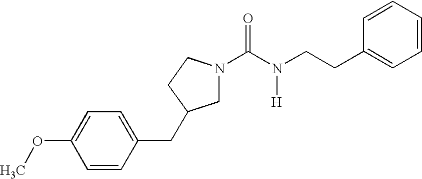 | 338.4 | 339 |
| 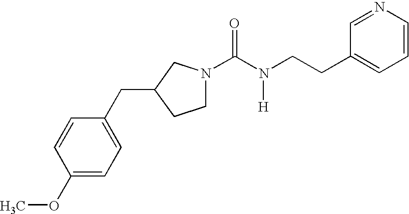 | 339.4 | 340 |
| 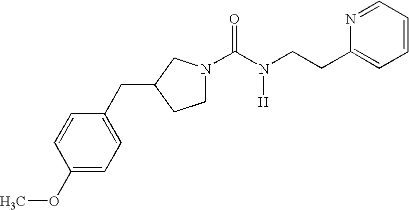 | 339.4 | 340 |
| 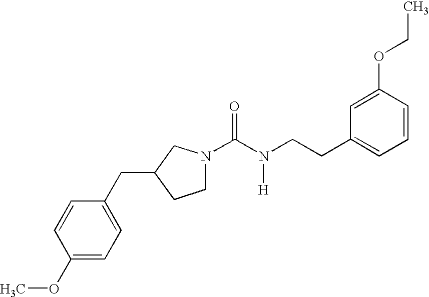 | 382.5 | 383 |
| 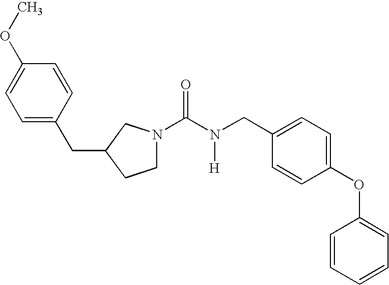 | 416.5 | 417 |
| 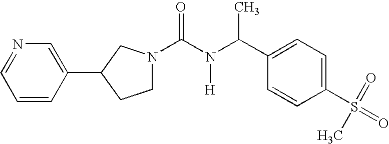 | 373.5 | 374 |
| 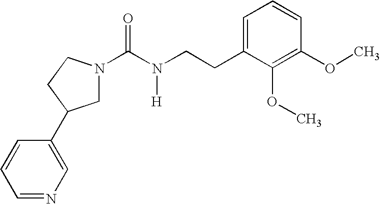 | 355.4 | 356 |
| 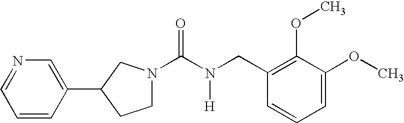 | 341.4 | 342 |
| 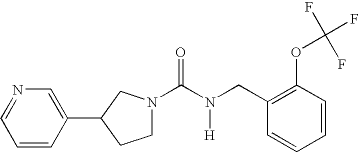 | 365.4 | 366 |
| 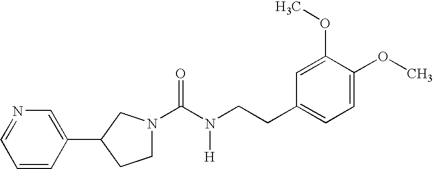 | 355.4 | 356 |
| 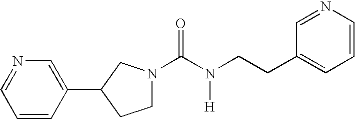 | 296.4 | 297 |
| 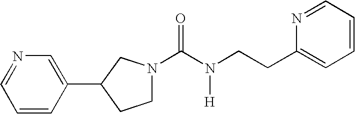 | 296.4 | 297 |
| 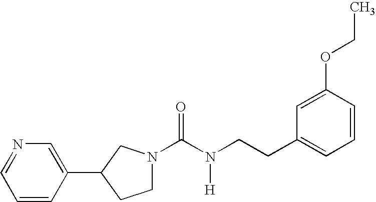 | 339.4 | 340 |
| 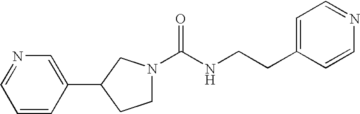 | 296.4 | 297 |
| 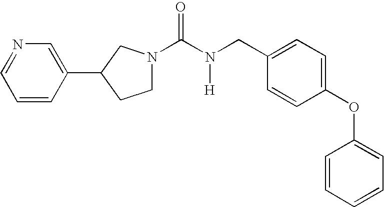 | 373.5 | 374 |
| 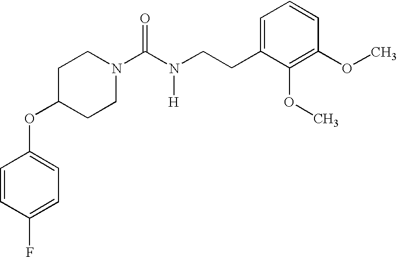 | 402.5 | 403 |
| 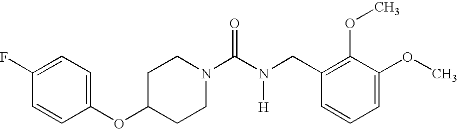 | 388.4 | 389 |
| 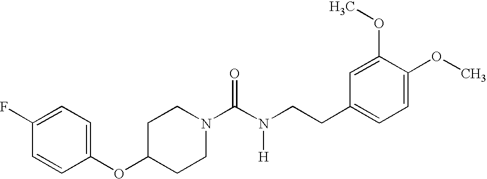 | 402.5 | 403 |
| 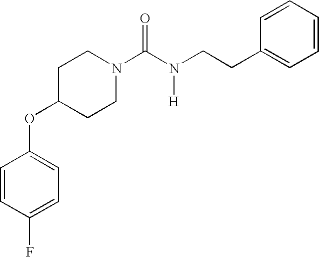 | 342.4 | 343 |
| 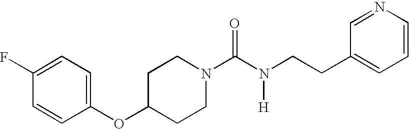 | 343.4 | 344 |
| 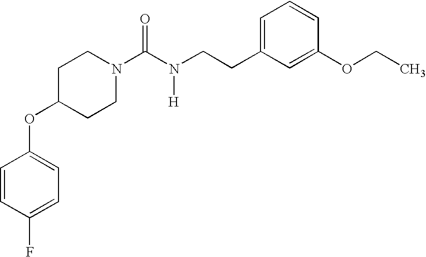 | 386.5 | 387 |
| 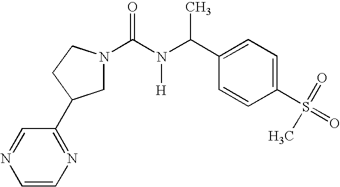 | 374.5 | 375 |
| 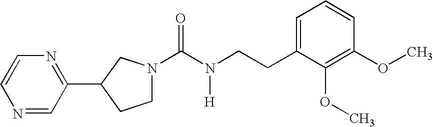 | 356.4 | 357 |
| 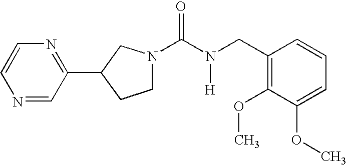 | 342.4 | 343 |
| 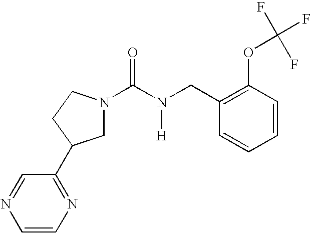 | 366.3 | 367 |
| 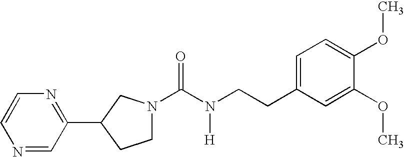 | 356.4 | 357 |
| 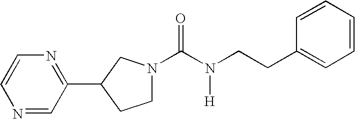 | 296.4 | 297 |
| 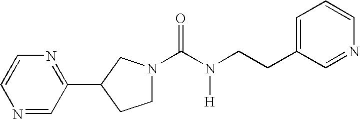 | 297.4 | 298 |
| 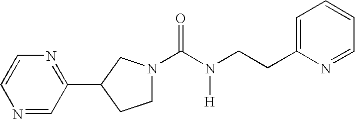 | 297.4 | 298 |
| 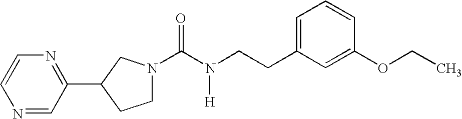 | 340.4 | 341 |
| 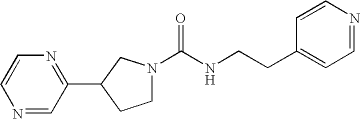 | 297.4 | 298 |
| 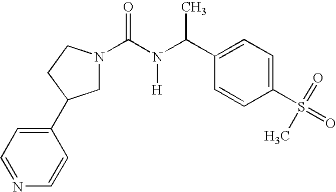 | 373.5 | 374 |
| 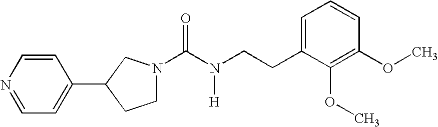 | 355.4 | 356 |
| 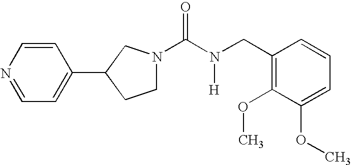 | 341.4 | 342 |
| 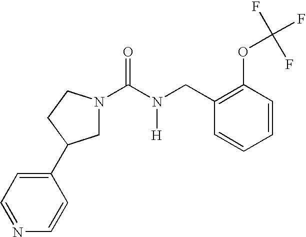 | 365.4 | 366 |
| 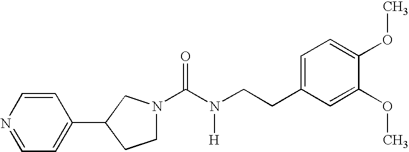 | 355.4 | 356 |
| 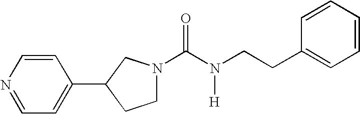 | 295.4 | 296 |
| 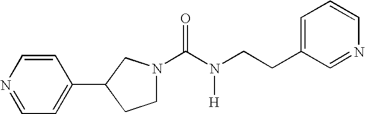 | 296.4 | 297 |
| 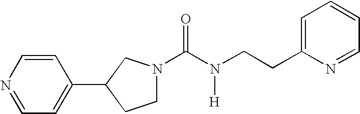 | 296.4 | 297 |
| 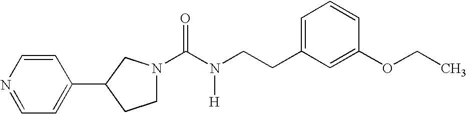 | 339.4 | 340 |
| 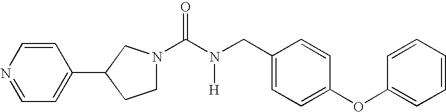 | 373.5 | 374 |
| 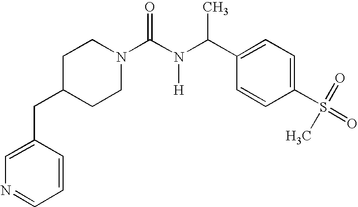 | 401.5 | 402 |
| 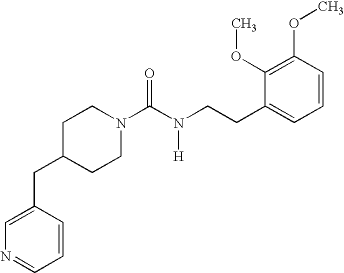 | 383.5 | 384 |
| 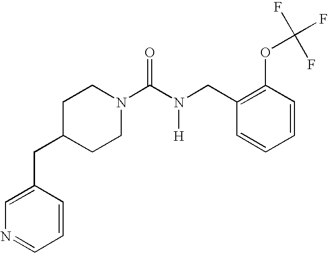 | 393.4 | 394 |
| 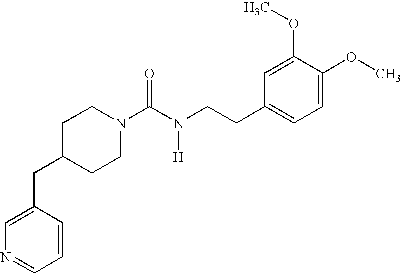 | 383.5 | 384 |
| 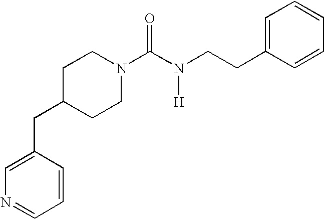 | 323.4 | 324 |
| 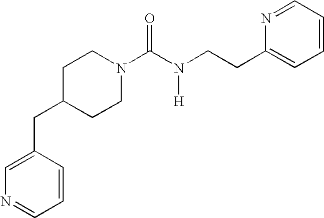 | 324.4 | 325 |
| 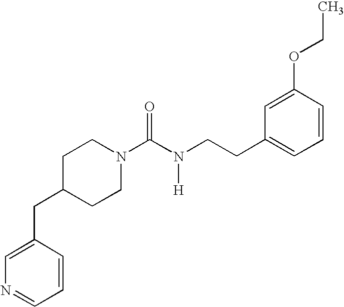 | 367.5 | 368 |
| 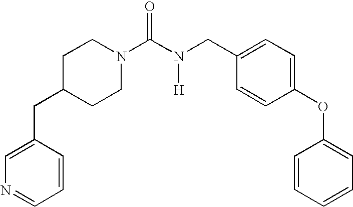 | 401.5 | 402 |
| 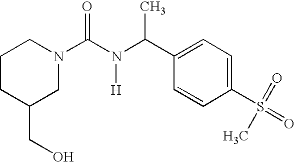 | 340.4 | 341 |
| 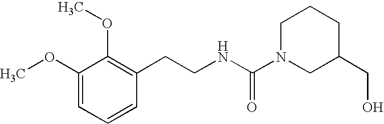 | 322.4 | 323 |
| 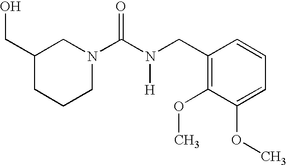 | 308.4 | 309 |
| 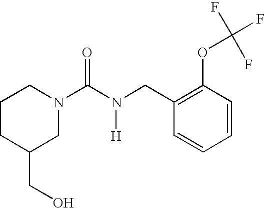 | 332.3 | 333 |
| 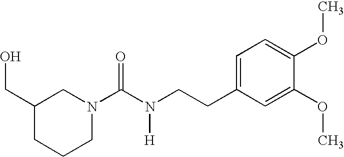 | 322.4 | 323 |
| 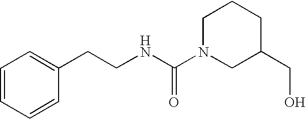 | 262.4 | 263 |
| 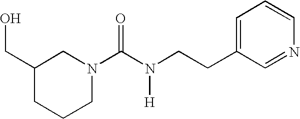 | 263.3 | 264 |
| 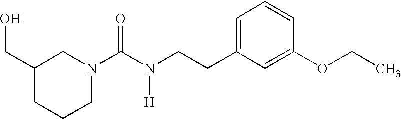 | 306.4 | 307 |
| 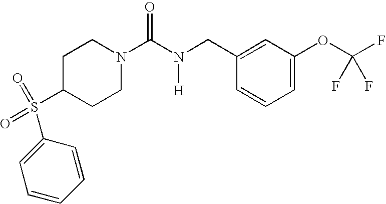 | 442.5 | 443 |
| 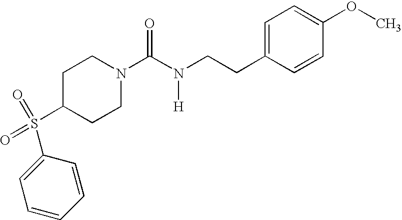 | 402.5 | 403 |
| 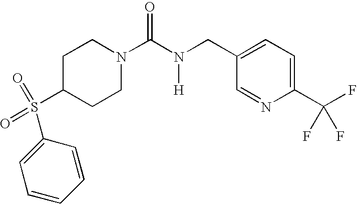 | 427.4 | 428 |
| 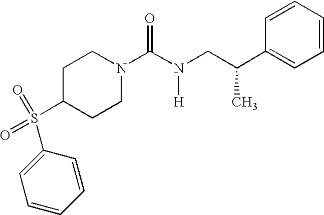 | 386.5 | 387 |
| 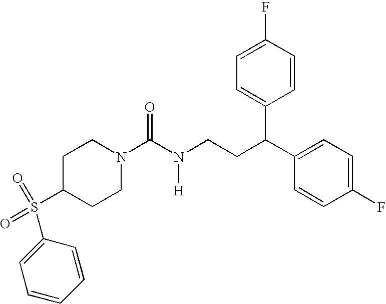 | 498.6 | 499 |
| 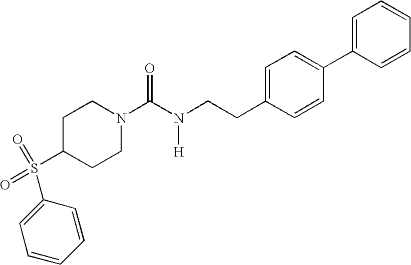 | 448.6 | 449 |
| 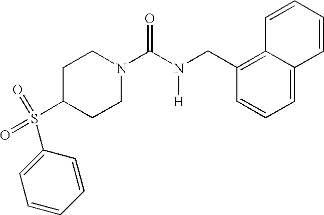 | 408.5 | 409 |
| 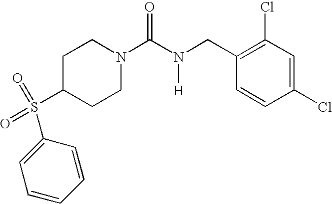 | 427.4 | 428 |
| 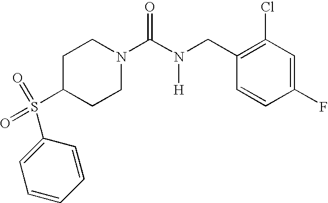 | 410.9 | 411 |
| 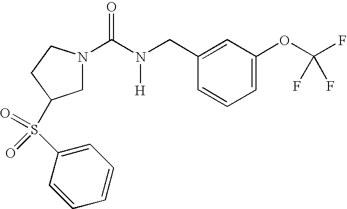 | 428.4 | 429 |
| 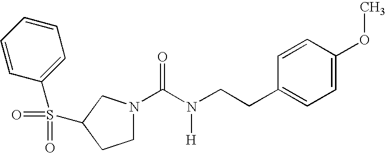 | 388.5 | 389 |
| 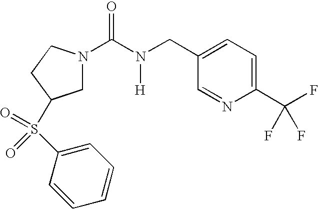 | 413.4 | 414 |
| 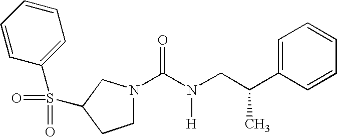 | 372.5 | 373 |
| 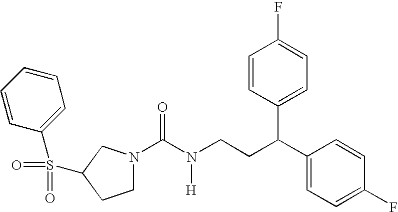 | 484.6 | 485 |
| 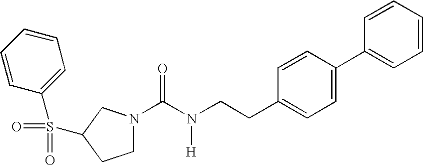 | 434.6 | 435 |
| 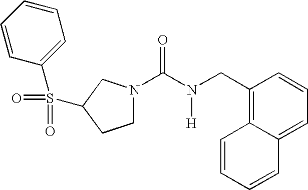 | 394.5 | 395 |
| 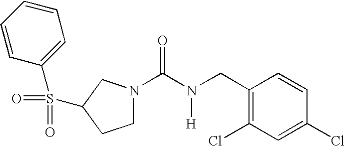 | 413.3 | 414 |
| 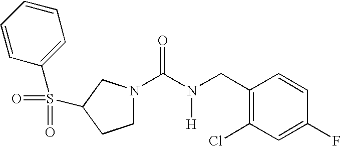 | 396.9 | 397 |
| 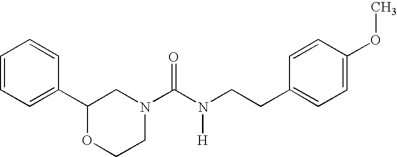 | 340.4 | 341 |
| 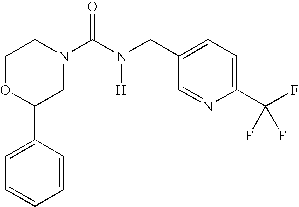 | 365.4 | 366 |
| 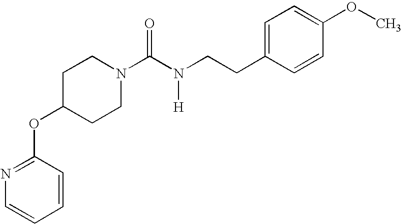 | 355.4 | 356 |
| 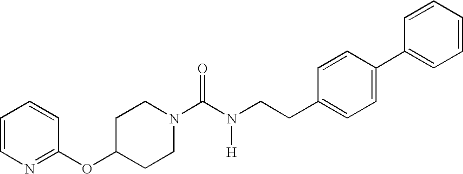 | 401.5 | 402 |
| 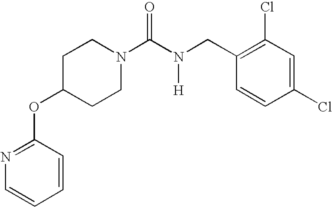 | 380.3 | 381 |
| 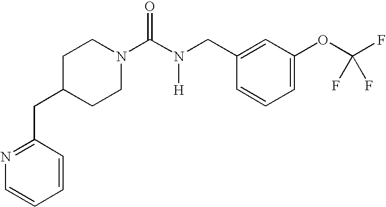 | 393.4 | 394 |
| 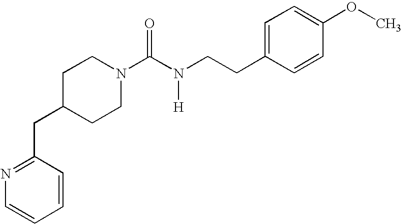 | 353.5 | 354 |
| 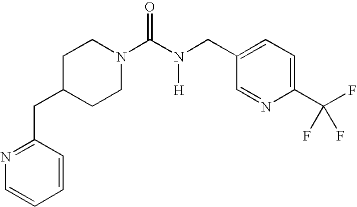 | 378.4 | 379 |
| 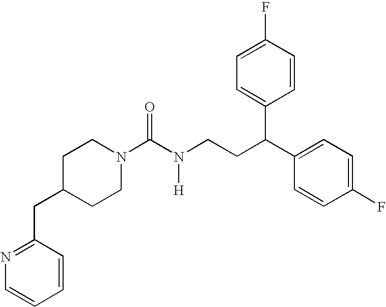 | 449.5 | 450 |
| 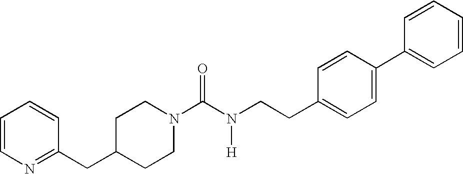 | 399.5 | 400 |
| 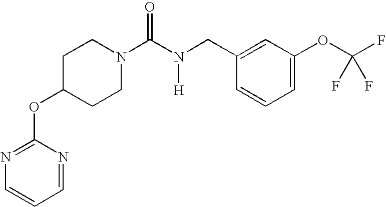 | 396.4 | 397 |
| 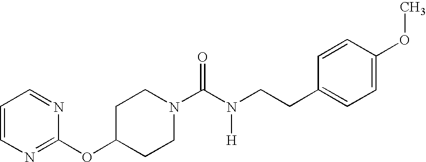 | 356.4 | 357 |
| 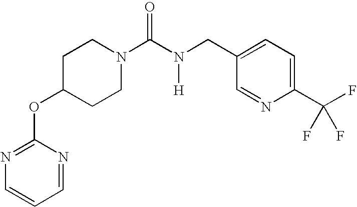 | 381.4 | 382 |
| 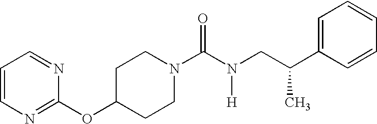 | 340.4 | 341 |
| 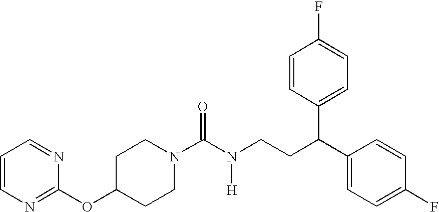 | 452.5 | 453 |
| 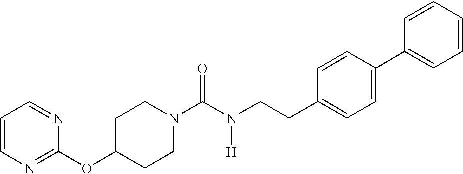 | 402.5 | 403 |
| 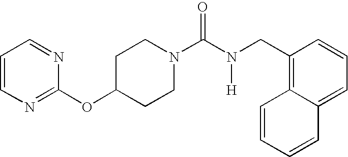 | 362.4 | 363 |
| 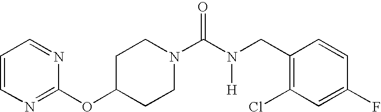 | 364.8 | 365 |
| 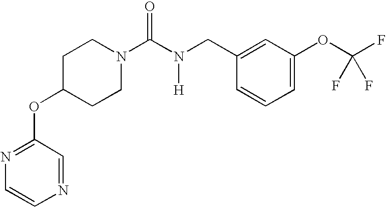 | 396.4 | 397 |
| 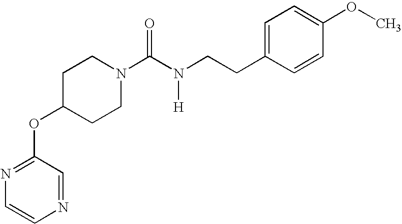 | 356.4 | 357 |
| 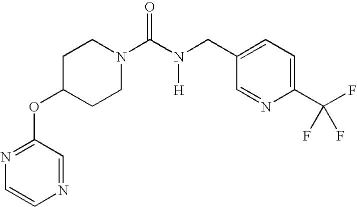 | 381.4 | 382 |
| 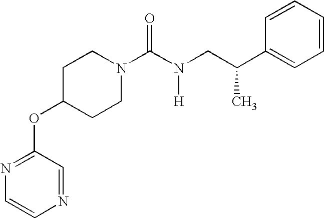 | 340.4 | 341 |
| 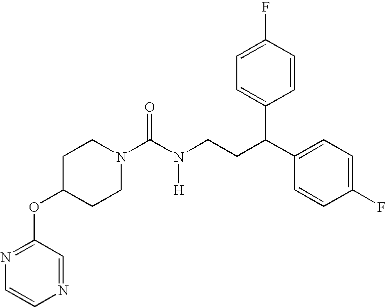 | 452.5 | 453 |
| 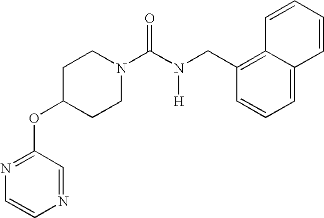 | 362.4 | 363 |
| 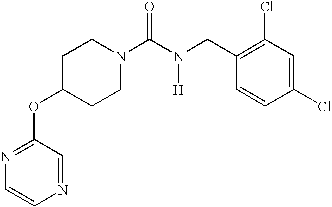 | 381.3 | 382 |
| 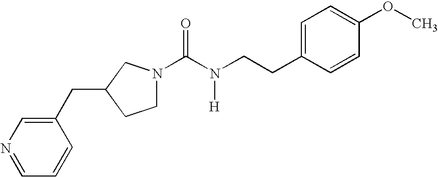 | 339.4 | 340 |
| 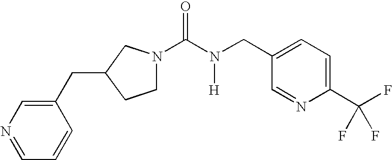 | 364.4 | 365 |
| 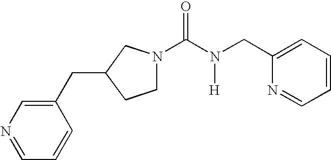 | 296.4 | 297 |
| 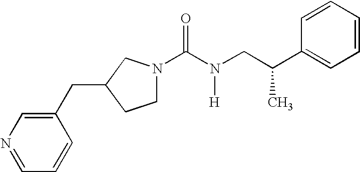 | 323.4 | 324 |
| 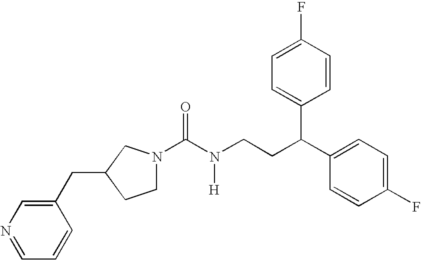 | 435.5 | 436 |
| 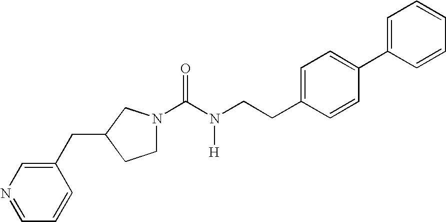 | 385.5 | 386 |
| 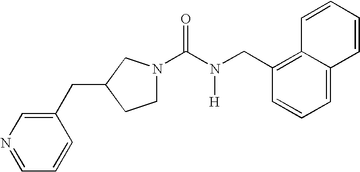 | 345.4 | 346 |
| 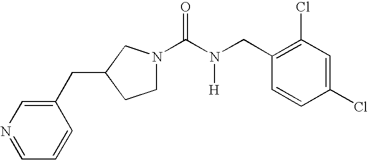 | 364.3 | 365 |
| 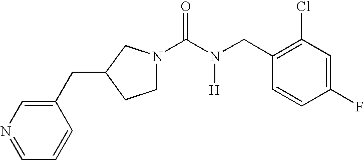 | 347.8 | 348 |
| 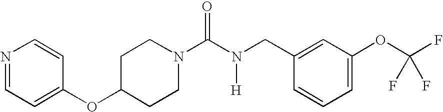 | 395.4 | 396 |
| 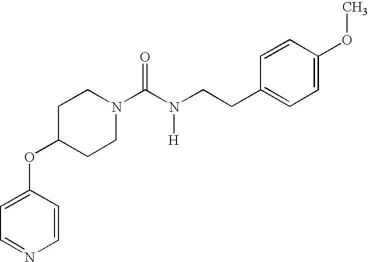 | 355.4 | 356 |
| 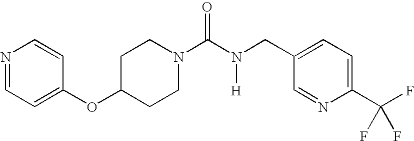 | 380.4 | 381 |
| 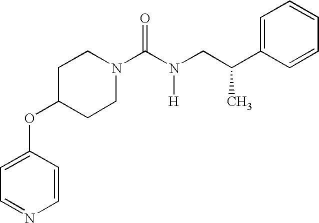 | 339.4 | 340 |
| 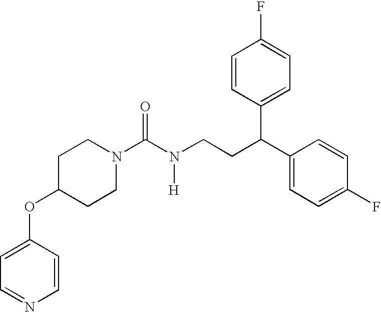 | 451.5 | 452 |
| 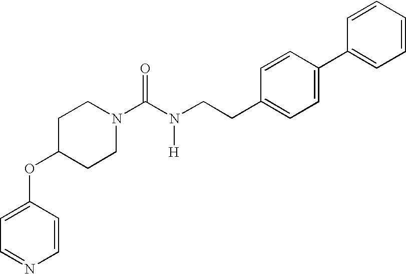 | 401.5 | 402 |
| 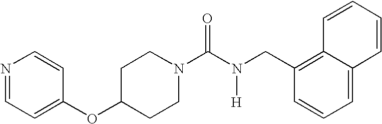 | 361.4 | 362 |
| 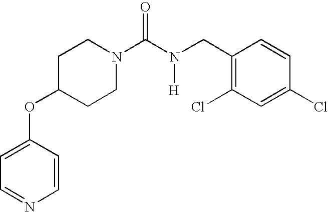 | 380.3 | 381 |
| 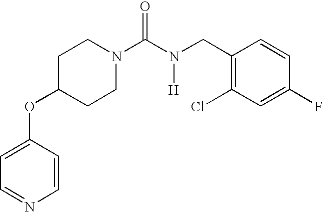 | 363.8 | 364 |
| 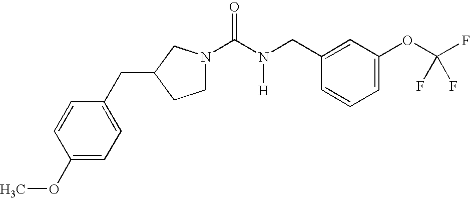 | 408.4 | 409 |
| 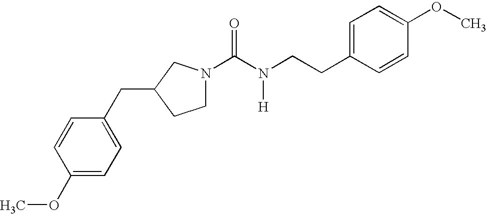 | 368.5 | 369 |
| 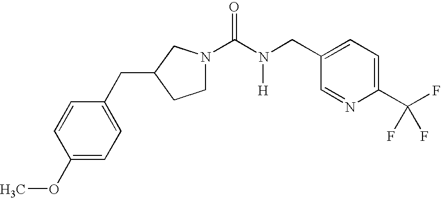 | 393.4 | 394 |
| 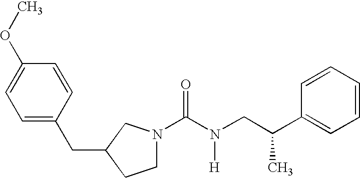 | 352.5 | 353 |
| 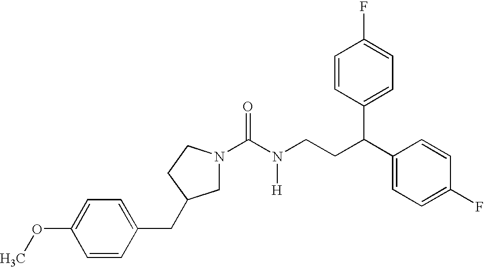 | 464.6 | 465 |
| 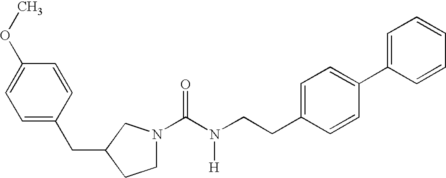 | 414.5 | 415 |
| 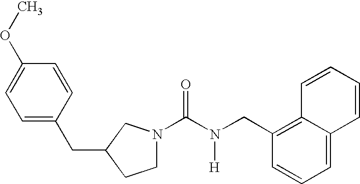 | 374.5 | 375 |
| 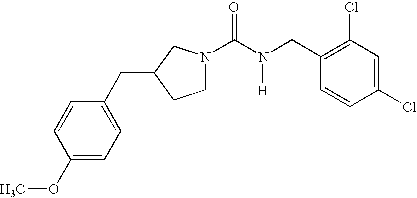 | 393.3 | 394 |
| 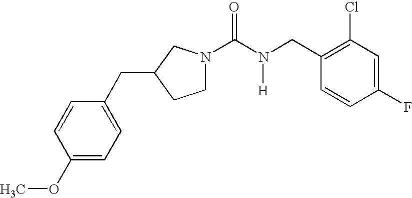 | 376.9 | 377 |
| 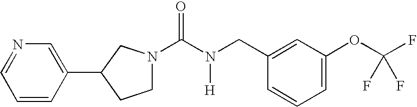 | 365.4 | 366 |
| 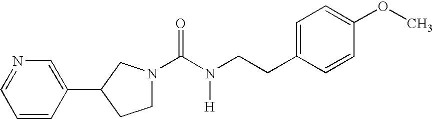 | 325.4 | 326 |
| 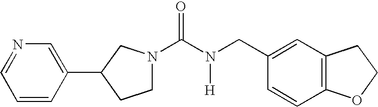 | 323.4 | 324 |
| 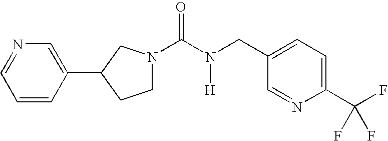 | 350.3 | 351 |
| 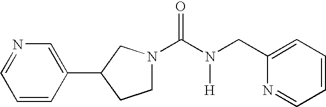 | 282.3 | 283 |
| 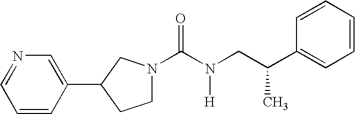 | 309.4 | 310 |
| 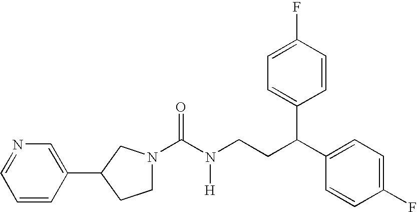 | 421.5 | 422 |
| 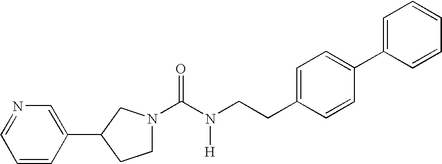 | 371.5 | 372 |
| 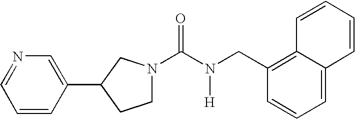 | 331.4 | 332 |
| 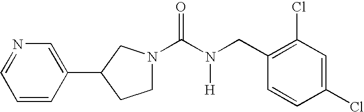 | 350.2 | 351 |
| 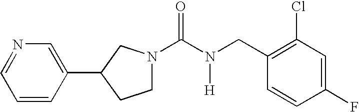 | 333.8 | 334 |
| 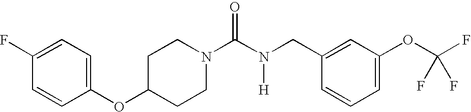 | 412.4 | 413 |
| 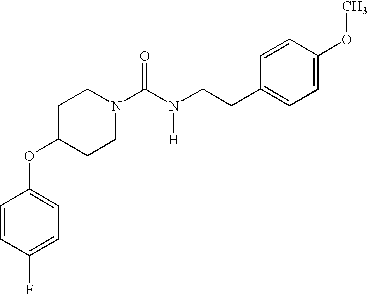 | 372.4 | 373 |
| 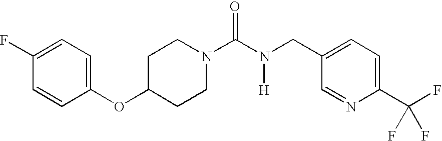 | 397.4 | 398 |
| 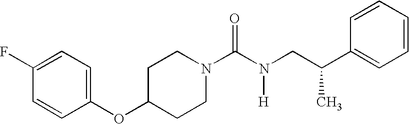 | 356.4 | 357 |
| 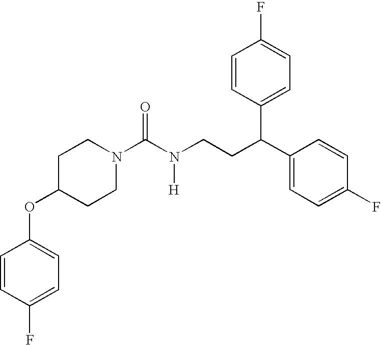 | 468.5 | 469 |
| 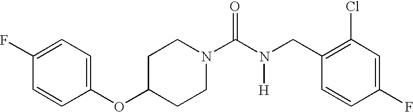 | 380.8 | 381 |
| 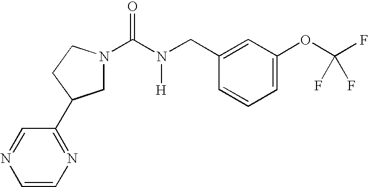 | 366.3 | 367 |
| 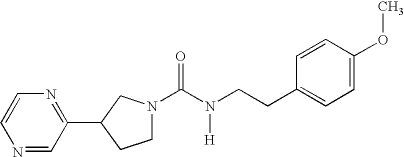 | 326.4 | 327 |
| 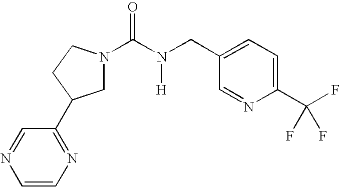 | 351.3 | 352 |
| 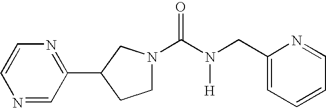 | 283.3 | 284 |
| 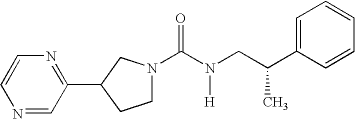 | 310.4 | 311 |
| 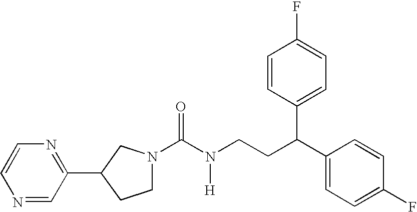 | 422.5 | 423 |
| 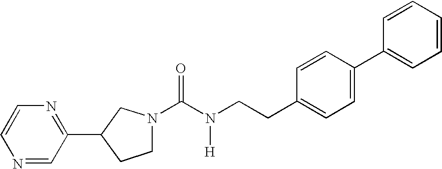 | 372.5 | 373 |
| 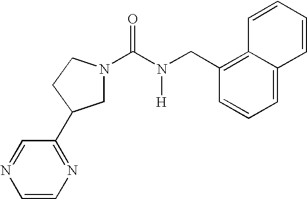 | 332.4 | 333 |
| 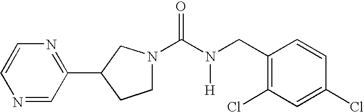 | 351.2 | 352 |
| 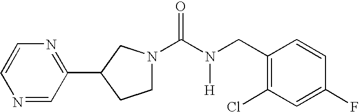 | 334.8 | 335 |
| 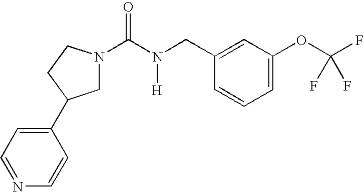 | 365.4 | 366 |
| 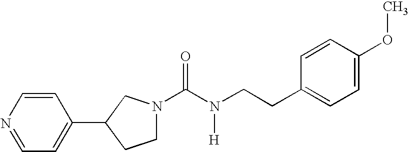 | 325.4 | 326 |
| 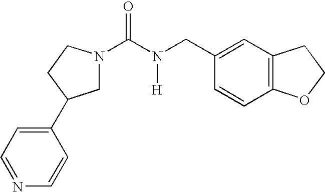 | 323.4 | 324 |
| 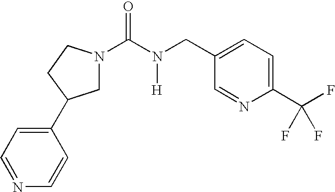 | 350.3 | 351 |
| 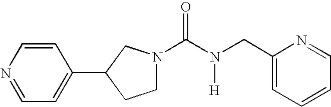 | 282.3 | 283 |
| 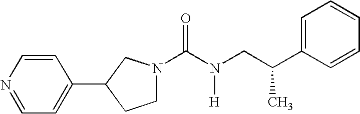 | 309.4 | 310 |
| 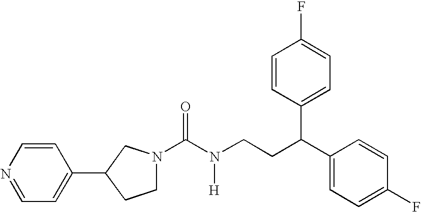 | 421.5 | 422 |
| 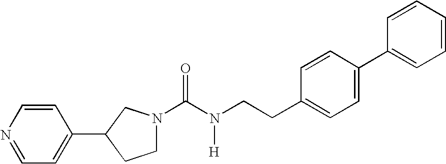 | 371.5 | 372 |
| 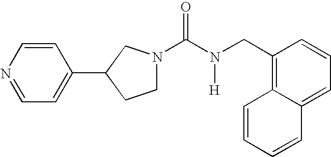 | 331.4 | 332 |
| 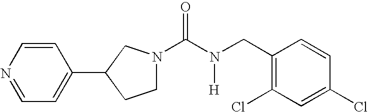 | 350.2 | 351 |
| 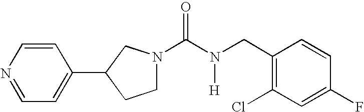 | 333.8 | 334 |
| 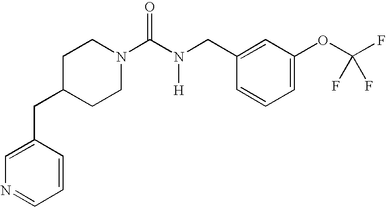 | 393.4 | 394 |
| 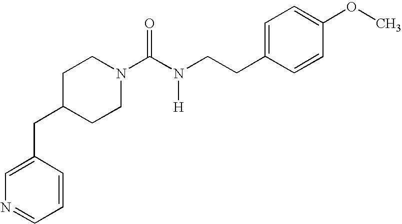 | 353.5 | 354 |
| 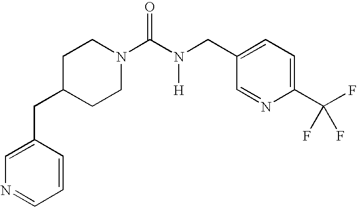 | 378.4 | 379 |
| 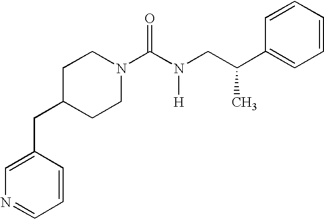 | 337.5 | 338 |
| 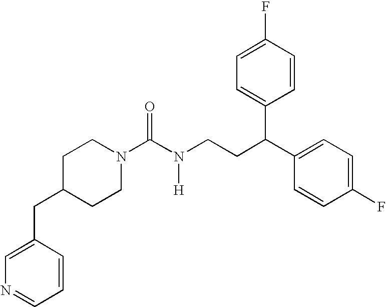 | 449.5 | 450 |
| 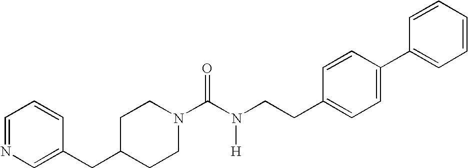 | 399.5 | 400 |
| 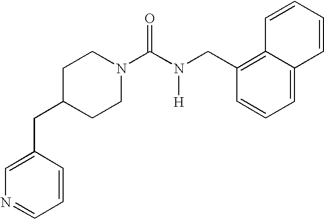 | 359.5 | 360 |
| 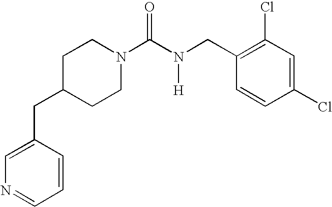 | 378.3 | 379 |
| 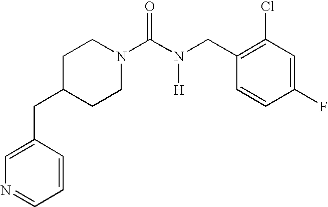 | 361.8 | 362 |
| 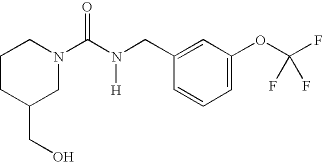 | 332.3 | 333 |
| 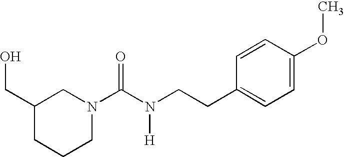 | 292.4 | 293 |
| 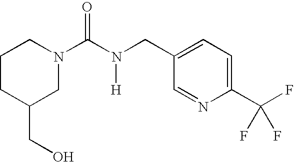 | 317.3 | 318 |
| 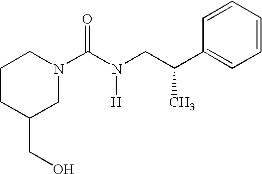 | 276.4 | 277 |
| 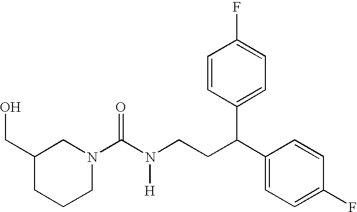 | 388.5 | 389 |
| 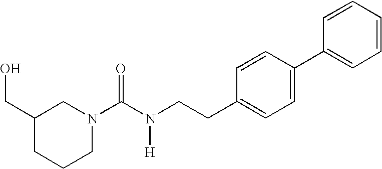 | 338.4 | 339 |
| 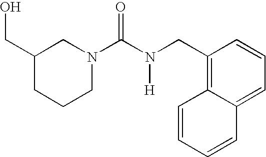 | 298.4 | 299 |
| 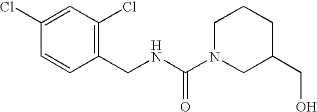 | 317.2 | 318 |
| 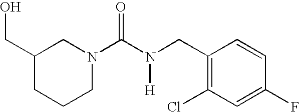 | 300.8 | 301 |

### Methods of Use

In accordance with the invention, there are provided methods of using the compounds as described herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

### In Vitro Assay for Inhibition of hsEH

This high throughput screen identifies compounds that inhibit the interaction of human soluble epoxide hydrolase (sEH) with a tetramethyl rhodamine (TAMRA)-labeled probe. The UHTS employs the Zymark Allegro modular robotic system to dispense reagents, buffers, and test compounds into either 96-well or 384-well black microtiter plates (from Costar). The assay buffer is: 20 mM TES, 200 mM NaCl, 0.05% w/v CHAPS, 1 mM TCEP, pH=7.0. Test compounds dissolved in neat DMSO at 5 mg/mL are diluted to 0.5 mg/mL in neat DMSO. The 0.5 mg/mL solutions are further diluted to 30 μg/mL in assay buffer containing DMSO such that the final concentration of DMSO is 30%. For 384-well format, a mixture of 10.35 nM human sEH and 2.59 nM probe is prepared in assay buffer and 60 μL is added to each well for a final sEH concentration of 10 nM and a final probe concentration of 2.5 nM. 2.1 μL of diluted test compound is then added to each well, where the final assay concentration will be 1 μg/mL test compound and 1% DMSO. The final volume in each well is 62.1 μL. Positive controls are reaction mixtures containing no test compound; negative controls (blanks) are reaction mixtures containing 3 μM BI00611349XX. For 96-well format, the final concentration of all reaction components remains the same. 135 μL sEH/probe mixture is added to wells containing 15 μL test compound so that the final well volume is 150 mL. After incubating the reaction for 30 minutes at room temperature, the plates are read for fluorescence polarization in the LJL Analyst set to 530 nm excitation, 580 nm emission, using the Rh 561 dichroic mirror.

### In Vitro Assay for Inhibition of rsEH

This screen identifies compounds that inhibit the interaction of rat soluble epoxide hydrolase (sEH) with a tetramethyl rhodamine (TAMRA)-labeled probe. The assay employs a Multimek, a Multidrop, and manual multi-channel pipettors to dispense reagents, buffers, and test compounds into 96-well black microtiter plates (Costar 3792). The assay buffer is: 20 mM TES, 200 mM NaCl, 0.05% w/v CHAPS, 1 mM TCEP, pH=7.0. Test compounds dissolved in neat DMSO at 10 mM are diluted to 1.5 mM in neat DMSO. The 1.5 mM solutions are serially diluted using 3-fold dilutions in neat DMSO in polypropylene plates. Assay buffer is added to the wells such that the compounds are diluted 10-fold and the DMSO concentration is 10%. A mixture of 11.1 nM rat sEH and 2.78 nM probe is prepared in assay buffer. 15 uL of diluted test compound is added to each well, where the final maximum assay concentration will be 3 uM test compound and 1% DMSO. 135 uL of sEH/probe mixture is added to each well for a final sEH concentration of 10 nM and a final probe concentration of 2.5 nM. The final volume in each well is 150 uL. Positive controls are reaction mixtures containing no test compound; negative controls (blanks) are reaction mixtures containing 3 uM BT00611349XX. After incubating the reaction for 30 minutes at room temperature, the plates are read for fluorescence polarization in the LJL Analyst set to 530 nm excitation, 580 nm emission, using the Rh 561 dichroic mirror.

1. A compound of the formula (I): 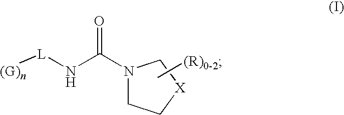
wherein:
G is carbocycle, heteroaryl or heterocyclyl optionally substituted by one or more Y;
n is 1 or 2 such that L can be substituted with one to two G;
L is a methylene or ethylene linking group optionally substituted by hydroxy, amino, lower alkoxy, lower alkylamino, lower alkylthio or 1-3 fluorine atoms;
X is a bond, methylene or ethylene;
R if present is chosen from:
i) —C(O)—R₁;
R₁ is chosen from —OH, —O(CH₂)₀₋₅—CH₃, —NR₂R₃, carbocycle, heteroaryl or heterocyclyl;
ii) carbocycle, heteroaryl or heterocyclyl optionally substituted by one or more R₄;
iii) —W-Q, wherein:
W is chosen from alkylene, O, S, NH—S(O)₂— and NH;
Q is chosen from OH, alkyl, carbocycle, heteroaryl and heterocyclyl optionally substituted by one or more R₅;
iv) lower alkyl;
Y is chosen from
halogen, lower alkyl, lower alkoxy each optionally halogenated, aryloxy, sulfone, nitrile, or Y is carbocycle optionally substituted by one to three oxo, lower acyl, halogen, nitrile, lower alkylS(O)ₘ—, lower alkylS(O)ₘ—NH—, lower alkoxycarbonyl, NR₂R₃—C(O)—, —NR₂R₃, lower alkyl, C₃₋₆ cycloalkylC₀₋₂alkyl, hydroxy, lower alkoxy or arylC₀₋₄ alkyl the aryl group being optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl or lower alkyl optionally substituted by lower alkylS(O)ₘ—, lower alkoxy, hydroxy or mono or diC₁₋₃ alkyl amino;
or R₂ and R₃ optionally combine with the nitrogen atom to which they are attached to form a heterocyclic ring;
each R₄ and R₅ are independently nitrile, hydroxy, lower alkylS(O)ₘ—, carboxy, halogen, lower alkoxy, arylC₀₋₄ alkyl, heteroaryl C₀₋₄ alkyl, heterocycle C₀₋₄alkyl, C₁₋₂ acyl, aroyl, lower alkyl optionally substituted by lower alkylS(O)ₘ—, lower alkoxy or hydroxy, —C(O)—NH₂ or —S(O)ₘ—NH₂ wherein each case the N atom is optionally substituted by lower-alkyl; each R₄ and R₅ are optionally halogenated;
m is 0, 1 or 2;
or the pharmaceutically acceptable salts thereof.

2. The compound according to claim 1, and wherein:
X is ethylene;
R if present is chosen from:
i) —C(O)—R₁;
R₁ is chosen from —OH, —NR₂R₃, phenyl, C3-6 cycloalkyl and heteroaryl chosen from pyrimidinyl, pyridinyl, pyridazinyl, pyrazinyl, pyranyl, pyrrolyl, pyrazolyl, imidazolyl, furanyl, oxazolyl, thienyl and thiazolyl;
ii) phenyl, heteroaryl or heterocyclyl optionally substituted by one or more R₄;
iii) —W-Q, wherein:
W is chosen from methylene, ethylene and O;
Q is chosen from OH, —O(CH₂)₀₋₂—CH₃, methyl, phenyl, heteroaryl chosen from pyrimidinyl, pyridinyl, pyridazinyl, pyrazinyl, pyranyl, pyrrolyl, pyrazolyl, imidazolyl, furanyl, oxazolyl, thienyl and thiazolyl, optionally substituted by one or more R₅;
iv) lower alkyl;
Y is chosen from
aryloxy, sulfone, nitrile, halogen, lower alkyl, lower alkoxy each optionally halogenated or Y is phenyl or C₃₋₆ cycloalkyl each optionally substituted by C₃₋₆ cycloalkylC₀₋₂alkyl or arylC₀₋₄ alkyl the cycloalkyl or aryl group being optionally substituted by one to three hydroxy, lower alkyl or lower alkoxy;
each R₂ and R₃ are independently hydrogen, phenylC₀₋₂ alkyl, heteroaryl C₀₋₂ alkyl, heterocycle C₀₋₂alkyl or lower alkyl optionally substituted by lower alkylS(O)ₘ—, lower alkoxy or hydroxy;
each R₄ and R₅ are independently nitrile, hydroxy, lower alkylS(O)ₘ—, carboxy, halogen, lower alkoxy, phenylC₀₋₂ alkyl, heteroaryl C₀₋₂ alkyl, heterocycle C₀₋₂alkyl, lower alkyl optionally substituted by lower alkylS(O)ₘ—, lower alkoxy or hydroxyl or hydroxy, —C(O)—NH₂ or —S(O)ₘ—NH₂ wherein each case the N atom is optionally substituted by lower-alkyl; each R₄ and R₅ are optionally halogenated.

3. The compound according to claim 2, and wherein:
G is phenyl, C3-8 cycloalkyl, bicycloheptane [2.2.1], bicyclo[2.2.1]5-heptene or adamantyl optionally substituted by one or more Y;
L is a methylene linking group optionally substituted by hydroxy, amino, lower alkoxy, lower alkylamino, lower alkylthio or 1-3 fluorine atoms;
R if present is chosen from:
i) —C(O)—R₁;
R₁ is chosen from —OH, —NR₂R₃, phenyl, C3-6 cycloalkyl and heteroaryl chosen from pyrimidinyl, pyridinyl, pyridazinyl and pyrazinyl;
ii) phenyl, morpholino, piperidinyl, benzimidazolyl or pyridinyl optionally substituted by one or more R₄;
iii) —W-Q, wherein:
W is chosen from methylene, ethylene and O;
Q is chosen from OH, —O(CH₂)₀₋₂—CH₃, methyl, phenyl, heteroaryl chosen from pyrimidinyl, pyridinyl, pyridazinyl and pyrazinyl, optionally substituted by one or more R₅;
iv) lower alkyl;
Y is chosen from
Cl, F, —CH₃, —O—CF₃, —O—CH₃, phenoxy or phenyl;
each R₂ and R₃ are independently hydrogen, pyridinylmethyl, tetrahydropyranylethyl, pyrrolidinylethyl, benzodioxanylmethyl, or lower alkyl optionally substituted by lower alkylS(O)ₘ— or lower alkoxy;
each R₄ and R₅ are independently Cl, F, lower alkoxy, phenyl and —CF₃.

4. A compound of the formula (Ia): 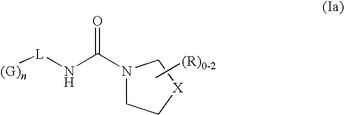
wherein for the Formula (Ia), the component 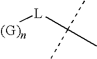
is chosen from A1-A67 in the table I below; in combination with any component 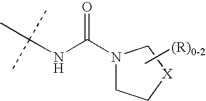
chosen from B1-B97 in the table I below;
TABLE I

| | |
|---|---|
| A | 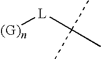 |
| A1 | 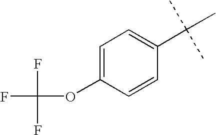 |
| A2 | 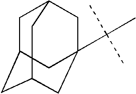 |
| A3 | 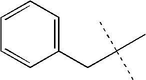 |
| A4 | 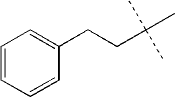 |
| A5 | 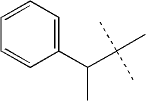 |
| A6 | 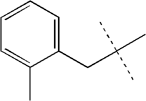 |
| A7 | 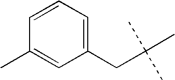 |
| A8 | 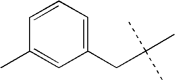 |
| A9 | 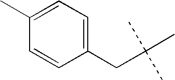 |
| A10 | 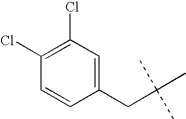 |
| A11 | 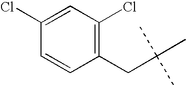 |
| A12 | 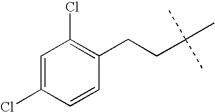 |
| A13 | 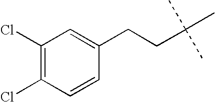 |
| A14 | 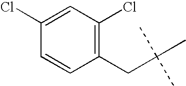 |
| A15 | 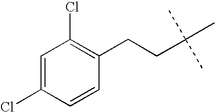 |
| A16 | 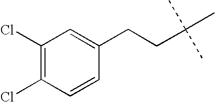 |
| A17 | 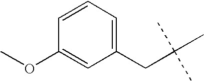 |
| A18 | 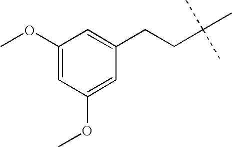 |
| A19 | 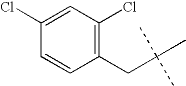 |
| A20 | 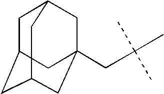 |
| A21 | 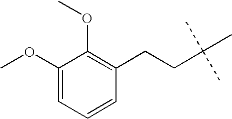 |
| A22 | 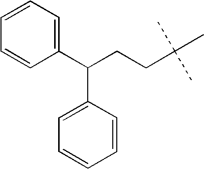 |
| A22 | 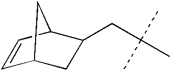 |
| A23 | 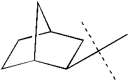 |
| A24 | 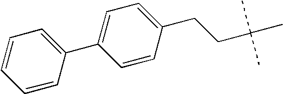 |
| A25 | 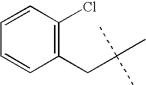 |
| A26 | 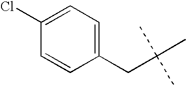 |
| A27 | 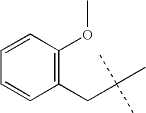 |
| A28 | 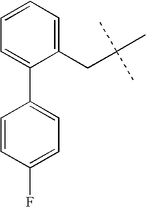 |
| A29 | 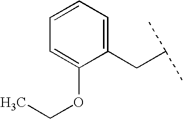 |
| A30 | 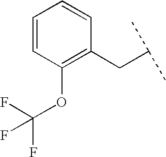 |
| A31 | 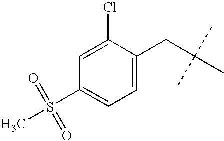 |
| A32 | 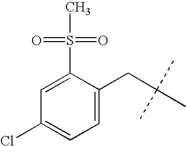 |
| A33 | 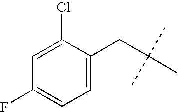 |
| A34 | 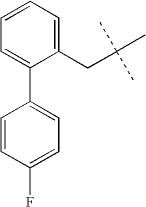 |
| A35 | 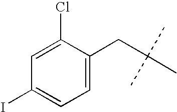 |
| A36 | 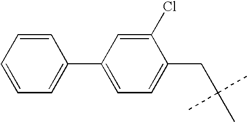 |
| A37 | 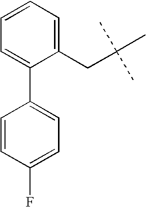 |
| A38 | 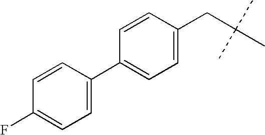 |
| A39 | 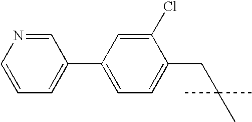 |
| A40 | 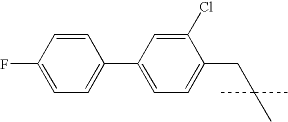 |
| A41 | 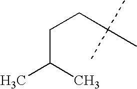 |
| A42 | 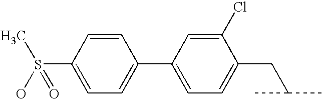 |
| A43 | 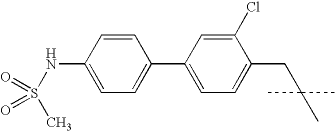 |
| A44 | 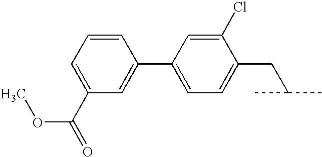 |
| A45 | 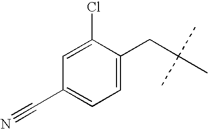 |
| A46 | 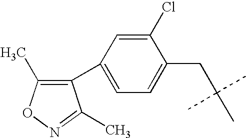 |
| A47 | 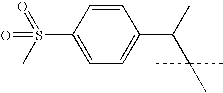 |
| A48 | 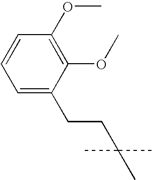 |
| A49 | 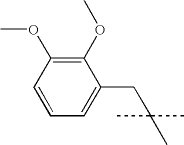 |
| A50 | 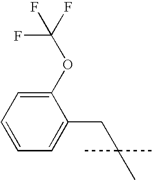 |
| A51 | 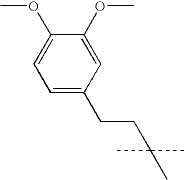 |
| A52 | 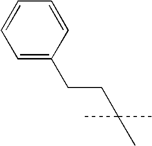 |
| A53 | 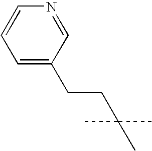 |
| A54 | 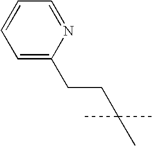 |
| A55 | 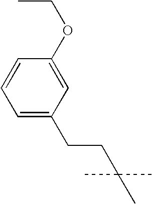 |
| A56 | 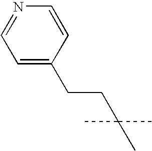 |
| A57 | 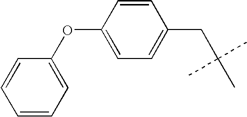 |
| A58 | 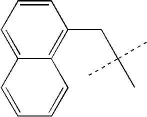 |
| A59 | 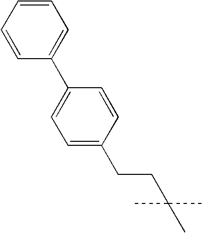 |
| A60 | 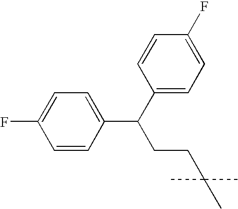 |
| A61 | 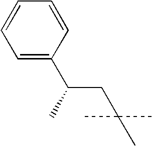 |
| A62 | 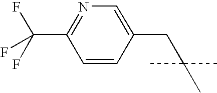 |
| A63 | 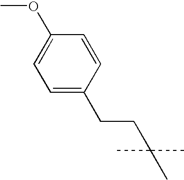 |
| A64 | 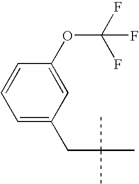 |
| A65 | 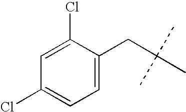 |
| A66 | 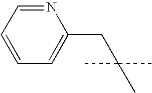 |
| A67 | 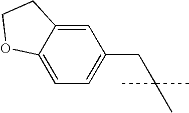 |
| B | 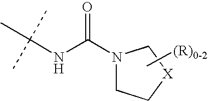 |
| B1 | 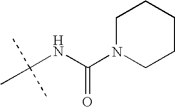 |
| B2 | 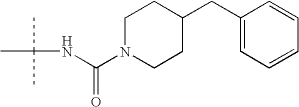 |
| B3 | 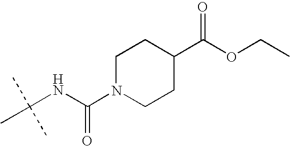 |
| B4 | 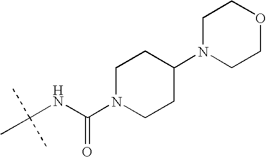 |
| B5 | 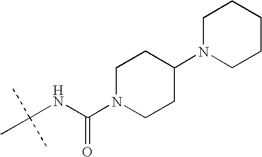 |
| B6 | 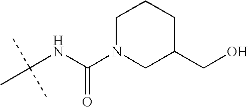 |
| B7 | 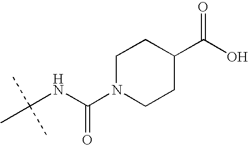 |
| B8 | 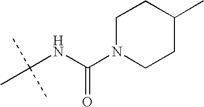 |
| B9 | 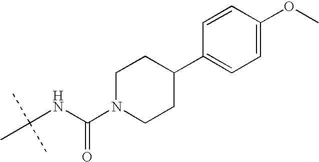 |
| B10 | 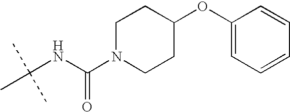 |
| B11 | 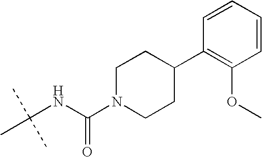 |
| B12 | 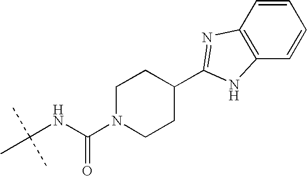 |
| B13 | 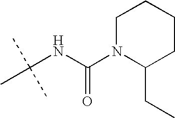 |
| B14 | 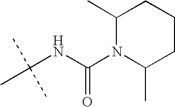 |
| B15 | 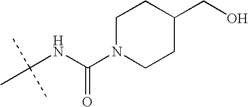 |
| B16 | 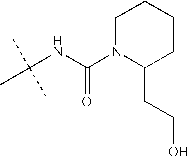 |
| B17 | 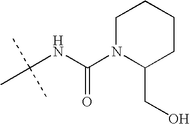 |
| B18 | 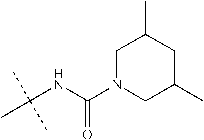 |
| B19 | 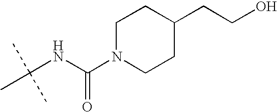 |
| B20 | 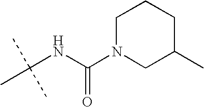 |
| B21 | 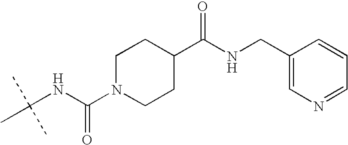 |
| B22 | 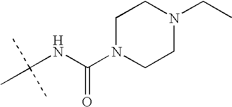 |
| B22 | 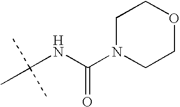 |
| B23 | 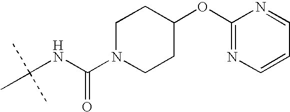 |
| B24 | 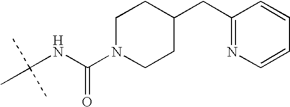 |
| B25 | 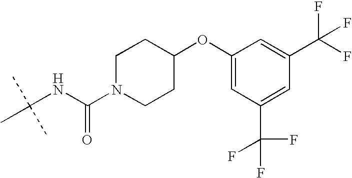 |
| B26 | 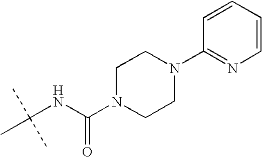 |
| B27 | 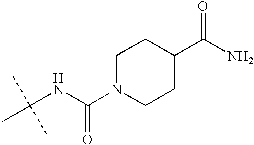 |
| B28 | 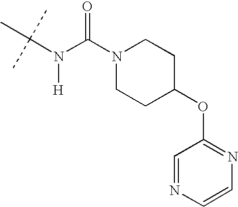 |
| B29 | 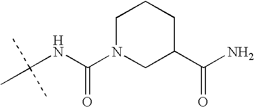 |
| B30 | 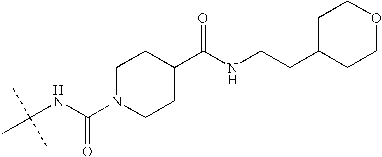 |
| B31 | 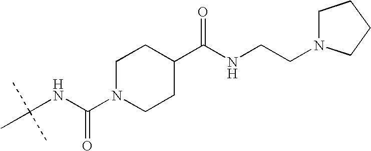 |
| B32 | 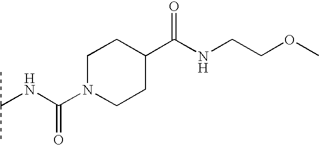 |
| B33 | 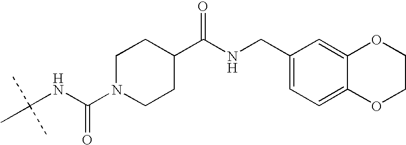 |
| B34 | 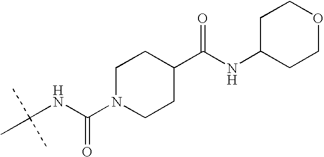 |
| B35 | 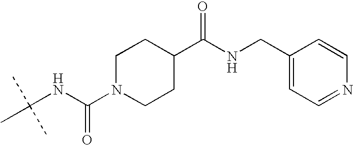 |
| B36 | 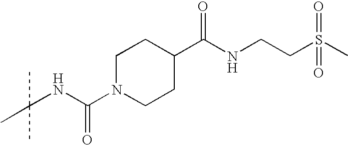 |
| B37 | 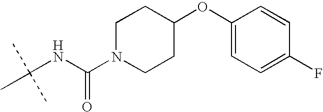 |
| B38 | 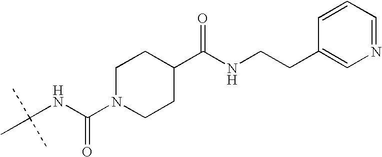 |
| B39 | 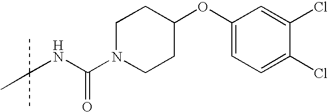 |
| B40 | 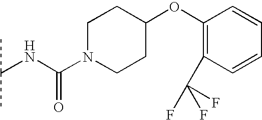 |
| B41 | 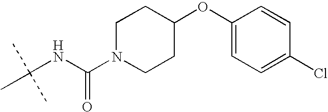 |
| B42 | 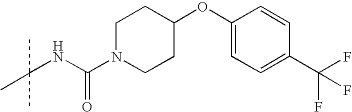 |
| B43 | 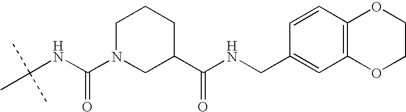 |
| B44 | 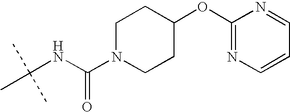 |
| B45 | 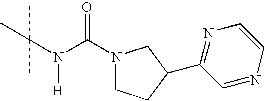 |
| B46 | 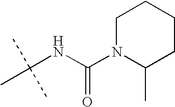 |
| B47 | 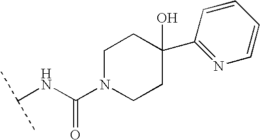 |
| B48 | 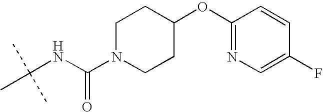 |
| B49 | 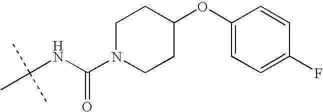 |
| B50 | 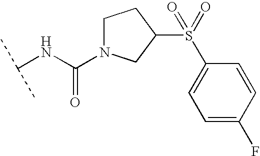 |
| B51 | 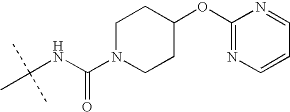 |
| B52 | 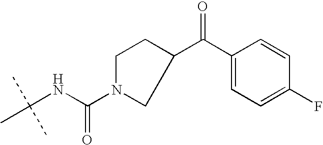 |
| B53 | 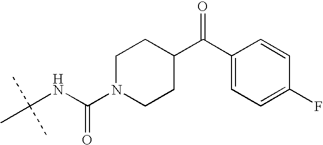 |
| B54 | 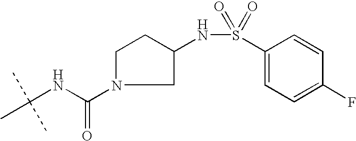 |
| B55 | 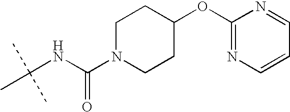 |
| B56 | 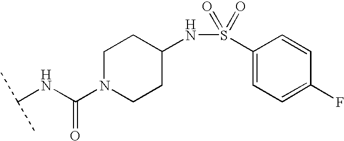 |
| B57 | 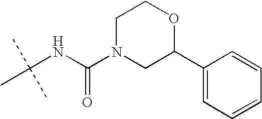 |
| B58 | 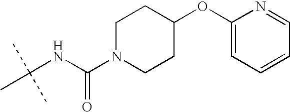 |
| B59 | 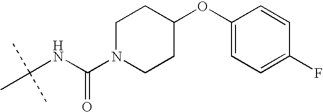 |
| B60 | 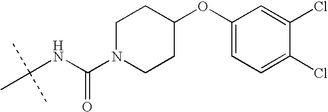 |
| B61 | 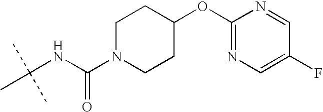 |
| B62 | 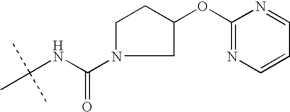 |
| B63 | 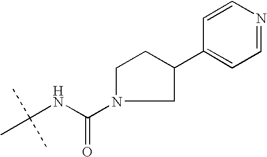 |
| B64 | 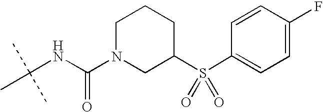 |
| B65 | 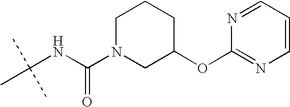 |
| B66 | 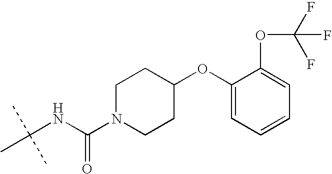 |
| B67 | 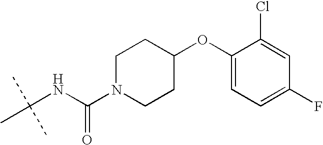 |
| B68 | 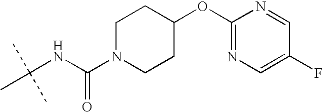 |
| B69 | 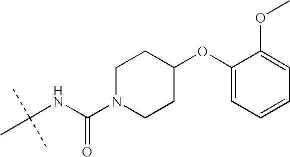 |
| B70 | 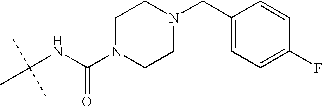 |
| B71 | 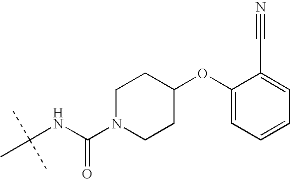 |
| B72 | 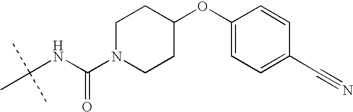 |
| B73 | 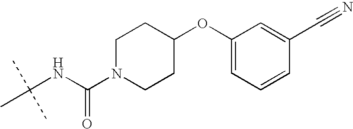 |
| B74 | 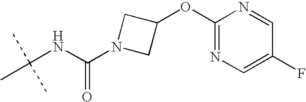 |
| B75 | 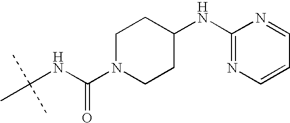 |
| B76 | 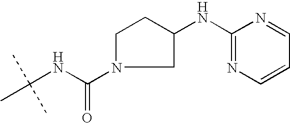 |
| B77 | 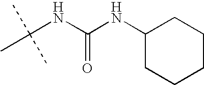 |
| B78 | 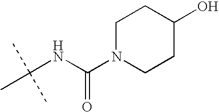 |
| B79 | 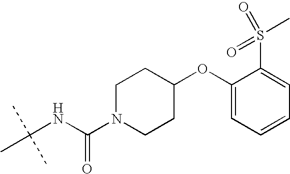 |
| B80 | 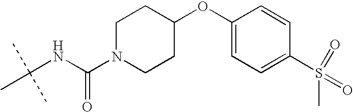 |
| B81 | 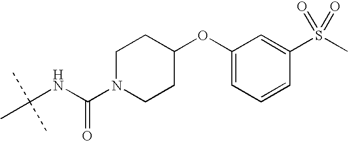 |
| B82 | 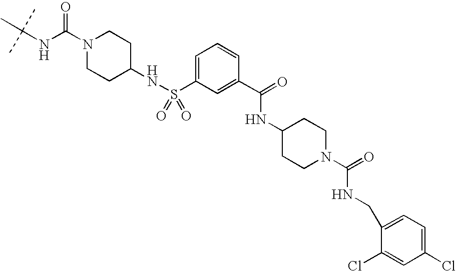 |
| B83 | 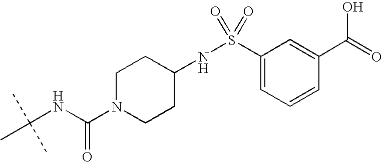 |
| B84 | 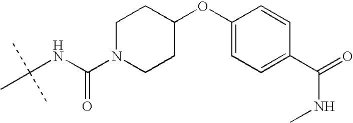 |
| B85 | 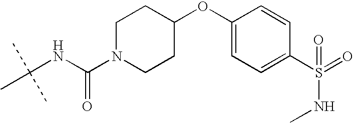 |
| B86 | 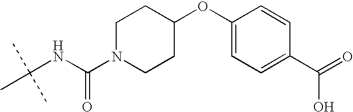 |
| B87 | 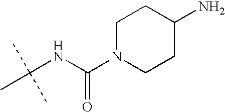 |
| B88 | 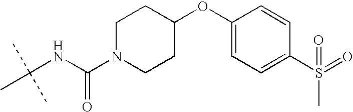 |
| B89 | 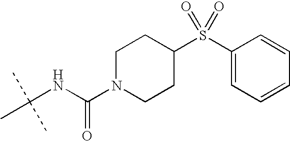 |
| B90 | 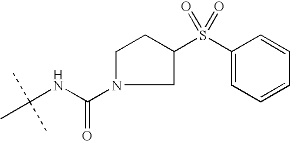 |
| B91 | 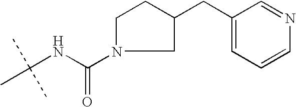 |
| B92 | 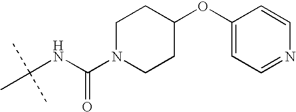 |
| B93 | 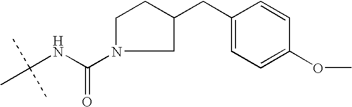 |
| B94 | 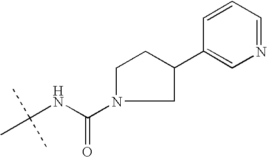 |
| B95 | 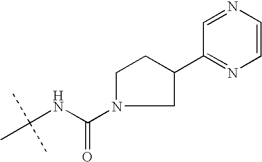 |
| B96 | 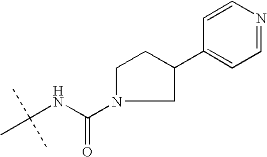 |
| B97 | 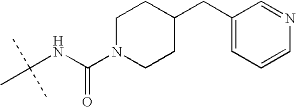 |

or the pharmaceutically acceptable salts thereof.

5. The compound according to claim 4, and wherein:
wherein for the Formula (Ia), the component 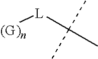
is chosen from A1-A41 in the table II below; in combination with any component 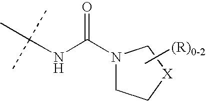
chosen from B1-B97 in the table II below;
TABLE II

| | |
|---|---|
| A | 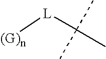 |
| A1 | 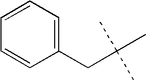 |
| A2 | 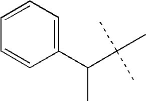 |
| A3 | 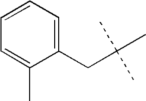 |
| A4 | 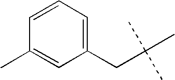 |
| A5 | 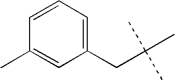 |
| A6 | 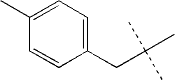 |
| A7 | 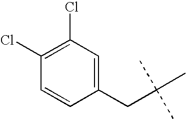 |
| A8 | 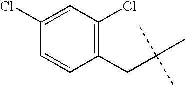 |
| A9 | 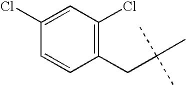 |
| A10 | 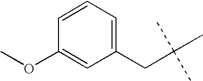 |
| A11 | 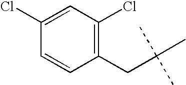 |
| A12 | 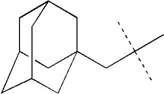 |
| A13 | 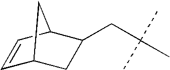 |
| A14 | 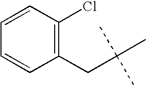 |
| A15 | 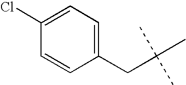 |
| A16 | 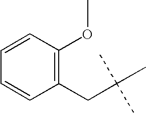 |
| A17 | 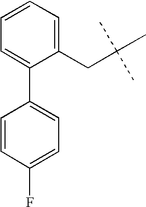 |
| A18 | 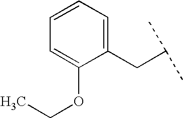 |
| A19 | 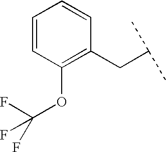 |
| A20 | 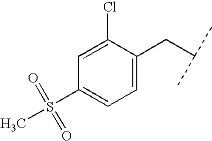 |
| A21 | 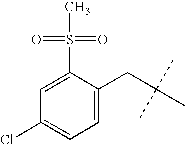 |
| A22 | 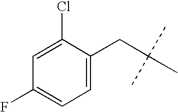 |
| A22 | 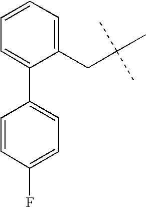 |
| A23 | 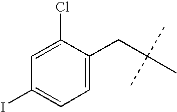 |
| A24 | 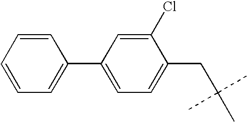 |
| A25 | 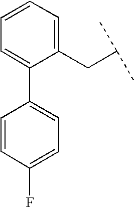 |
| A26 | 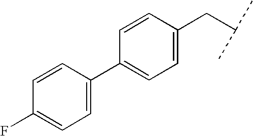 |
| A27 | 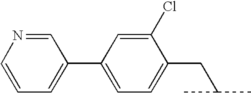 |
| A28 | 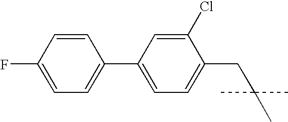 |
| A29 | 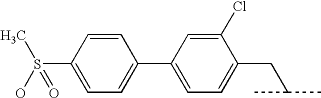 |
| A30 | 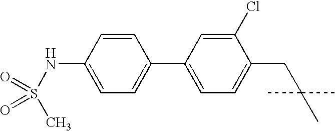 |
| A31 | 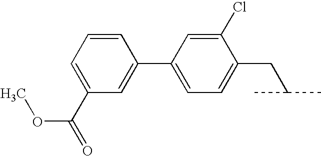 |
| A32 | 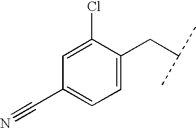 |
| A33 | 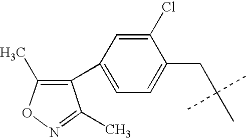 |
| A34 | 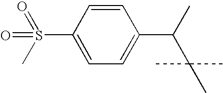 |
| A35 | 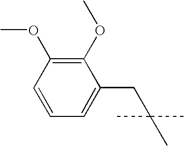 |
| A36 | 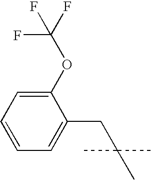 |
| A37 | 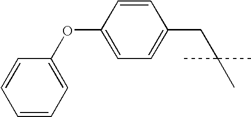 |
| A38 | 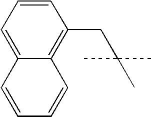 |
| A39 | 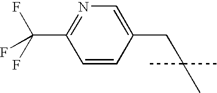 |
| A40 | 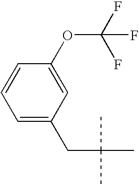 |
| A41 | 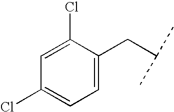 |
| B | 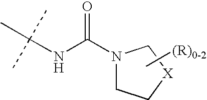 |
| B1 | 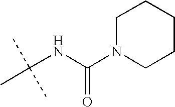 |
| B2 | 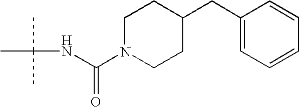 |
| B3 | 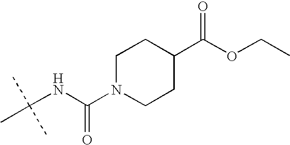 |
| B4 | 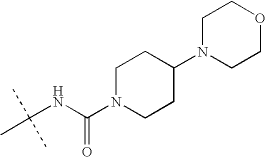 |
| B5 | 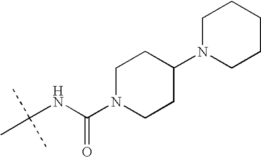 |
| B6 | 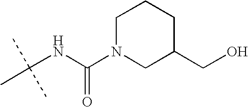 |
| B7 | 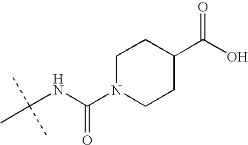 |
| B8 | 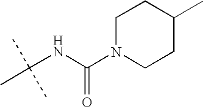 |
| B9 | 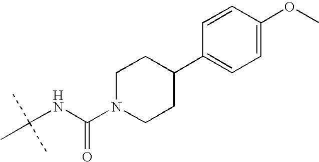 |
| B10 | 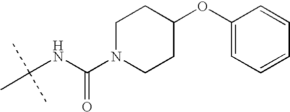 |
| B11 | 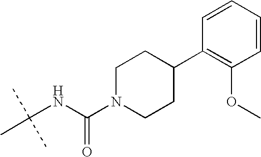 |
| B12 | 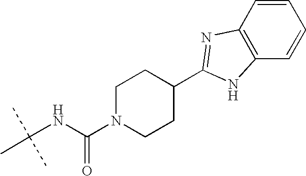 |
| B13 | 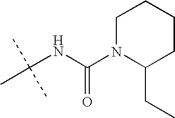 |
| B14 | 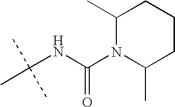 |
| B15 | 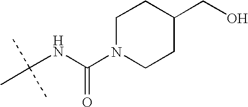 |
| B16 | 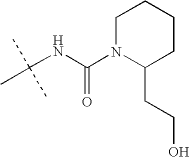 |
| B17 | 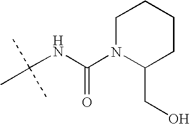 |
| B18 | 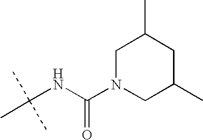 |
| B19 | 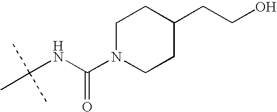 |
| B20 | 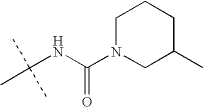 |
| B21 | 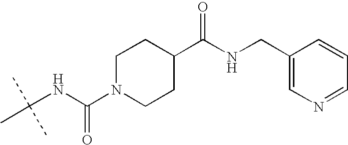 |
| B22 | 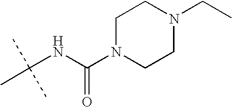 |
| B22 | 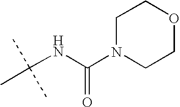 |
| B23 | 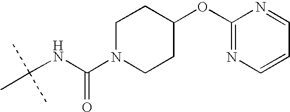 |
| B24 | 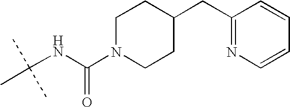 |
| B25 | 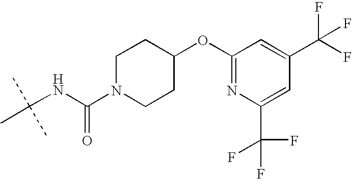 |
| A26 | 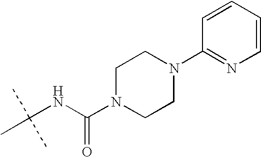 |
| B27 | 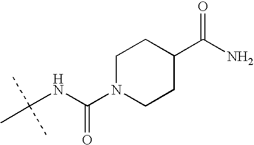 |
| B28 | 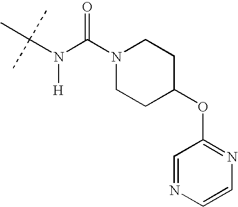 |
| B29 | 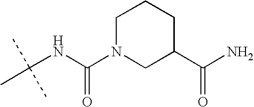 |
| B30 | 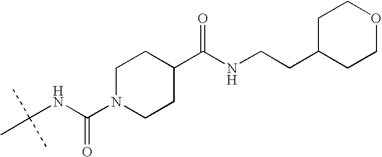 |
| B31 | 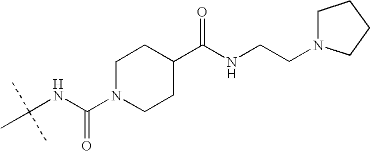 |
| B32 | 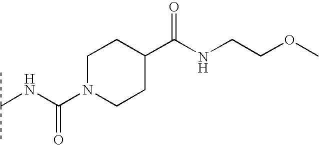 |
| B33 | 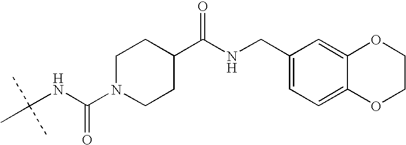 |
| B34 | 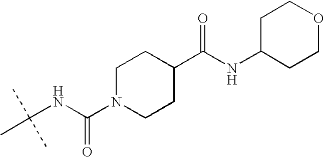 |
| B35 | 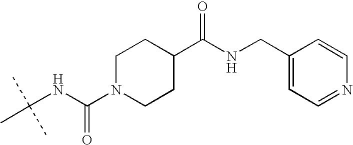 |
| B36 | 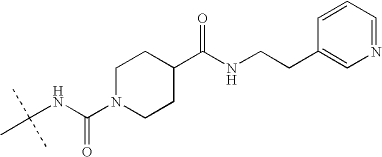 |
| B37 | 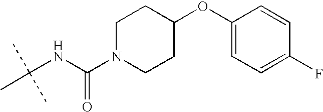 |
| B38 | 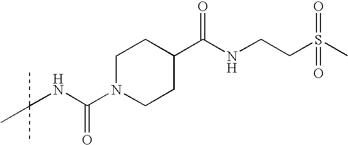 |
| B39 | 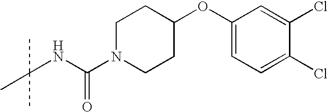 |
| B40 | 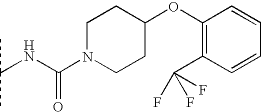 |
| B41 | 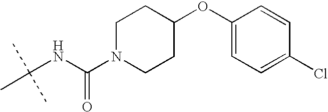 |
| B42 | 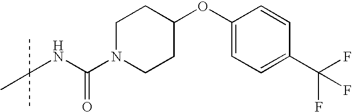 |
| B43 | 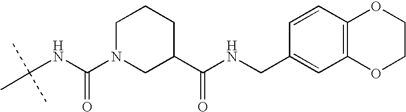 |
| B44 | 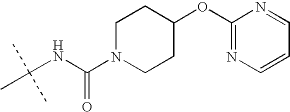 |
| B45 | 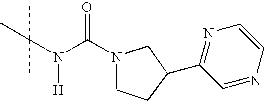 |
| B46 | 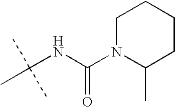 |
| B47 | 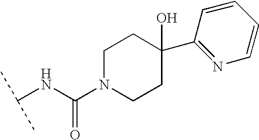 |
| B48 | 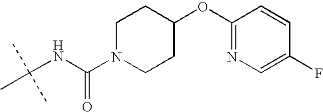 |
| B49 | 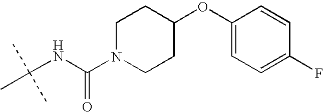 |
| B50 | 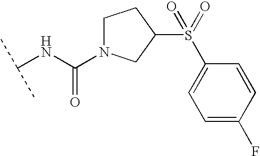 |
| B51 | 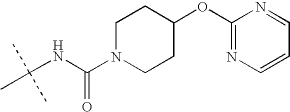 |
| B52 | 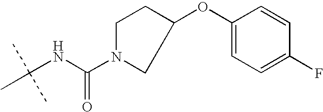 |
| B53 | 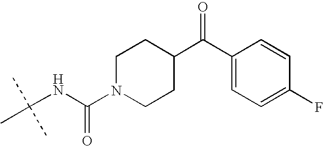 |
| B54 | 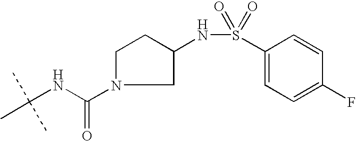 |
| B55 | 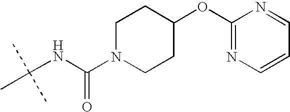 |
| B56 | 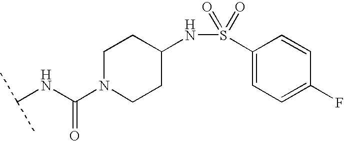 |
| B57 | 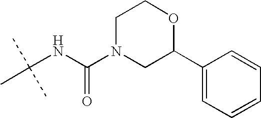 |
| B58 | 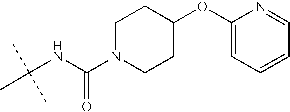 |
| B59 | 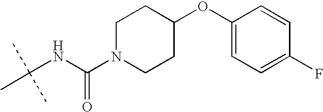 |
| B60 | 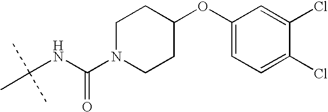 |
| B61 | 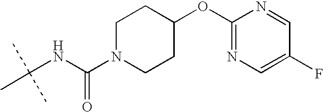 |
| B62 | 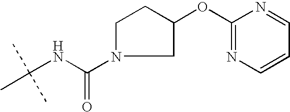 |
| B63 | 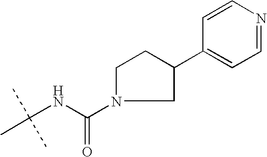 |
| B64 | 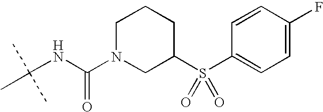 |
| B65 | 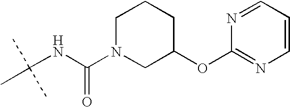 |
| B66 | 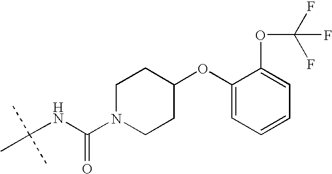 |
| B67 | 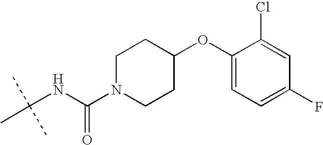 |
| B68 | 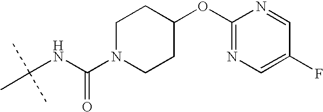 |
| B69 | 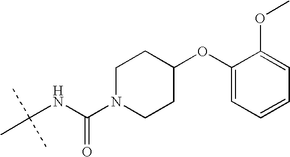 |
| B70 | 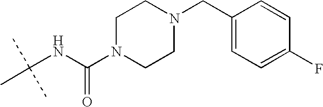 |
| B71 | 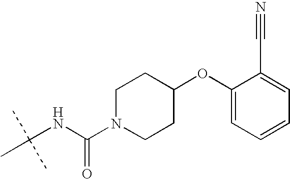 |
| B72 | 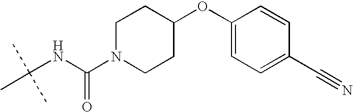 |
| B73 | 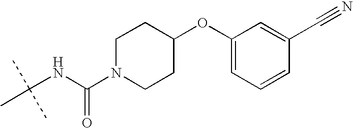 |
| B74 | 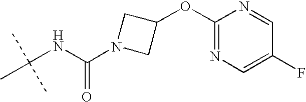 |
| B75 | 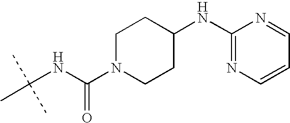 |
| B76 | 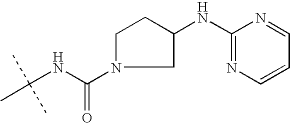 |
| B77 | 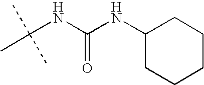 |
| B78 | 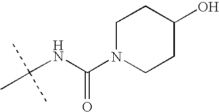 |
| B79 | 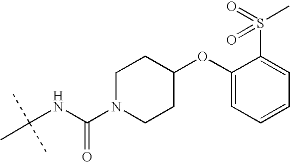 |
| B80 | 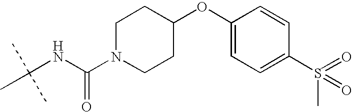 |
| B81 | 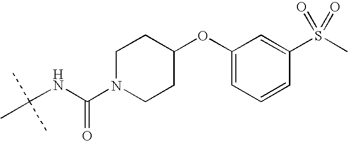 |
| B82 | 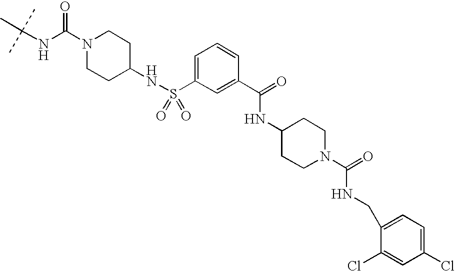 |
| B83 | 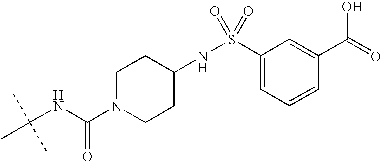 |
| B84 | 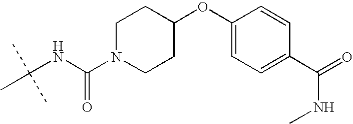 |
| B85 | 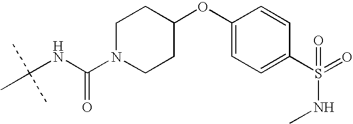 |
| B86 | 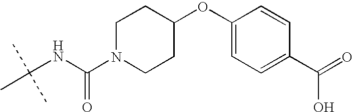 |
| B87 | 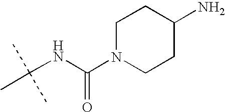 |
| B88 | 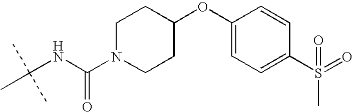 |
| B89 | 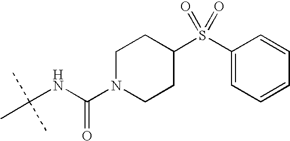 |
| B90 | 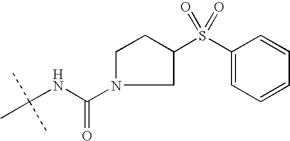 |
| B91 | 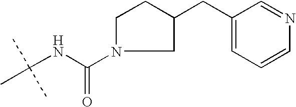 |
| B92 | 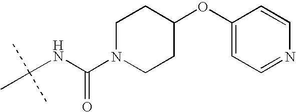 |
| B93 | 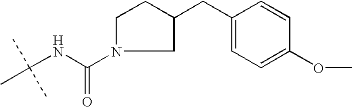 |
| B94 | 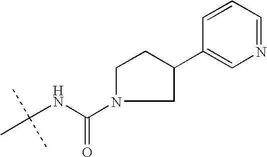 |
| B95 | 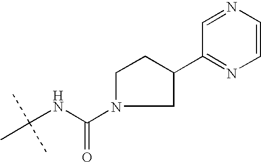 |
| B96 | 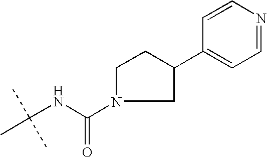 |
| B97 | 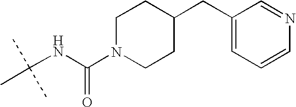 |

6. The compound according to claim 5, and wherein column B of table II is:

| | |
|---|---|
| B | 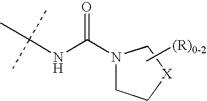 |
| B10 | 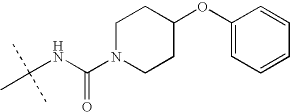 |
| B23 | 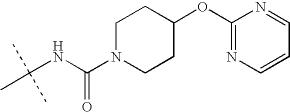 |
| B25 | 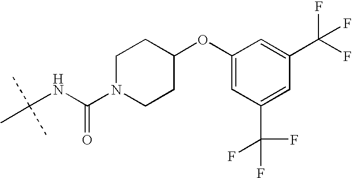 |
| B28 | 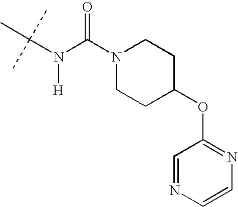 |
| B37 | 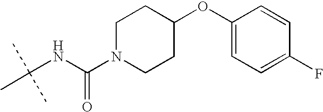 |
| B39 | 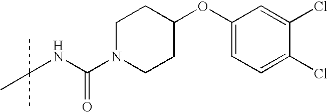 |
| B40 | 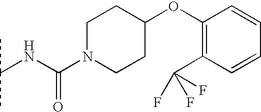 |
| B41 | 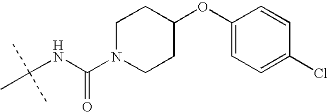 |
| B42 | 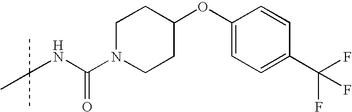 |
| B44 | 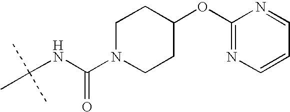 |
| B48 | 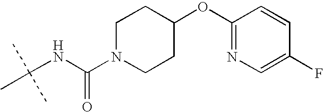 |
| B49 | 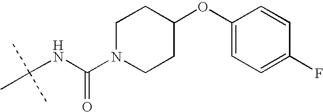 |
| B51 | 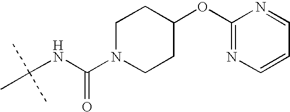 |
| B52 | 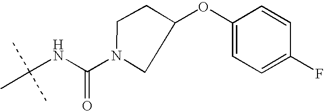 |
| B55 | 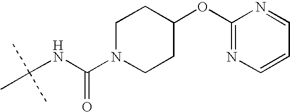 |
| B58 | 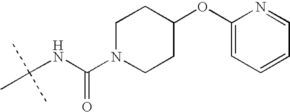 |
| B59 | 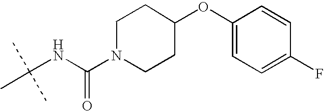 |
| B60 | 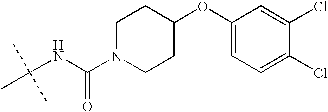 |
| B61 | 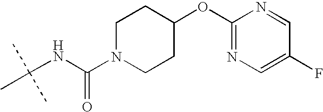 |
| B62 | 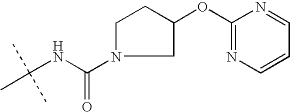 |
| B65 | 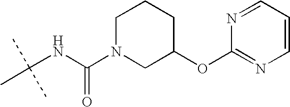 |
| B66 | 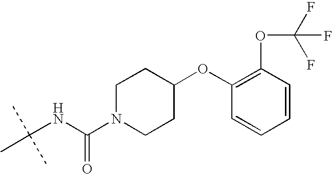 |
| B67 | 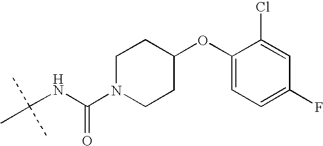 |
| B68 | 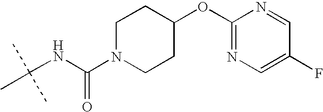 |
| B69 | 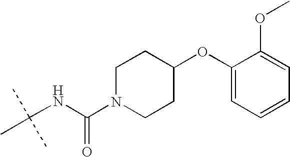 |
| B71 | 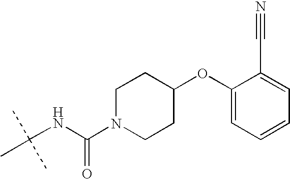 |
| B72 | 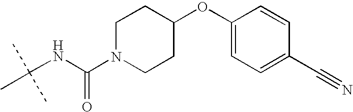 |
| B73 | 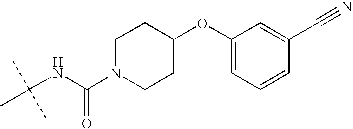 |
| B74 | 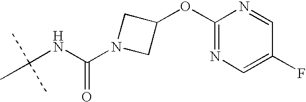 |
| B79 | 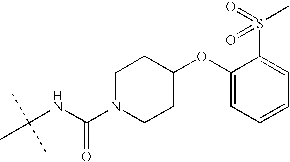 |
| B80 | 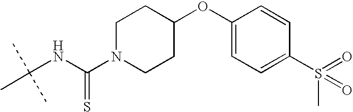 |
| B81 | 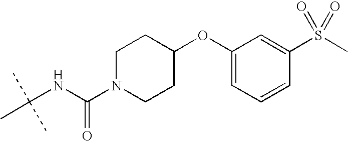 |
| B84 | 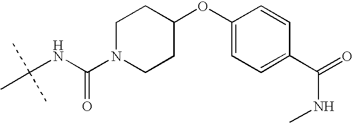 |
| B85 | 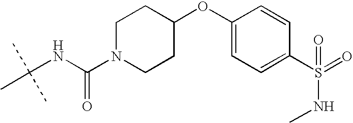 |
| B86 | 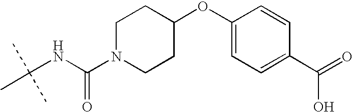 |
| B88 | 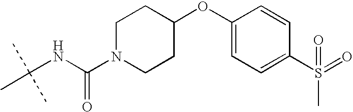 |
| B89 | 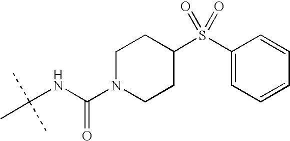 |
| B90 | 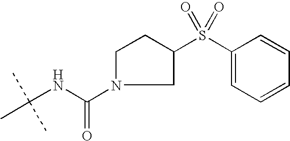 |
| B92 | 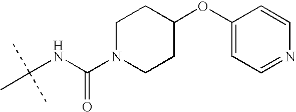 |

7. A compound chosen from: 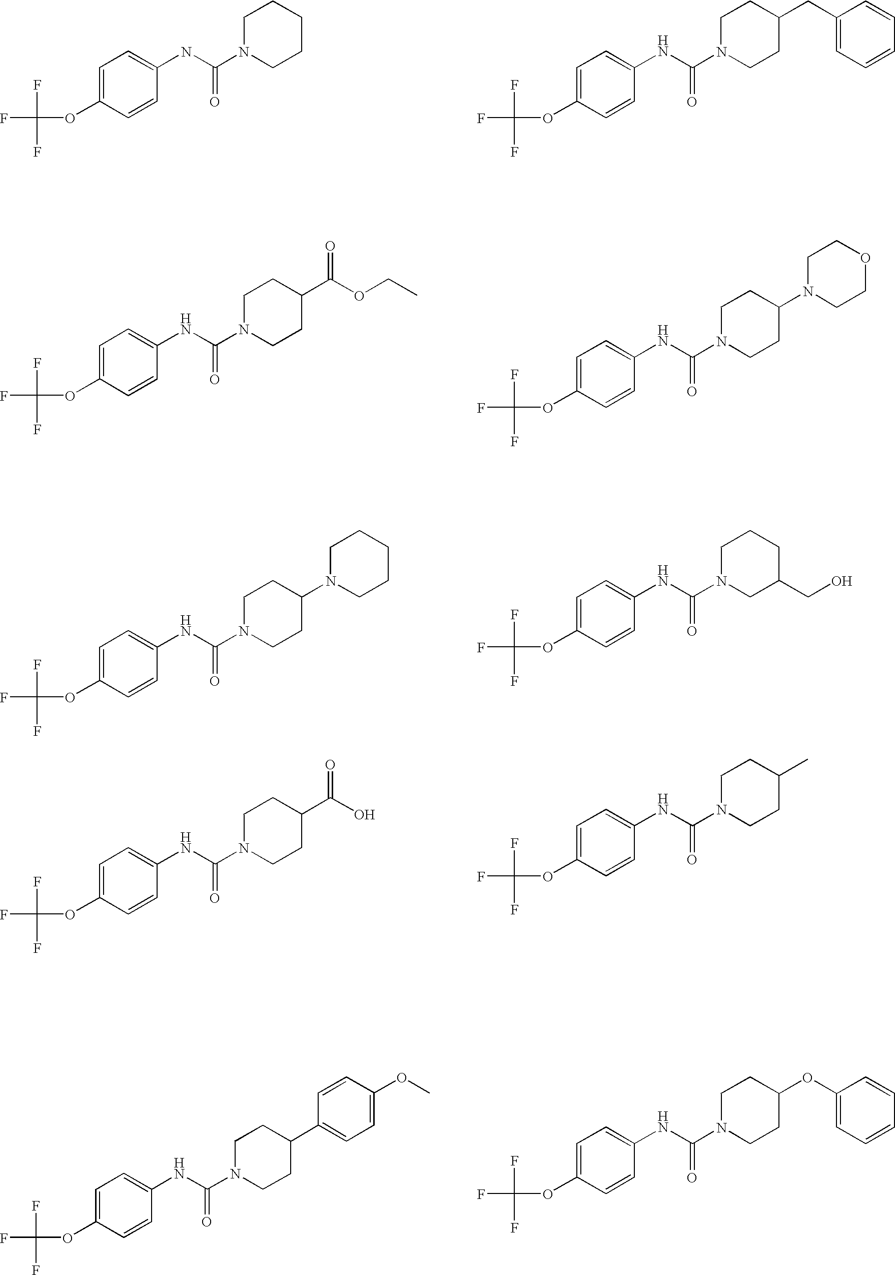 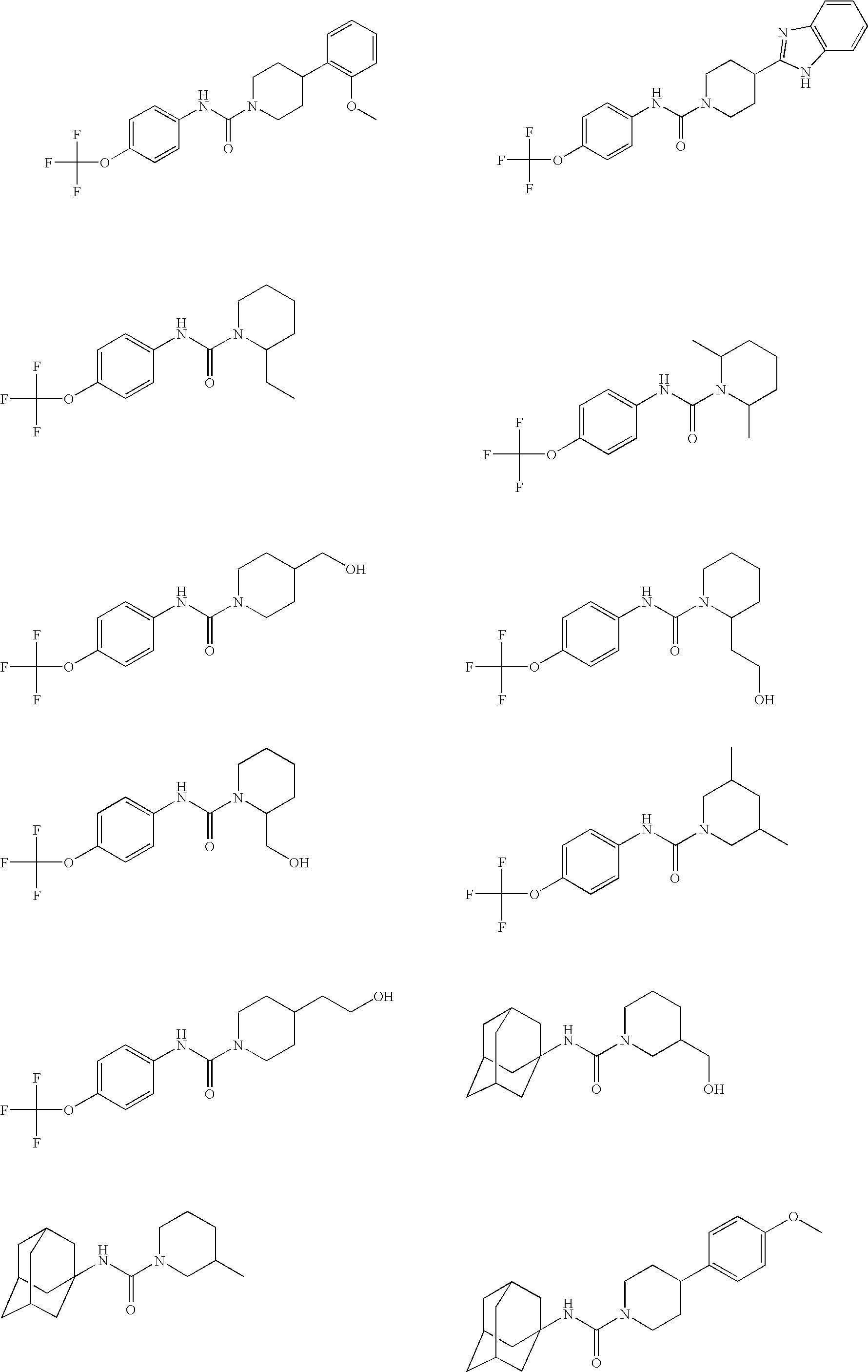 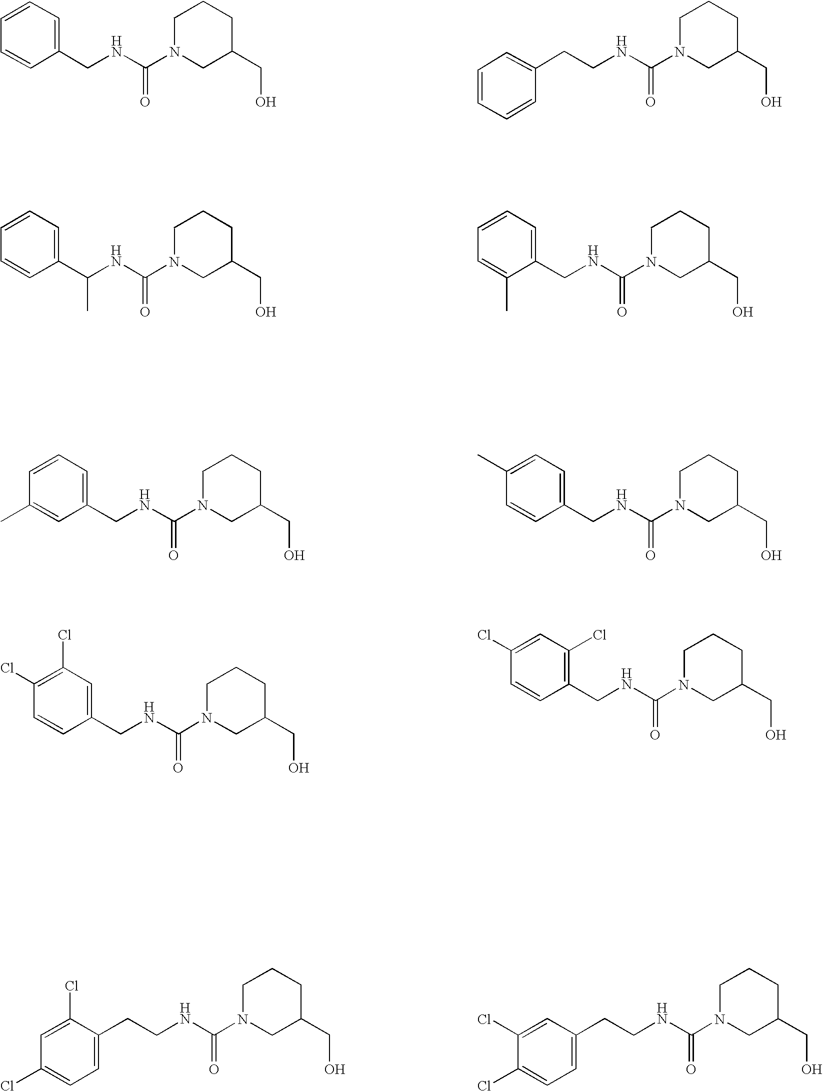 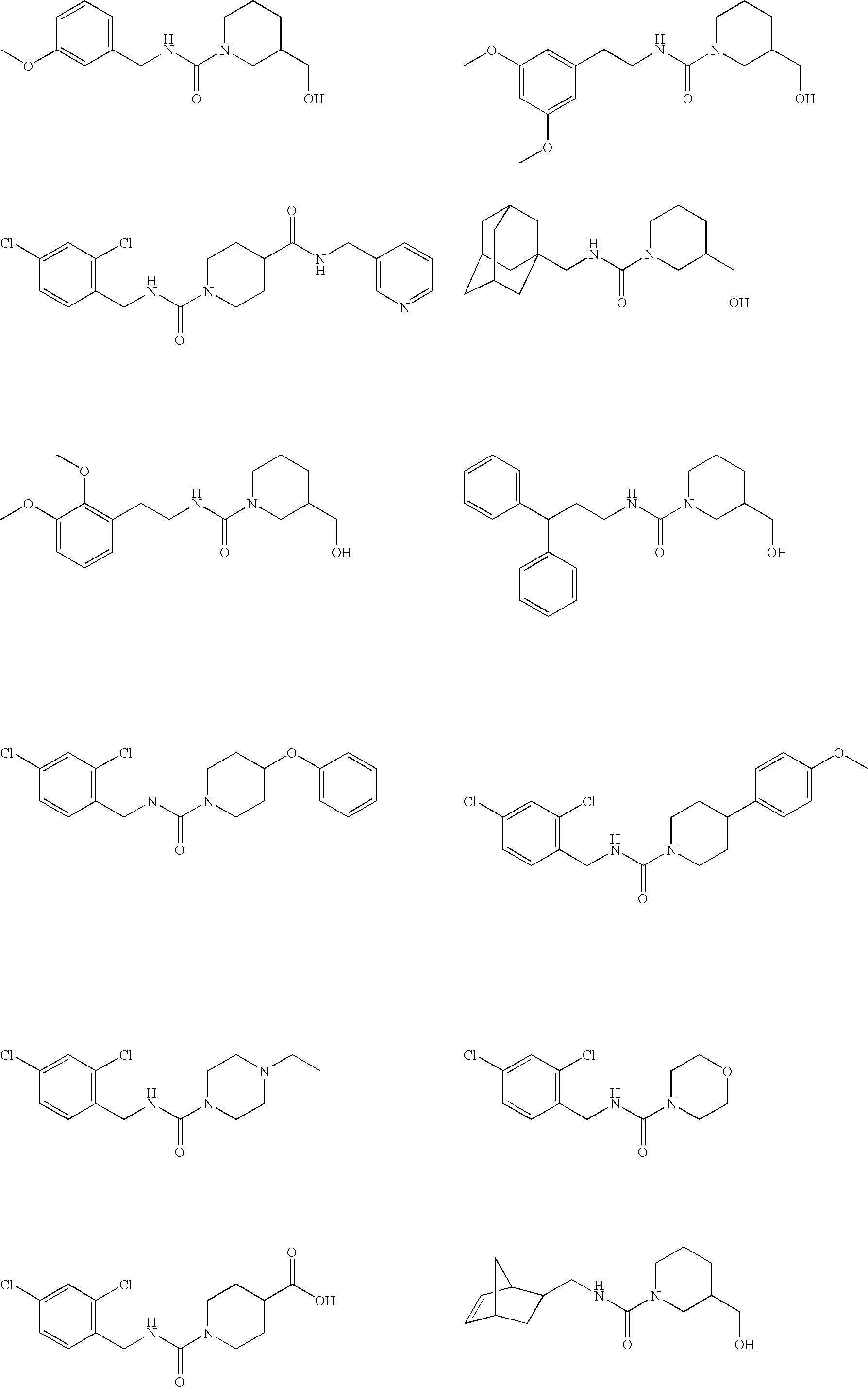 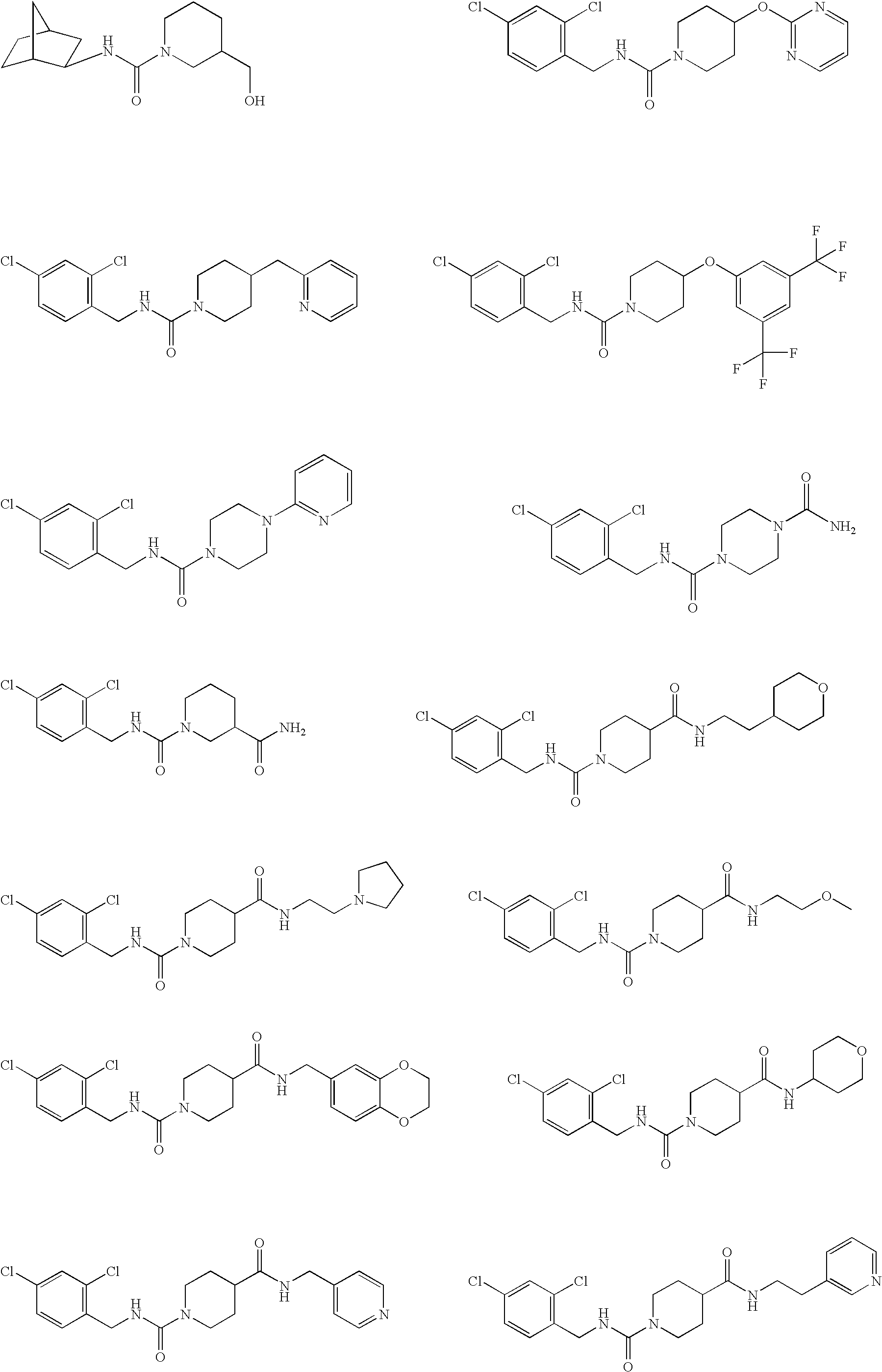 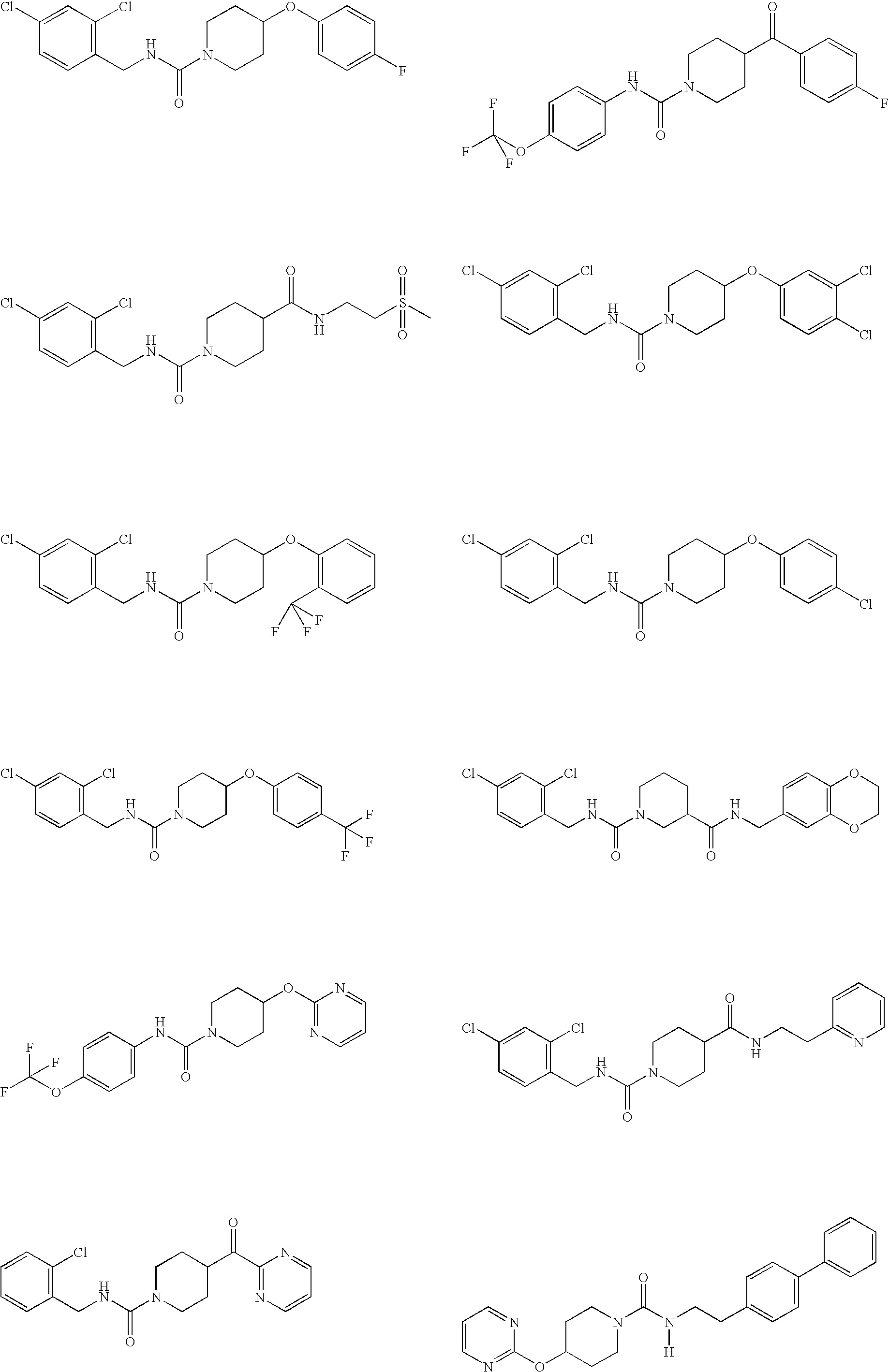 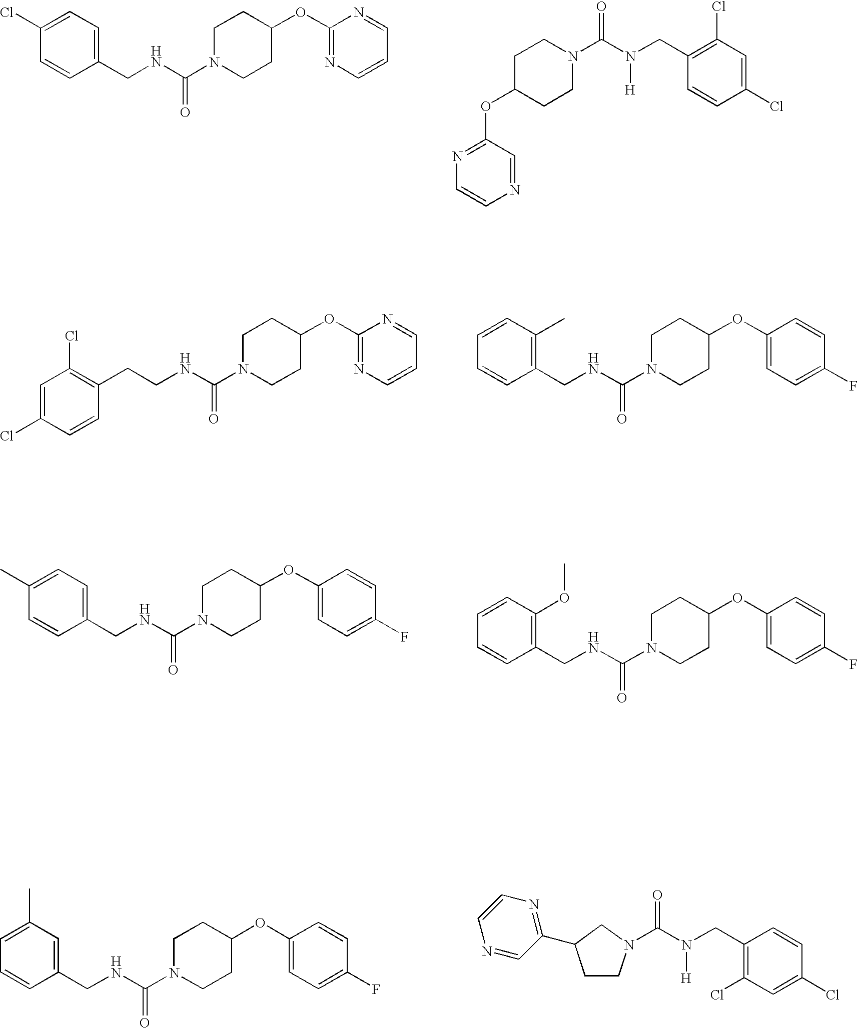 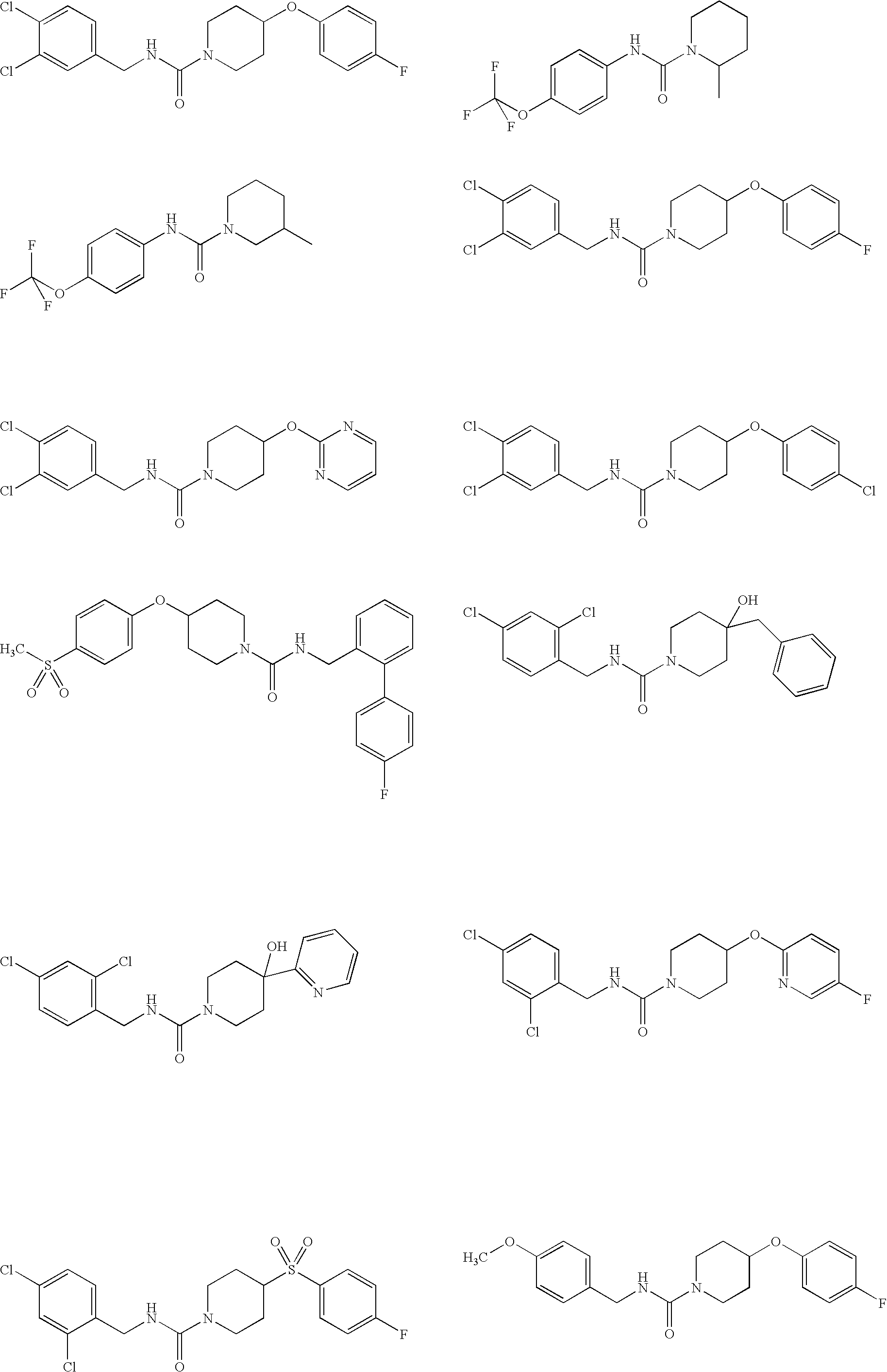 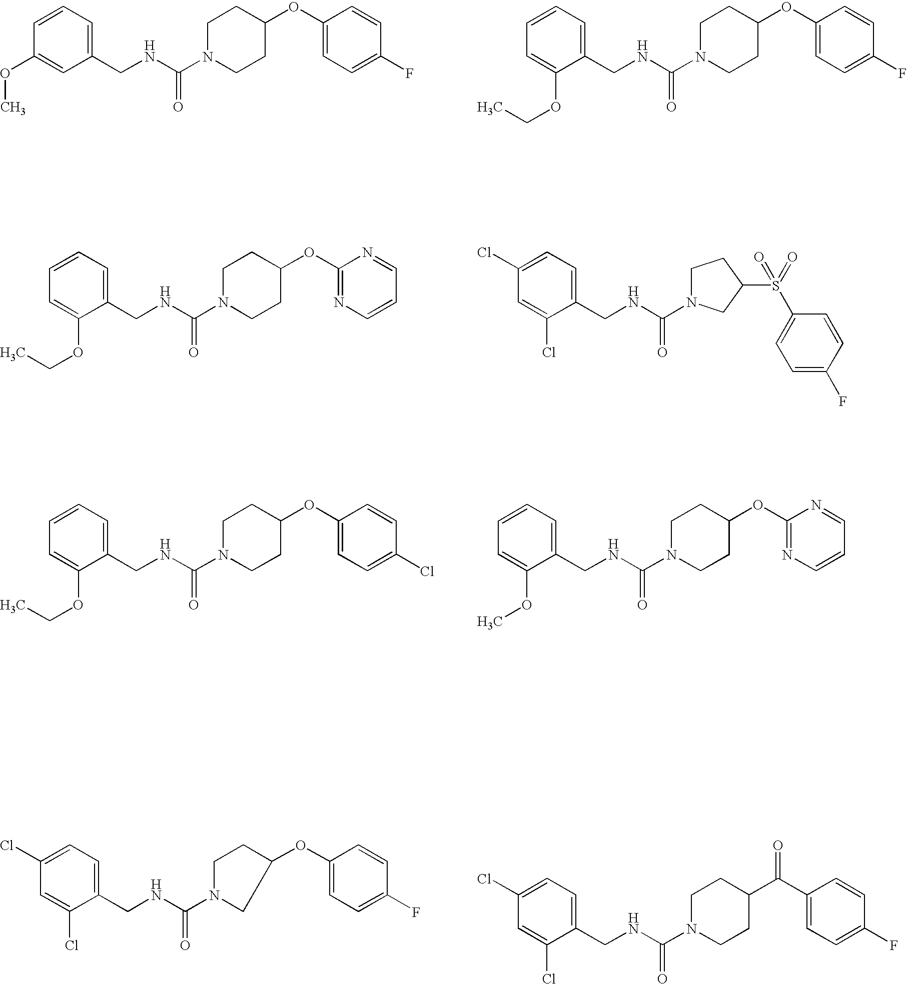 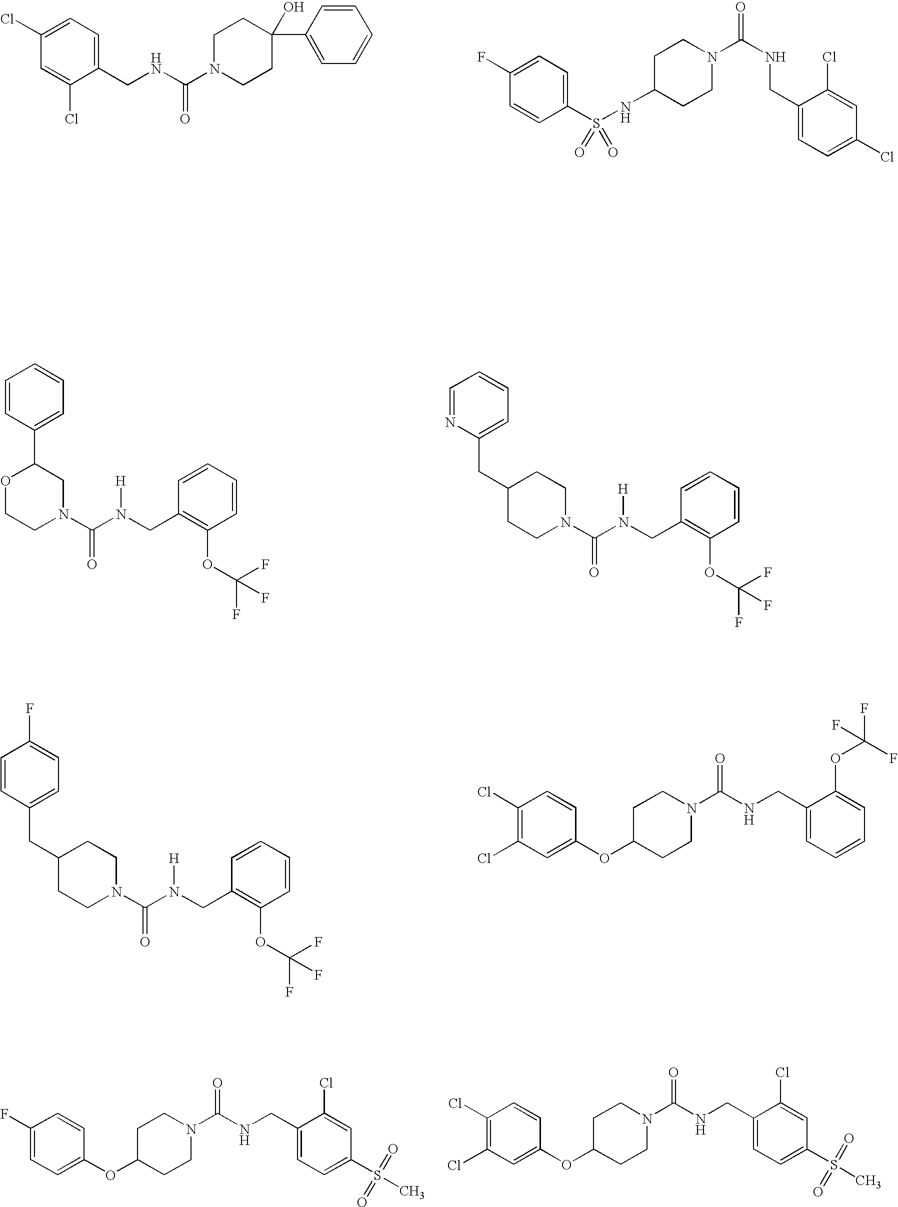 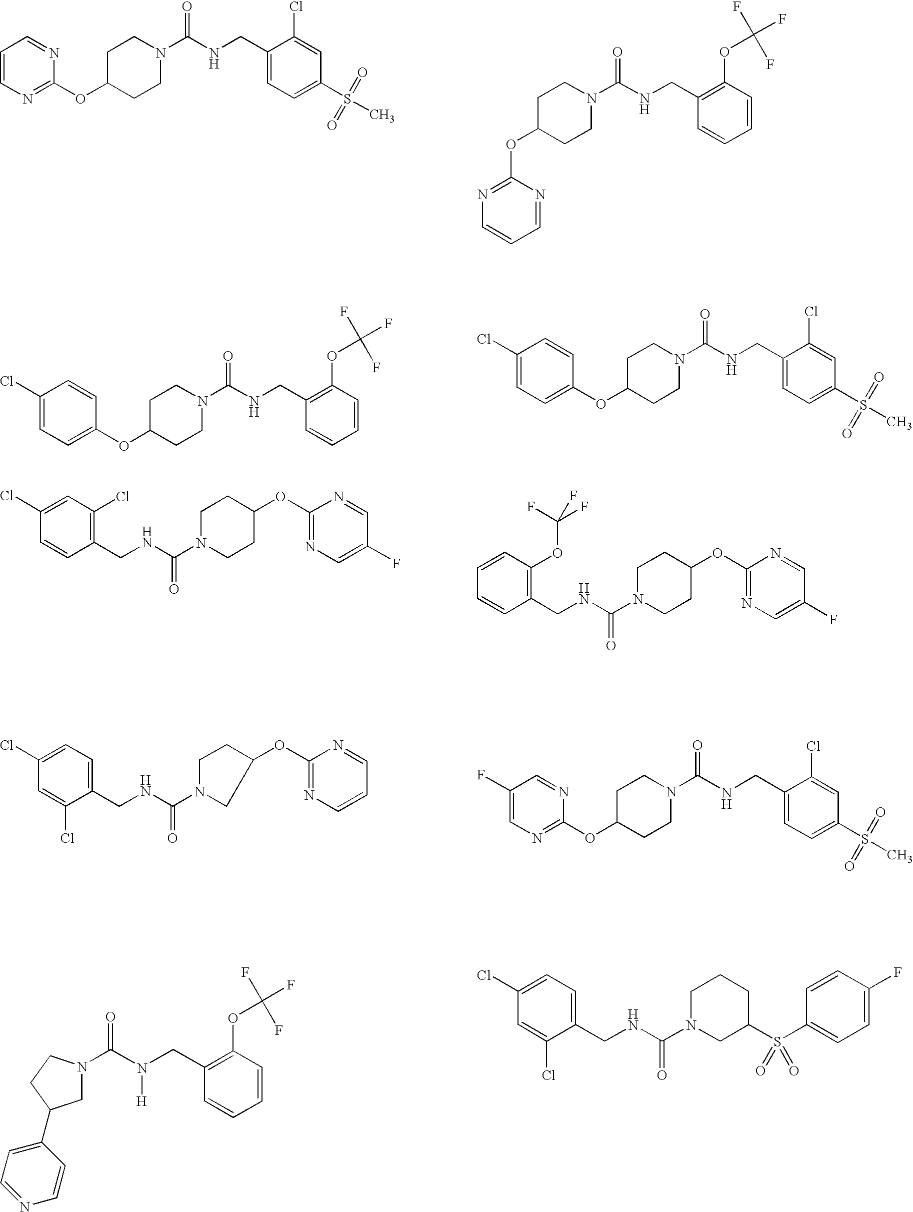 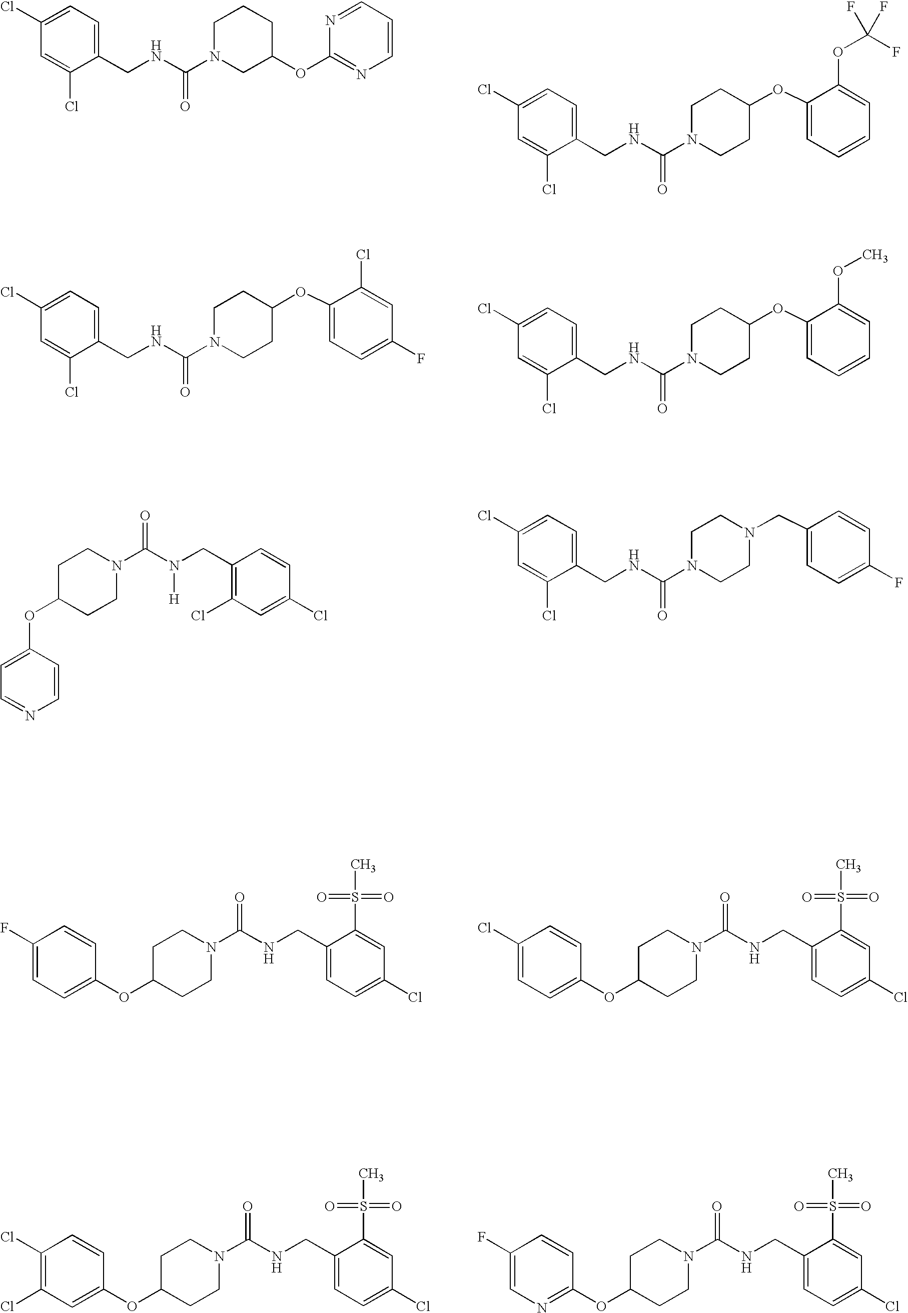 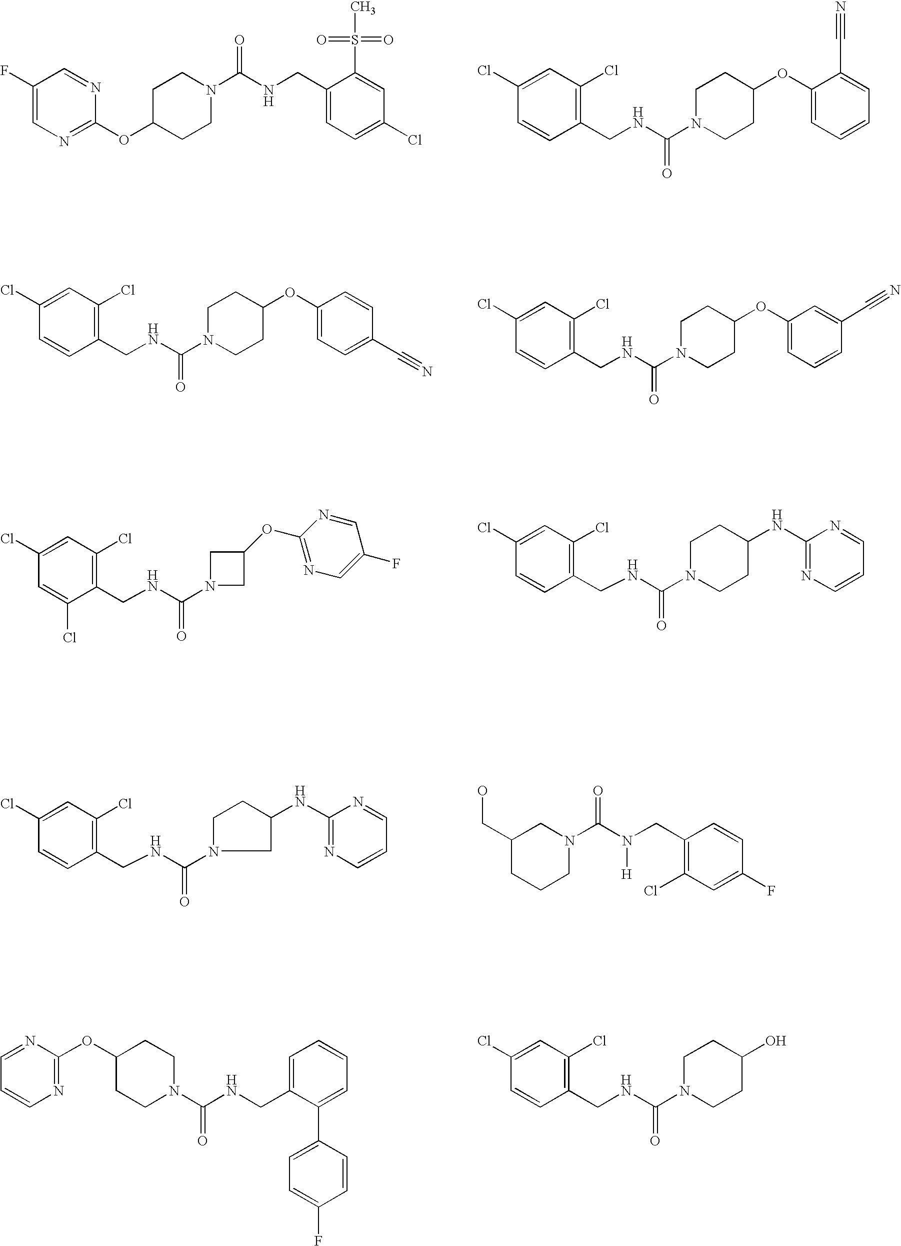 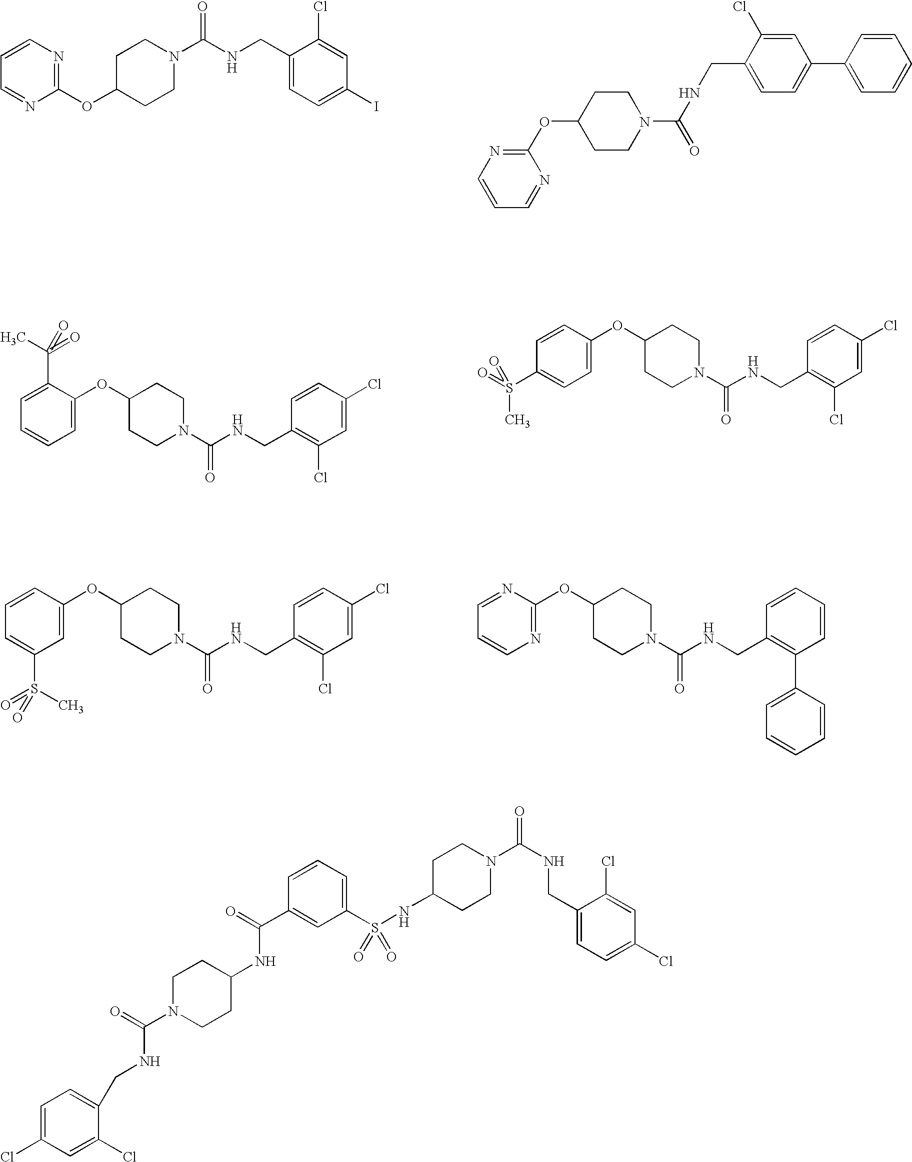 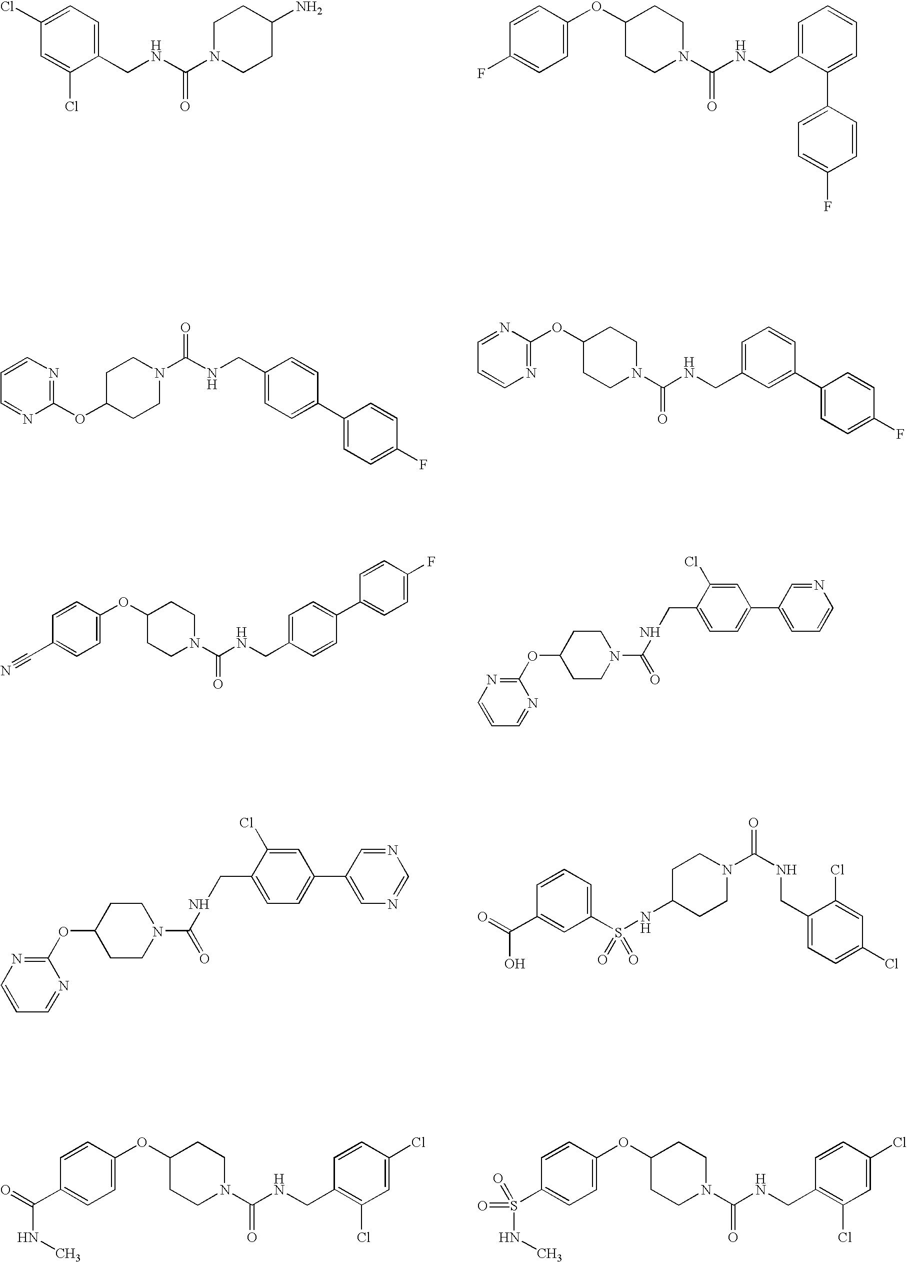 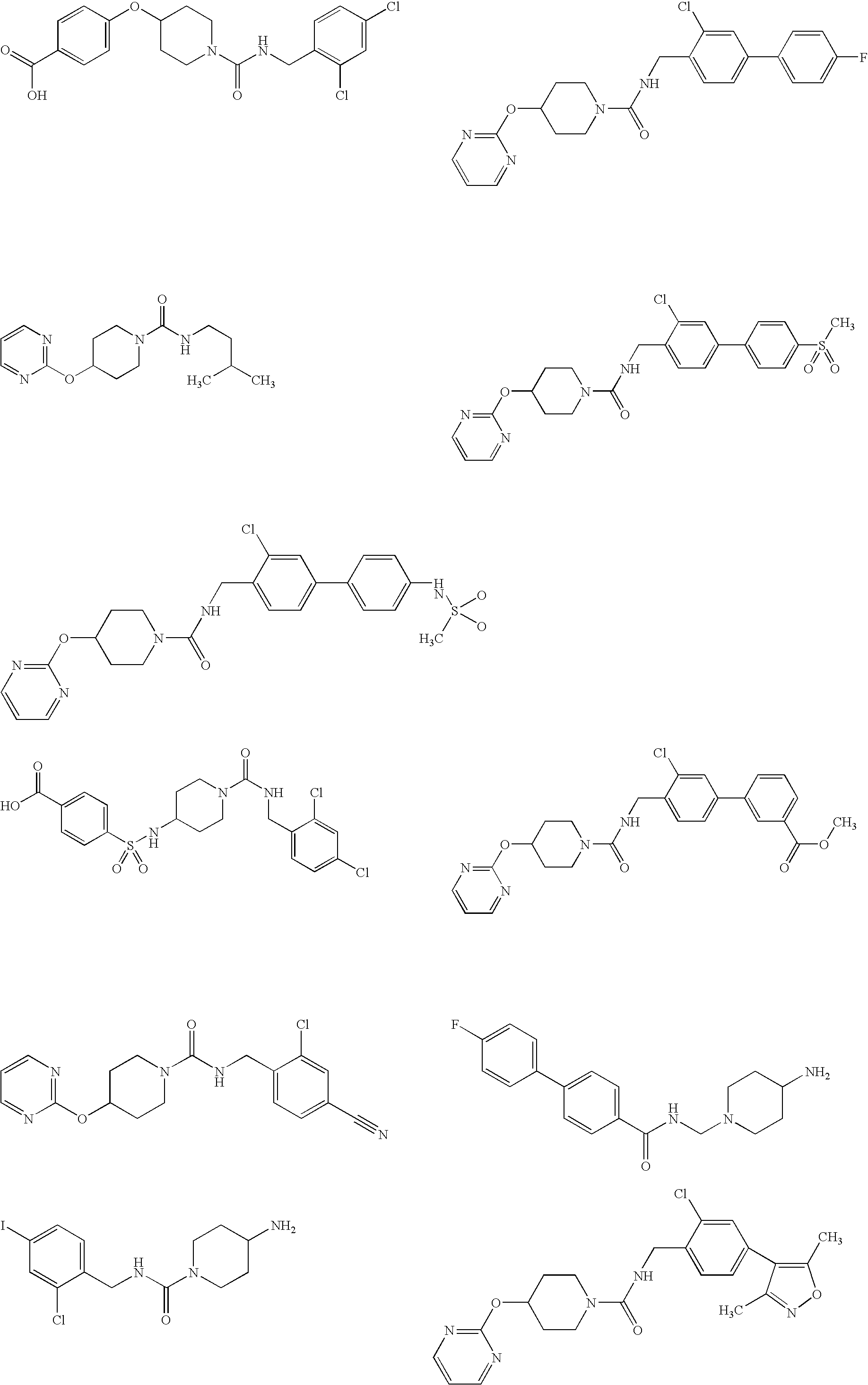 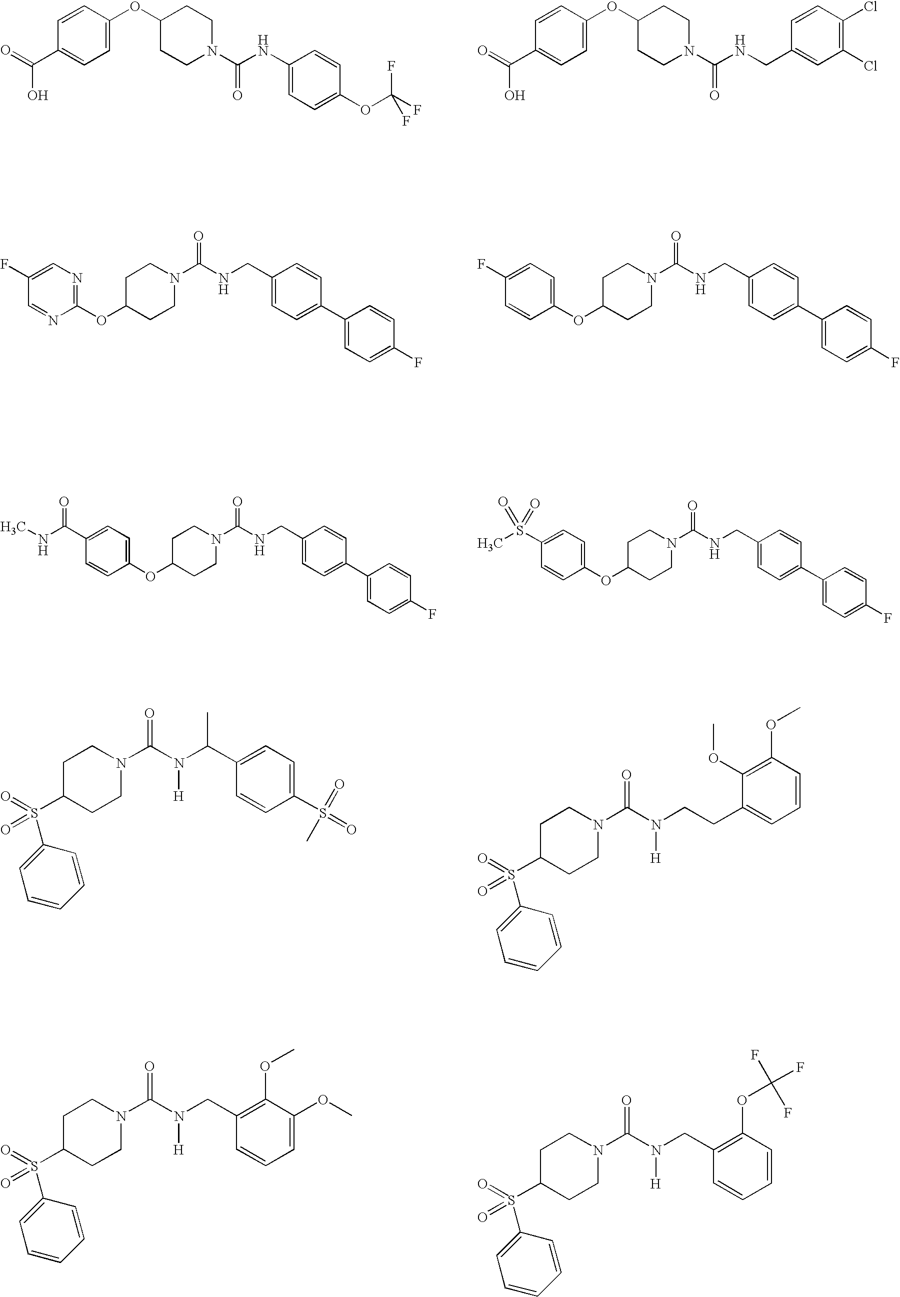 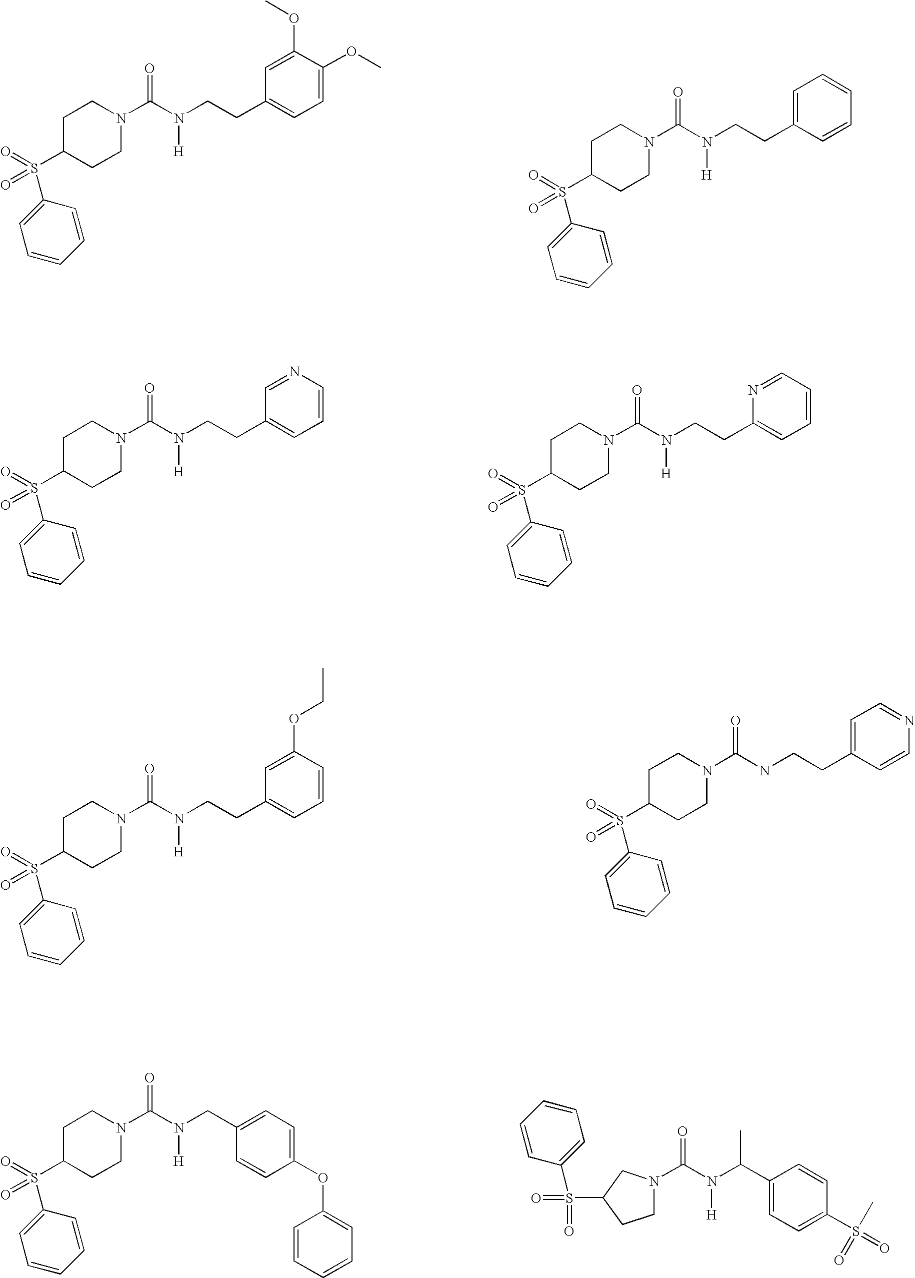 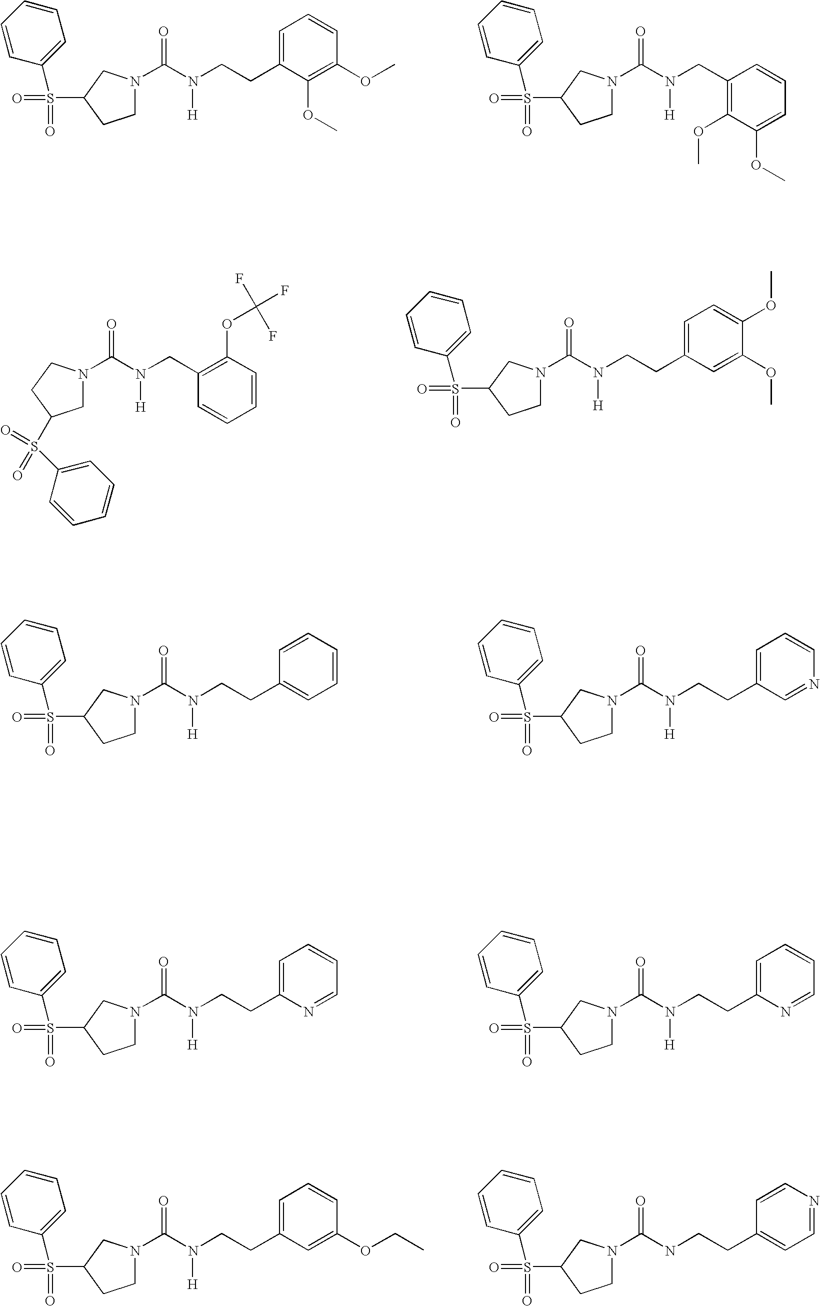 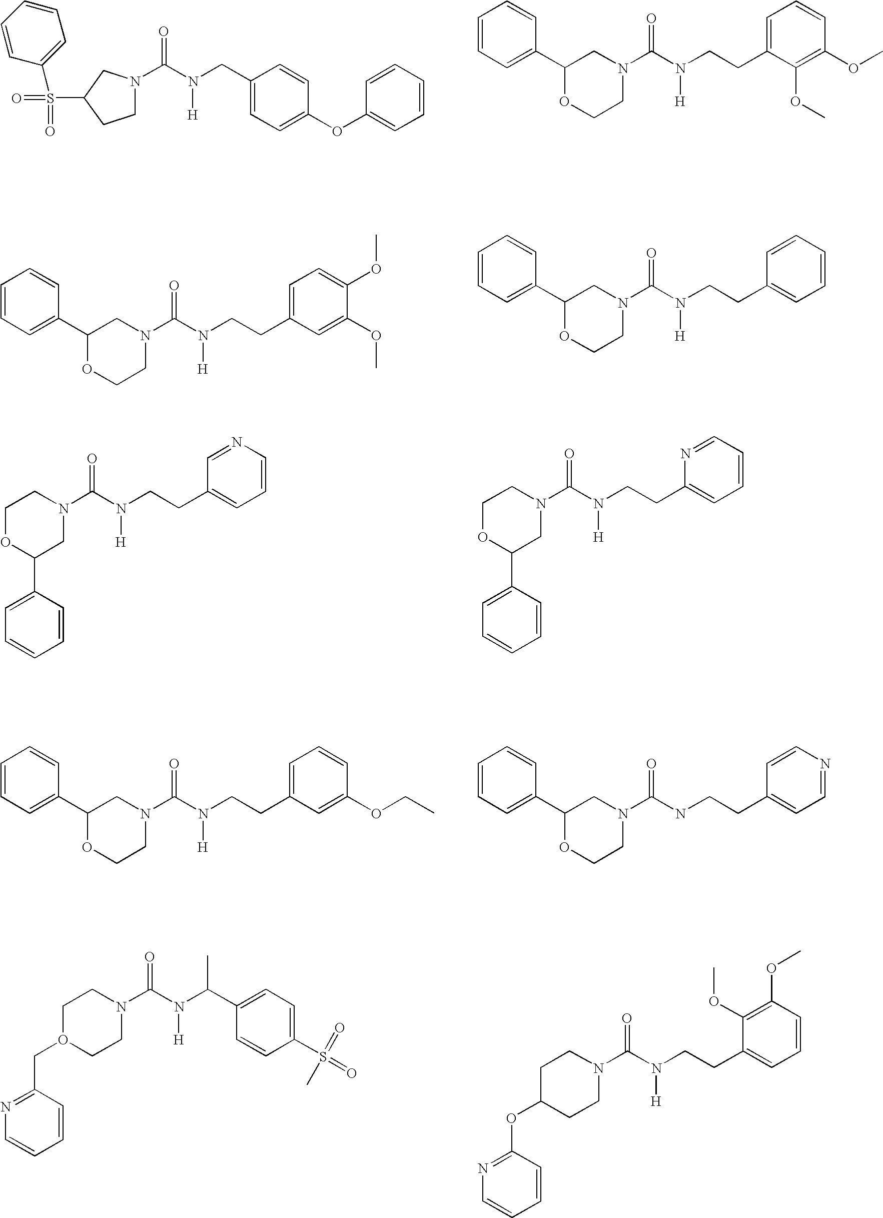 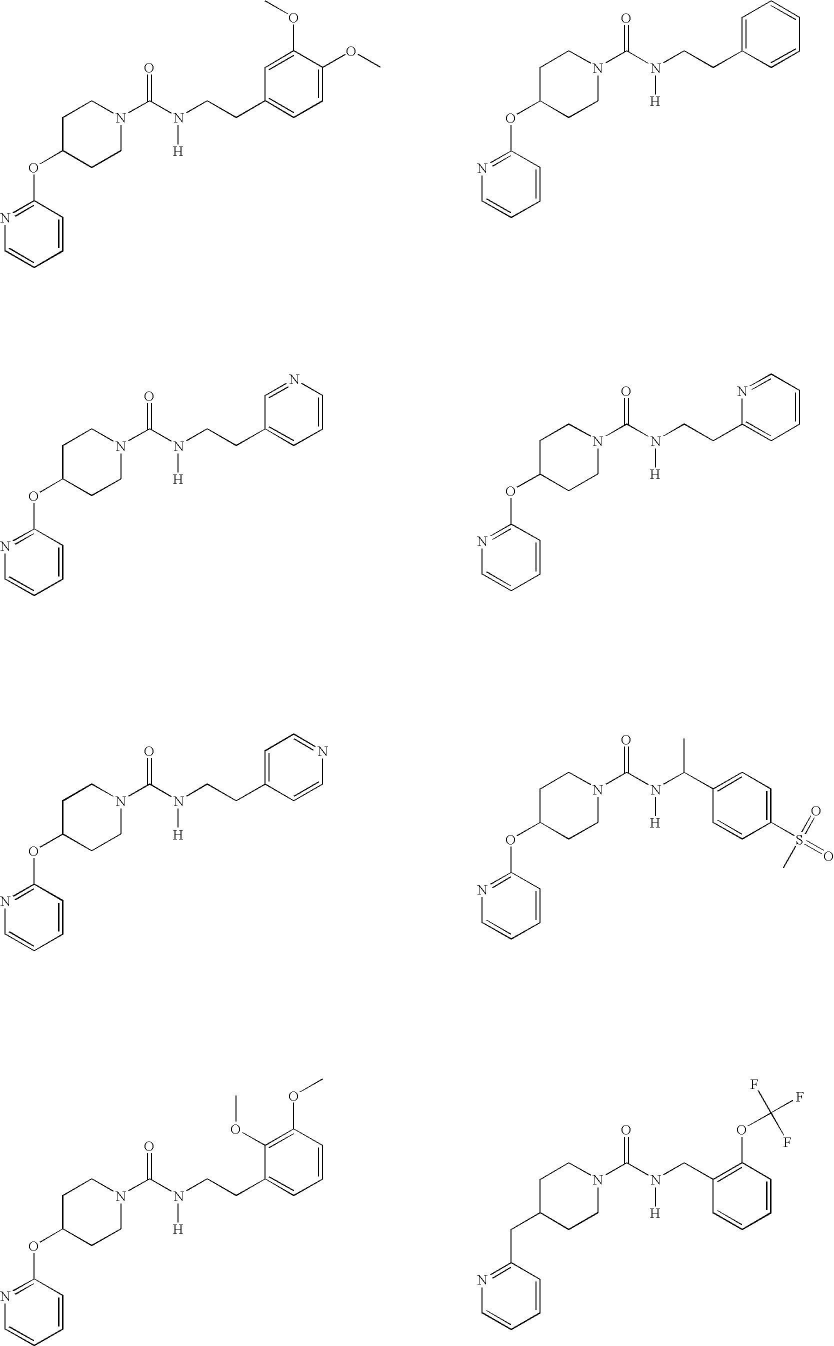 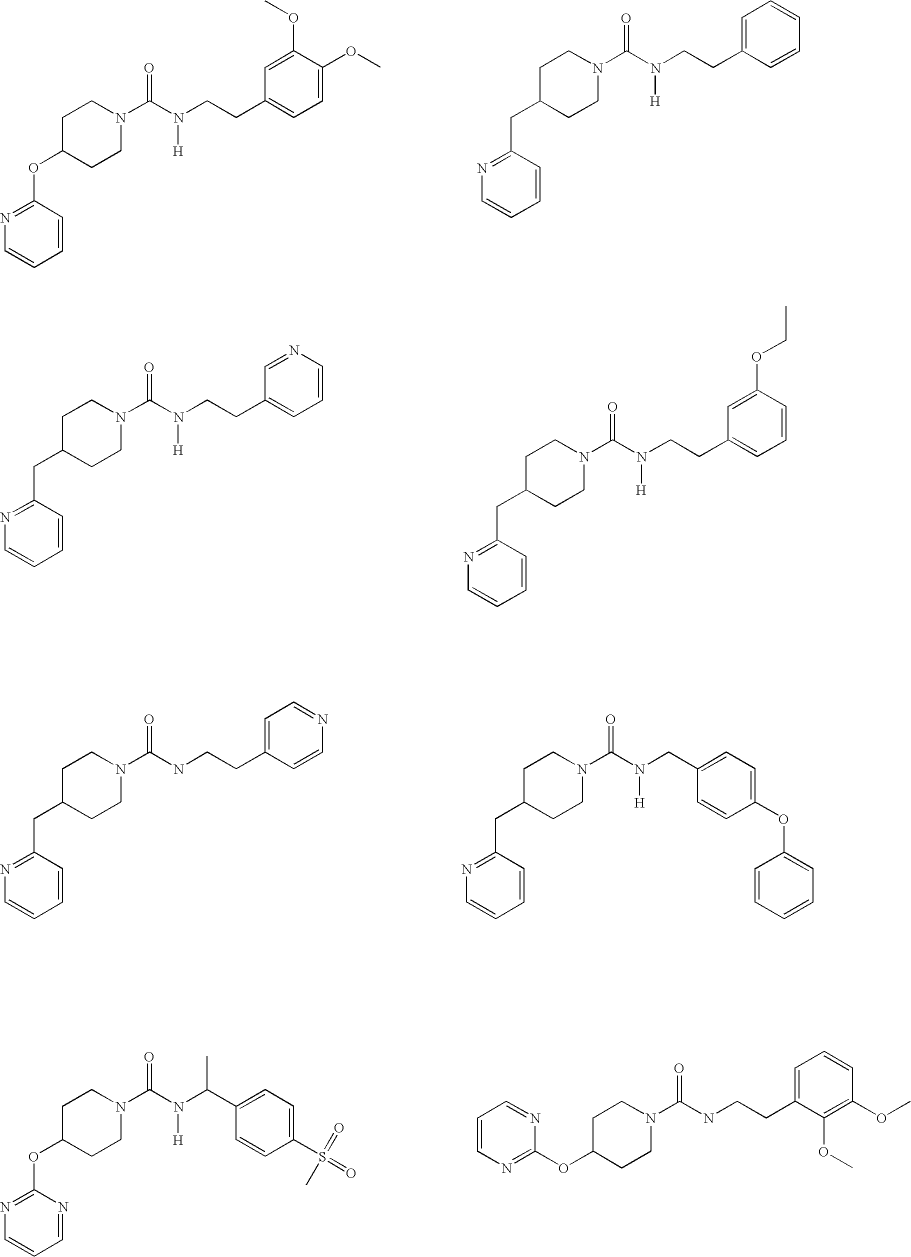 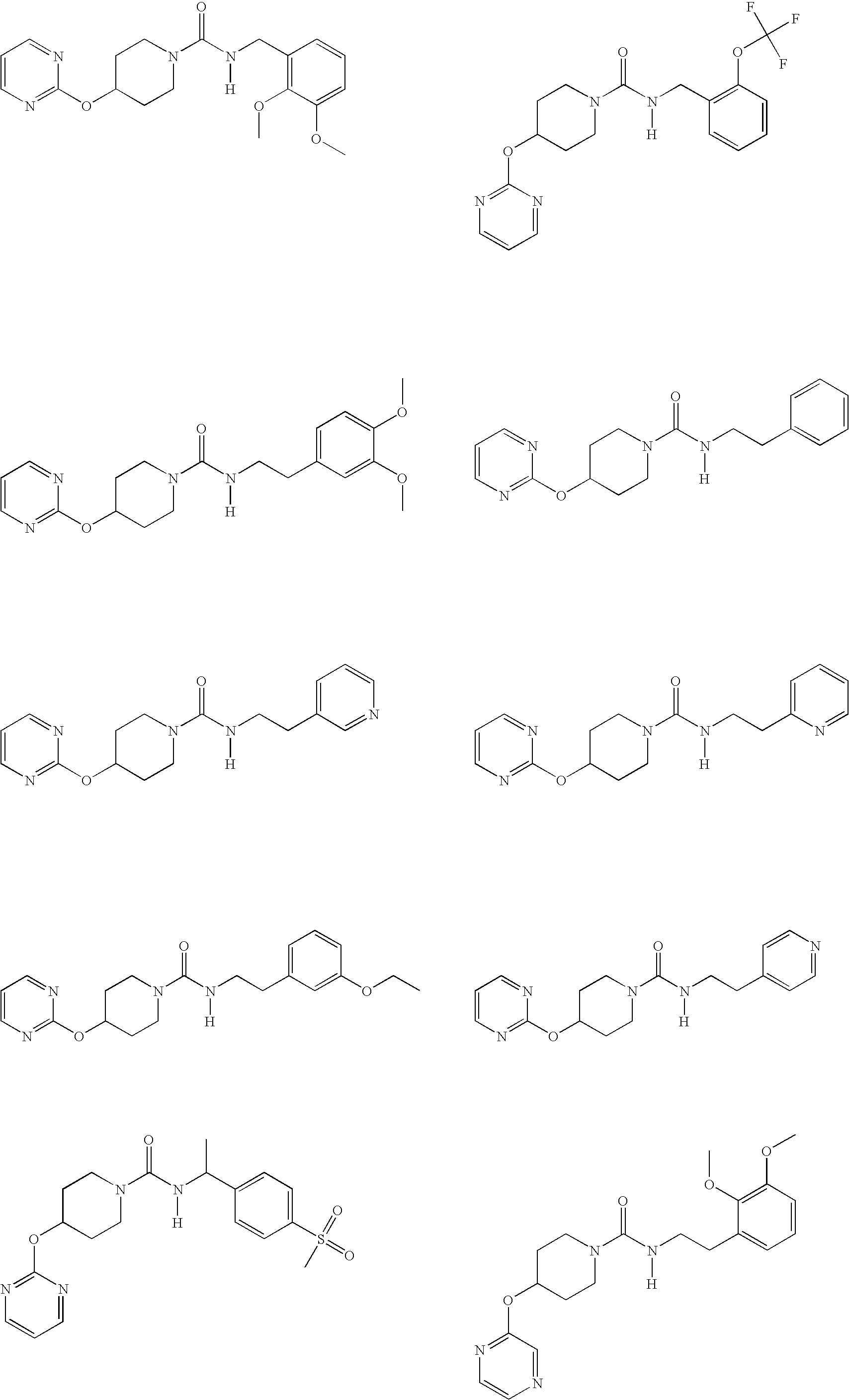 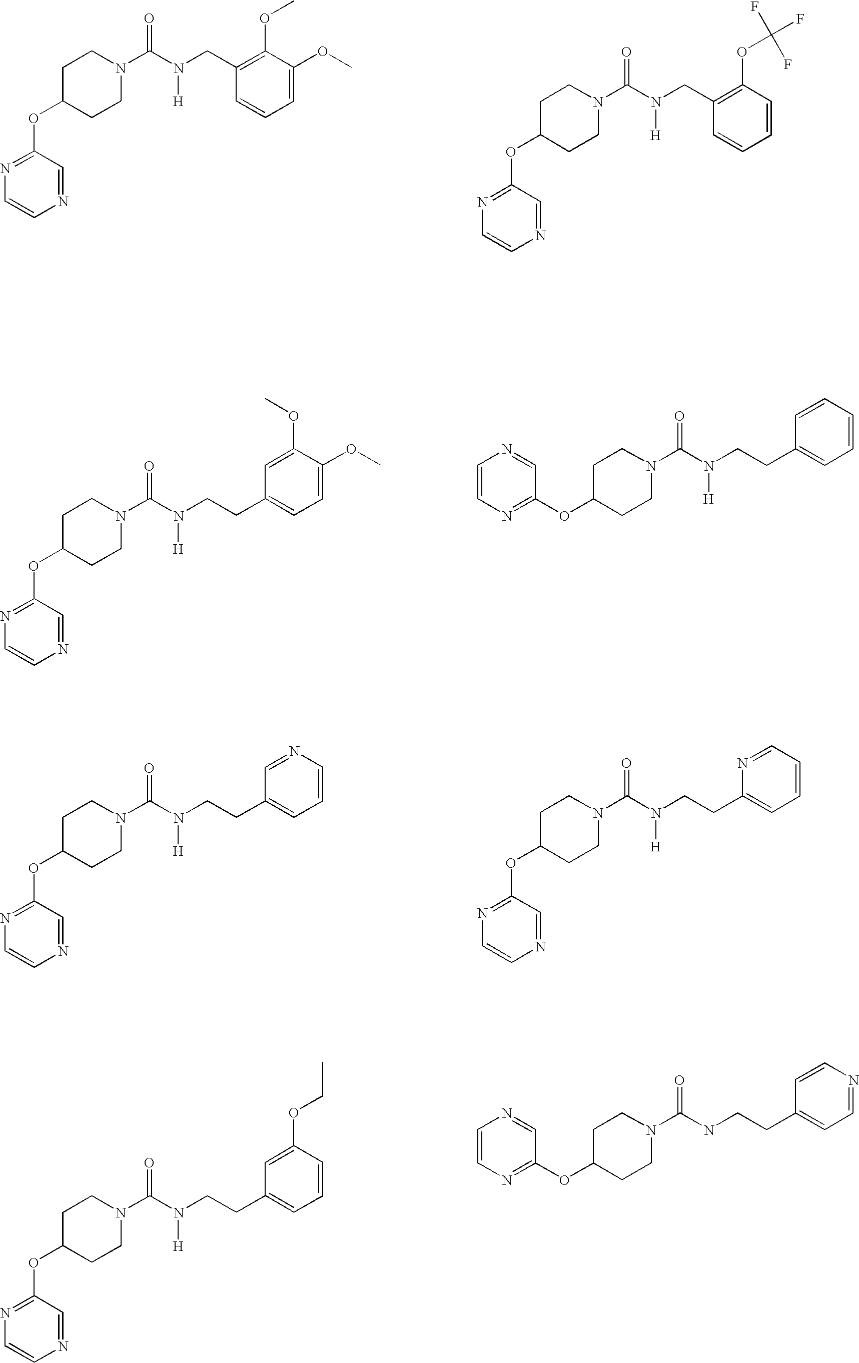 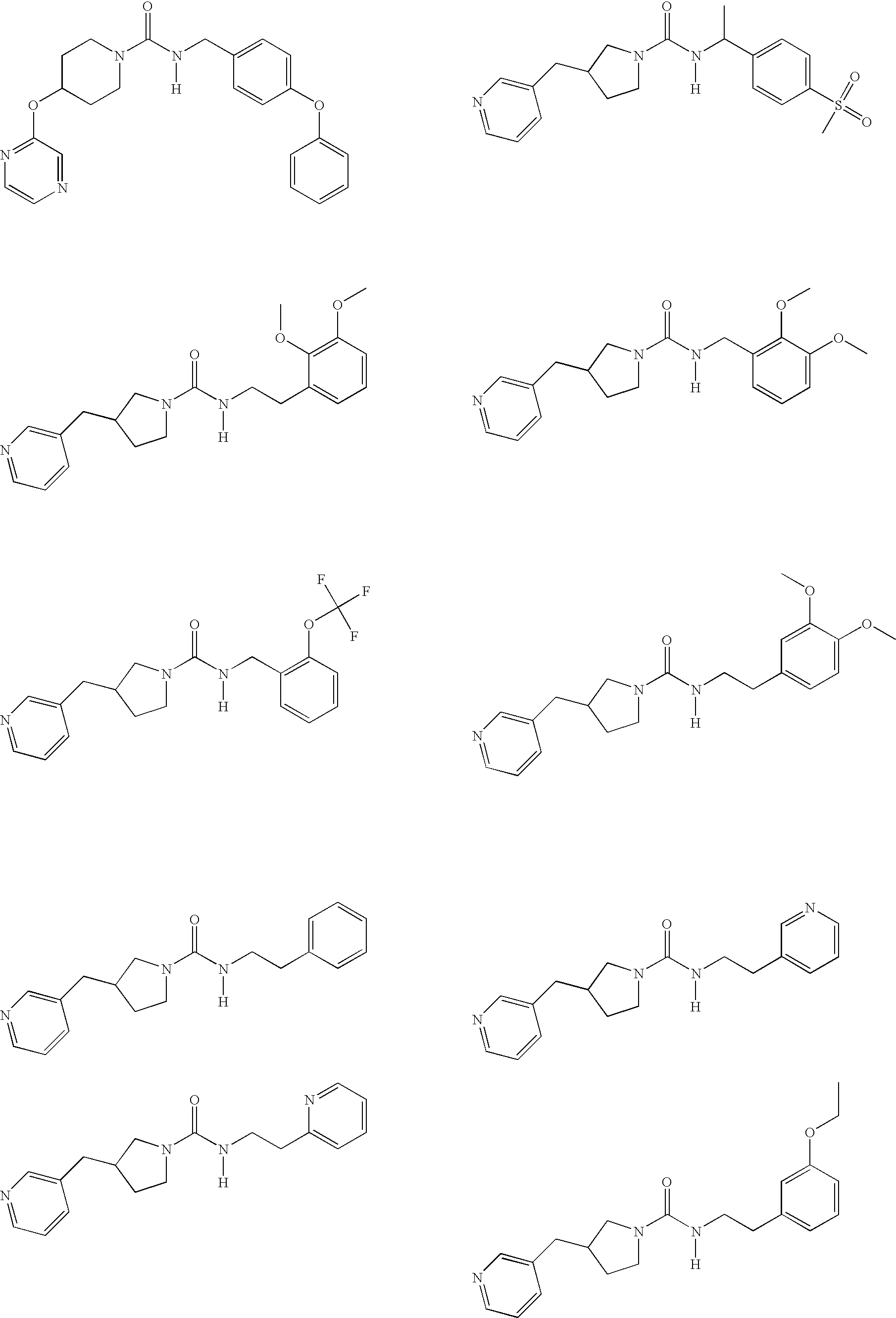 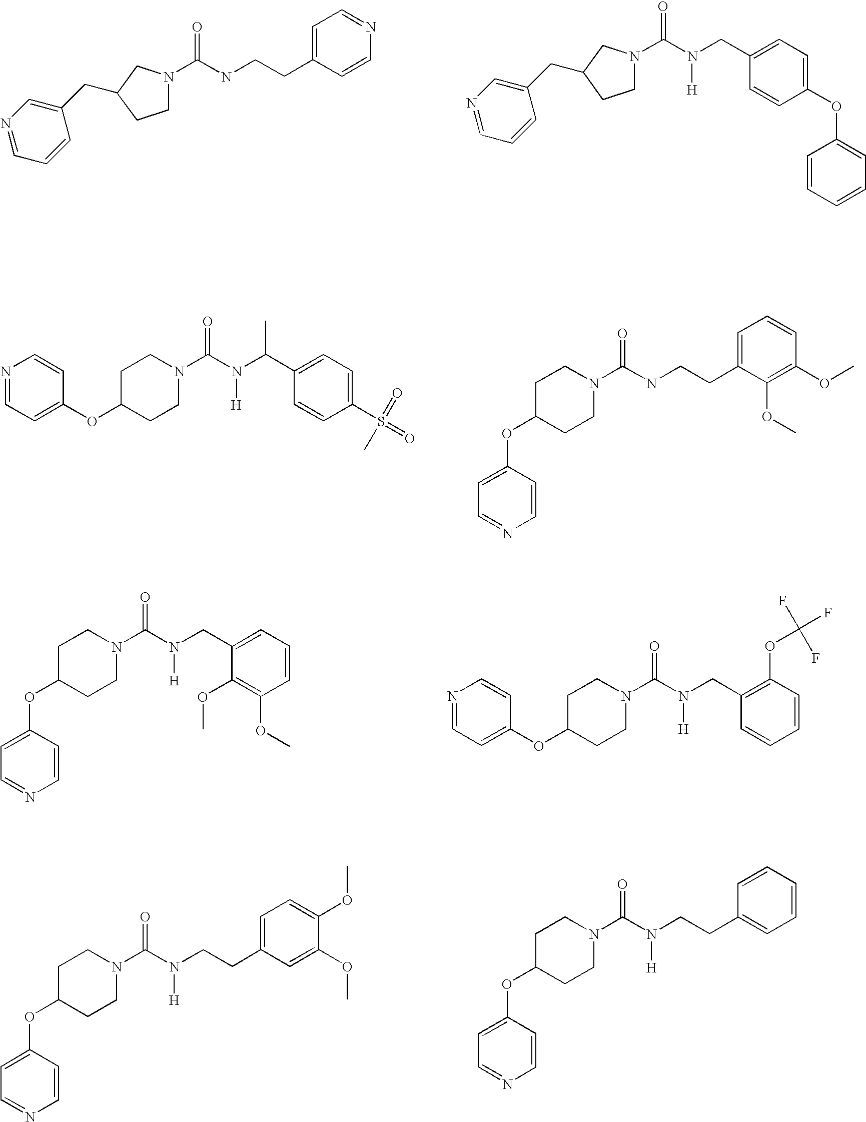 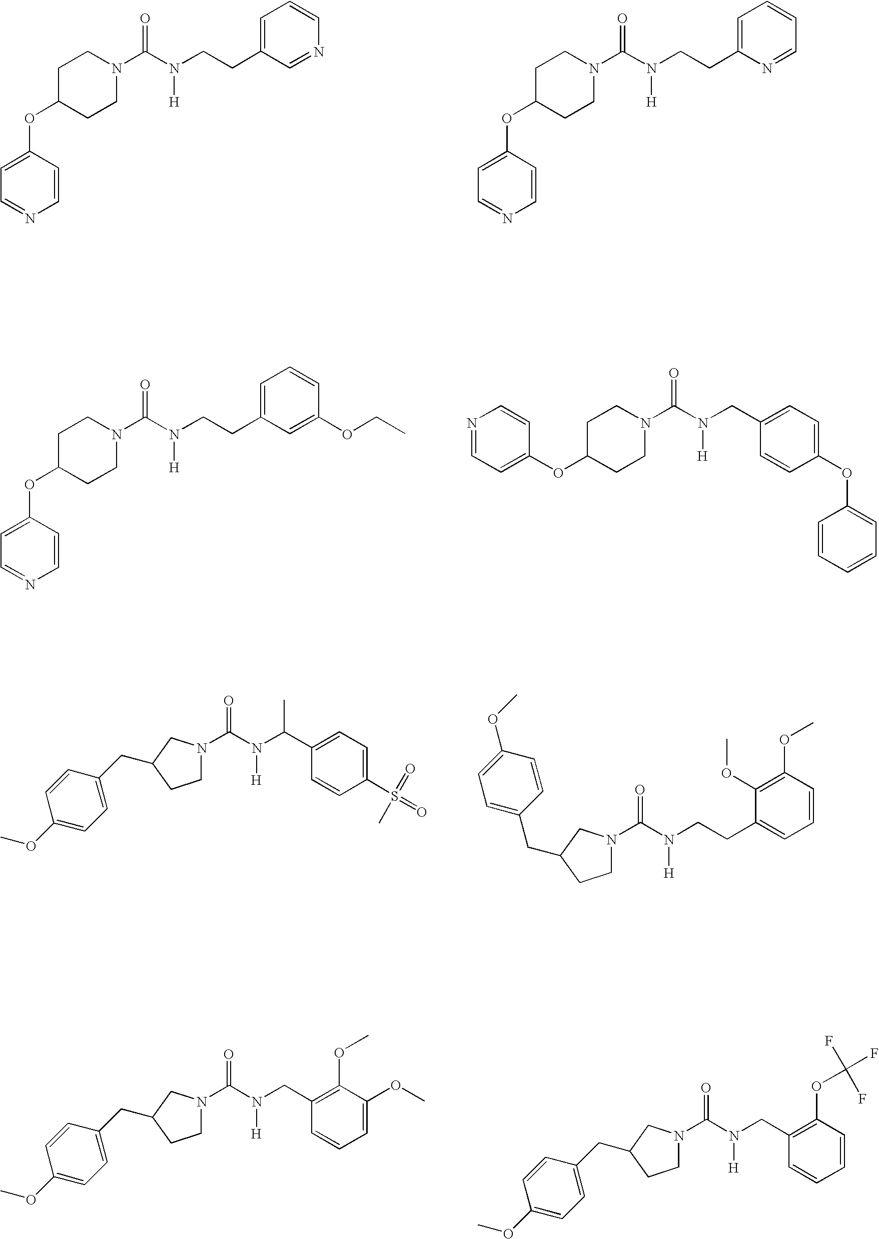 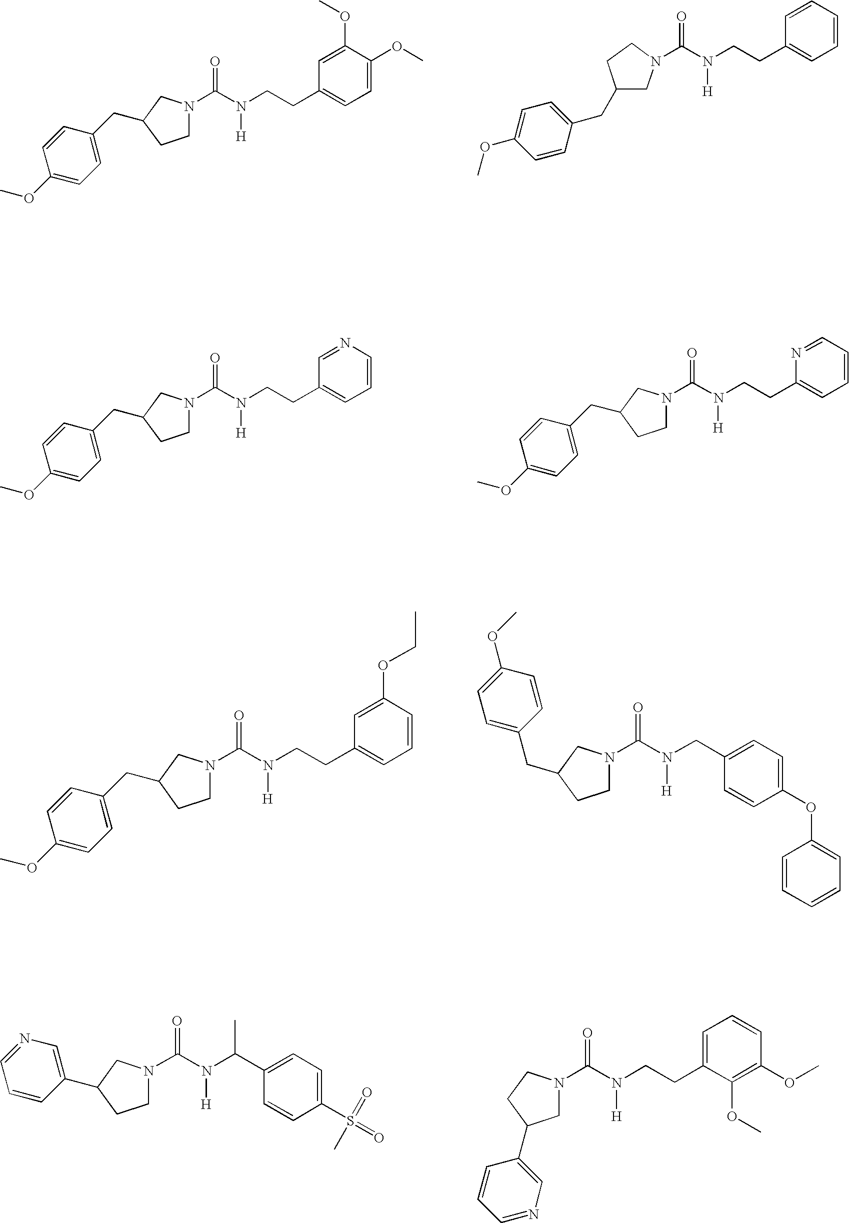 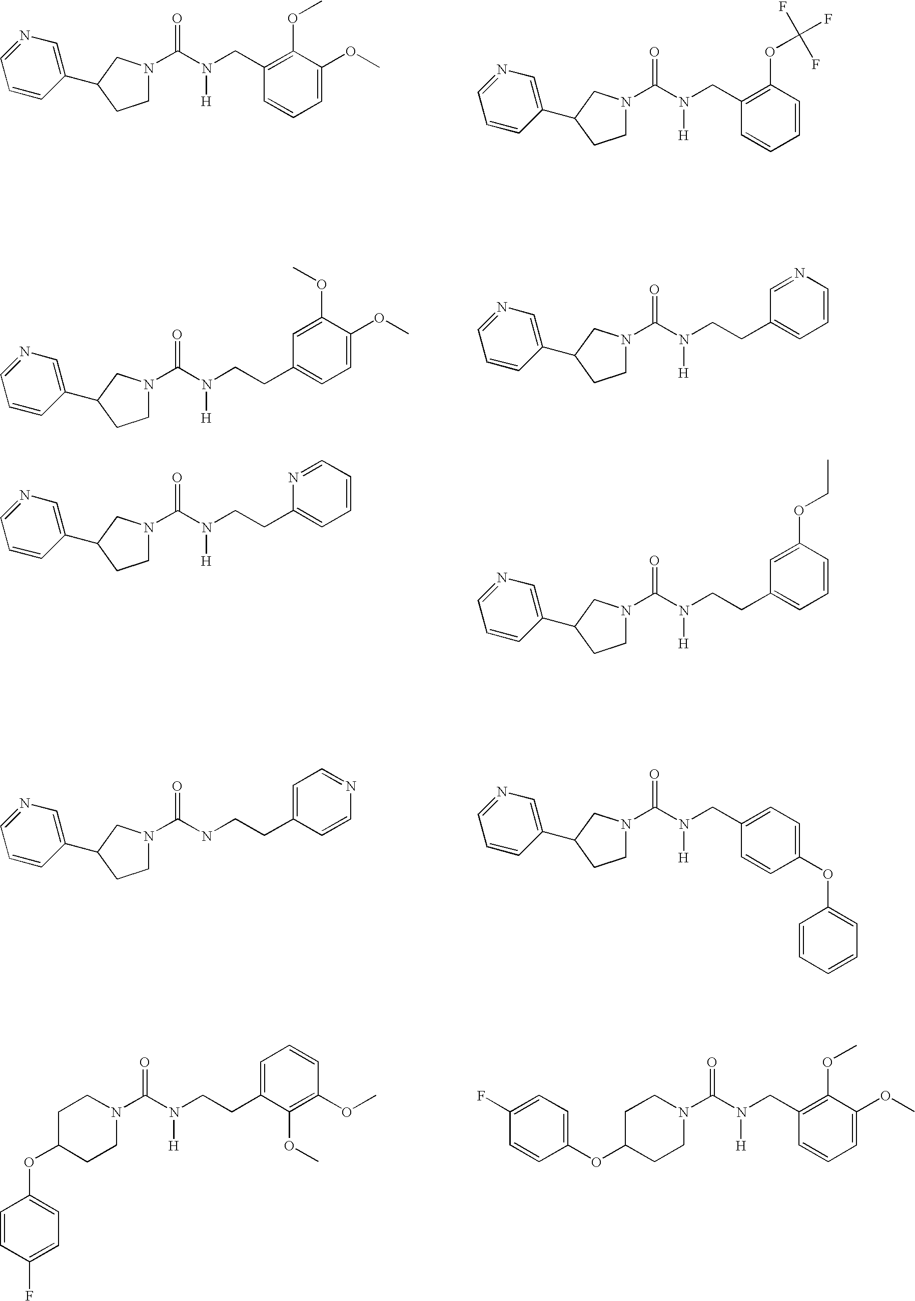 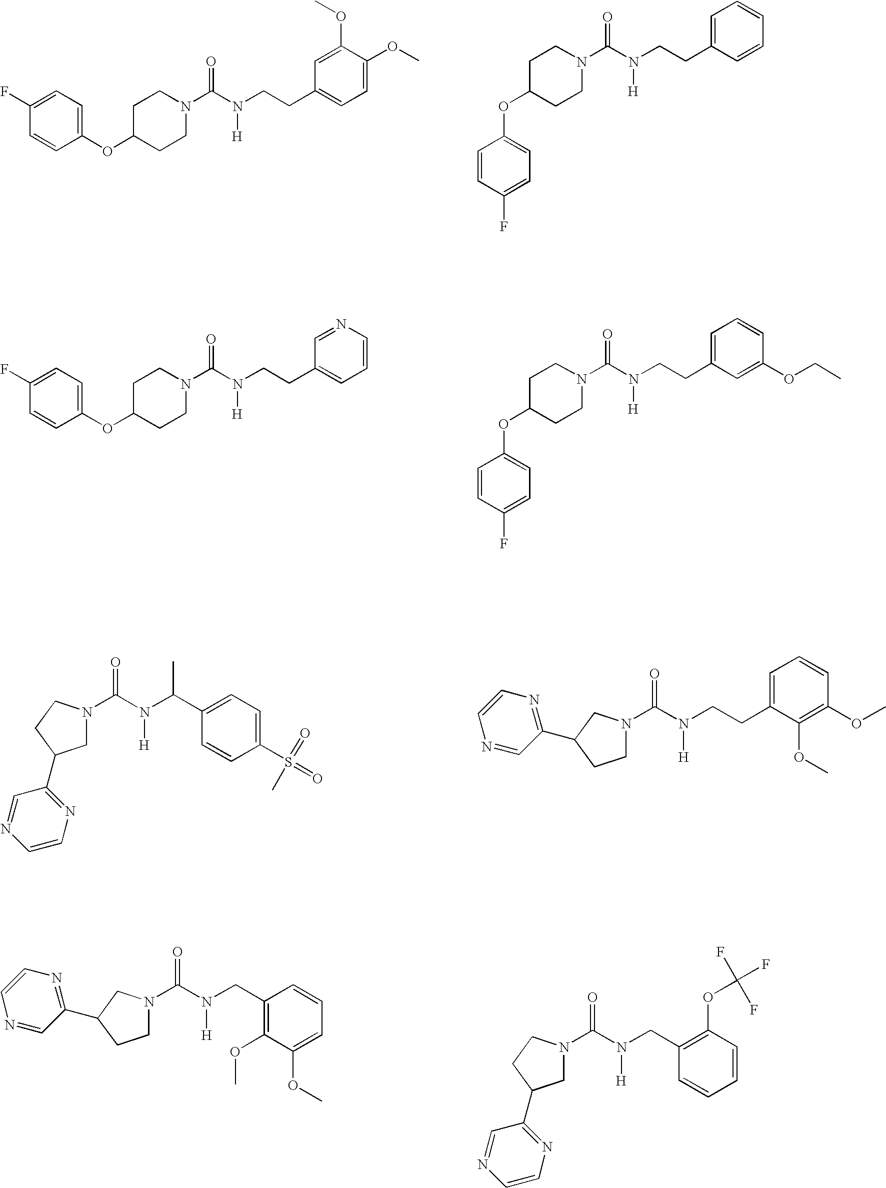 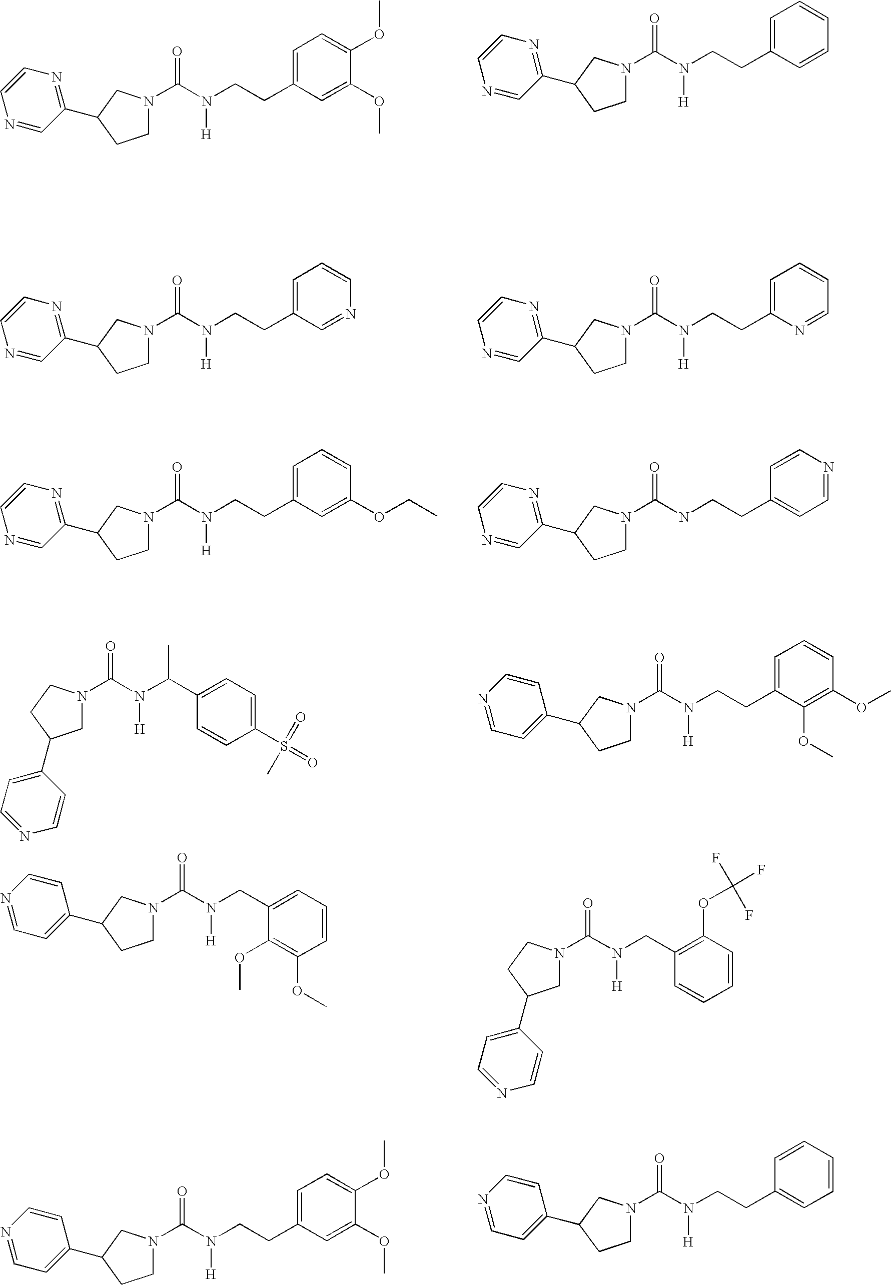 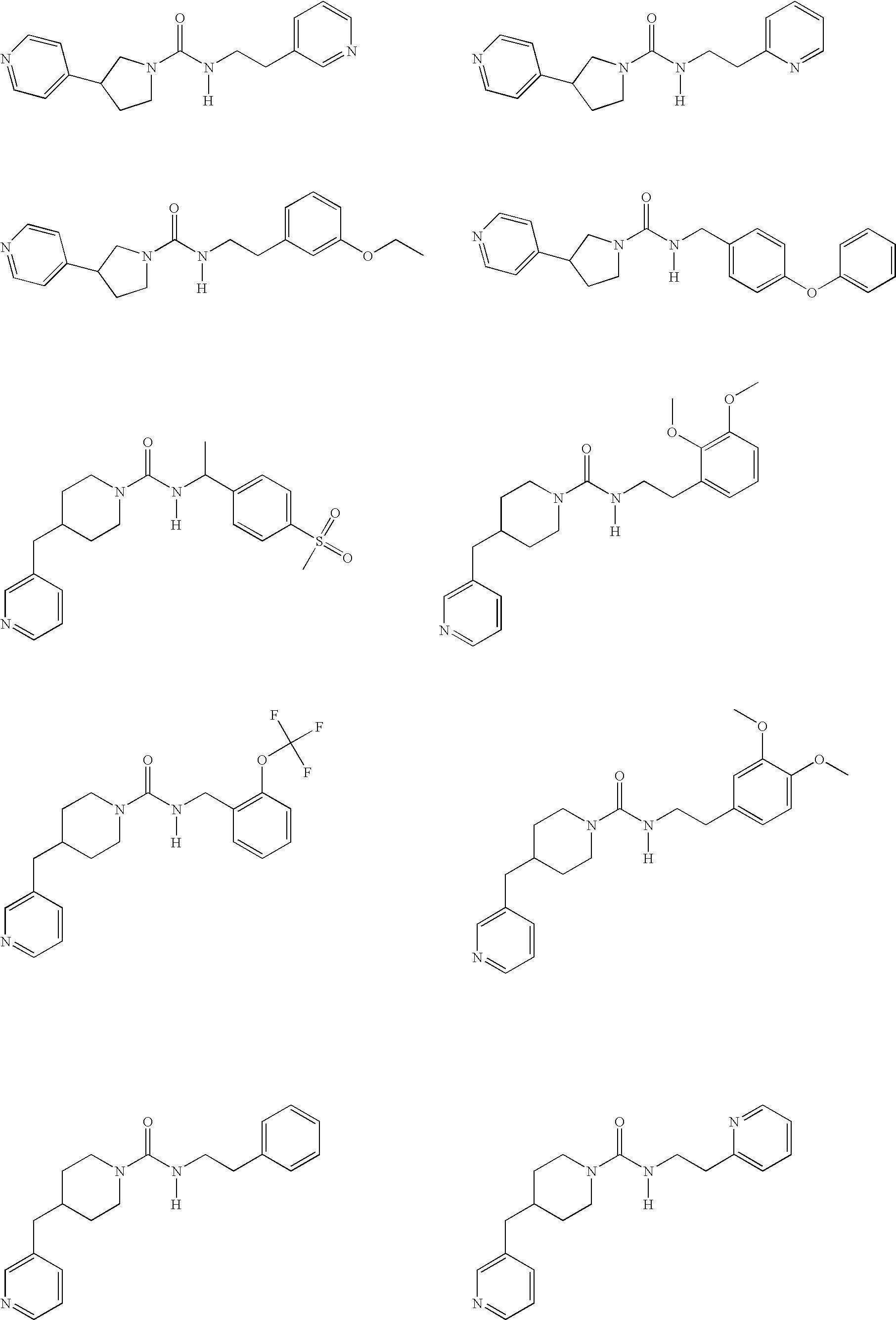 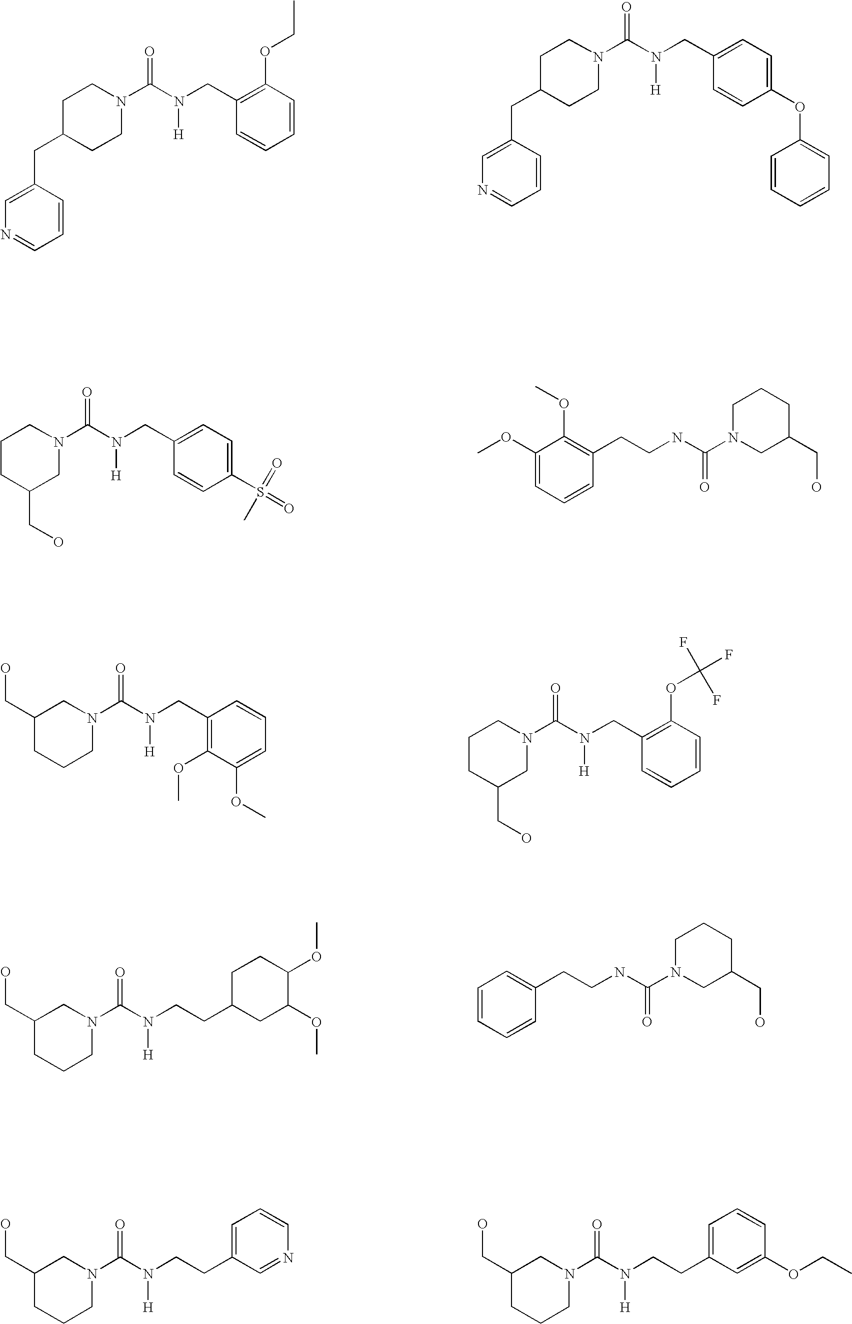 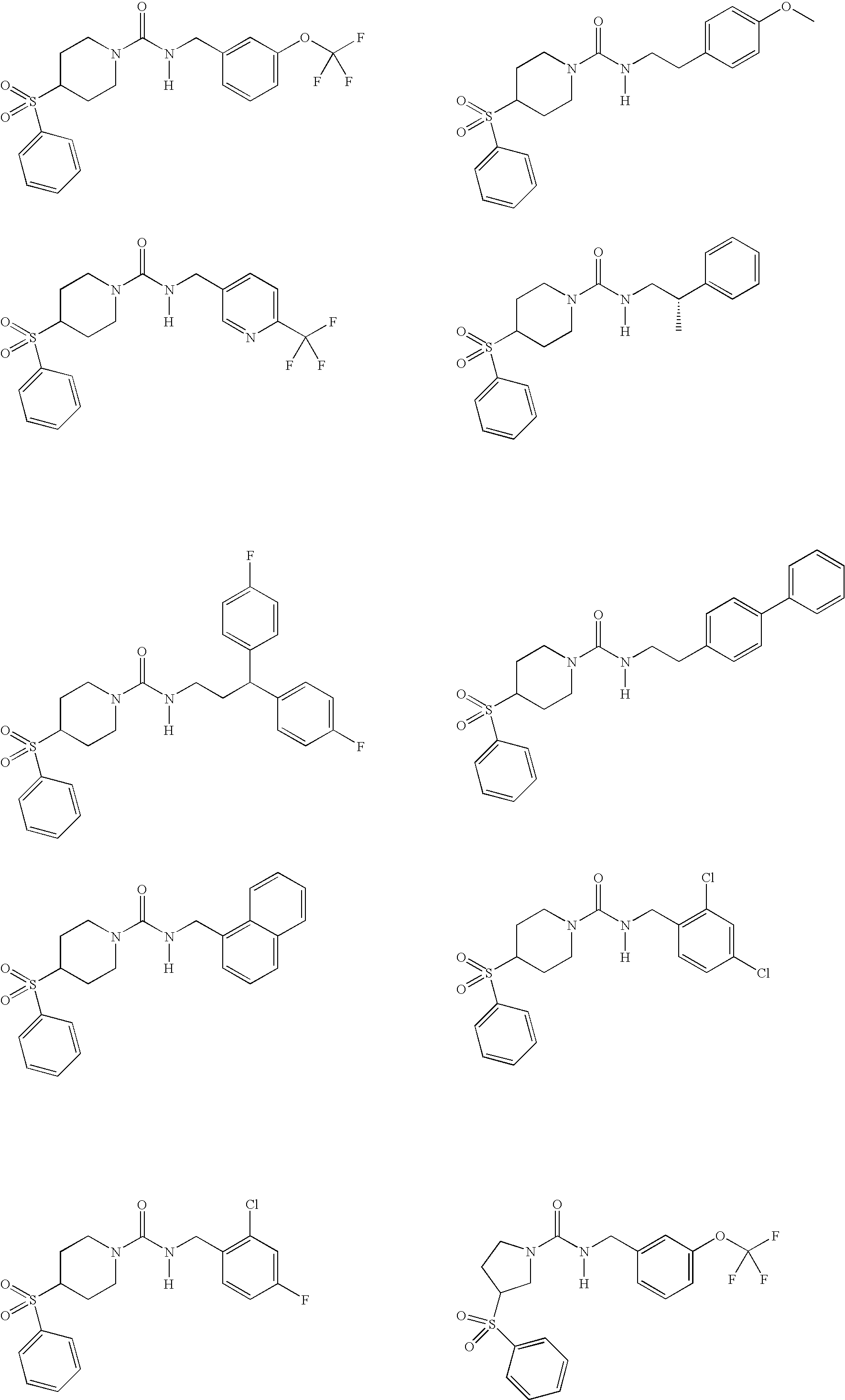 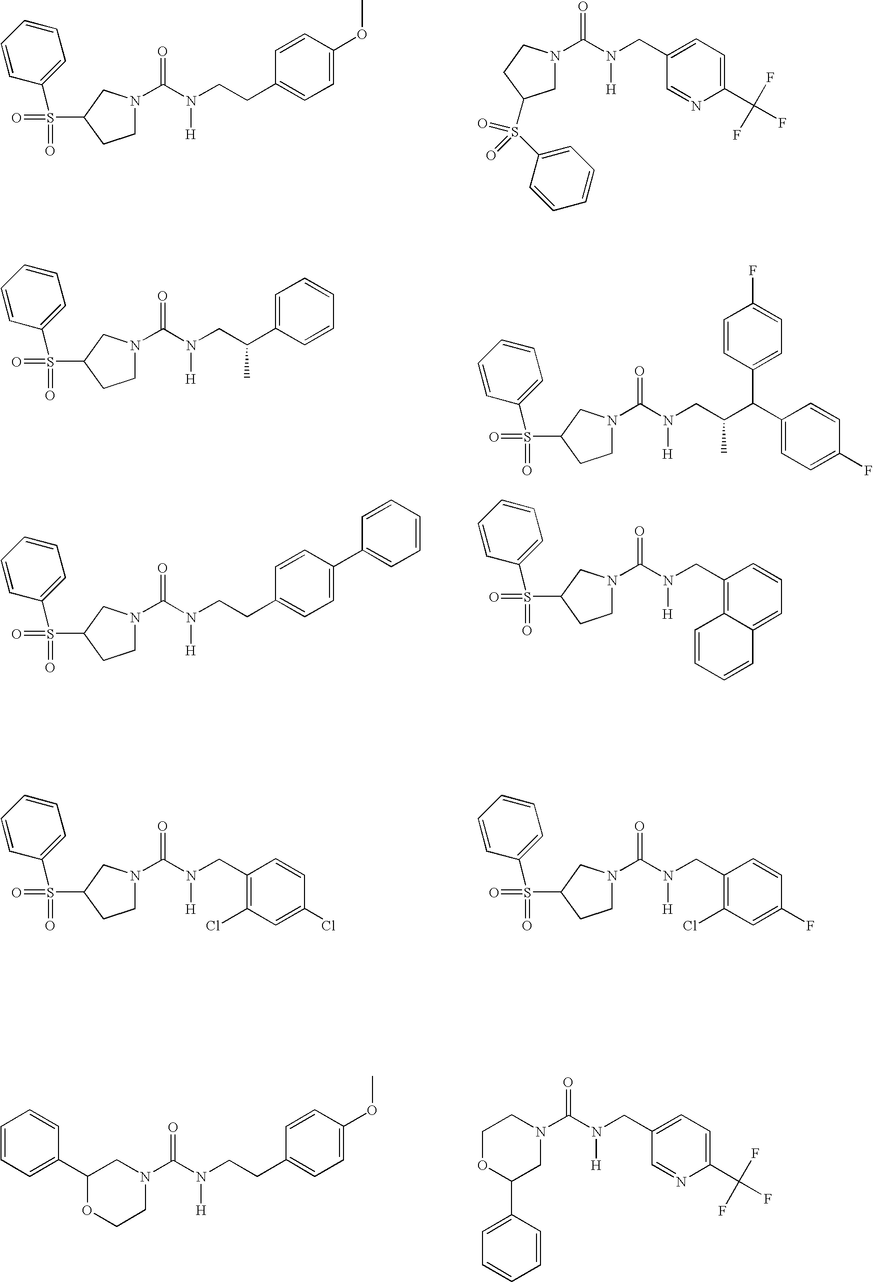 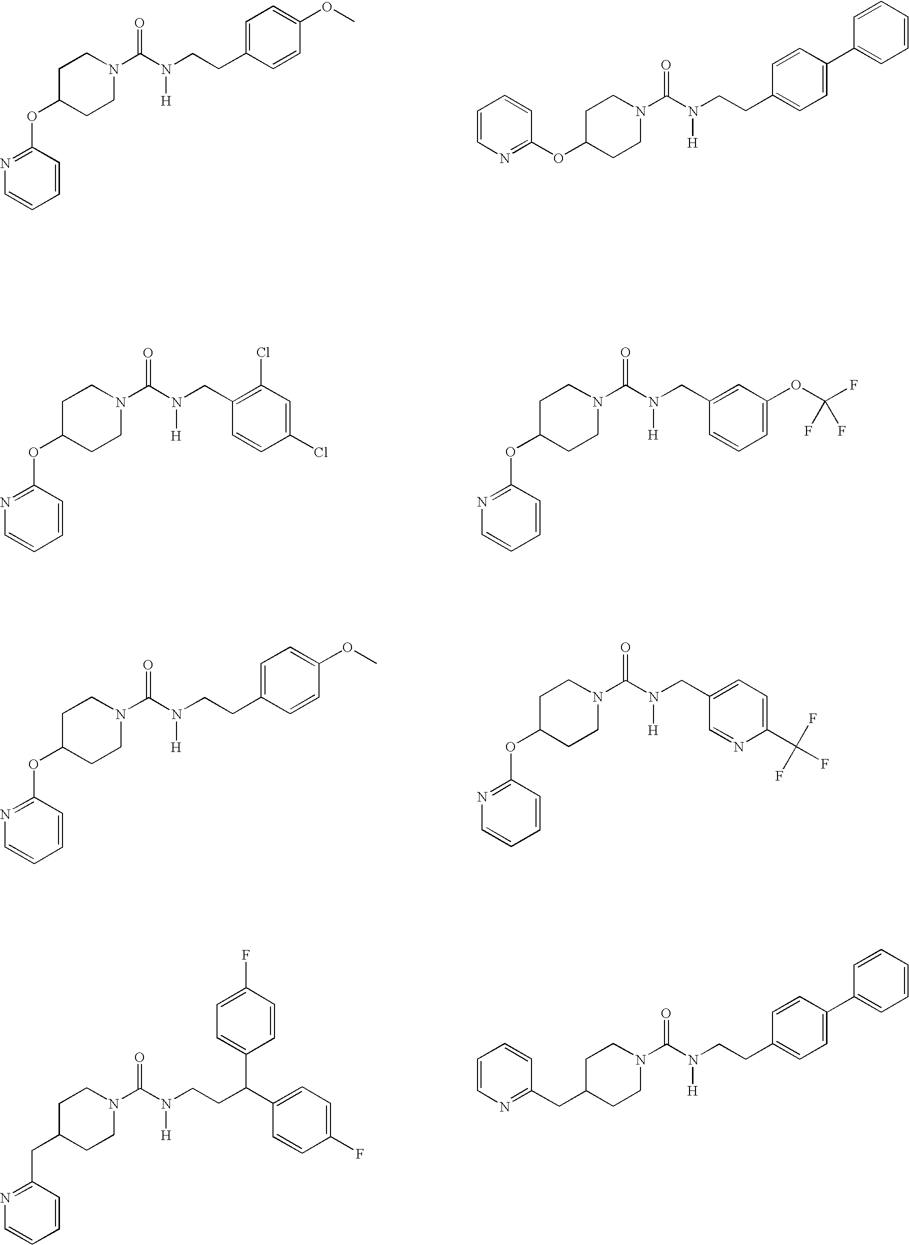 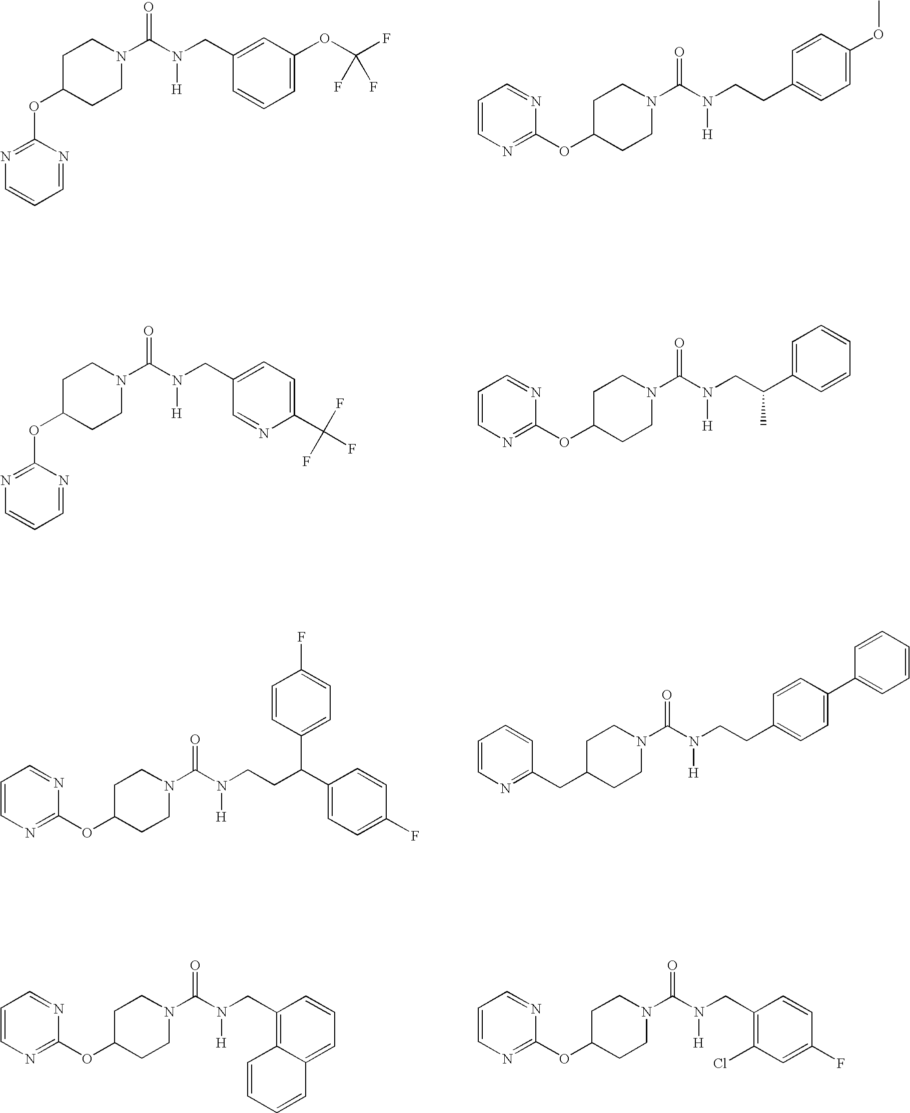 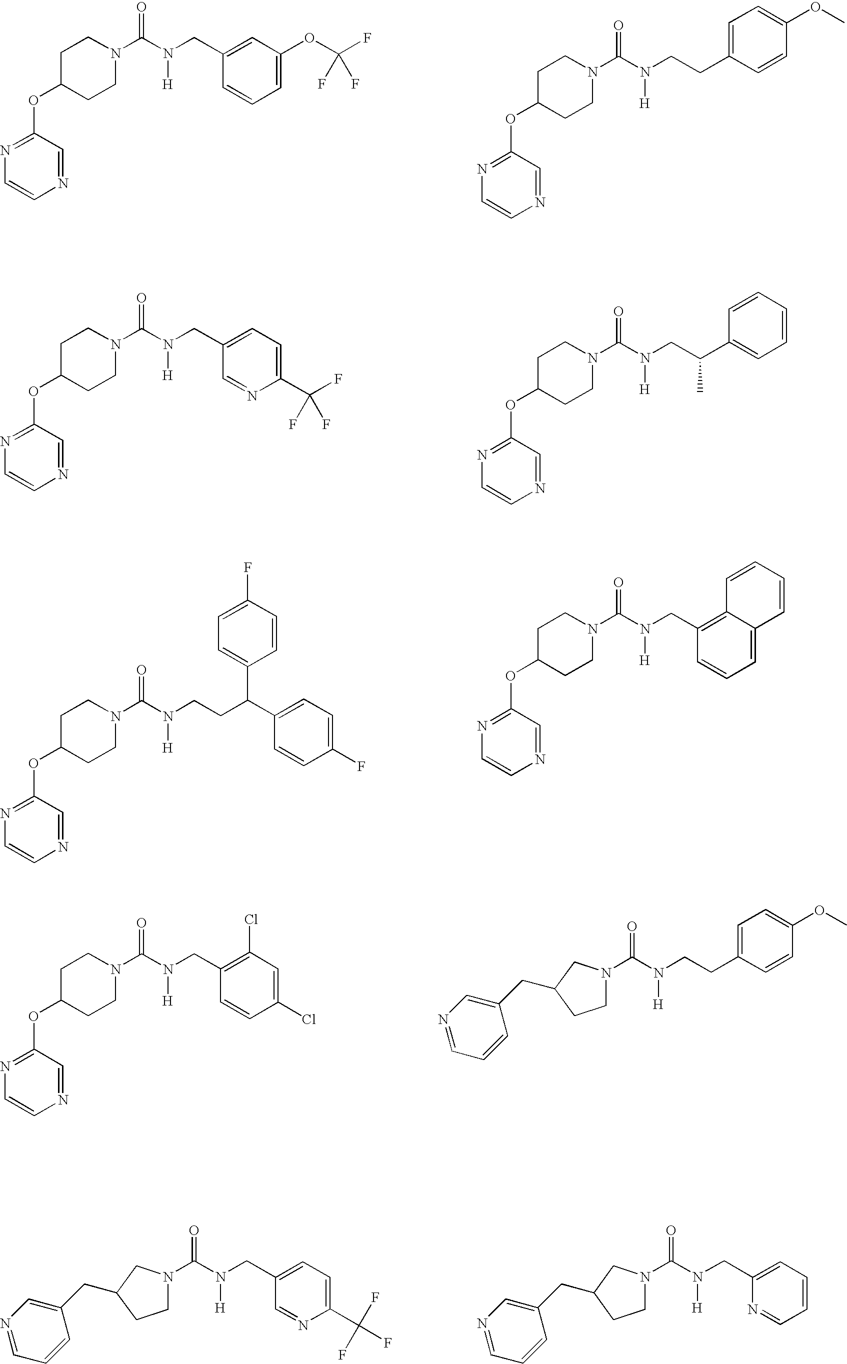 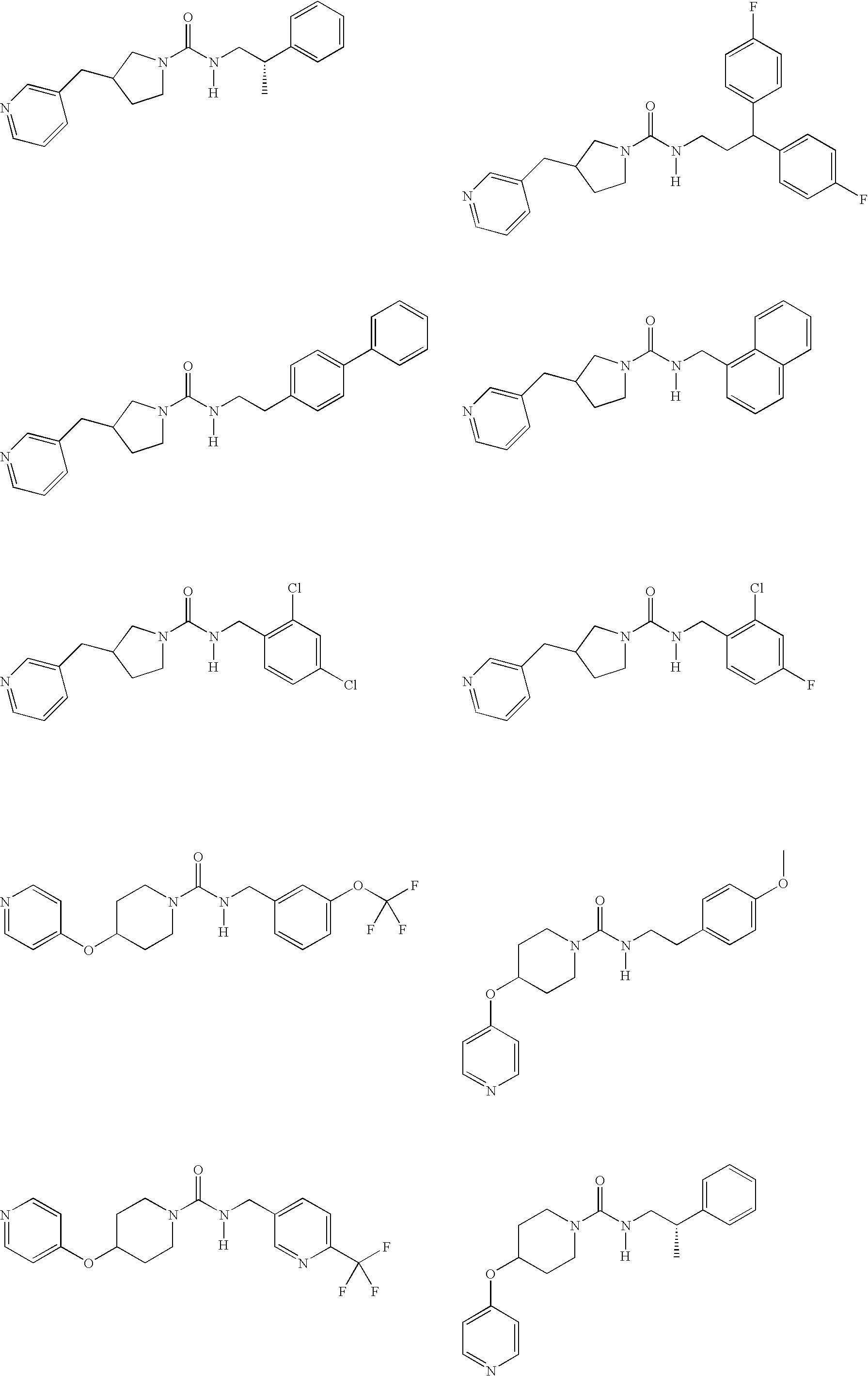 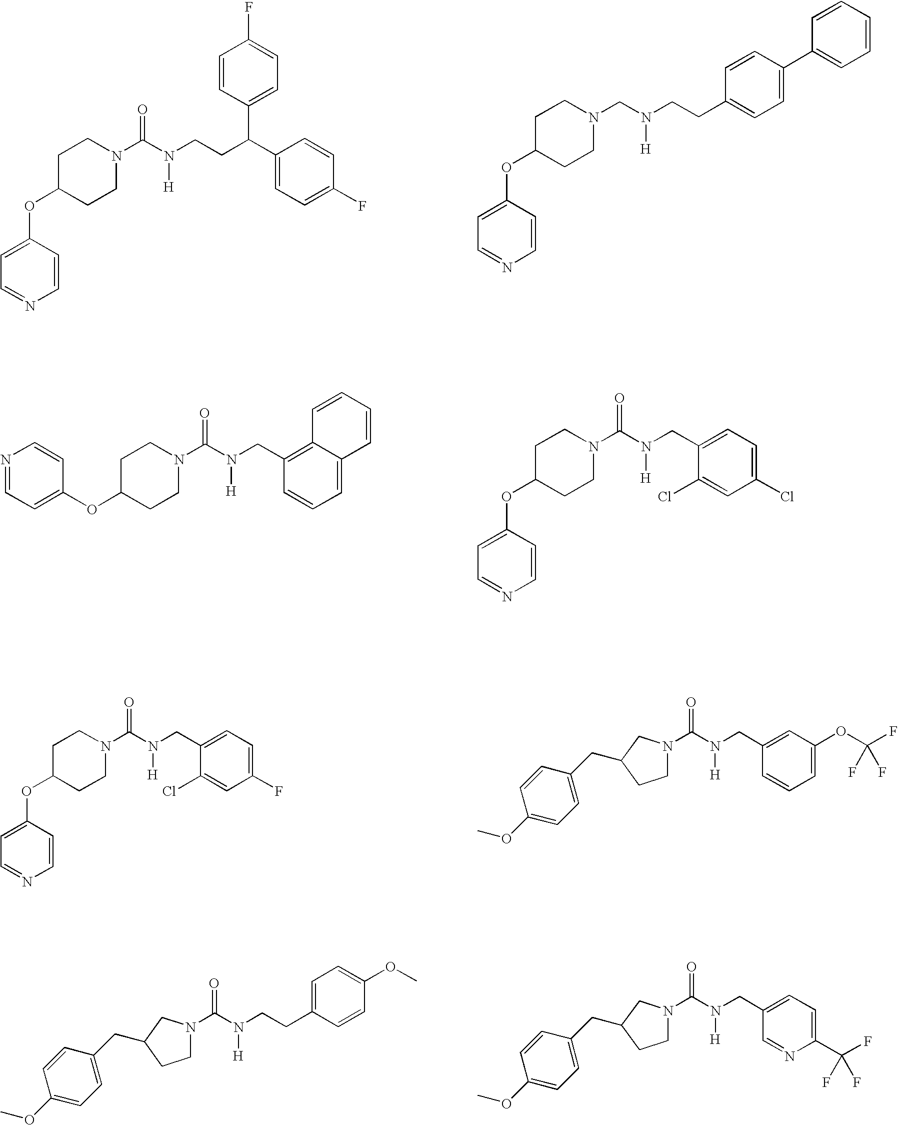 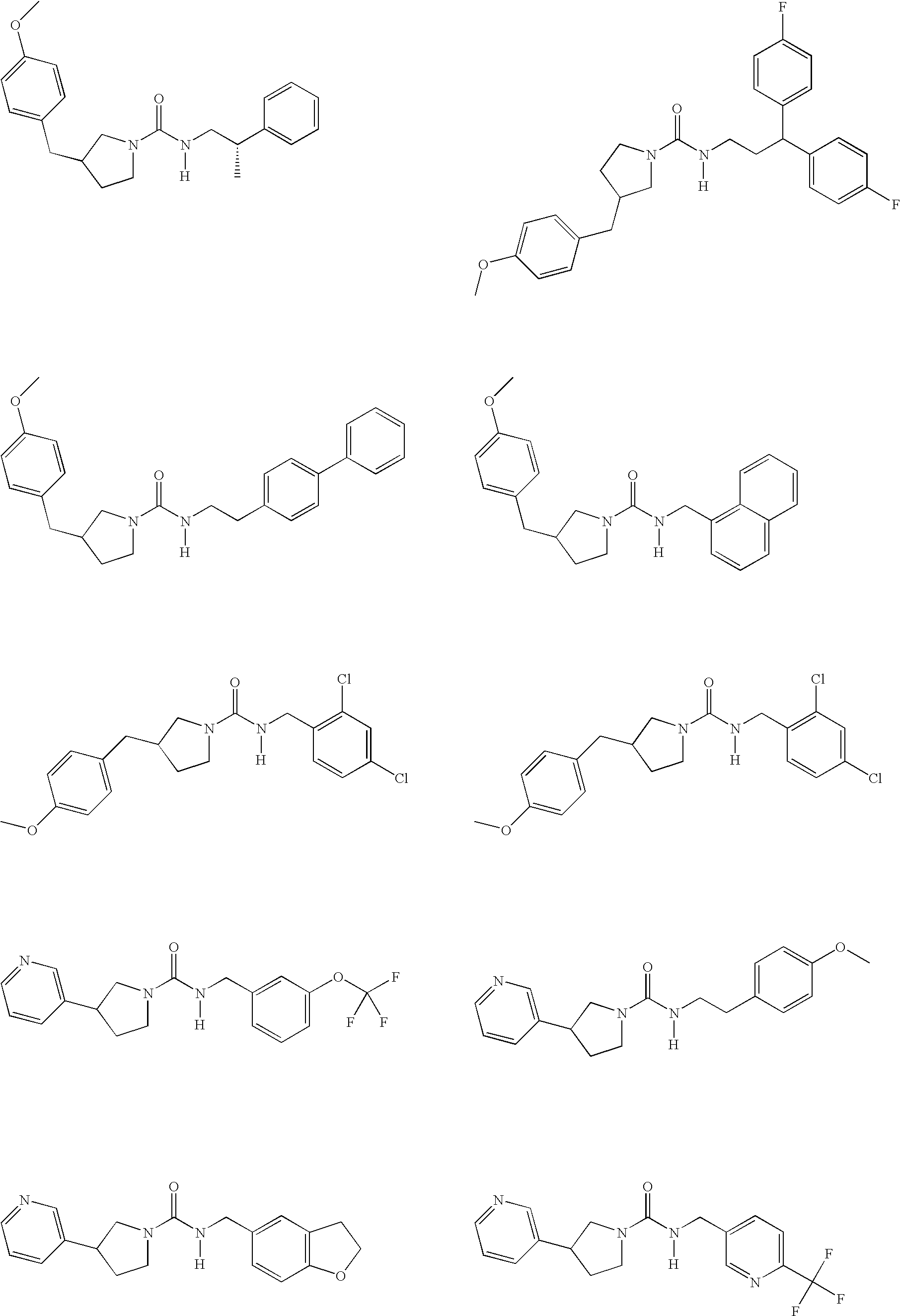 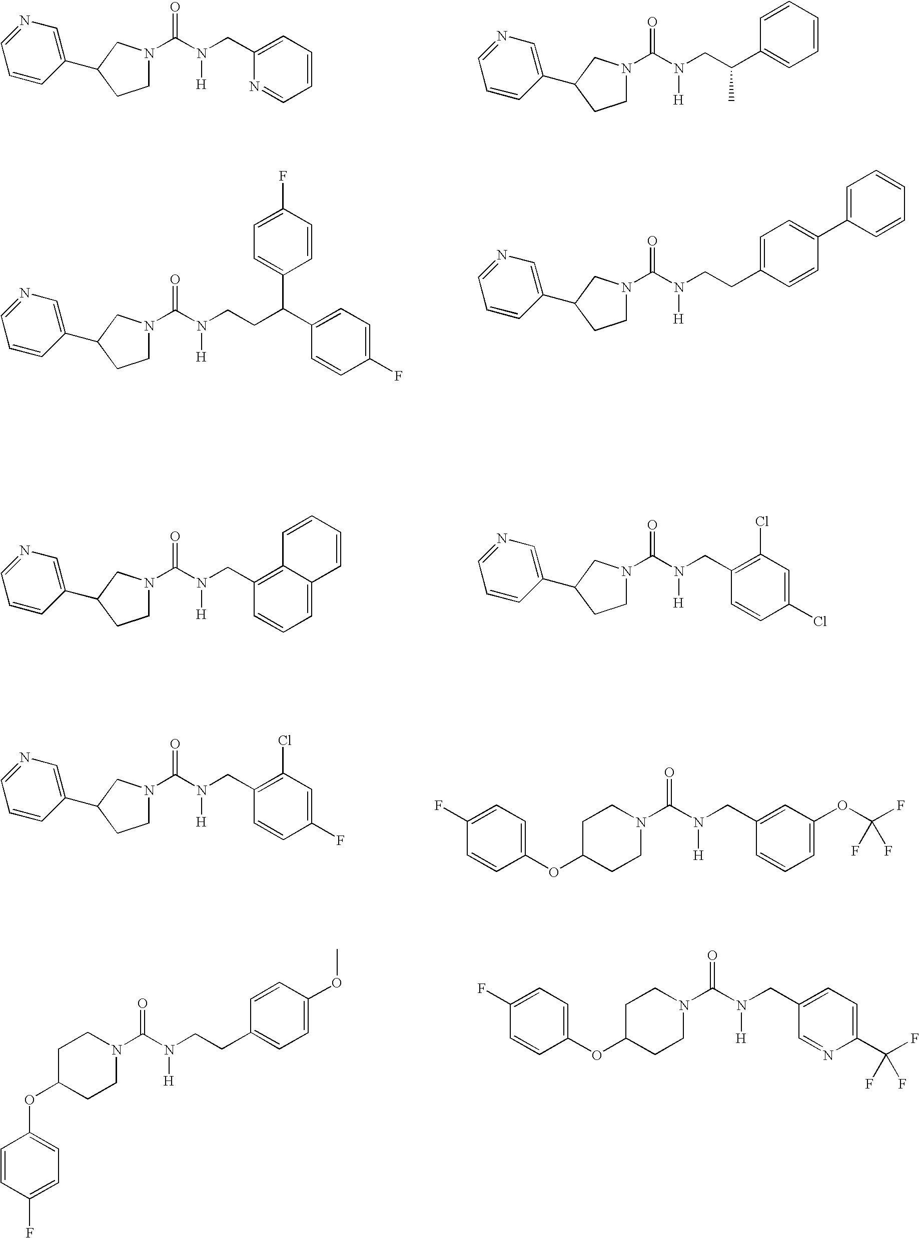 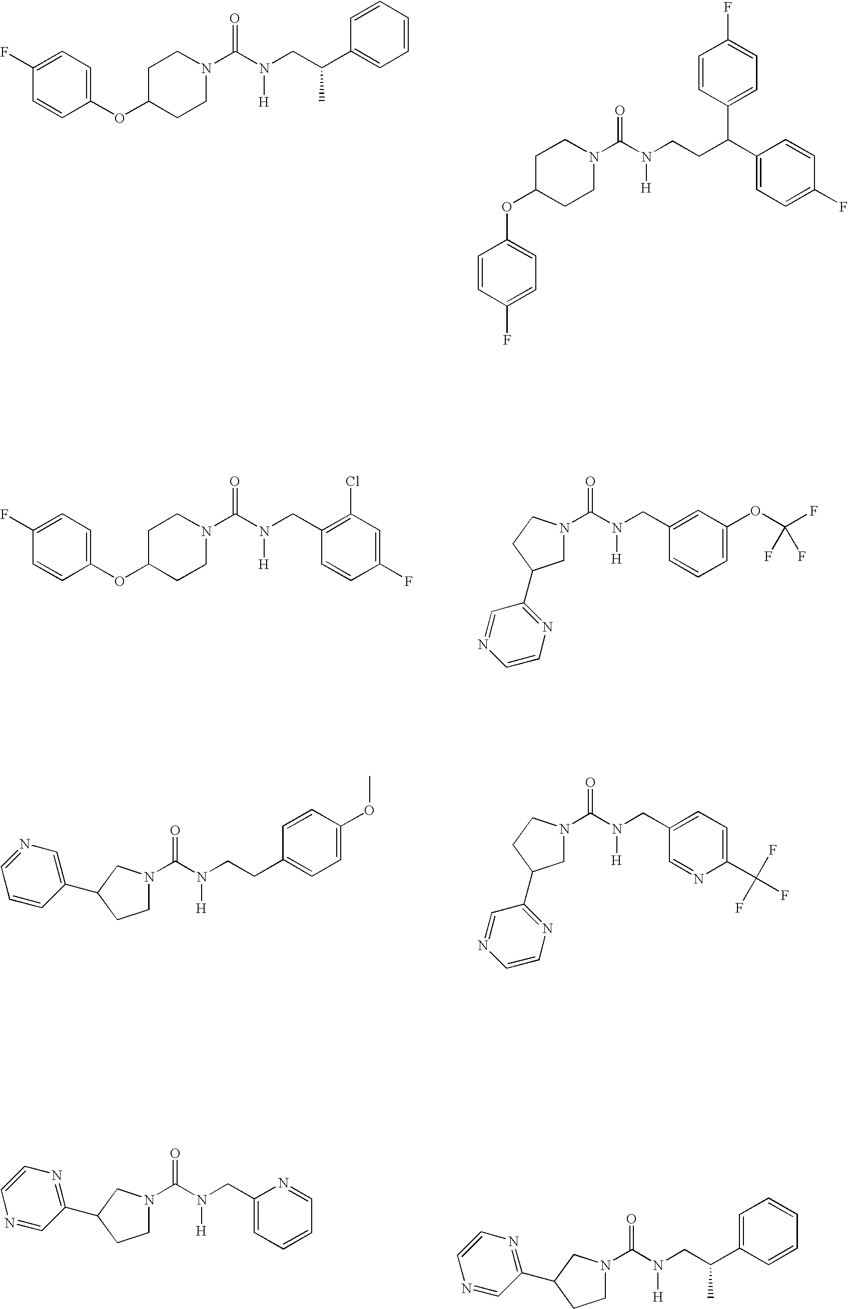 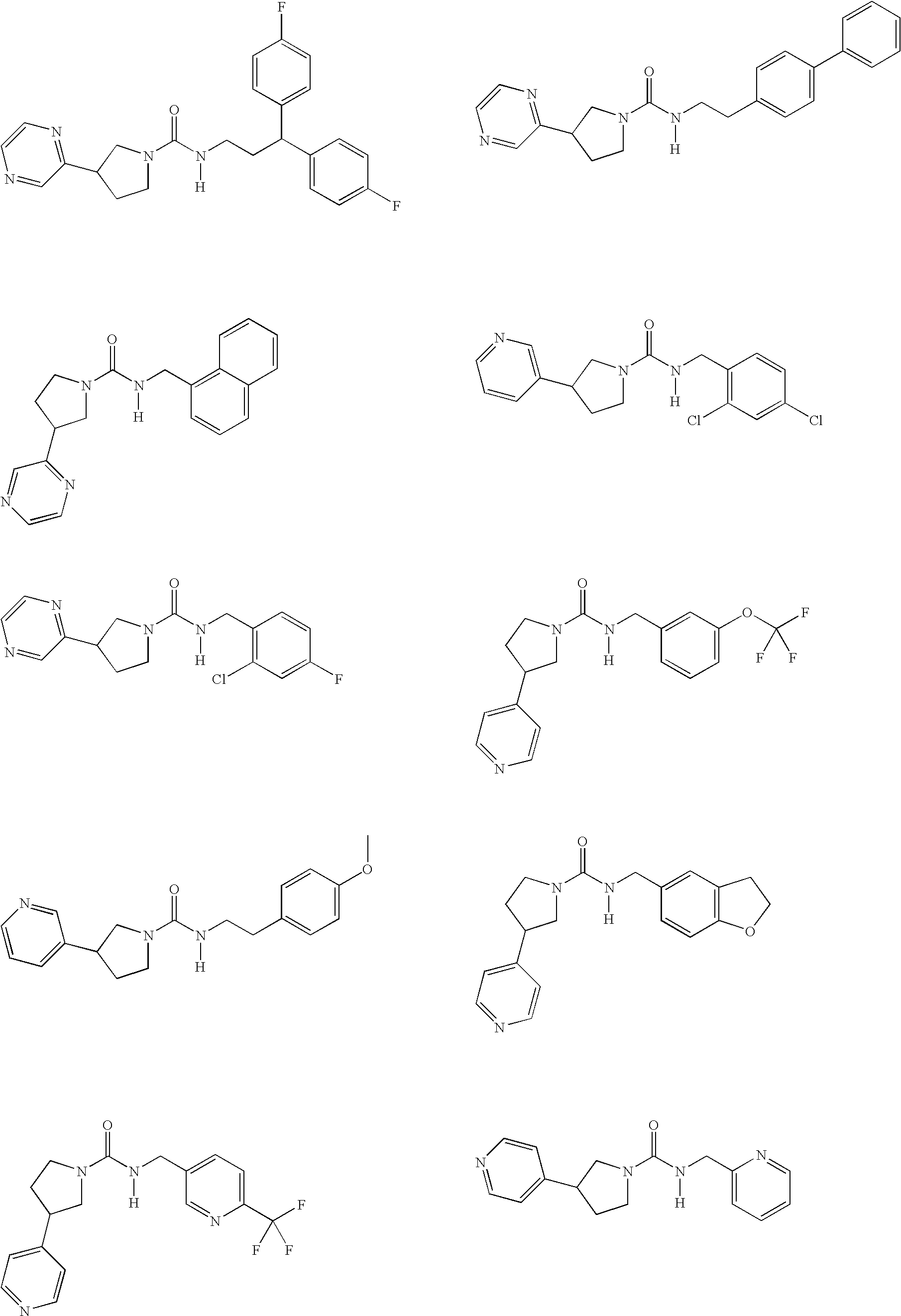 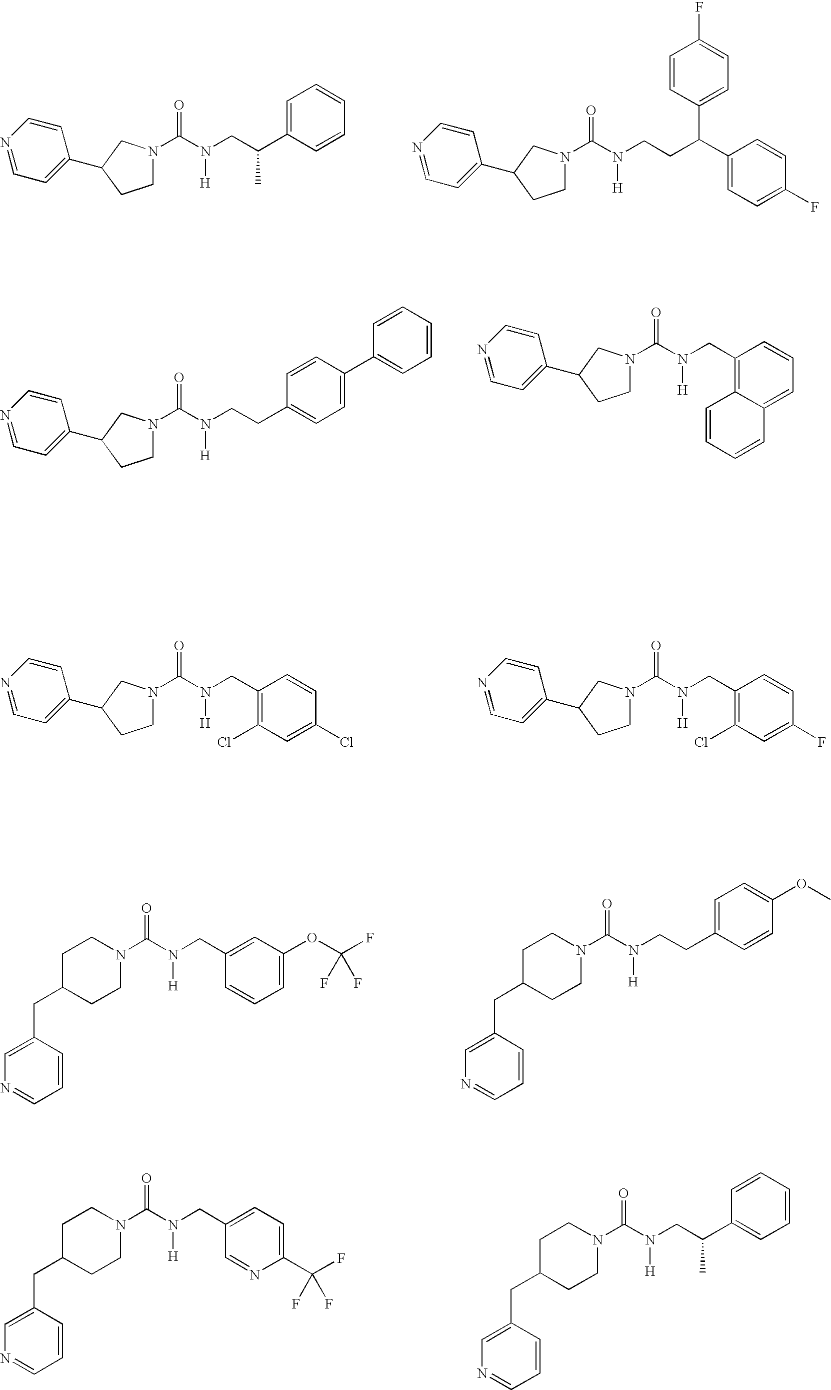 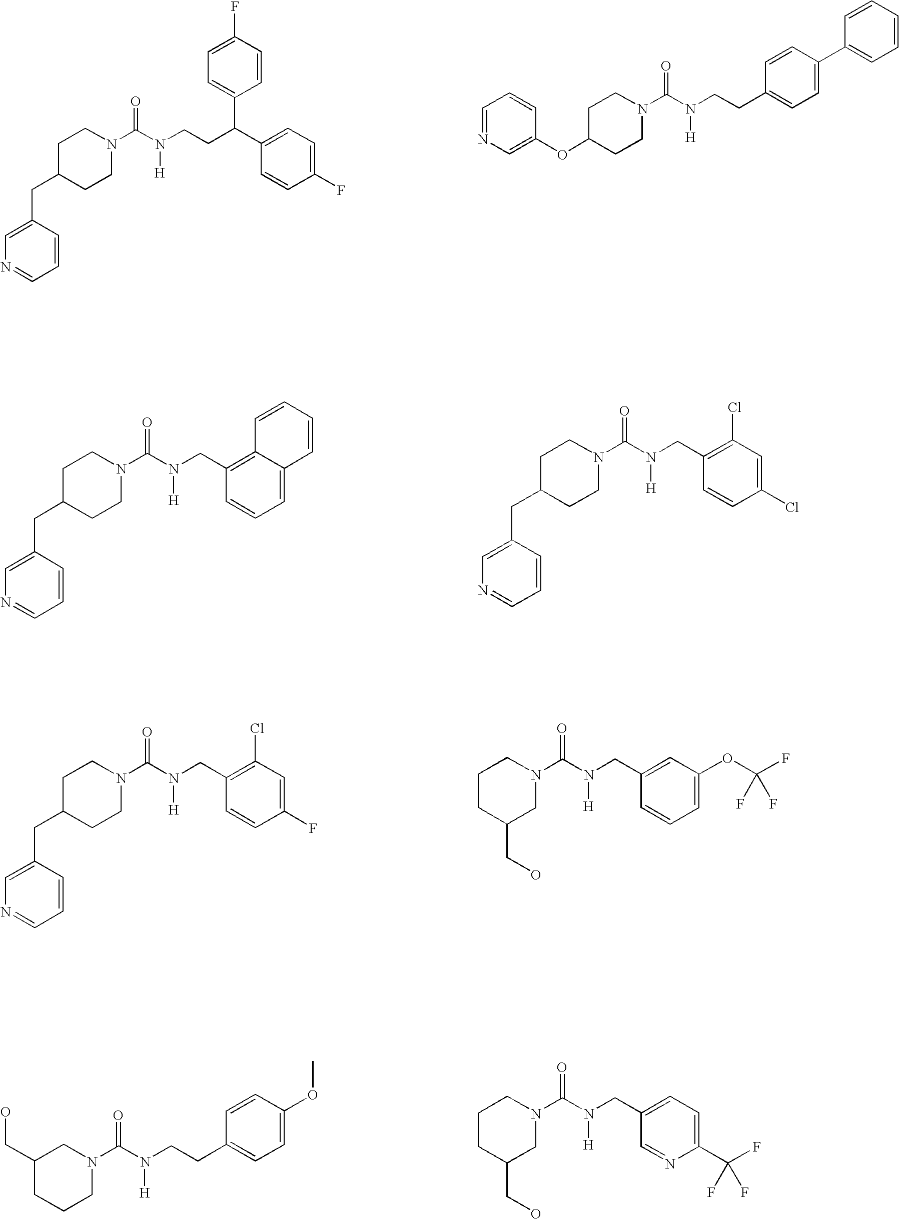 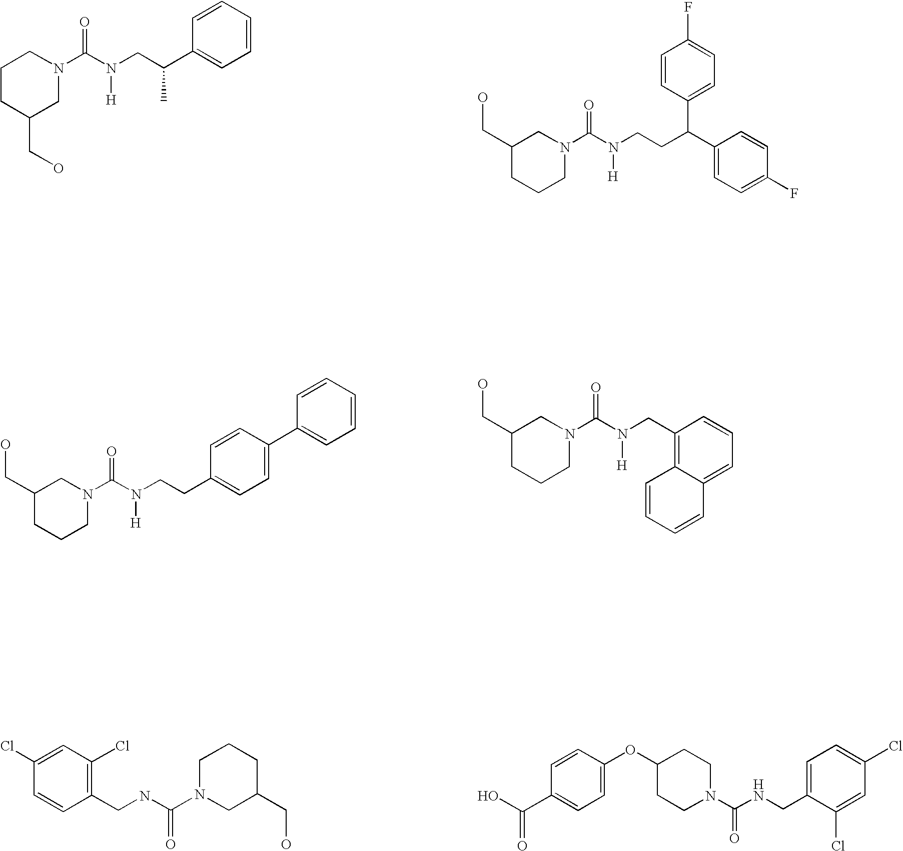
or the pharmaceutically acceptable salts thereof.

8. A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a pharmaceutically effective amount of a compound according to claim 1.

9. A pharmaceutical composition comprising a pharmaceutically effective amount of a compound according to claim 1 and one or more pharmaceutically acceptable carriers.
